SEQUENCE LISTING (96 sequences)

SEQ ID NO: 1 - DNA, 2694 bp, Caenorhabditis elegans
aagcgcccagaaatctctctctcgccgtctgcttctctgctccctcctacacgaagtttcaacacttcgctcgaacacaaccagggcgagaagacgttgaagaagataaattcttttaatcaaaaaaaaaaatgcacggaagcgggcacagtctaacgggcgctccgcatcaaattccgccgccacggacacagggagccgccaccggtggccaacaactctcggcgacagctaatcaatttgttgataaaatcgatccgttccataataaaagaggaacttctagacgattgcgcatcaacaacagcagccgatataatgttgattcggctcaagaactcgtacaattggcgctcatcaaagatactgctgcaaatgagcaaccggctctcgttatcgaaaagctcgtccaatgtcagcacgtattcgatttctatgatccagtagctcaactgaaatgcaaagaaatcaaaagagcagctctaaatgagctcatcgatcacattacatcgacaaaaggagcaattgttgagacaatttatccggctgtcattaaaatggttgccaagaatatattccgtgtacttccaccatcagaaaattgtgaatttgatccagaagaggatgaaccgactttagaagtctcgtggccacatttacagcttgtctacgagctcttcctgagattccttgagtcgccagatttccaagcttcaatcggtaaaaagtatattgatcagagatttgtgctcaagctgctcgatttattcgattcggaggatccacgtgaacgagactttttgaagacggttcttcatcgtatttacggaaaatttttgggccttcgggcttttattcgcaaacatatcaataatatgtttttgagattcgtatacgaaacggactcattcaacggtgtcggtgagcttctcgagattcttggctcaattataaacggatttgctcttccattgaagcaagagcacaaggttttccttgtaaaagttcttctaccattgcacaaaccgaaatgcttatcactgtatcatgcacagctcgcctactgcgtcgttcaattcatcgaaaaagactcatcactgactccacaagtttttgaggctctgctcaaattttggcctcgaacatgtagcagcaaggaggttatgttcctgggtgaagtggaggaaattctcgacattatcgaaccggaacaattcaaaaagattatcgatccattattccgtcaattggccaaatgtgtcagtagtccacattttcaagttgctgaacgggctctctacttttggaataatgaatatatattgtcattaattgaggatacaagcagtttggtgatgccgattatgttcccagcgctctatcggatttccaaagagcactggaatcaaacaattgttgcacttgtctataatgtactcaaaacttttatggaaatgaatggaaaactgtttgatgagcttacttctacgtacaaaggtgaacgattgcgggaaaagcaacgagaaaaggatcgtgatgctttttggaagaaaatggaagctcttgaattgaatccaccggctgaaggaaaagaagtgacaccttcgttgtttccagagaagttgactgattatttgaagaaggacggtccaaatatgacgccactgccagttgcaactgcaggtggcggtgataaatctccatctgtagtgaagaaatcgtcaacaggatcagaaacgacgacgccggctaaaaactcctatcaatcatttcttattcttcttcttcaaaaaaaaaaactcgatatcaatggtgatatatatgatgataagcacctcttctatttttccaaaatgctctccgtctctgcgtctccttcttatacacactctctcgtcttctgctgctggatcgaaaccattttatttgaaataaaatcaatcgataattgtacacacacaaatccccgaaaacccgctaaatcacacacattacccaccccaccccaaccaaaaaataaacgccatcattaatattatttttctctttttttattatttttccttttttcccccaaattttattgaatttcttccatttttcgccctcttttctgaaaattcgtgtttttgctcaccatgtcttcgtgtttttcttttccctctccacttttccctttctctctctctctcgtcgtcgttttccacatattttattccagatttttctctattcataatatttttgttgtatcccaaattctattatcgagttgcgaacaaatggtgatttttggcggaaaattagggtctcgccgcgaaaaaacaatttgctacagcacccctttaaaggcgcacgatagcgtcgagaccttttttaatcaaaaatcgatattttctccgcaactgggtaatattactgaactatttcagatttttaaatatacatttttttgtgccccgttttatttttttttgtatgtctgttgtctctagtagtatcaaatatcaataccaaaaataatgtatggaatattttttaatattagtgttgtaaagatccccctcctcctcaaaatgtcccaaaaattgcgaagaaacaaaaaaattcactctctccccctctttctctctctctctgtatgaattttttgtatttatatacacaaacatacacacacacatacacaccata

SEQ ID NO: 2 - protein, 633 aa, Caenorhabditis elegans
MHGSGHSLTGAPHQIPPPRTQGAATGGQQLSATANQFVDKIDPFHNKRGTSRRLRINNSSRYNVDSAQELVQLALIKDTAANEQPALVIEKLVQCQHVFDFYDPVAQLKCKEIKRAALNELIDHITSTKGAIVETIYPAVIKMVAKNIFRVLPPSENCEFDPEEDEPTLEVSWPHLQLVYELFLRFLESPDFQASIGKKYIDQRFVLKLLDLFDSEDPRERDFLKTVLHRIYGKFLGLRAFIRKHINNMFLRFVYETDSFNGVGELLEILGSIINGFALPLKQEHKVFLVKVLLPLHKPKCLSLYHAQLAYCVVQFIEKDSSLTPQVFEALLKFWPRTCSSKEVMFLGEVEEILDIIEPEQFKKIIDPLFRQLAKCVSSPHFQVAERALYFWNNEYILSLIEDTSSLVMPIMFPALYRISKEHWNQTIVALVYNVLKTFMEMNGKLFDELTSTYKGERLREKQREKDRDAFWKKMEALELNPPAEGKEVTPSLFPEKLTDYLKKDGPNMTPLPVATAGGGDKSPSVVKKSSTGSETTTPAKNSYQSFLILLLQKKKLDINGDIYDDKHLFYFSKMLSVSASPSYTHSLVFCCWIETILFEIKSIDNCTHTNPRKPAKSHTLPTPPQPKNKRHH

SEQ ID NO: 3 - DNA, 2710 bp, Homo sapiens
gggcggcgggtgccggtgcgcacggagccgagccggggctcccgttgcgctgcaccgcgttgggtcggagtcccaggacttcagcggagatccgcgcgctgcgacggccggtgcagagcccgccgagcgcccagtcccggcccggggctgagttgggggcatgctctagccgcccccccggagcccgggagagaacccaggagcgccgccgcccagccccagcgccccgagcggaaccgctgcgaaggggccctgaacggccgtcgccctccctacgggcagcccccgggggttggcgaccgaagtctaggttttcgagaaagccagggtgggaaccctaactggactcttcgggacccccaggaaggatctgaggcctgagccatcctcctttctaccctgtctgccccccaggactgggcagttgcaggaggccctgggggggggcccaggactgtggttgtgccccccccccaaaggccggacaggatgggaccaagttagtctgtccagtctcacccagcacctcccaggcccagagagaacccccggggctctgaaagcttgccctgccgcctgaccgccatggagacgaagctgccccctgcaagcacccccactagcccctcctcccccgggctgtcgcctgtgcccccacccgacaaggtggacggcttctcccgccgttccctccgcagagcccggccccgccgctcccacagctcctctcagttccgctatcagagcaaccagcaagagctcacaccgctgcccctgctcaaagatgtgccggcttccgagctgcacgagctgctgagccggaagctggcccagtgtggggtgatgtttgacttcttggactgtgtggccgacctcaaggggaaggaggtgaagcgggcagccctcaacgagctggtggagtgtgtggggagcacccggggtgtcctcatcgagcccgtctacccagacatcatccgcatgatctcagtgaatatcttccggactctgccgcccagtgagaaccctgaatttgaccctgaagaggatgagcccaatcttgagccttcgtggccacacctgcagctggtatatgagtttttcctgcgtttcttggagagcccagacttccagccctccgtggccaagagatatgtggatcaaaagtttgtcctgatgctcctggagctatttgatagtgaggatccccgggagcgtgagtacctcaagaccatcctgcaccgggtctatggcaagttcctgggtctccgggcctacatccgcaaacagtgcaaccacatcttcctccggttcatctatgaattcgagcacttcaatggtgtggctgagctgctggagatcctaggaagcatcatcaatggctttgcgctgcccctgaagacggagcacaagcagttcctggttcgcgtcctgatccccctgcactctgtcaagtcgctgtctgtcttccatgcccagctggcatactgtgtggtgcagttcctggagaaggatgccactctgacagagcacgtgatccgggggctgctcaaatactggccaaaaacctgcacccagaaggaggtgatgtttctgggggagatggaagagattcttgatgtcatcgagccctcccagtttgtgaagatccaggagcccctttttaagcaggtggctcgctgtgtttccagcccccatttccaggttgcagagcgggctctgtatttctggaacaatgagtatatcctaagcctcattgaggacaactgccacactgtgctgcctgctgtgtttgggaccctctaccaagtctccaaggagcactggaaccaaaccatcgtatcactgatctacaatgtgctcaagaccttcatggagatgaatgggaagctgtttgatgagctcacagcctcctacaagctggaaaagcagcaggagcagcagaaggcccaggagcgtcaggagttatggcaaggtctggaggagctgcggctacgccggctacaggggacccagggggccaaggaggcccccctccagcggcttacaccccaggtggccgccagtgggggtcagagctagacagcacctcagaaggggaaaagctaaacccagagctgtcagtccctctatcccttctcctgtccaggggcccagagagaaacacacctacccctggccttgccagagtggcttctgaggactccctgcccagcccagctttcactggggggagacgaggagaggcaatggtggtcttggcaacagaatgctcagcccctcgtggcaggacttgacaagggcaagcttgaccaggaagctgccatcagggatcttcccctgccccgcaaagctaggctccagctgcaggcgggctcccaccctctgctcctggccttgggcaagggcactcagcgcctcgcctgcccctgccttggccaatgcgaggtccttccttatccccaccatggggtccatggtctatttattctcgcccagctcaccctctacacagacactgtcctgggtgcacactcctcccttccctcgctgtgtacttccttgtcccctttttatttattgggcagggggagggggagggcacaggcaagaagagattcacagtgtcctggggtaagggggggttcacagtaatcatggtctactcctctttccgtggctgggggtagacttaataaagagagaaattcaa

SEQ ID NO: 4 - protein, 497 aa, Homo sapiens
METKLPPASTPTSPSSPGLSPVPPPDKVDGFSRRSLRRARPRRSHSSSQFRYQSNQQELTPLPLLKDVPASELHELLSRKLAQCGVMFDFLDCVADLKGKEVKRAALNELVECVGSTRGVLIEPVYPDIIRMISVNIFRTLPPSENPEFDPEEDEPNLEPSWPHLQLVYEFFLRFLESPDFQPSVAKRYVDQKFVLMLLELFDSEDPREREYLKTILHRVYGKFLGLRAYIRKQCNHIFLRFIYEFEHFNGVAELLEILGSIINGFALPLKTEHKQFLVRVLIPLHSVKSLSVFHAQLAYCVVQFLEKDATLTEHVIRGLLKYWPKTCTQKEVMFLGEMEEILDVIEPSQFVKIQEPLFKQVARCVSSPHFQVAERALYFWNNEYILSLIEDNCHTVLPAVFGTLYQVSKEHWNQTIVSLIYNVLKTFMEMNGKLFDELTASYKLEKQQEQQKAQERQELWQGLEELRLRRLQGTQGAKEAPLQRLTPQVAASGGQS

SEQ ID NO: 5 - protein, 607 aa, Caenorhabditis elegans
MLRSKKKDKENGKSEKKDKEKDKKSMKDDGAGSSKASTIPTIRTEDVGGDMIPADAPPPTNIGRTNTYGGGPVIPRRERRQSSSMFNISQNRELQRLPAIKDADPSERETLFIQKLRQCCVVFDFANDALSDLKFKEVKRAALNELVDHVSGAPKGSLSDAVYPEAIGMFSTNLFRPLSPPTNPIGAEFDPDEDEPTLEAAWPHLQLVYEFFLRFLECPDFQSQVAKRYIDQNFILRLLMIMDSEDPRERDFLKTTLHRIYGKFLGHRAYIRKQINNIFYSFIYETERHNGIAELLEILGSIINGFALPLKEEHKTFLLRVLLPLHKVKSLSVYHPQLAYCVVQFIEKDSSLTEPVISGMLRFWPKQHSPKEVMFLNELEEVLDVIEPNEFQKIMTPLFSQIARCVSSPHFQVAERALYYWNNEYVMSLVADNARVIIPIMFPVLFKNSKSHWNKTIHGLIYNALKMFMEMNQKLFDECSQAYQKDRVQEKTLNEEKERIWNNIEKQAMGNPQYVEVKALFARFNPDEIISSRQQNGVDENMKTSTVLSKDEILKNAVGVSSMKNDMDFGPNHKQSDFPPDEQTTRALGEYKRHDPFLKKVTSTDEQ

SEQ ID NO: 6 - DNA, 1845 bp, Caenorhabditis elegans
gcggtcggctccgcatccgcaatgttgagatcaaagaagaaggacaaggaaaatgggaaatcagagaaaaaggacaaagagaaggataagaaatcaatgaaagatgacggtgctggaagcagtaaggcatctacaattcctacgatccgaacagaagatgttggaggagatatgattcctgcagatgcaccaccaccaacaaatattggccgaacaaacacgtacggaggagggccagtgataccgcgaagagagcgccgccaatcaagtagcatgttcaacatttcacaaaacagagagctgcaaagacttccagctatcaaagatgcggatccgagcgagagagaaacactgttcattcaaaaacttcgacaatgctgcgttgtttttgactttgccaatgatgctctcagcgatttaaaattcaaagaagtcaaacgggcagctctaaatgaactcgtcgaccacgtttccggagctccaaaaggatcgctatctgatgctgtctatccagaagctatcggcatgttctccactaatcttttccgtcctttgagcccaccgaccaatccaattggtgctgaatttgaccccgatgaagacgaaccaacattagaagctgcctggccacatctgcagctcgtctacgagtttttcctgagattcctggagtgccccgattttcaatctcaagttgcaaaacgttacattgatcaaaatttcattcttcgtcttctcatgattatggacagtgaagatccacgtgaacgtgactttctgaagacaactttacatcgaatttatggaaagttcctcggacatcgtgcttacattcggaagcaaatcaacaacattttctactcgttcatctacgaaactgagaggcacaacgggattgctgaattactcgagattctaggtagcattatcaatggattcgctcttcccttgaaagaggaacacaaaactttcttgctccgagttcttcttccgctacacaaggtcaaatcattatctgtatatcatcctcaactcgcgtactgtgtcgtacagttcattgaaaaagactcatcactgacagagcctgtaatcagtgggatgctacgcttctggccaaaacaacatagtcccaaggaagtgatgttcctgaatgaattggaagaagtcctcgatgtgatagagccgaatgagttccaaaaaatcatgactccattgttctcacaaattgctcgctgtgtcagcagtccacacttccaagttgccgaacgagctctctactattggaataatgaatatgtgatgtcattagtagcggacaatgctcgtgtgataattccaataatgttccctgttcttttcaaaaacagcaagtcacattggaataaaacgattcatggacttatctacaacgccctcaagatgttcatggaaatgaatcaaaaactgtttgacgagtgttcccaagcttatcagaaagatcgagttcaagaaaagacattgaatgaagaaaaagaacgcatctggaacaatattgagaaacaggcaatgggaaacccgcaatatgttgaagttaaagcactatttgctcgatttaatccagatgaaatcatcagctcgcgtcagcagaatggagttgacgagaatatgaagacttcaactgtgcttagcaaggatgaaattctgaaaaatgctgtaggagtgagctcgatgaaaaacgatatggatttcggacccaaccacaaacaatccgatttccctccagatgagcaaacgacgagagctttaggcgagtacaagcgtcacgatccatttttgaagaaagtgactagcaccgacgaacagtga

SEQ ID NO: 7 - protein, 561 aa, Caenorhabditis elegans
MIPADAPPPTNIGRTNTYGGDLISGPVIPRRERRQSSSMFNISQNRELQRLPAIKDADPSERETLFIQKLRQCCVVFDFANDALSDLKFKEVKRAALNELVDHVSGAPKGSLSDAVYPEAIGMFSTNLFRPLSPPTNPIGAEFDPDEDEPTLEAAWPHLQLVYEFFLRFLECPDFQSQVAKRYIDQNFILRLLMIMDSEDPRERDFLKTTLHRIYGKFLGHRAYIRKQINNIFYSFIYETERHNGIAELLEILGSIINGFALPLKEEHKTFLLRVLLPLHKVKSLSVYHPQLAYCVVQFIEKDSSLTEPVISGMLRFWPKQHSPKEVMFLNELEEVLDVIEPNEFQKIMTPLFSQIARCVSSPHFQVAERALYYWNNEYVMSLVADNARVIIPIMFPVLFKNSKSHWNKTIHGLIYNALKMFMEMNQKLFDECSQAYQKDRVQEKTLNEEKERIWNNIEKQAMGNPQYVEVKALFARFNPDEIISSRQQNGVDENMKTSTVLSKDEILKNAVGVSSMKNDMDFGPNHKQSDFPPDEQTTRALGEYKRHDPFLKKVTSTDEQ

SEQ ID NO: 8 - DNA, 1760 bp, Caenorhabditis elegans
ggataaatttttttttgaatcacaatcattgatttttccattttcggccctcatatttatcatttctaggagatatgattcctgcagatgcaccaccaccaacaaatattggccgaacaaacacgtacggaggagatttaatttcagggccagtgataccgcgaagagagcgccgccaatcaagtagcatgttcaacatttcacaaaacagagagctgcaaagacttccagctatcaaagatgcggatccgagcgagagagaaacactgttcattcaaaaacttcgacaatgctgcgttgtttttgactttgccaatgatgctctcagcgatttaaaattcaaagaagtcaaacgggcagctctaaatgaactcgtcgaccacgtttccggagctccaaaaggatcgctatctgatgctgtctatccagaagctatcggcatgttctccactaatcttttccgtcctttgagcccaccgaccaatccaattggtgctgaatttgaccccgatgaagacgaaccaacattagaagctgcctggccacatctgcagctcgtctacgagtttttcctgagattcctggagtgccccgattttcaatctcaagttgcaaaacgttacattgatcaaaatttcattcttcgtcttctcatgattatggacagtgaagatccacgtgaacgtgactttctgaagacaactttacatcgaatttatggaaagttcctcggacatcgtgcttacattcggaagcaaatcaacaacattttctactcgttcatctacgaaactgagaggcacaacgggattgctgaattactcgagattctaggtagcattatcaatggattcgctcttcccttgaaagaggaacacaaaactttcttgctccgagttcttcttccgctacacaaggtcaaatcattatctgtatatcatcctcaactcgcgtactgtgtcgtacagttcattgaaaaagactcatcactgacagagcctgtaatcagtgggatgctacgcttctggccaaaacaacatagtcccaaggaagtgatgttcctgaatgaattggaagaagtcctcgatgtgatagagccgaatgagttccaaaaaatcatgactccattgttctcacaaattgctcgctgtgtcagcagtccacacttccaagttgccgaacgagctctctactattggaataatgaatatgtgatgtcattagtagcggacaatgctcgtgtgataattccaataatgttccctgttcttttcaaaaacagcaagtcacattggaataaaacgattcatggacttatctacaacgccctcaagatgttcatggaaatgaatcaaaaactgtttgacgagtgttcccaagcttatcagaaagatcgagttcaagaaaagacattgaatgaagaaaaagaacgcatctggaacaatattgagaaacaggcaatgggaaacccgcaatatgttgaagttaaagcactatttgctcgatttaatccagatgaaatcatcagctcgcgtcagcagaatggagttgacgagaatatgaagacttcaactgtgcttagcaaggatgaaattctgaaaaatgctgtaggagtgagctcgatgaaaaacgatatggatttcggacccaaccacaaacaatccgatttccctccagatgagcaaacgacgagagctttaggcgagtacaagcgtcacgatccatttttgaagaaagtgactagcaccgacgaacagtga

SEQ ID NO: 9 - protein, 557 aa, Caenorhabditis elegans
MIPADAPPPTNIGRTNTYGGGPVIPRRERRQSSSMFNISQNRELQRLPAIKDADPSERETLFIQKLRQCCVVFDFANDALSDLKFKEVKRAALNELVDHVSGAPKGSLSDAVYPEAIGMFSTNLFRPLSPPTNPIGAEFDPDEDEPTLEAAWPHLQLVYEFFLRFLECPDFQSQVAKRYIDQNFILRLLMIMDSEDPRERDFLKTTLHRIYGKFLGHRAYIRKQINNIFYSFIYETERHNGIAELLEILGSIINGFALPLKEEHKTFLLRVLLPLHKVKSLSVYHPQLAYCVVQFIEKDSSLTEPVISGMLRFWPKQHSPKEVMFLNELEEVLDVIEPNEFQKIMTPLFSQIARCVSSPHFQVAERALYYWNNEYVMSLVADNARVIIPIMFPVLFKNSKSHWNKTIHGLIYNALKMFMEMNQKLFDECSQAYQKDRVQEKTLNEEKERIWNNIEKQAMGNPQYVEVKALFARFNPDEIISSRQQNGVDENMKTSTVLSKDEILKNAVGVSSMKNDMDFGPNHKQSDFPPDEQTTRALGEYKRHDPFLKKVTSTDEQ

SEQ ID NO: 10 - DNA, 1845 bp, Caenorhabditis elegans; identical to SEQ ID NO: 6

SEQ ID NO: 11 - protein, 497 aa, Mus musculus
METKLPPASTPTSPSSPGLSPVPPPDKVDGFSRRSLRRARPRRSHSSSQFRYQSNQQELTPLPLLKDVPASELHELLSRKLAQCGVMFDFLDCVADLKGKEVKRAALNELVECVGCTRGVLIEPVYPDIIRMISVNIFRTLPPSENPEFDPEEDEPNLEPSWPHLQLVYEFFLRFLESPDFQPSVAKRYVDQKFVLMLLELFDSEDPREREYLKTILHRVYGKFLGLRAYIRKQCNHIFLRFIYELEHFNGVAELLEILGSIINGFALPLKTEHKQFLVRVLIPLHSVKSLSVFHAQLAYCVVQFLEKDATLTEHVIRGLLKYWPKTCTQKEVMFLGEMEEILDVIEPSQFVKIQEPLFKQVARCVSSPHFQVAERALYFWNNEYILSLIEDNCHTVLPAVFGTLYQVSKEHWNQTIVSLIYNVLKTFMEMNGKLFDELTASYKLEKQQEQQKAQERQELWRGLEELRLRRLQGTQGAKEAPVPRPTPQVAASGGQS

SEQ ID NO: 12 - DNA, 2708 bp, Mus musculus
gtcatcctgagcagctgggcggcgggtgccggtgcgcagcgagccggggctcccgctgcgctgcaccgcgctgatctgagtcccgggatatctgccgagggtcgcgtgctgccggggcccagcccagagcccaccgagtgcccgaccccttttggggctgagctgggggcatgctccagcacccccagagcctgggagcgaacccaggagcgccgccgcccagccccagcgccccgagcggcagaaccaccgctaaggacccttgaaccgtcgtctcttccaccgaggcaacctctagggtcggcgaccaaagtctgggtttctgagaaaagagccagcgtgggaaccctgactggaactcttctggatccccaggaaagacctgagccattgcctttctaccttacctgcccccccaggactgggcagttgccagaggccctgggggggtcaggactgtggccgtgcctcccccaccccctgaaacatgctggacaggatgggtcccagttagtgtgtgtggcctcattcaacttactccagacccacaaggagccccctgggctctgaaagattgacccgtgttgtccactggccatggagacgaagctgccccctgcgagcacccccacaagcccctcctccccagggctgtctccagtgccaccaccagacaaggtggatggcttctcccgcaggtccctccgcagggcccggccccgtcgctcacacagctcttctcagttccgctatcagagcaaccagcaagagctcactccactgcccctgctcaaagatgtgccagcctctgagttgcatgagttgctgagccggaaactggcccaatgtggggtgatgtttgacttcttggactgcgtggctgacctcaaggggaaggaggtgaagcgtgcagccctcaatgaactggtggaatgtgtgggttgcacccggggtgtgctcatcgagcccgtctacccagacatcatccgcatgatatcagtaaatatcttccggaccctgccacccagcgagaaccctgagtttgaccctgaagaagatgagcccaaccttgagccctcgtggccacatctgcagctggtatatgagtttttcctgcgtttcttggagagtccagatttccagccctctgtggccaagagatacgtggatcaaaagtttgtcctaatgctcctggagctatttgacagcgaggacccccgggaacgtgagtacctcaagaccatcttgcatcgggtgtacggcaagttcctgggtctccgggcctacatccgcaaacagtgcaaccacatcttcctccggttcatctacgagctggaacacttcaatggtgtggctgagctcttagagatcttaggaagcatcatcaatggctttgcgctgcccctgaagactgagcacaagcagttcctggttcgagtcctgatccccttgcactctgtcaagtcactatctgtttttcatgctcagctggcatactgtgtggtgcagttcctggagaaggatgcaaccttgacagagcatgttatccgggggcttctcaaatactggcctaaaacctgcacccagaaggaggtgatgttcctgggggagatggaagagattcttgatgtcatcgagccctcccagtttgtgaagatccaggagcccctcttcaagcaggtggctcgctgtgtctccagcccccatttccaggttgcagagcgggctctgtatttctggaacaatgagtacatcctgagcctcattgaggacaactgccacactgtgctgcctgcggtatttgggaccctctaccaagtgtccaaggagcactggaatcaaaccatcgtgtccctgatctacaacgtgctcaagactttcatggagatgaacggaaagctgtttgacgagctcacagcctcctacaagctggaaaaacaacaggagcagcagaaggcccaggagcggcaggagctatggcgaggcttggaggaactgcggctacgccggctacaggggacccaaggggccaaggaagcccccgtcccacggcctacgccccaggtggctgccagtgggggtcagagctagatacacctagaacaggagatgctaaacccagagctatcagcccctccatccctctgcccaggggcccagtgaggcccacgcctccccgtggccttgccggagtggctctaggactccctaccagccccgtgggaacagctttcacggagggggaaacaagaaggcaagatggtagtcttggcagcagaactctcaggcctttgtggcaagattctggcaagactagaccagggcaagtatgcaactgggaagctgccatcagggatcctcccctgccctacacagccaggctccaggcggcagctaggcctctacccctgctcccagcctggggcatggacactcagcgcctggccagggccttcctcatccccaccatgggggcacagtctatttattctgcccagctcacccccaacaagaccttgtccagggacattctcctatcctctccctggccctgactcccttatcccctttttatttattgggcagggggaggggtgagggcacaggcaagaagattcacattgtcctggggtgaggggggaggtcacagtaatcatggtctgcccccttcacctggctgggggcagatttaataaagagcgaaactc

SEQ ID NO: 13 - protein, 497 aa, Rattus norvegicus
METKLPPASTPTSPSSPGLSPVPPPDKVDGFSRRSLRRARPRRSHSSSQFRYQSNQQELTPLPLLKDVPASELHELLSRKLAQCGVMFDFLDCVADLKGKEVKRAALNELVECVGSTRGVLIEPVYPDIIRMISVNIFRTLPPSENPEFDPEEDEPNLEPSWPHLQLVYEFFLRFLESPDFQPSVAKRYVDQKFVLMLLELFDSEDPREREYLKTILHRVYGKFLGLRAYIRKQCNHIFLRFIYELEHFNGVAELLEILGSIINGFALPLKTEHKQFLVRVLIPLHSVKSLSVFHAQLAYCVVQFLEKDATLTEHVIRGLLKYWPKTCTQKEVMFLGEMEEILDVIEPSQFVKIQEPLFKQVARCVSSPHFQVAERALYFWNNEYILSLIEDNCHTVLPAVFGTLYQVSKEHWNQTIVSLIYNVLKTFMEMNGKLFDELTASYKLEKQQEQQKAQERQELWRGLEELRLRRLQGTQGAKEAPVPRPTPQVAASGGQS

SEQ ID NO: 14 - DNA, 2213 bp, Rattus norvegicus
caaggagccccttgggctctgaaagattgacttgctgccctctggccatggagacgaagctgccccctgcaagcacccccacaagcccctcctccccggggctgtctccagtgccaccaccagacaaggtggatggcttctcccgccgatccctccgcagggcccggccccgacgctcacacagctcttctcagttccgctatcagagcaaccagcaagagctcactccactgcccctgctcaaagatgtgccagcctctgagttacatgagttgctaagccggaaactggcccaatgtggggtgatgtttgacttcttggactgtgtggctgacctcaagggcaaggaggtgaagcgtgcagccctcaatgaacttgtggaatgtgtggggagcacccggggggtcctcattgagcctgtctacccagacatcatccgcatgatatcagtaaatatcttccggaccctgccgcccagtgagaaccctgaatttgaccctgaagaagatgagcccaaccttgagccttcgtggccacatctacagctggtatatgagtttttcctgcggttcttggagagtccagatttccagccctctgtggccaagagatacgtggatcaaaagtttgttctgatgctcctggagctatttgacagcgaggacccccgggaacgtgagtacctcaagaccatcttgcatcgggtatacggcaagttcctgggtctccgggcctacatccgcaaacagtgcaaccacatcttcctccggttcatctatgagctggagcactttaatggtgtggctgagctgttagagatcttaggaagcatcatcaatggctttgcgctgcccctgaagactgagcacaaacagttcctggttcgagtcctcatccccttgcactctgtcaagtcactgtctgtttttcatgctcagctggcatactgtgtggtgcagttcctggagaaggatgcgaccttgacagagcatgttatccgggggcttctcaaatactggcctaaaacctgcacccagaaggaggtgatgttcctgggggagatggaagagattctcgatgtgatcgagccctcccagtttgtgaagatccaggagcctctcttcaagcaggtggctcgctgtgtctccagcccccatttccaggttgcagagcgggctctgtatttctggaacaatgagtacatcctgagcctcattgaggacaactgccacactgtgctgcctgcggtatttgggaccctctaccaagtgtccaaggagcactggaatcaaaccatcgtgtccctgatctacaacgtgctcaagactttcatggagatgaatgggaagctgtttgatgagcttacagcctcctacaagctagaaaaacagcaggagcagcagaaggcccaggagcggcaggaactttggcgaggcttggaggaactgcggttacgccggctacaggggacccaaggggccaaggaagcccctgtcccaaggcctacgccccaggtggctgccagtgggggtcaaagctagataaatctagaacaggagaagctagacccagagctatcagcccctccatcccttctgcccaggggcccagggaggcccatgcctccccgtggccttgccagcgtggctctaggactccctaccagccccatgggaacagctttcatggaggggagaccagaaggcaagatggtagtcttggcagcagaactctcaggcccttgtggcaagattctggcaagactagaccagggcaagtcttcgactgggaagctgccatcggggatcctcccctgccctacacagctaggctccaggcggcagctgggcttctccccctgctcctggcttgggccatggacactcagcacctggcttggcctgccttggtctggtcagggccttcctcatccccatcatgggggcacagtctatttattctgcccagctcaccttcaacaagacattgtccagggacatcttcctctcctcccccttgccctgaacttccttatcccctttttatttattgggcagggggaggggtgagggcacaggcaagaagagagtcacattgtcctggggtgagggggggtcacagtaatcatggtctgctcccttcacctggctagggggcagacttaataaagagcgaaactcaaaaaaaaaaaaaaaaaaaaaaaaaaaaaaagg

SEQ ID NO: 15 - protein, 486 aa, Homo sapiens
MSSSSPPAGAASAAISASEKVDGFTRKSVRKAQRQKRSQGSSQFRSQGSQAELHPLPQLKDATSNEQQELFCQKLQQCCILFDFMDSVSDLKSKEIKRATLNELVEYVSTNRGVIVESAYSDIVKMISANIFRTLPPSDNPDFDPEEDEPTLEASWPHIQLVYEFFLRFLESPDFQPSIAKRYIDQKFVQQLLELFDSEDPRERDFLKTVLHRIYGKFLGLRAFIRKQINNIFLRFIYETEHFNGVAELLEILGSIINGFALPLKAEHKQFLMKVLIPMHTAKGLALFHAQLAYCVVQFLEKDTTLTEPVIRGLLKFWPKTCSQKEVMFLGEIEEILDVIEPTQFKKIEEPLFKQISKCVSSSHFQVAERALYFWNNEYILSLIEENIDKILPIMFASLYKISKEHWNPTIVALVYNVLKTLMEMNGKLFDDLTSSYKAERQREKKKELEREELWKKLEELKLKKALEKQNSAYNMHSILSNTSAE

SEQ ID NO: 16 - DNA, 3145 bp, Homo sapiens
cgcagagggccggggctacggggcagcgccccgggcgatgaggggccggcgttgaccgggaagagcgggcaccgcggcagtggctccgaggggacccgcgatggcagcgccctgagaggaggctccaggcagggcgggctgcgctggcagcggccgctgaggtgctggccggccggctggctggcgacgggggcagaagcgacgagaggcgcgctcggcacccgcacccccgtgcccccgcctcagttgtctaaacttcgggctctcttccacccgctctgcgcgcccagagtcaacaacttcttcacccccctccgcccccgcccttccctccgtcagccccgggagctcgccgcgcgccggggaccaggaacctccagcgctgagatgtggccgtgaggcgttggcgggcggcgaggagaagctcggcggcgtcccggggccggagggccgtggggccggggcgcaggggcgcgagcaccccgcgcctctcccccgcctcctcctgccgtctccgccgctgcccgtgccttgcaagcagcagccggagctgccaagcgtcagggccgcggagatgtcgtcgtcgtcgccgccggcgggggctgccagcgccgccatctcggcctcggagaaagtggacggcttcacccggaaatcggtccgcaaggcgcagaggcagaagcgctcccagggctcgtcgcagtttcgcagccagggcagccaggcagagctgcacccgctgccccagctcaaagatgccacttcaaatgaacaacaagagcttttctgtcagaagttgcagcagtgttgtatactgtttgatttcatggactctgtttcagacttgaagagcaaagaaattaaaagagcaacactgaatgaactggttgagtatgtttcaactaatcgtggtgtaattgttgaatcagcgtattctgatatagtaaaaatgatcagtgctaacatcttccgtacacttcctccaagtgataatccagattttgatccagaagaggatgaacccacgcttgaggcctcttggcctcacatacagttggtatatgaattcttcttgagatttttggagagccctgatttccagcctagcattgcaaaacgatacattgatcagaaattcgtacaacagctcctggagctttttgatagtgaagatcccagagaacgtgacttcctgaagactgttctgcaccgaatttatgggaaatttcttggattaagagcattcatcagaaaacaaattaacaacattttcctcaggtttatatatgaaacagaacatttcaatggtgttgctgaacttcttgaaatattaggaagtattatcaatggctttgcattgccactgaaagcagaacataaacaatttctaatgaaggttcttattcctatgcatactgcaaaaggattagctttgtttcatgctcagctagcatattgtgttgtacagttcctggagaaagatacaacactaacagagccagtgatcagaggactgctgaaattttggccaaaaacctgcagtcagaaagaggtgatgtttttaggagaaattgaagaaatcttagatgtcattgaaccaacacagttcaaaaaaattgaagagccacttttcaagcagatatccaagtgtgtatccagttctcattttcaggttgcagaaagggcattgtacttctggaataacgaatatattcttagtttgattgaggagaacattgataaaattctgccaattatgtttgccagtttgtacaaaatttccaaagaacactggaatccgaccattgtagcactggtatacaatgtgctgaaaaccctaatggaaatgaatggcaagcttttcgatgaccttactagctcatacaaagctgaaagacagagagagaaaaagaaggaattggaacgtgaagaattatggaaaaaattagaggagctaaagctaaagaaagctctagaaaaacagaatagtgcttacaacatgcacagtattctcagcaatacaagtgccgaataaaaaaaaagcctcccacctctgccggataggcagagttttgtatgcttttttgaaatatgtaaaaattacaaaacaaacctcatcagtataatataattaaaaggccaattttttctggcaactgtaaatggaaaaatatatggactaaacgtagccctgtgctgtatcatggccatagtatattgtaacctttgtctaatcattggatttattgtgtcacttctgaagtttcacagaaatgaatgaattttatcatctatgatatgagtgagataattatgggagtggtaagaattatgacttgaattcttctttgattgtgttgcacatagatatggtagtctgctctgtatatttttcccttttataatgtgcttttcacactgctgcaaaccttagttacatcctaggaaaaaatacttcctaaaataaaactaaggtatcatccttacccttctctttgtctcacccagaaatatgatggggggaattacctgccctaacccctccctcaataaatacattactgtactctggaatttaggcaaaaccttaaatctccaggctttttaaagcacaaaatataaataaaagctgggaaagtaaaccaaaattcttcagattgttcctcatgaatatcccccttcctctgcaattctccagagtggtaacagatgggtagaggcagctcaggtgaattacccagcttgcctctcaattcattcctcctcttcctctcaaaggctgaaggcagggcctttccagtcctcacaacctgtccttcacctagtccctcctgacccagggatggaggctttgagtcccacagtgtggtgatacagagcactagttgtcactgcctggctttatttaaaggaactgcagtaggcttcctctgtagagctctgaaaaggttgactatatagaggtcttgtatgtttttacttggtcaagtatttctcacatcttttgttatcagagtaccattccaatctcttaacttgcagttgtgtggaaaactgttttgtaatgaaagatcttcattgggggattgagcagcatttaataaagtctatgtttgtattttgccttaaaaaaaaaaaaaaaaaa

SEQ ID NO: 17 - protein, 486 aa, Mus musculus
MSSPSPPAPVACAAISASEKVDGFTRKSVRKAQRQKRSQGSSQFRSQGSQAELHPLPQLKDATSNEQQELFCQKLQQCCVLFDFMDSVSDLKSKEIKRATLNELVEYVSTNRGVIVESAYSDIVKMISANIFRTLPPSDNPDFDPEEDEPTLEASWPHIQLVYEFFLRFLESPDFQPSIAKRYIDQKFVQQLLELFDSEDPRERDFLKTVLHRIYGKFLGLRAFIRKQINNIFLRFIYETEHFNGVAELLEILGSIINGFALPLKAEHKQFLMKVLIPMHTAKGLALFHAQLAYCVVQFLEKDTTLTEPVIRGLLKFWPKTCSQKEVMFLGEIEEILDVIEPTQFKKIEEPLFKQISKCVSSSHFQVAERALYFWNNEYILSLIEENIDKILPIMFASLYKISKEHWNQTIVALVYNVLKTLMEMNGKLFDDLTSSYKAERQREKKKELEREELWKKLEELQLKKALEKQNNAYNMHSIRSSTSAK

SEQ ID NO: 18 - DNA, 3082 bp, Mus musculus
cccccctcacccgaaccagccaccctctcaagttgtagcagttgcttcccgggcgtgctccgtgggcggccggtgggcgcgggaggctgagcgagggcgacgctccagggatccaaggatccaaggatcggggtacgggtggccgccccgggcgatgaggggccatcgctgacaggagaacgagcgctcggcagcggctcgcgcggacccgcgatggaagcgccccgggaggaggctcccggccgggcggacagcgcgggcggcagccgctgaagacctgtccggactccgcgcgctaaggggccgcgaggcgcgcccggcgtccgcacccgcgcgctcgccccagccgtccaaacttcgggctcgcctccgcccgccctcgtcgccgggcgtcagcaacttgtgcggcccgcgcgcccccgccctcccctccgccagccccgggaggcggccgcgcggcgcggggatgcggccgtgaggcgctgtcgggtgaggagcagctcgcggagcgcgagggcggcgcggccggggcccggggcgcaggggcgcaggcccgccgtgcccgtcccccgccgattgcccgggccagccgccgcgggaggcgccgatcgcccgggtcgccgagatgtcgtcgccgtcgccgcccgcaccagttgcctgcgccgccatctcggcctcggagaaagtggacggcttcacccggaaatcggtgcgcaaggcgcagaggcagaagcgctctcagggctcgtcgcagttccgcagccagggcagccaggcggagctgcaccccctgccccagctcaaagatgccacttcaaatgaacagcaagagcttttctgccagaagctacaacagtgctgtgtactgtttgatttcatggactctgtctcagacttgaagagcaaagaaattaaaagagcgacgctgaatgaactggttgagtatgtttcaactaatcgtggtgtaattgttgaatcagcgtattctgatatagtaaaaatgatcagtgctaacatcttccggacacttcctccaagtgataatccagattttgacccggaagaggatgagcccacacttgaggcctcttggcctcacatacagttggtgtatgaattcttcttgagattcttggagagtcctgatttccagcccagcattgcaaagcgatacattgatcagaagtttgtccaacagctcctggagctttttgatagtgaagatccacgggagcgcgacttcctgaagaccgtcctgcatcggatttacggcaagttccttggcctgagagcgttcatcagaaagcaaattaacaacattttcctcaggtttatatatgaaacggaacatttcaatggtgtggctgagctcctggaaatattgggaagtatcatcaatggctttgcattgccactgaaagcagagcataagcagtttctaatgaaggttcttattcctatgcatactgcaaaaggattggccttgtttcacgcacagctggcgtactgtgttgtgcagttcctggagaaagacacaacgctgacagagccggtgatcagaggactgctgaaattttggccaaaaacatgcagtcagaaagaggtgatgtttttaggagaaattgaagagatcttagatgtcattgaaccaacacaattcaaaaaaattgaagagccgctttttaagcagatatccaagtgcgtctccagctctcatttccaggttgcagaaagagcactgtacttctggaataatgaatatattcttagtttgattgaagagaatattgataaaatcctgccaatcatgtttgccagcttgtataaaatttccaaagaacactggaatcagactattgtagcactggtgtacaatgtgctgaaaaccctcatggagatgaacggcaagctttttgacgaccttactagttcctacaaagccgaaagacagagagagaagaagaaagaactggaacgggaagagttgtggaaaaaactagaggagctgcagctgaagaaggctctagagaaacagaacaatgcttacaacatgcacagtattcgcagcagtaccagtgccaaataaagatcagctccccctgctgggcggtcgttttgtacacttttttttttttttgaatatataaaaacttcagagcagacctcatcagtataatataattaggaggccagtttttcctggcaagcgtaaaagcgaaagaattatggactaaaacatagccctgtgctgtatcacggccacagtatattgtaaactctgtctaatcatggattgtgtcactgtctctgttgagtgaggtgatcgtgggagtggcaagcgtgtgttgcgacttgagcccggttgtgctgcacacacagatgaagccgtcctctgcacacttcctttatcatgtgttttcaccgtgctgcacaccttggtgctgcacaccttgagtacatctgaggaaagagcctcgtaagataagcggaggggttgcccttccctcacctctcctagagaggtgtgggcaggggacaagagcccagcctcattaaagacactgccatactctgggttttacaacatctgacatttccaggcttctgaagcacaaagtattaaagttgggggggggaagtaaaccaaaattctgatgttcccaaatccccccttcagcagcggctccccggagcgtgtgcggagcagcacaggccacgggtggacccgaggctcacctccttcattcctctcctccaaggctggaggcagggccttcccagtcctcaccctgccagtccccaggcctgcctgcctgcagggtggagctctgggtccctcccacagtgtgatgcagactgctagctgtcactgcctggctttatttaaaggaactgcagcaggtgtcctcagagctgactatgtagaagctttgtctgtttttacctggtcaggtatttttcacactgttgttaccagtaccattccagcctcttgccttgcagttgtatggaaaactgttttataatgagagatctttactgaggattgagcagcatttaataaagtctatgtttgtatttt

SEQ ID NO: 19 - protein, 524 aa, Homo sapiens
MLTCNKAGSRMVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQYTVYSQASTMSIPVAMETDGPLFEDVQMLRKTVKDEAHQAQKDPKKDRPLARRKSELPQDPHTKKALEAHCRADELASQDGR

SEQ ID NO: 20 - DNA, 4167 bp, Homo sapiens
agttccctccagctgcagagagcttcagtttgtctttttttttttaaactaaaatggaggctggtttcttgccttaaggagcccattgcctttcccgctgaagtctagatgttgacatgtaataaagcgggcagcaggatggtggtggatgcggccaactccaatgggcctttccagcccgtggtccttctccatattcgagatgttcctcctgctgatcaagagaagctttttatccagaagttacgtcagtgttgcgtcctctttgactttgtttctgatccactaagtgacctaaagtggaaggaagtaaaacgagctgctttaagtgaaatggtagaatatatcacccataatcggaatgtgatcacagagcctatttacccagaagtagtccatatgtttgcagttaacatgtttcgaacattaccaccttcctccaatcctacgggagcggaatttgacccggaggaagatgaaccaacgttagaagcagcctggcctcatctacagcttgtttatgaatttttcttaagatttttagagtctccagatttccaacctaatatagcgaagaaatatattgatcagaagtttgtattgcagcttttagagctctttgacagtgaagatcctcgggagagagattttcttaaaaccacccttcacagaatctatgggaaattcctaggcttgagagcttacatcagaaaacagataaataatatattttataggtttatttatgaaacagagcatcataatggcatagcagagttactggaaatattgggaagtataattaatggatttgccttaccactaaaagaagagcacaagattttcttattgaaggtgttactacctttgcacaaagtgaaatctctgagtgtctaccatccccagctggcatactgtgtagtgcagtttttagaaaaggacagcaccctcacggaaccagtggtgatggcacttctcaaatactggccaaagactcacagtccaaaagaagtaatgttcttaaacgaattagaagagattttagatgtcattgaaccatcagaatttgtgaagatcatggaacccctcttccggcagttggccaaatgtgtctccagcccacacttccaggtggcagagcgagctctctattactggaataatgaatacatcatgagtttaatcagtgacaacgcagcgaagattctgcccatcatgtttccttccttgtaccgcaactcaaagacccattggaacaagacaatacatggcttgatatacaacgccctgaagctcttcatggagatgaaccaaaagctatttgatgactgtacacaacagttcaaagcagagaaactaaaagagaagctaaaaatgaaagaacgggaagaagcatgggttaaaatagaaaatctagccaaagccaatccccagtacacagtgtatagtcaagccagcaccatgagcattccggttgcaatggagacagatgggcctttatttgaagatgtgcagatgctgagaaagacagtgaaggacgaggctcatcaggcacagaaagatccgaagaaggaccgtcctcttgcacgccgcaagtccgagctgcctcaggacccccacaccaagaaagccttggaagctcactgcagggccgatgagctggcctcccaggacggccgctagcctccggggcgccgcgtcggggccgggcccgccagttcttttccggattctgtagaaaatacatacttcctgtgccataccaatcagttacactcaaagctttcttggaccccgttccgtaggcaataacgtgcgtccgcctcagcgcgagattaggagttcaaacaatggtgacttcccagagcccgctggcagagccgcgggttgacgacggtgtcctcgcagtgtcgccgccaccccagcgtagtccaagtcagactatttcacaaagtcagagcgataggaaagcaccctgcccttcatcttcatgttctcccaaatggaacttaggatcttttaacataggtggttctgtgataacatcagtgttttccaaatcaaaggaacgctttaaaaaataggacctattttttaagactttacagcctttgaaatggtttccacgtgattgttacgccagcagttctcgttttgtttgtttttcaatctcagtgaaatggctctttgctttcgagttctcacgcaacgtactgggcaaatgacaatcctcagccgctggtattttctaaggggtctcttcactttgatgagtgacatgaacaccgtgtctccttctcttgtgtgtacctaaagccatatttccaagtctgtggtactccaggattccaggagtaagcctgtagaagagatttattttaaaagagattgctctgaaatttatcttaaaagagcttgctctgtctaccttgacagaaattggagttttaaaattatgtgttaatatttttatttgcagatttcgtttccgtcaacttaaacattgttgcccttcaacaaggctcttgaattaataaaattatagtctctaagaattccacattttatggaaagttagagcaaaatcattttgagttaagccagttcttagcctaatgcaaactgcagcgcctttaagcataaagtaacacaacagcattgcacggggccggcactgccgctgccttcactgaaggctgcagtgctgttctgagagcttggaggaggcaccagcgaggatgacgtttagtggagctctttctgttgaaaagagctcacgttatcaacaccttgtaaggaaaatacagtgtctgagttttcatcggtcttcacatgctgctatatattccacagagttccttgcatgtactgagcttttgttttagatggaatagcacaaggagaaaaatctttaaacttagtgctttgtctattctttatttctctcagggtggccagtattttgacttatttatcctgcttgaaagctacttgagatgtgtactgctattctaaacacgtgatctagtttctttcatctctggcataagattatataacttaatgttaagtgtcttgaggcataaaagacaaaatgtggcttattttaggatctgttttttcatcgaggtctcgggtatcctttcaaagatagtgagaagcagacactgctccttgtgcagctctggtacctcctgcccactgctgtcacttcaagccactggcaatgcttctgtcctcgtgtcttggaggaaaatcacctggggggaggggacttcttgtggtaagagcaagtgcaggtatgaaatgcgaagattgccccagctaaaagtggacaagtccgctttgtgagatgaatacttcctgagaaacttgacaagtatctctccattttaccattatgaaaactatcattaaaaaaaacagtttagatgccttctccttttgagggaaaaagggtgctttttattgtataaagcagcgtcttatgtattttgatataccattgtttgaacttccgtctttagctgatagattctcaaatatccttgattttggatgttcagtatgtttgtgagagaggtttctgggaagactctctttttgccctcgggaaaaagcaaaatatcaatgtttgggtgactgtgtaaagctcagtgtgtaagaacatctttttgtctaggttttctttctgctctttattgaagacaaacactcaccaaaaagaaaaataaaagttttcagagaaactaattttctttggcaagagtattacttaatattttggcctcctaaagtttccctagttagtactcggactcctgtgctaattgtcagcttacatatcattgtatagagactgtttattctgtaccaaactgatttcaaaagtactacattgaaaataaaccggtgactgtttttcttcataaagttctgcgtttggcatcttcactctttccaaaatgtatctgtacatcagaaatgtcactattccaagtgtctttttagtgtggctttagtatggcttccttttaatattgtacatacattgtatctttgttttatggtaataagtaataaaaatgtagacttcatattttgtacaaaatgtcctatgtacagaataaaaaagttcatagaaacagcaaaaaaaaaaaaaaaaaa

SEQ ID NO: 21 - protein, 384 aa, Homo sapiens
MLTCNKAGSRMVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNK

SEQ ID NO: 22 - DNA, 1460 bp, Homo sapiens
agttccctccagctgcagagagcttcagtttgtctttttttttttaaactaaaatggaggctggtttcttgccttaaggagcccattgcctttcccgctgaagtctagatgttgacatgtaataaagcgggcagcaggatggtggtggatgcggccaactccaatgggcctttccagcccgtggtccttctccatattcgagatgttcctcctgctgatcaagagaagctttttatccagaagttacgtcagtgttgcgtcctctttgactttgtttctgatccactaagtgacctaaagtggaaggaagtaaaacgagctgctttaagtgaaatggtagaatatatcacccataatcggaatgtgatcacagagcctatttacccagaagtagtccatatgtttgcagttaacatgtttcgaacattaccaccttcctccaatcctacgggagcggaatttgacccggaggaagatgaaccaacgttagaagcagcctggcctcatctacagcttgtttatgaatttttcttaagatttttagagtctccagatttccaacctaatatagcgaagaaatatattgatcagaagtttgtattgcagcttttagagctctttgacagtgaagatcctcgggagagagattttcttaaaaccacccttcacagaatctatgggaaattcctaggcttgagagcttacatcagaaaacagataaataatatattttataggtttatttatgaaacagagcatcataatggcatagcagagttactggaaatattgggaagtataattaatggatttgccttaccactaaaagaagagcacaagattttcttattgaaggtgttactacctttgcacaaagtgaaatctctgagtgtctaccatccccagctggcatactgtgtagtgcagtttttagaaaaggacagcaccctcacggaaccagtggtgatggcacttctcaaatactggccaaagactcacagtccaaaagaagtaatgttcttaaacgaattagaagagattttagatgtcattgaaccatcagaatttgtgaagatcatggaacccctcttccggcagttggccaaatgtgtctccagcccacacttccaggtggcagagcgagctctctattactggaataatgaatacatcatgagtttaatcagtgacaacgcagcgaagattctgcccatcatgtttccttccttgtaccgcaactcaaagacccattggaacaagtaagaaagaactggctgccatctttttcagtcattttaaaatatggcacgttttactgctacttcagtaagaataaatatcagaattttaaatatcaattaaaaaacaagaaggtcaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - protein, 449 aa, Homo sapiens
MLTCNKAGSRMVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQVLKKRIT

SEQ ID NO: 24 - DNA, 1586 bp, Homo sapiens
agttccctccagctgcagagagcttcagtttgtctttttttttttaaactaaaatggaggctggtttcttgccttaaggagcccattgcctttcccgctgaagtctagatgttgacatgtaataaagcgggcagcaggatggtggtggatgcggccaactccaatgggcctttccagcccgtggtccttctccatattcgagatgttcctcctgctgatcaagagaagctttttatccagaagttacgtcagtgttgcgtcctctttgactttgtttctgatccactaagtgacctaaagtggaaggaagtaaaacgagctgctttaagtgaaatggtagaatatatcacccataatcggaatgtgatcacagagcctatttacccagaagtagtccatatgtttgcagttaacatgtttcgaacattaccaccttcctccaatcctacgggagcggaatttgacccggaggaagatgaaccaacgttagaagcagcctggcctcatctacagcttgtttatgaatttttcttaagatttttagagtctccagatttccaacctaatatagcgaagaaatatattgatcagaagtttgtattgcagcttttagagctctttgacagtgaagatcctcgggagagagattttcttaaaaccacccttcacagaatctatgggaaattcctaggcttgagagcttacatcagaaaacagataaataatatattttataggtttatttatgaaacagagcatcataatggcatagcagagttactggaaatattgggaagtataattaatggatttgccttaccactaaaagaagagcacaagattttcttattgaaggtgttactacctttgcacaaagtgaaatctctgagtgtctaccatccccagctggcatactgtgtagtgcagtttttagaaaaggacagcaccctcacggaaccagtggtgatggcacttctcaaatactggccaaagactcacagtccaaaagaagtaatgttcttaaacgaattagaagagattttagatgtcattgaaccatcagaatttgtgaagatcatggaacccctcttccggcagttggccaaatgtgtctccagcccacacttccaggtggcagagcgagctctctattactggaataatgaatacatcatgagtttaatcagtgacaacgcagcgaagattctgcccatcatgtttccttccttgtaccgcaactcaaagacccattggaacaagacaatacatggcttgatatacaacgccctgaagctcttcatggagatgaaccaaaagctatttgatgactgtacacaacagttcaaagcagagaaactaaaagagaagctaaaaatgaaagaacgggaagaagcatgggttaaaatagaaaatctagccaaagccaatccccaggtactaaaaaagagaataacatgaaaacgcccagggttacttgaatgtttttataagataggaatatatgtcttcaccatggggggggtctcgatttcactaacgttgtatatgaaaatgtctgcaataaaaagtacttttaaactttgtaa

SEQ ID NO: 25 - protein, 485 aa, Homo sapiens
MLTCNKAGSRMVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQAQKDPKKDRPLARRKSELPQDPHTKKALEAHCRADELASQDGR

SEQ ID NO: 26 - DNA, 4050 bp, Homo sapiens
agttccctccagctgcagagagcttcagtttgtctttttttttttaaactaaaatggaggctggtttcttgccttaaggagcccattgcctttcccgctgaagtctagatgttgacatgtaataaagcgggcagcaggatggtggtggatgcggccaactccaatgggcctttccagcccgtggtccttctccatattcgagatgttcctcctgctgatcaagagaagctttttatccagaagttacgtcagtgttgcgtcctctttgactttgtttctgatccactaagtgacctaaagtggaaggaagtaaaacgagctgctttaagtgaaatggtagaatatatcacccataatcggaatgtgatcacagagcctatttacccagaagtagtccatatgtttgcagttaacatgtttcgaacattaccaccttcctccaatcctacgggagcggaatttgacccggaggaagatgaaccaacgttagaagcagcctggcctcatctacagcttgtttatgaatttttcttaagatttttagagtctccagatttccaacctaatatagcgaagaaatatattgatcagaagtttgtattgcagcttttagagctctttgacagtgaagatcctcgggagagagattttcttaaaaccacccttcacagaatctatgggaaattcctaggcttgagagcttacatcagaaaacagataaataatatattttataggtttatttatgaaacagagcatcataatggcatagcagagttactggaaatattgggaagtataattaatggatttgccttaccactaaaagaagagcacaagattttcttattgaaggtgttactacctttgcacaaagtgaaatctctgagtgtctaccatccccagctggcatactgtgtagtgcagtttttagaaaaggacagcaccctcacggaaccagtggtgatggcacttctcaaatactggccaaagactcacagtccaaaagaagtaatgttcttaaacgaattagaagagattttagatgtcattgaaccatcagaatttgtgaagatcatggaacccctcttccggcagttggccaaatgtgtctccagcccacacttccaggtggcagagcgagctctctattactggaataatgaatacatcatgagtttaatcagtgacaacgcagcgaagattctgcccatcatgtttccttccttgtaccgcaactcaaagacccattggaacaagacaatacatggcttgatatacaacgccctgaagctcttcatggagatgaaccaaaagctatttgatgactgtacacaacagttcaaagcagagaaactaaaagagaagctaaaaatgaaagaacgggaagaagcatgggttaaaatagaaaatctagccaaagccaatccccaggcacagaaagatccgaagaaggaccgtcctcttgcacgccgcaagtccgagctgcctcaggacccccacaccaagaaagccttggaagctcactgcagggccgatgagctggcctcccaggacggccgctagcctccggggcgccgcgtcggggccgggcccgccagttcttttccggattctgtagaaaatacatacttcctgtgccataccaatcagttacactcaaagctttcttggaccccgttccgtaggcaataacgtgcgtccgcctcagcgcgagattaggagttcaaacaatggtgacttcccagagcccgctggcagagccgcgggttgacgacggtgtcctcgcagtgtcgccgccaccccagcgtagtccaagtcagactatttcacaaagtcagagcgataggaaagcaccctgcccttcatcttcatgttctcccaaatggaacttaggatcttttaacataggtggttctgtgataacatcagtgttttccaaatcaaaggaacgctttaaaaaataggacctattttttaagactttacagcctttgaaatggtttccacgtgattgttacgccagcagttctcgttttgtttgtttttcaatctcagtgaaatggctctttgctttcgagttctcacgcaacgtactgggcaaatgacaatcctcagccgctggtattttctaaggggtctcttcactttgatgagtgacatgaacaccgtgtctccttctcttgtgtgtacctaaagccatatttccaagtctgtggtactccaggattccaggagtaagcctgtagaagagatttattttaaaagagattgctctgaaatttatcttaaaagagcttgctctgtctaccttgacagaaattggagttttaaaattatgtgttaatatttttatttgcagatttcgtttccgtcaacttaaacattgttgcccttcaacaaggctcttgaattaataaaattatagtctctaagaattccacattttatggaaagttagagcaaaatcattttgagttaagccagttcttagcctaatgcaaactgcagcgcctttaagcataaagtaacacaacagcattgcacggggccggcactgccgctgccttcactgaaggctgcagtgctgttctgagagcttggaggaggcaccagcgaggatgacgtttagtggagctctttctgttgaaaagagctcacgttatcaacaccttgtaaggaaaatacagtgtctgagttttcatcggtcttcacatgctgctatatattccacagagttccttgcatgtactgagcttttgttttagatggaatagcacaaggagaaaaatctttaaacttagtgctttgtctattctttatttctctcagggtggccagtattttgacttatttatcctgcttgaaagctacttgagatgtgtactgctattctaaacacgtgatctagtttctttcatctctggcataagattatataacttaatgttaagtgtcttgaggcataaaagacaaaatgtggcttattttaggatctgttttttcatcgaggtctcgggtatcctttcaaagatagtgagaagcagacactgctccttgtgcagctctggtacctcctgcccactgctgtcacttcaagccactggcaatgcttctgtcctcgtgtcttggaggaaaatcacctggggggaggggacttcttgtggtaagagcaagtgcaggtatgaaatgcgaagattgccccagctaaaagtggacaagtccgctttgtgagatgaatacttcctgagaaacttgacaagtatctctccattttaccattatgaaaactatcattaaaaaaaacagtttagatgccttctccttttgagggaaaaagggtgctttttattgtataaagcagcgtcttatgtattttgatataccattgtttgaacttccgtctttagctgatagattctcaaatatccttgattttggatgttcagtatgtttgtgagagaggtttctgggaagactctctttttgccctcgggaaaaagcaaaatatcaatgtttgggtgactgtgtaaagctcagtgtgtaagaacatctttttgtctaggttttctttctgctctttattgaagacaaacactcaccaaaaagaaaaataaaagttttcagagaaactaattttctttggcaagagtattacttaatattttggcctcctaaagtttccctagttagtactcggactcctgtgctaattgtcagcttacatatcattgtatagagactgtttattctgtaccaaactgatttcaaaagtactacattgaaaataaaccggtgactgtttttcttcataaagttctgcgtttggcatcttcactctttccaaaatgtatctgtacatcagaaatgtcactattccaagtgtctttttagtgtggctttagtatggcttccttttaatattgtacatacattgtatctttgttttatggtaataagtaataaaaatgtagacttcatattttgtacaaaatgtcctatgtacagaataaaaaagttcatagaaacagcaaaaaaaaaaaaaaaaaa

SEQ ID NO: 27 - protein, 452 aa, Mus musculus
MLTCNKAGSGMVVDAASSNGPFQPVALLHIRDVPPADQEKLFIQKLRQCCVLFDFVSDPLSDLKWKEVKRAALSEMVEYITHNRNVITEPIYPEAVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQVLKKRVTREC

SEQ ID NO: 28 - DNA, 2377 bp, Mus musculus
cgaagcagccagttccctccagctgcagagagcttcagtttgtctttttttttttttaaactaaaatggaggctggtttcttgccttaaggagtacagcgcccttcccgctggagcctagatgttgacatgtaataaagcgggcagcgggatggtggtggatgcggccagctccaacgggcctttccagcccgtggcccttctccacattcgagatgttcctcctgcggatcaagagaagctttttatccagaagctacgccagtgttgtgtcctctttgactttgtctctgacccactgagtgacctgaagtggaaggaagtaaagcgcgctgcgctgagcgagatggtggagtatatcacccacaaccggaacgtgatcacggagcccatttaccccgaggccgtccacatgtttgcagttaacatgttccgaaccttgccaccttcctccaatcccacgggagcagaattcgacccagaagaggatgaaccaacgttagaagcagcctggcctcatctgcagcttgtttatgaatttttcttaagatttttagagtctccagatttccaacccaatatagcaaagaaatatattgatcagaagtttgtattgcagcttctagagctgtttgacagcgaggatcctcgggagagagattttctaaaaaccaccctgcacagaatctatgggaagttcttaggcctgcgtgcttacatcaggaaacagatcaataatatattttataggtttatctatgagacagagcatcacaatggcatagcggagttactggagatcctgggaagtataattaatggatttgccttaccactgaaggaggaacacaagattttcctgctgaaggtgttgctgcccttgcacaaagtgaagtccctgagtgtctaccatccccagctggcgtactgtgtcgtgcagtttttagagaaggacagcaccctcactgaaccagtggtaatggcacttctcaaatactggccaaagactcacagtccaaaagaagtaatgttcttaaatgaattagaagaaattttagatgtaattgaaccatcagagtttgtgaagatcatggagcctcttttccgacagttagccaaatgtgtttccagccctcacttccaggtggccgagcgggcgctctattactggaacaacgagtacatcatgagtttaatcagtgacaacgcagcgaagattctgcccatcatgtttccgtccttataccgcaactcaaagacccactggaacaagacaatacacggcttgatatacaacgccctgaaactcttcatggagatgaaccaaaaactcttcgatgactgcactcagcagttcaaagcagagaaactcaaagagaagctaaaaatgaaagagcgagaagaagcatgggttaaaatagaaaatctagccaaagcgaatccccaggtactaaaaaagagagtaactcgggagtgttgaggctttgcgtgaatgtctgagatagggcctggctccaccccaggaagggaggccaacgtcactaacactgtatgtgcaaatgtccgcaataaaacactttccaactttgtaacttcctcttgtataagtacttatttgccacacgtaacttttaccacagaatcgatttttttctcttctttttaaaagtaagatgtgatgtggtaaagagaacaccaggatgtaacctctaagattgtaatgtcctttcttgctcgaatgtcatagatgctgtcacttgaaccgtgttcctccgttttattctcatacatgagagggatgggggggaggcagatgaagaatgctgaaaactaactgaattctgccctgctcacactaactgttcctctaccctagccgatgcagtgtgctcccgcaaggcgaagccaggcagatggtcctgtctgtcagagctgcaggtgactcagcagcctctgtccagggttggcttttaggctggcagagcctgggacagctttgcagccatagctctttggtgttgcctccgctggtccgaatgatcagttattggggtgttgcctctgctggtccgaatgatcagttattggggtgttgcctctgctgctccgaatgatcaatgttactgggtgtttcttttctccttgttgcgtgtgtctgattataacagccacttgatgaaatgtctgaccttctccacactaagatctctcaggcttctctcagctctccagggaagaaaatatcttactaaacagtgtatttctttttggttgagaagtgtgtattaagtgtgtacataaattattatgtaagttggatgtgggtttttttaatctttgtcatttaataaaaacaatatatgaagataaaaaaaaaa

SEQ ID NO: 29 - protein, 524 aa, Mus musculus
MLTCNKAGSGMVVDAASSNGPFQPVALLHIRDVPPADQEKLFIQKLRQCCVLFDFVSDPLSDLKWKEVKRAALSEMVEYITHNRNVITEPIYPEAVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQYAVYSQASAVSIPVAMETDGPQFEDVQMLKKTVSDEARQAQKELKKDRPLVRRKSELPQDPHTEKALEAHCRASELLSQDGR

SEQ ID NO: 30 - DNA, 4224 bp, Mus musculus
cgaagcagccagttccctccagctgcagagagcttcagtttgtctttttttttttttaaactaaaatggaggctggtttcttgccttaaggagtacagcgcccttcccgctggagcctagatgttgacatgtaataaagcgggcagcgggatggtggtggatgcggccagctccaacgggcctttccagcccgtggcccttctccacattcgagatgttcctcctgcggatcaagagaagctttttatccagaagctacgccagtgttgtgtcctctttgactttgtctctgacccactgagtgacctgaagtggaaggaagtaaagcgcgctgcgctgagcgagatggtggagtatatcacccacaaccggaacgtgatcacggagcccatttaccccgaggccgtccacatgtttgcagttaacatgttccgaaccttgccaccttcctccaatcccacgggagcagaattcgacccagaagaggatgaaccaacgttagaagcagcctggcctcatctgcagcttgtttatgaatttttcttaagatttttagagtctccagatttccaacccaatatagcaaagaaatatattgatcagaagtttgtattgcagcttctagagctgtttgacagcgaggatcctcgggagagagattttctaaaaaccaccctgcacagaatctatgggaagttcttaggcctgcgtgcttacatcaggaaacagatcaataatatattttataggtttatctatgagacagagcatcacaatggcatagcggagttactggagatcctgggaagtataattaatggatttgccttaccactgaaggaggaacacaagattttcctgctgaaggtgttgctgcccttgcacaaagtgaagtccctgagtgtctaccatccccagctggcgtactgtgtcgtgcagtttttagagaaggacagcaccctcactgaaccagtggtaatggcacttctcaaatactggccaaagactcacagtccaaaagaagtaatgttcttaaatgaattagaagaaattttagatgtaattgaaccatcagagtttgtgaagatcatggagcctcttttccgacagttagccaaatgtgtttccagccctcacttccaggtggccgagcgggcgctctattactggaacaacgagtacatcatgagtttaatcagtgacaacgcagcgaagattctgcccatcatgtttccgtccttataccgcaactcaaagacccactggaacaagacaatacacggcttgatatacaacgccctgaaactcttcatggagatgaaccaaaaactcttcgatgactgcactcagcagttcaaagcagagaaactcaaagagaagctaaaaatgaaagagcgagaagaagcatgggttaaaatagaaaatctagccaaagcgaatccccagtatgcagtgtatagtcaagccagcgccgtgagcattccggtcgcaatggagacagatgggcctcagtttgaagatgtgcagatgctgaaaaagacagtgagcgacgaggctcgtcaggcacagaaggagctgaagaaggatcgtcccctcgtgcgccgcaagtccgagctgcctcaggacccccacaccgagaaagccttggaagctcactgcagagccagtgagctgctctcccaggacgggcgctagcgtctggagcagcacgccgagctgggcctgtcggttctcttctggatgctgtagaaaagacatactgtgtgtgccataccagtcagttacactcaatgtcaaagcttcctctgaccccgttctgtaggcaataatgcacgtccgcctcagctcgagattaggagttcaaacaatggtggcttctctggccctgctggccaagcagggggctgggggagtcggtgacagtgtcatcaccatgttgtcaccaccccagcacagccctgagtgagatccaaagtcagagctgtaggaaagcaccctgactgtcaccttctcgtacccagtagaccctgtggtcctctgaaatagggattctgtggtaggtaacaccgatgctgtggcaaatcaaacgcacatggtaaaaggtgtctcctccctttacaaccctgacgctgtccccacgcttgctgtgccagccgcccgtggtggttggttaggtctctcagagatggctcctcgctttcccgttctagctgcctgccgggaaagaggccaacttttgcgctggggccgtccacagtgtttctttcctttggttaatggcacggtcaccatgtcctgcttaccttaatgtctaccgaaagccatatttcatagtctgtggtagaggccagggttccacctcagcgcaagggagggttctgagaagtgcttcctcagcccaccttgaggagaaccgccacgttggaatgacgtgttcatgttagttgcagaatcagtttttcacctacttacacatgatccttcaacaaggctctcgagtggataaagatcacaggatctaagaattccacattggtagaaagtgacagtaaactcagccccttggcctagtgcagcctgcagctgcctgtggcctgactgcgggccgcactgtgtgggcgccctcagtgagcctttctttctgttggaaaaagcacatgtcaccaagctcgcgaggaaaggcgcggtgcccgcgttcgcaccagtctccacatgctgctccggccttcacagaattcttgcatggattaagcttctgttttagatgggatagcacgaggagatacatccttaaacttagtgctttgtcttcttgtcctctcagggtggccagtattttgacttatttatcctgctcaaaagctgcttgaaatgtgtgctgctctctgaacagtggcgagtcttctcttgtctccagcatatatataactcagtgttacatgtcttgatgcataagataggttaaaaaaaaagaagaagaagaaaaatggggcttcttttaaaacctgattttaatcgtggtctcagggatccgtgaaaaagttgtgaaatcagacattgctctctcgcggccttggaggcgggtcccacggggaagaagggcatgtgcagggagggagagccagcacggctagtcaaaaaggcggccccagctgagggtgaacaggttggccacttgggtgagaacacttcctgagaaacttgacaagtacctatccactttaccattatgaaatctataattaaaaaaaaaaagttgagatgccttctccttttgagggaaaaagggtgcttttattgtataaagcagtgtctctgtgttttgatagcccactgtttgaactctcgtctttagctggtagagtctcagatatccctgacgtgtggggcattccgagtgtggtgagaggtcccaagaggctcctttgccctgcgtgaagagcatgctatcagtgtacgggtgattgtgcaaagctcagcgcgggggagcatcttctgctctaggttttatttctgctctttattgaagacaaacattcgccaataaagaaaagggaaaaaaatgttttgagagaaactaattttctttggcaaaagtattacttaaatttttggcctattaaggttcccctagttagtactcggattccccatgctaattgttcagcttgtatgttgttaagacactgttgattctgtaccagactggtttcaaaacaaaaaaagtaccacgttgaaaataaactggtgactgtttttcttcatatggctctagtttggcaccttcactctttccaaaacgtgtctgtgcgccagagatgtcacagttcaagtgtctttctagtgtggctttgtatggcttcctttgaacattgtacatacattgtatctttgttttatggtaataagtaataaaaaatgtagacttcgtattttgtacaaaatgtcctatgtacagaataaaaaaaagttcatagaaacagcaaaaataggttaagtggcacagttatttttctttagaaaatatctgtaactttatgctttagtgaaacgttaagtaccaacatattttttaacattttgtaattcaaaacttttttgttttgacattgtttatgaagaaagacttcatgcacttgccatttaatatgctcttttatctaattttaaagaactctttaaaatggtgtattatatggactaaataaagaacatgtgaatttt

SEQ ID NO: 31 - protein, 485 aa, Mus musculus
MLTCNKAGSGMVVDAASSNGPFQPVALLHIRDVPPADQEKLFIQKLRQCCVLFDFVSDPLSDLKWKEVKRAALSEMVEYITHNRNVITEPIYPEAVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQAQKELKKDRPLVRRKSELPQDPHTEKALEAHCRASELLSQDGR

SEQ ID NO: 32 - DNA, 4107 bp, Mus musculus
cgaagcagccagttccctccagctgcagagagcttcagtttgtctttttttttttttaaactaaaatggaggctggtttcttgccttaaggagtacagcgcccttcccgctggagcctagatgttgacatgtaataaagcgggcagcgggatggtggtggatgcggccagctccaacgggcctttccagcccgtggcccttctccacattcgagatgttcctcctgcggatcaagagaagctttttatccagaagctacgccagtgttgtgtcctctttgactttgtctctgacccactgagtgacctgaagtggaaggaagtaaagcgcgctgcgctgagcgagatggtggagtatatcacccacaaccggaacgtgatcacggagcccatttaccccgaggccgtccacatgtttgcagttaacatgttccgaaccttgccaccttcctccaatcccacgggagcagaattcgacccagaagaggatgaaccaacgttagaagcagcctggcctcatctgcagcttgtttatgaatttttcttaagatttttagagtctccagatttccaacccaatatagcaaagaaatatattgatcagaagtttgtattgcagcttctagagctgtttgacagcgaggatcctcgggagagagattttctaaaaaccaccctgcacagaatctatgggaagttcttaggcctgcgtgcttacatcaggaaacagatcaataatatattttataggtttatctatgagacagagcatcacaatggcatagcggagttactggagatcctgggaagtataattaatggatttgccttaccactgaaggaggaacacaagattttcctgctgaaggtgttgctgcccttgcacaaagtgaagtccctgagtgtctaccatccccagctggcgtactgtgtcgtgcagtttttagagaaggacagcaccctcactgaaccagtggtaatggcacttctcaaatactggccaaagactcacagtccaaaagaagtaatgttcttaaatgaattagaagaaattttagatgtaattgaaccatcagagtttgtgaagatcatggagcctcttttccgacagttagccaaatgtgtttccagccctcacttccaggtggccgagcgggcgctctattactggaacaacgagtacatcatgagtttaatcagtgacaacgcagcgaagattctgcccatcatgtttccgtccttataccgcaactcaaagacccactggaacaagacaatacacggcttgatatacaacgccctgaaactcttcatggagatgaaccaaaaactcttcgatgactgcactcagcagttcaaagcagagaaactcaaagagaagctaaaaatgaaagagcgagaagaagcatgggttaaaatagaaaatctagccaaagcgaatccccaggcacagaaggagctgaagaaggatcgtcccctcgtgcgccgcaagtccgagctgcctcaggacccccacaccgagaaagccttggaagctcactgcagagccagtgagctgctctcccaggacgggcgctagcgtctggagcagcacgccgagctgggcctgtcggttctcttctggatgctgtagaaaagacatactgtgtgtgccataccagtcagttacactcaatgtcaaagcttcctctgaccccgttctgtaggcaataatgcacgtccgcctcagctcgagattaggagttcaaacaatggtggcttctctggccctgctggccaagcagggggctgggggagtcggtgacagtgtcatcaccatgttgtcaccaccccagcacagccctgagtgagatccaaagtcagagctgtaggaaagcaccctgactgtcaccttctcgtacccagtagaccctgtggtcctctgaaatagggattctgtggtaggtaacaccgatgctgtggcaaatcaaacgcacatggtaaaaggtgtctcctccctttacaaccctgacgctgtccccacgcttgctgtgccagccgcccgtggtggttggttaggtctctcagagatggctcctcgctttcccgttctagctgcctgccgggaaagaggccaacttttgcgctggggccgtccacagtgtttctttcctttggttaatggcacggtcaccatgtcctgcttaccttaatgtctaccgaaagccatatttcatagtctgtggtagaggccagggttccacctcagcgcaagggagggttctgagaagtgcttcctcagcccaccttgaggagaaccgccacgttggaatgacgtgttcatgttagttgcagaatcagtttttcacctacttacacatgatccttcaacaaggctctcgagtggataaagatcacaggatctaagaattccacattggtagaaagtgacagtaaactcagccccttggcctagtgcagcctgcagctgcctgtggcctgactgcgggccgcactgtgtgggcgccctcagtgagcctttctttctgttggaaaaagcacatgtcaccaagctcgcgaggaaaggcgcggtgcccgcgttcgcaccagtctccacatgctgctccggccttcacagaattcttgcatggattaagcttctgttttagatgggatagcacgaggagatacatccttaaacttagtgctttgtcttcttgtcctctcagggtggccagtattttgacttatttatcctgctcaaaagctgcttgaaatgtgtgctgctctctgaacagtggcgagtcttctcttgtctccagcatatatataactcagtgttacatgtcttgatgcataagataggttaaaaaaaaagaagaagaagaaaaatggggcttcttttaaaacctgattttaatcgtggtctcagggatccgtgaaaaagttgtgaaatcagacattgctctctcgcggccttggaggcgggtcccacggggaagaagggcatgtgcagggagggagagccagcacggctagtcaaaaaggcggccccagctgagggtgaacaggttggccacttgggtgagaacacttcctgagaaacttgacaagtacctatccactttaccattatgaaatctataattaaaaaaaaaaagttgagatgccttctccttttgagggaaaaagggtgcttttattgtataaagcagtgtctctgtgttttgatagcccactgtttgaactctcgtctttagctggtagagtctcagatatccctgacgtgtggggcattccgagtgtggtgagaggtcccaagaggctcctttgccctgcgtgaagagcatgctatcagtgtacgggtgattgtgcaaagctcagcgcgggggagcatcttctgctctaggttttatttctgctctttattgaagacaaacattcgccaataaagaaaagggaaaaaaatgttttgagagaaactaattttctttggcaaaagtattacttaaatttttggcctattaaggttcccctagttagtactcggattccccatgctaattgttcagcttgtatgttgttaagacactgttgattctgtaccagactggtttcaaaacaaaaaaagtaccacgttgaaaataaactggtgactgtttttcttcatatggctctagtttggcaccttcactctttccaaaacgtgtctgtgcgccagagatgtcacagttcaagtgtctttctagtgtggctttgtatggcttcctttgaacattgtacatacattgtatctttgttttatggtaataagtaataaaaaatgtagacttcgtattttgtacaaaatgtcctatgtacagaataaaaaaaagttcatagaaacagcaaaaataggttaagtggcacagttatttttctttagaaaatatctgtaactttatgctttagtgaaacgttaagtaccaacatattttttaacattttgtaattcaaaacttttttgttttgacattgtttatgaagaaagacttcatgcacttgccatttaatatgctcttttatctaattttaaagaactctttaaaatggtgtattatatggactaaataaagaacatgtgaatttt

SEQ ID NO: 33 - protein, 602 aa, Homo sapiens
MPYKLKKEKEPPKVAKCTAKPSSSGKDGGGENTEEAQPQPQPQPQPQAQSQPPSSNKRPSNSTPPPTQLSKIKYSGGPQIVKKERRQSSSRFNLSKNRELQKLPALKDSPTQEREELFIQKLRQCCVLFDFVSDPLSDLKFKEVKRAGLNEMVEYITHSRDVVTEAIYPEAVTMFSVNLFRTLPPSSNPTGAEFDPEEDEPTLEAAWPHLQLVYEFFLRFLESPDFQPNIAKKYIDQKFVLALLDLFDSEDPRERDFLKTILHRIYGKFLGLRAYIRRQINHIFYRFIYETEHHNGIAELLEILGSIINGFALPLKEEHKMFLIRVLLPLHKVKSLSVYHPQLAYCVVQFLEKESSLTEPVIVGLLKFWPKTHSPKEVMFLNELEEILDVIEPSEFSKVMEPLFRQLAKCVSSPHFQVAERALYYWNNEYIMSLISDNAARVLPIMFPALYRNSKSHWNKTIHGLIYNALKLFMEMNQKLFDDCTQQYKAEKQKGRFRMKEREEMWQKIEELARLNPQYPMFRAPPPLPPVYSMETETPTAEDIQLLKRTVETEAVQMLKDIKKEKVLLRRKSELPQDVYTIKALEAHKRAEEFLTASQEAL

SEQ ID NO: 34 - DNA, 2975 bp, Homo sapiens
gagacgccgagcgggccgagtgcggccgagcaaagccggagccggagcggggccgcaggagacgggccgggtccggacgggccgagatgccctataaactgaaaaaggagaaggagccccccaaggttgccaaatgcacagccaagcctagcagctcgggcaaggatggtggaggcgagaacactgaggaggcccagccgcagccccagccccagccccagccccaagcccagtctcagccaccgtcatccaacaagcgtcccagcaatagcacgccgccccccacgcagctcagcaaaatcaagtactcaggggggccccagattgtcaagaaggagcgacggcaaagctcctcccgcttcaacctcagcaagaatcgggagctgcagaagcttcctgccctgaaagattcgccaacccaggagcgggaggagctgtttatccagaagctacgccagtgctgtgtcctctttgacttcgtgtcagacccactcagtgacctcaaattcaaggaggtgaagcgggcaggactcaacgagatggtggagtacatcacccatagccgtgatgttgtcactgaggccatttaccctgaggctgtcaccatgttttcagtgaacctcttccggacgctgccaccttcatcgaatcccacaggggctgagtttgacccagaggaagatgagcccaccctggaagctgcttggccacatctccagctcgtgtatgagttcttcttacgtttccttgagtctcctgatttccagccaaacatagccaagaagtacatcgaccagaagtttgtacttgctctcctagacctatttgacagtgaggatcctcgagagcgggacttcctcaagaccattttgcatcgcatctatggcaagtttttggggctccgggcttatatccgtaggcagatcaaccacatcttctacaggttcatctacgagacggagcatcacaacgggattgctgagctcctggagatcctgggcagcatcatcaatggctttgccctgccccttaaagaagagcacaagatgttcctcatccgtgtcctacttccccttcacaaggtcaagtccctgagtgtctaccaccctcagctggcatactgtgtggtacaattcctggagaaggagagcagtctgactgagccggtaattgtgggacttctcaagttttggcccaagacccacagccccaaggaggtgatgttcttgaatgagctggaggagattctggacgtcattgaaccttctgagttcagcaaagtgatggaacccctcttccgccagctggccaagtgtgtctctagcccccatttccaggtggcagagcgtgctctctattactggaacaatgagtacatcatgagcctgataagtgacaatgctgcccgagtcctccccatcatgttccctgcactctacaggaactccaagagccactggaacaagacaatccatggactgatctataatgccctgaagttgtttatggaaatgaatcagaagctgtttgatgactgcacacaacaatacaaggcagagaagcagaagggccggttccgaatgaaggaaagggaagagatgtggcaaaaaatcgaggagctggcccggcttaatccccagtatcccatgttccgagcccctccaccactgccccctgtgtactcgatggagacagagacccccacagctgaggacatccagcttctgaagaggactgtggagactgaggctgttcagatgctaaaagacatcaagaaggagaaagtgctgctgcggaggaagtcggagctgccccaggacgtgtacaccatcaaggcactggaggcgcacaagcgggcggaagagttcctaactgccagccaggaggctctctgacccctcacgttcctaccacagggccacagcccacacagccctgggacactgccctggccctccatactctgctccctactggctgtcttgggggaaggcagcgcctctctagctactcaagggagggggatgtgggcacttgaagcagggacacccacagaatggtccctcttctccccaaaaggtgttcatgcctccctgtggctagtacaggctgagcactaagatgcttagtgctcagacaacctggggatgcctgtcccctacctgctcctcacccacagctacctgaggctgctctgagaagtacacacaggaatacatacgctcctctattcttcccttcatcctcatttgaacgccaggtatctcccctcctctctctcccctgcagaggcatgtagggaacagcaggagattattctcaccaaagttatgtcaagccccattggtcccagagtagctgaagggaagccaacccccctgcagcacaaataggccccccagtcccagccgtgtgctggcagatagggttgtattatttcttcttaccccatgcctgaccaagggaggtcaaaaggagaaaagtataggctgtggacaataactgatgaatatagggcccagatggaccaagtggggccggggagggatgaataaacaccctaccccgtgccctgtctttggtgagcagcagccctggggtcacagacatggaagggaccaccctggggctgactgcttttctgtgctgttggttcccaaaactagaaagaaggaagcagggagcggtgccccaagcatggctcctgccaacacctatttatttccttgtttgtgctatgctgggcaggccttctcttgtcccttataggtaccttggaggggccaggggctgaggaaggccggacccaggttccaggggcgcaggcagtgcggcttttggctgtgtacatagggtgctttattctccacagagtgatacatgctaaggtgggttgggcttggaccgatgtccccatatgtacagaactgaataaagtgggtctctgagaaaaaaaaaaaaaaaaaaaaaaaaaa

SEQ ID NO: 35 - protein, 570 aa, Homo sapiens
MPYKLKKEKEPPKVAKCTAKPSSSGKDGGGENTEEAQPQPQPQPQPQAQSQPPSSNKRPSNSTPPPTQLSKIKYSGGPQIVKKELFIQKLRQCCVLFDFVSDPLSDLKFKEVKRAGLNEMVEYITHSRDVVTEAIYPEAVTMFSVNLFRTLPPSSNPTGAEFDPEEDEPTLEAAWPHLQLVYEFFLRFLESPDFQPNIAKKYIDQKFVLALLDLFDSEDPRERDFLKTILHRIYGKFLGLRAYIRRQINHIFYRFIYETEHHNGIAELLEILGSIINGFALPLKEEHKMFLIRVLLPLHKVKSLSVYHPQLAYCVVQFLEKESSLTEPVIVGLLKFWPKTHSPKEVMFLNELEEILDVIEPSEFSKVMEPLFRQLAKCVSSPHFQVAERALYYWNNEYIMSLISDNAARVLPIMFPALYRNSKSHWNKTIHGLIYNALKLFMEMNQKLFDDCTQQYKAEKQKGRFRMKEREEMWQKIEELARLNPQYPMFRAPPPLPPVYSMETETPTAEDIQLLKRTVETEAVQMLKDIKKEKVLLRRKSELPQDVYTIKALEAHKRAEEFLTASQEAL

SEQ ID NO: 36 - DNA, 2871 bp, Homo sapiens
gagacgccgagcgggccgagtgcggccgagcaaagccggagccggagcggggccgcaggagacgggccgggtccggacgggccgagatgccctataaactgaaaaaggagaaggagccccccaaggttgccaaatgcacagccaagcctagcagctcgggcaaggatggtggaggcgagaacactgaggaggcccagccgcagccccagccccagccccagccccaagcccagtctcagccaccgtcatccaacaagcgtcccagcaatagcacgccgccccccacgcagctcagcaaaatcaagtactcaggggggccccagattgtcaagaaggagctgtttatccagaagctacgccagtgctgtgtcctctttgacttcgtgtcagacccactcagtgacctcaaattcaaggaggtgaagcgggcaggactcaacgagatggtggagtacatcacccatagccgtgatgttgtcactgaggccatttaccctgaggctgtcaccatgttttcagtgaacctcttccggacgctgccaccttcatcgaatcccacaggggctgagtttgacccagaggaagatgagcccaccctggaagctgcttggccacatctccagctcgtgtatgagttcttcttacgtttccttgagtctcctgatttccagccaaacatagccaagaagtacatcgaccagaagtttgtacttgctctcctagacctatttgacagtgaggatcctcgagagcgggacttcctcaagaccattttgcatcgcatctatggcaagtttttggggctccgggcttatatccgtaggcagatcaaccacatcttctacaggttcatctacgagacggagcatcacaacgggattgctgagctcctggagatcctgggcagcatcatcaatggctttgccctgccccttaaagaagagcacaagatgttcctcatccgtgtcctacttccccttcacaaggtcaagtccctgagtgtctaccaccctcagctggcatactgtgtggtacaattcctggagaaggagagcagtctgactgagccggtaattgtgggacttctcaagttttggcccaagacccacagccccaaggaggtgatgttcttgaatgagctggaggagattctggacgtcattgaaccttctgagttcagcaaagtgatggaacccctcttccgccagctggccaagtgtgtctctagcccccatttccaggtggcagagcgtgctctctattactggaacaatgagtacatcatgagcctgataagtgacaatgctgcccgagtcctccccatcatgttccctgcactctacaggaactccaagagccactggaacaagacaatccatggactgatctataatgccctgaagttgtttatggaaatgaatcagaagctgtttgatgactgcacacaacaatacaaggcagagaagcagaagggccggttccgaatgaaggaaagggaagagatgtggcaaaaaatcgaggagctggcccggcttaatccccagtatcccatgttccgagcccctccaccactgccccctgtgtactcgatggagacagagacccccacagctgaggacatccagcttctgaagaggactgtggagactgaggctgttcagatgctaaaagacatcaagaaggagaaagtgctgctgcggaggaagtcggagctgccccaggacgtgtacaccatcaaggcactggaggcgcacaagcgggcggaagagttcctaactgccagccaggaggctctctgacccctcacgttcctaccacagggccacagcccacacagccctgggacactgccctggccctccatactctgctccctactggctgtcttgggggaaggcagcgcctctctagctactcaagggagggggatgtgggcacttgaagcagggacacccacagaatggtccctcttctccccaaaaggtgttcatgcctccctgtggctagtacaggctgagcactaagatgcttagtgctcagacaacctggggatgcctgtcccctacctgctcctcacccacagctacctgaggctgctctgagaagtacacacaggaatacatacgctcctctattcttcccttcatcctcatttgaacgccaggtatctcccctcctctctctcccctgcagaggcatgtagggaacagcaggagattattctcaccaaagttatgtcaagccccattggtcccagagtagctgaagggaagccaacccccctgcagcacaaataggccccccagtcccagccgtgtgctggcagatagggttgtattatttcttcttaccccatgcctgaccaagggaggtcaaaaggagaaaagtataggctgtggacaataactgatgaatatagggcccagatggaccaagtggggccggggagggatgaataaacaccctaccccgtgccctgtctttggtgagcagcagccctggggtcacagacatggaagggaccaccctggggctgactgcttttctgtgctgttggttcccaaaactagaaagaaggaagcagggagcggtgccccaagcatggctcctgccaacacctatttatttccttgtttgtgctatgctgggcaggccttctcttgtcccttataggtaccttggaggggccaggggctgaggaaggccggacccaggttccaggggcgcaggcagtgcggcttttggctgtgtacatagggtgctttattctccacagagtgatacatgctaaggtgggttgggcttggaccgatgtccccatatgtacagaactgaataaagtgggtctctgagaaaaaaaaaaaaaaaaaa

SEQ ID NO: 37 - protein, 496 aa, Homo sapiens
MPYKLKKEKELFIQKLRQCCVLFDFVSDPLSDLKFKEVKRAGLNEMVEYITHSRDVVTEAIYPEAVTMFSVNLFRTLPPSSNPTGAEFDPEEDEPTLEAAWPHLQLVYEFFLRFLESPDFQPNIAKKYIDQKFVLALLDLFDSEDPRERDFLKTILHRIYGKFLGLRAYIRRQINHIFYRFIYETEHHNGIAELLEILGSIINGFALPLKEEHKMFLIRVLLPLHKVKSLSVYHPQLAYCVVQFLEKESSLTEPVIVGLLKFWPKTHSPKEVMFLNELEEILDVIEPSEFSKVMEPLFRQLAKCVSSPHFQVAERALYYWNNEYIMSLISDNAARVLPIMFPALYRNSKSHWNKTIHGLIYNALKLFMEMNQKLFDDCTQQYKAEKQKGRFRMKEREEMWQKIEELARLNPQYPMFRAPPPLPPVYSMETETPTAEDIQLLKRTVETEAVQMLKDIKKEKVLLRRKSELPQDVYTIKALEAHKRAEEFLTASQEAL

SEQ ID NO: 38 - DNA, 2649 bp, Homo sapiens
gagacgccgagcgggccgagtgcggccgagcaaagccggagccggagcggggccgcaggagacgggccgggtccggacgggccgagatgccctataaactgaaaaaggagaaggagctgtttatccagaagctacgccagtgctgtgtcctctttgacttcgtgtcagacccactcagtgacctcaaattcaaggaggtgaagcgggcaggactcaacgagatggtggagtacatcacccatagccgtgatgttgtcactgaggccatttaccctgaggctgtcaccatgttttcagtgaacctcttccggacgctgccaccttcatcgaatcccacaggggctgagtttgacccagaggaagatgagcccaccctggaagctgcttggccacatctccagctcgtgtatgagttcttcttacgtttccttgagtctcctgatttccagccaaacatagccaagaagtacatcgaccagaagtttgtacttgctctcctagacctatttgacagtgaggatcctcgagagcgggacttcctcaagaccattttgcatcgcatctatggcaagtttttggggctccgggcttatatccgtaggcagatcaaccacatcttctacaggttcatctacgagacggagcatcacaacgggattgctgagctcctggagatcctgggcagcatcatcaatggctttgccctgccccttaaagaagagcacaagatgttcctcatccgtgtcctacttccccttcacaaggtcaagtccctgagtgtctaccaccctcagctggcatactgtgtggtacaattcctggagaaggagagcagtctgactgagccggtaattgtgggacttctcaagttttggcccaagacccacagccccaaggaggtgatgttcttgaatgagctggaggagattctggacgtcattgaaccttctgagttcagcaaagtgatggaacccctcttccgccagctggccaagtgtgtctctagcccccatttccaggtggcagagcgtgctctctattactggaacaatgagtacatcatgagcctgataagtgacaatgctgcccgagtcctccccatcatgttccctgcactctacaggaactccaagagccactggaacaagacaatccatggactgatctataatgccctgaagttgtttatggaaatgaatcagaagctgtttgatgactgcacacaacaatacaaggcagagaagcagaagggccggttccgaatgaaggaaagggaagagatgtggcaaaaaatcgaggagctggcccggcttaatccccagtatcccatgttccgagcccctccaccactgccccctgtgtactcgatggagacagagacccccacagctgaggacatccagcttctgaagaggactgtggagactgaggctgttcagatgctaaaagacatcaagaaggagaaagtgctgctgcggaggaagtcggagctgccccaggacgtgtacaccatcaaggcactggaggcgcacaagcgggcggaagagttcctaactgccagccaggaggctctctgacccctcacgttcctaccacagggccacagcccacacagccctgggacactgccctggccctccatactctgctccctactggctgtcttgggggaaggcagcgcctctctagctactcaagggagggggatgtgggcacttgaagcagggacacccacagaatggtccctcttctccccaaaaggtgttcatgcctccctgtggctagtacaggctgagcactaagatgcttagtgctcagacaacctggggatgcctgtcccctacctgctcctcacccacagctacctgaggctgctctgagaagtacacacaggaatacatacgctcctctattcttcccttcatcctcatttgaacgccaggtatctcccctcctctctctcccctgcagaggcatgtagggaacagcaggagattattctcaccaaagttatgtcaagccccattggtcccagagtagctgaagggaagccaacccccctgcagcacaaataggccccccagtcccagccgtgtgctggcagatagggttgtattatttcttcttaccccatgcctgaccaagggaggtcaaaaggagaaaagtataggctgtggacaataactgatgaatatagggcccagatggaccaagtggggccggggagggatgaataaacaccctaccccgtgccctgtctttggtgagcagcagccctggggtcacagacatggaagggaccaccctggggctgactgcttttctgtgctgttggttcccaaaactagaaagaaggaagcagggagcggtgccccaagcatggctcctgccaacacctatttatttccttgtttgtgctatgctgggcaggccttctcttgtcccttataggtaccttggaggggccaggggctgaggaaggccggacccaggttccaggggcgcaggcagtgcggcttttggctgtgtacatagggtgctttattctccacagagtgatacatgctaaggtgggttgggcttggaccgatgtccccatatgtacagaactgaataaagtgggtctctgagaaaaaaaaaaaaaaaaaa

SEQ ID NO: 39 - protein, 594 aa, Mus musculus
MSYKLKKDKEPSKLAKGTAKPSSSSKDGGGENTDEAQPQPQSQSPSSNKRPSNSTPPPTQLSKIKYSGGPQIVKKERRQSSFPFNLNKNRELQKLPALKDSPTQEREELFIQKLRQCCVLFDFVSDPLSDLKCKEVKRAGLNEMVEYITHSRDVVTEAIYPEAVTMFSVNLFRTLPPSSNPTGAEFDPEEDEPTLEAAWPHLQLVYEFFLRFLESPDFQPNIAKKYIDQKFVLALLDLFDSEDPRERDFLKTILHRIYGKFLGLRAYIRRQINHIFYRFIYETEHHNGIAELLEILGSIINGFALPLKEEHKVFLVRVLLPLHKVKSLSVYHPQLAYCVVQFLEKESSLTEPVIVGLLKFWPKTHSPKEVMFLNELEEILDVIEPSEFSKVMEPLFRQLAKCVSSPHFQVAERALYYWNNEYIMSLISDNAARILPIMFPALYRNSKSHWNKTIHGLIYNALKLFMEMNQKLFDDCTQQYKAEKQKGRFRMKEREEMWQKIEELARLNPQYPMFRAPPPLPPVYSMETETPTAEDIQLLKRTVETEAVQMLKDIKKDKVLLRRKSELPQDVYTIKALEAHKRAEEFLTASQEAL

SEQ ID NO: 40 - DNA, 2871 bp, Mus musculus
gtggcgaagagacgccgagcgggccgagtgtggccgagcagagccggagcggggccgcaggagccgggccgggtgtggaccggccgagatgtcctataaactgaagaaggataaggaaccctccaagcttgccaaaggcacagccaagcccagcagctcaagcaaggatggtggaggggagaacaccgatgaggcccagccccagccccagtctcagtcaccatcatccaacaagcgacccagcaacagtacaccacccccaacacaactcagcaaaatcaagtactcagggggaccccagattgtcaagaaagaacgacgccaaagctccttccccttcaacctcaacaagaaccgggagctacaaaaacttcctgccttgaaagactcaccaacccaggaacgtgaggagctgtttatccagaagctacgccagtgctgtgtcctctttgacttcgtgtcagacccactcagtgacctcaaatgcaaagaggtgaagcgggcaggactcaatgagatggtggagtatatcacccacagccgtgatgttgtcactgaggccatctaccctgaggctgtcaccatgttttcagtgaatctcttccggacgctgcctccttcatcgaatcccacaggagcagagtttgaccctgaggaagatgagccaaccttggaagctgcctggccgcatctccagctcgtgtacgagtttttcttacgtttcttggagtctccagatttccagccaaatatagccaagaagtacattgaccagaagtttgtacttgctctcctggacctattcgatagtgaagaccctagagagcgggattttctcaagaccattttgcatcgcatctatggcaagtttttggggctccgggcttatattcgtaggcagatcaaccacatcttctacaggtttatctatgagactgagcatcacaatgggattgcggagctgctggagatcctgggcagcatcatcaatggctttgccttgccccttaaggaagaacacaaggtgtttctcgtccgtgtcctgcttccacttcacaaagtcaagtctctgagtgtataccaccctcagttggcgtactgtgtggtgcagttcctggagaaggagagcagtctcactgagccggtgattgtgggacttctcaagttttggcctaagacccacagccccaaggaggtgatgtttctgaacgagctggaggagattctagatgtcattgaaccctcggagttcagtaaagtcatggagccgctcttccgccagcttgccaaatgtgtttccagcccccatttccaggtggcggagcgcgccctctactactggaacaatgagtacatcatgagcctcatcagcgacaatgctgcccgcatcctccccatcatgtttcctgcactctataggaactccaaaagccactggaacaagacaatccatggactcatctacaatgccctgaagctcttcatggagatgaaccagaagctgtttgacgactgcacacagcagtacaaggcggagaagcagaagggcaggttccgaatgaaggagagagaggagatgtggcagaagatcgaggagctggcccggctcaatccccagtaccctatgtttcgggctcctccgccactgccccctgtgtactccatggagacagagacgcccacagcagaggacattcagctcctgaagaggacggtggagacagaggccgtgcagatgctgaaggacatcaagaaggacaaagtgctgctccggaggaagtcagagctgccacaggacgtgtacaccatcaaggcactagaggcgcacaagcgggcagaagagttcctgactgccagccaggaggccctctgaccccctcacccttcccttcccacaggattccagcccactcagccctgggatgcagcccagccctccaccctctgctcctcatcggctgacctggggcagaacggcacctctctggctactctagaggatgcaggcactggaagcgggatgcccagagcgctccctcttctcccctaaacgtgttcatgcctccctgtggctagtacagacaggctgagcactgagtgctcagtgcttagacaacctgggggtgcctgtccccctgctcctaaccccacagctgcctgaaactgttctgagaaatacacacaggaatcacatgtccctctctacccccttaatcctcatctgaagttgaggtgtctctcctctccctgcagagtacaacaggagactgtcaccaaagtcacgttaagctccgttggccccttgagcgaagagctggagggaaccaatccccagcagcacaaataggcccctagacccagctttatgcaggcgggggtggtagtgtgtttctttgtatcctatacctaaccaagacaacacagaggacaaacaagttacggacactagatgactagtgggcccggtggggagaaagaaccatctgccttgccctctctggcaagcagcagtcctgggatcacacatttgcaagggaccaccctgtggctgactgctttcccttgtgctcttggttcccagagctataaagaaggaggcaggagcagtgctccaagcatggctccctgctgtgcctgtttatttcctggtttctgctgacgctgggctgggcgggtcctcacctgtaccctctaggcgctcagaaacaaaagcctggggagggctggaccaggattcaggagcacaggcggtgtggcttctggctgtgtacacagggtgctttattctccacagagtgatacatgctaaggtgggctgggcttgggccgatgtccccatatgtacagaactgaataaagtgggtctctgag

SEQ ID NO: 41 - protein, 467 aa, Homo sapiens
MSSAPTTPPSVDKVDGFSRKSVRKARQKRSQSSSQFRSQGKPIELTPLPLLKDVPSSEQPELFLKKLQQCCVIFDFMDTLSDLKMKEYKRSTLNELVDYITISRGCLTEQTYPEVVRMVSCNIFRTLPPSDSNEFDPEEDEPTLEASWPHLQLVYEFFIRFLESQEFQPSIAKKYIDQKFVLQLLELFDSEDPRERDYLKTVLHRIYGKFLGLRAFIRKQINNIFLRFVYETEHFNGVAELLEILGSIINGFALPLKAEHKQFLVKVLIPLHTVRSLSLFHAQLAYCIVQFLEKDPSLTEPVIRGLMKFWPKTCSQKEVMFLGELEEILDVIEPSQFVKIQEPLFKQIAKCVSSPHFQVAERALYYWNNEYIMSLIEENSNVILPIMFSSLYRISKEHWNPAIVALVYNVLKAFMEMNSTMFDELTATYKSDRQREKKKEKEREELWKKLEDLELKRGLRRDGIIPT

SEQ ID NO: 42 - DNA, 3366 bp, Homo sapiens
ggttgcgcgtgtgccatggactcagccgcccggtgatattgacaataggagagagaaaggggcattgactgggacccaccgcgggtagcgaaaggtggctctggcagcggcggctccagctcctgcggctcctcctccttattctgtccccttctcttgctgccgctgcagatccagtcttcctccctcccttccccccctccccacgtcgtcgccgccgccgccgggtccggggcaacgagctgaggcgccgcccgccaggaatgtgagcgaggagccaccggcggagccgcaacggggtcggtgccgatttgatgggacgggcccgcgggggaggatcgtgaggccgccgccgccaccggaacgctgaggttcgggtccggccgtgaggcctagaggctccgccgccgcggaaccggagggaccccgtaccggacagccgtcgccccaggctccccgcagctgcccggacctccccctgcacgtcccggtcccgccgcccgcccccgctgcggccacctcgcccgtctcccgcccctccaagccacagatcatctttggattcttccccagaagcttcaagtagggatatgtcctcagcaccaactactcctccatcagtggataaagtagacggattttctcggaagtccgtcagaaaagccagacagaagaggtcgcaaagttcctcacagtttaggtctcaaggcaagcctattgagttaacacctctgccgctgctaaaagacgttccatcctcagagcagcctgaactgttcctaaagaaacttcagcagtgctgtgtcatttttgacttcatggacacgctatctgatcttaaaatgaaagaatacaagcgctccactcttaatgaactggtggactacattacaataagcagaggctgtttgacagagcagacttaccctgaagtagttagaatggtatcttgcaatatattcagaactctccctcctagtgacagcaatgaatttgatccagaagaagatgaacctacccttgaggcatcgtggccacacttacagcttgtatatgaatttttcatacgatttttggaaagccaagaattccaacccagcattgccaaaaaatatatagatcagaaatttgtattacagcttctggagctatttgacagcgaagaccctcgggaacgggactacttaaaaacagtcttacacagaatttatggcaagtttcttggtcttagagcatttatccgaaaacagattaacaatatttttctaaggtttgtttatgaaacagaacacttcaatggtgtagctgaactgctggaaatattaggaagtattatcaatggctttgctttacctcttaaggcagaacacaaacagtttctggtgaaagtattgatccctttacacactgtcaggagcttatcactcttccatgcacagctggcatattgtatagtacagtttctggagaaagatccttcactcacagaaccagttattagggggttaatgaaattttggcctaaaacatgtagtcaaaaagaggtcatgttccttggggaactggaagaaatattggatgtgattgaaccttcacaatttgttaaaatccaagaacctttgtttaaacaaatcgccaagtgtgtatctagcccccattttcaggtggcagaaagagcactctattattggaataatgaatacatcatgagtttgatagaagaaaactctaacgtcatccttcccatcatgttttccagcctttataggatttcaaaagaacattggaatccggctattgtggcgttggtgtacaatgtgttgaaggcatttatggaaatgaacagcaccatgtttgacgagctgacagccacatacaagtcagatcgtcagcgtgagaaaaagaaagaaaaggagcgtgaagaattgtggaaaaaattggaggatctggagttaaagagaggtcttagacgtgatggaataattccaacttaacaaaaacaatgacaacaacattactaacctgtggagtcacacgtttatgtagtagaagatggagcaacagttttctgtattgtgcaactttacagtagatttcacctttgtttcattattacagcagcactgtatatacctgtctctaagtaaaggaaaaaacaaaataaggacttcaatccaaagtttggacagtagatggacttctcagaactttgcaaacataatcattgttctcaccctcttttaaaaaaaaaaatcggtcttcaaagatctgttgatgaaattgctatgttaaaattccattatcgggagttccttatttatcactagcagagagtatgatacaattttcaaatgtgaacaatcttaaatttagcttgtctttctgctaagctgttaaatgtatttatagtaaaggaagaaaaaaagactgtcatttccttataagtttgtgtaacatcctcctctggataacttgactgtaatttaacatctttttcttttgcacatcttcctgagttgaatgtccacgtggaatggggtcatgaattataaaagtccctgataaaagttttgtttactggggtgaacatctttccagtaaccaggtagtcctggtactcctttagttttaaaattaggagttaagagagaagaggtgataaacatagtagggaagggaatatcggattcatgcatcagtttatggtgaatccaaatcaatgtcttgaatcctttgaaaacaggcactgggacatcacaggcttcagtacctgaccagtattagttgcatatatcattgaacacacataccagagatgttttagaaatgtgagaaaaacatccttttggaccatttgaaataagaaagacaaacactaaacaatacaaccatgaaattgatcaccgggattgcaaatctaattgggaaaagagttgagcaaacagcttggactgtttggagttgttgccttactttttaatatgtatttataaagtattccagcaaaagaggatgtagcctctgggaaaaaacaaacatgttacagtgttttttgtagattctcgttctatatctcatcacagcgccagccctgtttttagccggaaaggattcaggataaacattattatgcattctgaattggatgcatattcctaactactgtatttgttaccaaaagtggttctacaaatgctactgaaaaaaatctggaaattcctaatgtcctgagtattaataataaagtttaaaaatgcttttatatcaaaggtgcatcgtgaccaaattgtttaagaaaaaaaaacaaaaaaaacaaaatctagggctgtattttatatatatatatatatatatatatatatatat

SEQ ID NO: 43 - protein, 467 aa, Mus musculus
MSSAPTTPPSVDKVDGFSRKSVRKARQKRSQSSSQFRSQGKPIELTPLPLLKDVPTSEQPELFLKKLQQCCVIFDFMDTLSDLKMKEYKRSTLNELVDYITISRGCLTEQTYPEVVRMVSCNIFRTLPPSDSNEFDPEEDEPTLEASWPHLQLVYEFFIRFLESQEFQPSIAKKYIDQKFVLQLLELFDSEDPRERDYLKTVLHRIYGKFLGLRAFIRKQINNIFLRFVYETEHFNGVAELLEILGSIINGFALPLKAEHKQFLVKVLIPLHTVRSLSLFHAQLAYCIVQFLEKDPSLTEPVIRGLMKFWPKTCSQKEVMFLGELEEILDVIEPSQFVKIQEPLFKQIAKCVSSPHFQVAERALYYWNNEYIMSLIEENSNVILPIMFSSLYRISKEHWNPAIVALVYNVLKAFMEMNSTMFDELTATYKSDRQREKKKEKEREELWKKLEDLELKRGLRRDGIIPT

SEQ ID NO: 44 - DNA, 4841 bp, Mus musculus
gtgatattgacaataggagagagaaaggggcattgacggggaccctgcgcgggtagcgaacggcggctctggcagcggcggctcaggcggctccaactcttcctcccgactcccgcgcttgctgccgctgcaaatccaggcttcctccctcctccccctccccgcgtcgccgccgccgccgccgccgccaccaccgccgccgggtccggtgcaacgagcagaggcgccgcccgccgggaatgtgaacgaagagccaccggccgcgccgcaaccgggtcggtgccgatttgatgggacgggcccgcgggggaggctcgtgaggccgccgctgccactgccggagcgctgaggttaggggccagccgagaggcctagaaacactgccgctaccgcggaaccggagggacgtcgccccggacggccgtagtagcccggggctctccgccgctgcctggacctccccccgtgcgtcccgggccgtcgcccgcccccgccgcggcccctagctcgcagcccactccgcgcagccacagatcatcttccgattctttcccaggagcttcaagtagggatatgtcctcagcaccaactactcctccatcagtggataaagtagacggattttctcggaagtccgtcagaaaagccagacagaagaggtcgcaaagctcctcccagttcaggtctcaaggcaagcccattgagctcacgcctctgccactgctgaaagacgttccaacctcagagcagcctgaactgttcctaaagaaacttcagcagtgctgtgtcatttttgacttcatggacacgctatctgatcttaaaatgaaagaatacaagcgctccactcttaatgaactggtggactacattacaataagcagaggctgtctgacagagcagacttaccctgaagtagttagaatggtatcttgcaatatattcagaactctccctcctagtgacagcaatgaattcgacccagaagaagatgaacctaccctcgaggcatcatggccccacttacagcttgtatatgaatttttcataagatttttggaaagccaagaattccaacccagcattgccaaaaaatacatagatcagaaatttgtattacagcttttggagctatttgacagtgaagaccctcgagaacgggactatttaaaaacagtcttgcacaggatttatggcaagtttctcgggcttagagcatttatccgaaaacaaattaacaatatttttctaaggtttgtttatgaaacagaacacttcaatggtgtagctgaactgctggaaatattaggaagtattatcaatggctttgctttaccactcaaggcagaacacaaacagttcctggtgaaagtgttgatccctctacacactgtcaggagcttgtcactctttcatgcacagctggcgtattgtatagtacagtttctggagaaagacccttcccttacagaaccagttattagggggctaatgaaattttggcctaaaacatgcagtcaaaaagaggtcatgttcctcggggagctggaagaaatcttggatgtgattgaaccgtcacaatttgttaaaatccaagaacctttgtttaaacaaattgccaagtgtgtttctagcccccattttcaggtggcagaaagggcactctattattggaataatgaatacatcatgagtttgatagaagaaaactctaacgtcatccttcccatcatgttctccagcctttataggatttcaaaagagcattggaatccggccattgtggcattggtgtacaacgtgttgaaggcatttatggaaatgaacagcactatgtttgatgagctgacagccacatacaagtcagatcgtcagcgtgagaaaaagaaagaaaaggagcgagaagaactgtggaaaaaattggaggatctggagttaaagagaggtcttagacgcgatgggataattccaacttaacaacagcctgacagcgacacactaacccgtgggtcacacgcttatgtagtagaaggatggagcaacagttttctgtattgtgcaactttacagtagatttcacatttgtctcattattacagcagcactgtatatacctgtctctaagtaaaggaaaacataaggacttcaaagtgtggacagtagatggactctcagaactttgcaaacataatcattgttctaaccctctttaaaaacaaacaaacaaacaaaaggtctacagagatctgtcggggaaattgctatgttaaaattccattatcaggagttccttatttatcactagcagagagtatgatacaattttcaaatgtgaacaatcttaaatttagcttgtctttctgctaagctgttaaatgtatttatagtaaaggaagaaaagattgtcatttctttataagtttgtatagcgccctcctctggacatgactgtaacgtgatatcctttccttttgcacatcttcatgagtcaagtgtccacatggaccggggctgtgagtcagaggtctctgaggaaaggttgtctgctggatgaacatcttcccagctgccaggtcctggtactcctttagttttcagattaggagtaaaaagaagaggtgataagcatggtagcgaagggaatgttggacttcatatcagatatggtgagtccaaatcagtgtcttgaatcctttgaaaacaggcagtgggacgcgcaggcttcagcacctgcagcattagctgcagacataagtgtgcatccagaccagagacaacttagaaatgtcagagaacatcctttcggatcctgtggagtaagagagagagcactaagcgatagaaccgtgaagccgagcaccaggaccgcgcatccacttgggaaccgagttgagcaaacagcatgaactgtttgcagttgatgccttacttcctaatgtgtacttacaaagtatttcagcagaagaggatgcagggtgcagcctctgggaggaacatcctccttctctagctcaccacagcgccagccctgtttttagccagaaaggattcaggatcaacattatgcgttctgaattggatgcatattcctaactactgtatttgttaccaaaagtggttctacaaatgctactgaaaaaaatctggaaattcctaatgtcctgagtattaataataaagtttaaaaaatgcttttatatcaaaggtgcatcgtgaccaaattgtttaagttaaaaacaaaacaaaaccaaaccaacccgagggctgtgctctagctgggcttactgtctctgtactgggtccccctgggtaggtaagttgctgatgtacaggtgtgcagtatgtattcacccgatgtgttgcatcgctgtgtattggcaagtgtgtgttgtggggtttgtttccaggaagctttttcttgttagagacaggacagtggtgagcccataaccccgagccatcacatccagcagtgttcatggcatagagccgactctgtgttcatatgttgagttgtgattatagctaacatgaacacacatgagctattgttgctcttagtagaaatacagtataaatatgggcttgtatattttcttccttccttttaaatgtaaagtagaatatatagagctttgttgtttccaaggaaaaggaggggaaaagccactgacctatttcacttatgaagtatttccagattgtcagtcacaatgtgtacataaatgtaagctaattatggatattttcatttaggtacctttgttctcttgttttttaaaggtatgcgtaccaccaagtccaagatggttcagtggaataaataggactcttaaagagcgttaaggagggttccatctgaggacctggaaagtgtgagcagagctgcatgttcacatattgatgctaagaagcaaaccatagcacatctcccttagctgtcttagccgtttccatcttcattacacatatctttgatagtgtagaccttgcttgctgtggtccctctggggatgacatgtgctcatttgacatgaggaaggtcaggacctctttctgcaccgtgtcctgtcctcagagctaagggtcagtgttgtctgttggtcattcccaactgagaggaatgaagctaagctttagactatttaaagatgtgatgagagctgaatgtcagaactcacagggcaatctctgagaagaaacactcatttcccttttaaccaaaagtgacaatgtgacctctgtcatgttgtgtgtggtgaggagaagctagcccaggtgacttgattgttgcactatgtgacatatttaaggacatacttgctgcatttgcagcagcccccctgtctgacctccctgggcaatcctgctgtcactcaatgtgagttaactgtgacaaactcagctctgtgttatattttgtttgcttcatagtttccagaactatatataaatatattttttcaatagcaaatattgtagttagtgagtggtgaccacttcaaccttatccctaccatattggtttcagggatgggactaagaatgtccttggaataaaaaaattaaaatataaccctgtgatttagaatgatgccgaggttgcaggttgcagcctgcagcagctgtgtgagcgaagtgaccgtctcctctggatcaagcgtccactcctcattgtgcccaggccccagtgctccttcatcactgtttagttattgtccgctgtggttttgattatattttgctcttcctgacattgactgtttttttttaataaaagaaaataaaaagtaaatgaataacccctgaaaaaaaaaaaaaaaaaaaaaaaaaaaaaaaaa

SEQ ID NO: 45 - DNA, 22 bp, Artificial Sequence
  /note=“Description of Artificial Sequence Synthetic primer”
agagcgtctatgattagtattg

SEQ ID NO: 46 - DNA, 22 bp, Artificial Sequence
  /note=“Description of Artificial Sequence Synthetic primer”
tactttctgtggaactccggag

SEQ ID NO: 47 - DNA, 22 bp, Artificial Sequence
  /note=“Description of Artificial Sequence Synthetic primer”
agtttctgactgcacatttggc

SEQ ID NO: 48 - DNA, 22 bp, Artificial Sequence
  /note=“Description of Artificial Sequence Synthetic primer”
agtacacagagtgtgataatgc

SEQ ID NO: 49 - DNA, 21 bp, Artificial Sequence
  /note=“Description of Artificial Sequence Synthetic primer”
gcaaactccatcacaaaacag

SEQ ID NO: 50 - DNA, 21 bp, Artificial Sequence
  /note=“Description of Artificial Sequence Synthetic primer”
cacgtagtatgacgcaaattg

SEQ ID NO: 51 - DNA, 21 bp, Artificial Sequence
  /note=“Description of Artificial Sequence Synthetic primer”
tcaggcttcaaatctctggaa

SEQ ID NO: 52 - DNA, 20 bp, Artificial Sequence
  /note=“Description of Artificial Sequence Synthetic primer”
aaacaggcaaatacggcatc

SEQ ID NO: 53 - DNA, 20 bp, Artificial Sequence
  /note=“Description of Artificial Sequence Synthetic primer”
cacgtcatggcagaaagaaa

SEQ ID NO: 54 - DNA, 20 bp, Artificial Sequence
  /note=“Description of Artificial Sequence Synthetic primer”
ctggcagtcactaacacacg

SEQ ID NO: 55 - DNA, 20 bp, Artificial Sequence
  /note=“Description of Artificial Sequence Synthetic primer”
accctcatgcctactgtcag

SEQ ID NO: 56 - DNA, 35 bp, Artificial Sequence
  /note=“Description of Artificial Sequence Synthetic primer”
agctcgggtaccgatggtctcaaagggtgaagaag

SEQ ID NO: 57 - DNA, 102 bp, Artificial Sequence
  /note=“Description of Artificial Sequence Synthetic primer”
tacgaatgaattcttaatcatcgtccttatagtccattccgctagctccaccctgaaaatacaaattctctccagctcccttatacaattcatccatgccac

SEQ ID NO: 58 - DNA, 25 bp, Artificial Sequence
  /note=“Description of Artificial Sequence Synthetic primer”
caactttgtatagaaaagttgaacg

SEQ ID NO: 59 - DNA, 38 bp, Artificial Sequence
  /note=“Description of Artificial Sequence Synthetic primer”
ataatggtacctcaactttgtacaagaaagttgaacga

SEQ ID NO: 60 - DNA, 20 bp, Artificial Sequence
  /note=“Description of Artificial Sequence Synthetic primer”
tgttttgtgccaagcttcag

SEQ ID NO: 61 - DNA, 20 bp, Artificial Sequence
  /note=“Description of Artificial Sequence Synthetic primer”
agttccgtgtgctcttgctc

SEQ ID NO: 62 - DNA, 20 bp, Artificial Sequence
  /note=“Description of Artificial Sequence Synthetic primer”
ggacgacccccattaatttt

SEQ ID NO: 63 - DNA, 36 bp, Artificial Sequence
  /note=“Description of Artificial Sequence Synthetic primer”
atctgaaagctttgaaaaatcatttattgggatttg

SEQ ID NO: 64 - DNA, 17 bp, Artificial Sequence
  /note=“Description of Artificial Sequence Synthetic primer”
ggagttctcgccgtccg

SEQ ID NO: 65 - DNA, 21 bp, Artificial Sequence
  /note=“Description of Artificial Sequence Synthetic primer”
gtcgaatgggagatctgggag

SEQ ID NO: 66 - DNA, 23 bp, Artificial Sequence
  /note=“Description of Artificial Sequence Synthetic primer”
agtgtgactgcaaaaacaagcaa

SEQ ID NO: 67 - DNA, 23 bp, Artificial Sequence
  /note=“Description of Artificial Sequence Synthetic primer”
tccactgcattcacatttgtctc

SEQ ID NO: 68 - DNA, 20 bp, Artificial Sequence
  /note=“Description of Artificial Sequence Synthetic primer”
gggattggccacttcaaaag

SEQ ID NO: 69 - DNA, 20 bp, Artificial Sequence
  /note=“Description of Artificial Sequence Synthetic primer”
cgtcgtcgaggacattgaca

SEQ ID NO: 70 - DNA, 21 bp, Artificial Sequence
  /note=“Description of Artificial Sequence Synthetic primer”
aagagatcgttcactcgccag

SEQ ID NO: 71 - DNA, 20 bp, Artificial Sequence
  /note=“Description of Artificial Sequence Synthetic primer”
agccaagtcgacgtcctttg

SEQ ID NO: 72 - DNA, 20 bp, Artificial Sequence
  /note=“Description of Artificial Sequence Synthetic primer”
ctcttgccccatcaaccatg

SEQ ID NO: 73 - DNA, 20 bp, Artificial Sequence
  /note=“Description of Artificial Sequence Synthetic primer”
cttgcttggagatccacatc

SEQ ID NO: 74 - DNA, 21 bp, Artificial Sequence
  /note=“Description of Artificial Sequence Synthetic primer”
atggacgagaagttgttcacc

SEQ ID NO: 75 - DNA, 21 bp, Artificial Sequence
  /note=“Description of Artificial Sequence Synthetic primer”
cagtgactggacatcgaacct

SEQ ID NO: 76 - DNA, 22 bp, Artificial Sequence
  /note=“Description of Artificial Sequence Synthetic primer”
atgagtgcctacggaagtatgg

SEQ ID NO: 77 - DNA, 21 bp, Artificial Sequence
  /note=“Description of Artificial Sequence Synthetic primer”
cagggctcttatgtggtccag

SEQ ID NO: 78 - DNA, 23 bp, Artificial Sequence
  /note=“Description of Artificial Sequence Synthetic primer”
attgaagagccgctttttaagca

SEQ ID NO: 79 - DNA, 21 bp, Artificial Sequence
  /note=“Description of Artificial Sequence Synthetic primer”
tgagggttttcagcacattgt

SEQ ID NO: 80 - DNA, 19 bp, Artificial Sequence
  /note=“Description of Artificial Sequence Synthetic primer”
gggcctacatccgcaaaca

SEQ ID NO: 81 - DNA, 20 bp, Artificial Sequence
  /note=“Description of Artificial Sequence Synthetic primer”
ggatcaggactcgaaccagg

SEQ ID NO: 82 - DNA, 22 bp, Artificial Sequence
  /note=“Description of Artificial Sequence Synthetic primer”
acgtctcaagcaatcactggag

SEQ ID NO: 83 - DNA, 20 bp, Artificial Sequence
  /note=“Description of Artificial Sequence Synthetic primer”
tttcgtcgcttctatgtgcg

SEQ ID NO: 84 - DNA, 29 bp, Artificial Sequence
  /note=“Description of Artificial Sequence Synthetic primer”
gatatcactcttttagaggagcttgtacg

SEQ ID NO: 85 - DNA, 22 bp, Artificial Sequence
  /note=“Description of Artificial Sequence Synthetic primer”
ctcctcagcgtgaatgactgtt

SEQ ID NO: 86 - DNA, 20 bp, Artificial Sequence
  /note=“Description of Artificial Sequence Synthetic primer”
ccattgcacaaaccgaaatg

SEQ ID NO: 87 - DNA, 22 bp, Artificial Sequence
  /note=“Description of Artificial Sequence Synthetic primer”
gtaggcgagctgtgcatgatac

SEQ ID NO: 88 - DNA, 20 bp, Artificial Sequence
  /note=“Description of Artificial Sequence Synthetic primer”
caaacacgtacggaggaggg

SEQ ID NO: 89 - DNA, 17 bp, Artificial Sequence
  /note=“Description of Artificial Sequence Synthetic primer”
attggcggcgctctctt

SEQ ID NO: 90 - DNA, 20 bp, Artificial Sequence
  /note=“Description of Artificial Sequence Synthetic primer”
caacgggagacaaaggagga

SEQ ID NO: 91 - DNA, 26 bp, Artificial Sequence
  /note=“Description of Artificial Sequence Synthetic primer”
cactctgatctcgttggaaaattaca

SEQ ID NO: 92 - protein, 11 aa, Artificial Sequence
  /note=“Description of Artificial Sequence Synthetic peptide”
TSTFCGTPEYK

SEQ ID NO: 93 - protein, 16 aa, Artificial Sequence
  /note=“Description of Artificial Sequence Synthetic peptide”
CSYGDKTSTFSGTPEY

SEQ ID NO: 94 - protein, 16 aa, Artificial Sequence
  /note=“Description of Artificial Sequence Synthetic peptide”
CSYGDKTSTFCGTPEY

SEQ ID NO: 95 - protein, 15 aa, Artificial Sequence
  /note=“Description of Artificial Sequence Synthetic peptide”
CSNFTQFSFHNVMGS

SEQ ID NO: 96 - protein, 15 aa, Artificial Sequence
  /note=“Description of Artificial Sequence Synthetic peptide”
CSNFTQFSFHNVMGS

(19) United States
(12) Patent Application Publication
(10) Pub. No.: US 2011/0286930 A1
(43) Pub. Date: Nov. 24, 2011

(54) METHODS OF IDENTIFYING MODULATORS OF THE PP2A B56 BETA REGULATORY SUBUNIT

(75) Inventors: Heidi A. Tissenbaum, Wayland, MA (US); Srivatsan Padmanabhan, Shrewsbury, MA (US); Arnab Mukhopadhyay, New Delhi (IN); Sri Devi Narasimhan, Allston, MA (US)

(73) Assignee: UNIVERSITY OF MASSACHUSETTS, Boston, MA (US)

(21) Appl. No.: 13/120,613

(22) PCT Filed: Sep. 24, 2009
(86) PCT No.: PCT/US09/58205

§ 371 Date: Jul. 11, 2011

Related U.S. Application Data
(60) Provisional application No. 61/194,146, filed on Sep. 24, 2008.
(60) Provisional application No. 61/206,914, filed on Feb. 5, 2009.
(60) Provisional application No. 61/208,888, filed on Feb. 26, 2009.

(51) Int. Cl. A61K 49/00; C12Q 1/02; A61P 3/04; A61P 35/00; A61K 31/713; A61P 3/10

(52) U.S. Cl. 424/9.2; 514/44 A; 435/6.13

(57) ABSTRACT

The present invention is based at least in part on the discovery that the PP2A B56 regulatory subunit plays a role in regulating insulin signaling by directly regulating phosphorylation of AKT-I. Accordingly, the present invention features methods of identifying modulators of the PP2A B56 regulatory subunit in assays featuring organisms and/or cells. Further featured are therapeutic methods for the use of PP2A B56 regulatory subunit modulators to enhance longevity, to prevent or treat cancer, to prevent or reduce obesity and to prevent or treat type II diabetes.

### CROSS REFERENCE TO RELATED APPLICATIONS

This application is related and claims priority to U.S. provisional application Ser. No. 61/194,146 filed Sep. 24, 2008; U.S. provisional application Ser. No. 61/206,914 filed Feb. 5, 2009; and U.S. provisional application Ser. No. 61/208,888 filed Feb. 26, 2009. The entire contents of each of the foregoing applications are incorporated herein by this reference.

### GOVERNMENT RIGHTS

This invention was made at least in part with government support under grant no. AG025891 awarded by the National Institute on Aging. The government may have certain rights in this invention.

### BACKGROUND OF THE INVENTION

The insulin/IGF-1-like signaling (IIS) pathway is an evolutionarily conserved neuro-endocrine pathway that regulates multiple biological processes including metabolism, development, stress resistance and lifespan (Antebi, 2007; Barbieri et al., 2003; Kenyon, 2005; Wolff and Dillin, 2006). In Caenorhabditis elegans (C. elegans), the insulin-like receptor DAF-2 (Kimura et al., 1997) signals through a PI 3-kinase (AGE-1/AAP-1) (Morris et al., 1996; Wolkow et al., 2002) signaling cascade that activates the downstream serine/threonine kinases PDK-1, AKT-1, AKT-2 and SGK-1 (Hertweck et al., 2004; Paradis et al., 1999; Paradis and Ruvkun, 1998). These kinases in turn function to negatively regulate the forkhead transcription factor (FOXO), DAF-16 (Lin et al., 1997; Ogg et al., 1997).

Reduction-of-function mutations in serine/threonine kinases upstream of DAF-16 lead to changes in lifespan, development, metabolism and/or stress resistance (Antebi, 2007; Kenyon, 2005; Wolff and Dillin, 2006). Importantly, loss-of-function mutations in daf-16 completely suppress these phenotypes (Antebi, 2007; Kenyon, 2005; Mukhopadhyay et al., 2006; Wolff and Dillin, 2006). Thus DAF-16 is a major downstream target of the IIS pathway. Regulation of DAF-16 by AKT-1, AKT-2 and SGK-1 results in its nuclear exclusion and sequestration in the cytosol (Lin et al., 2001) (Hertweck et al., 2004; Lee et al., 2001). In contrast, under low signaling conditions, active DAF-16 enters the nucleus and transactivates or represses its direct target genes (Henderson and Johnson, 2001; Hertweck et al., 2004; Lee et al., 2001; Lin et al., 2001; Oh et al., 2006). Strikingly, this negative regulation of FOXO/DAF-16 is conserved across species. In mammals, the Akt and SGK kinases can phosphorylate and negatively regulate FOXO (Brunet et al., 1999; Brunet et al., 2001; Calnan and Brunet, 2008).

Although regulation of the IIS pathway by serine/threonine protein kinases has been extensively studied, little is known about the phosphatases acting in this pathway. In C. elegans, the lipid phosphatase DAF-18 (homologous to mammalian Phosphatase and Tensin Homolog, PTEN) is the only phosphatase that has been identified and characterized as a negative regulator of the IIS pathway (Gil et al., 1999; Mihaylova et al., 1999; Ogg and Ruvkun, 1998; Rouault et al., 1999). The increased lifespan of daf-2 mutant worms is suppressed by loss-of-function mutation in daf-18 (Dorman et al., 1995; Larsen et al., 1995). There exists, therefore, a clear need in the art for the identification of additional regulators of the IIS pathway and, in particular, phosphatases that regulate the IIS pathway.

### SUMMARY OF THE INVENTION

The present invention is based at least in part on the discovery that the PP2A B56 regulatory subunit (e.g., PPTR-1 or B56β) plays a role in regulating insulin signaling by directly regulating phosphorylation of AKT-1. Accordingly, the present invention features methods of identifying modulators of the PP2A B56 regulatory subunit in assays featuring organisms and/or cells Further featured are therapeutic methods for the use of PP2A B56 regulatory subunit modulators to enhance longevity, to prevent or treat cancer, to prevent or reduce obesity and to prevent or treat type II diabetes.

Other features and advantages of the invention will be apparent from the following detailed description and claims.

### BRIEF DESCRIPTION OF THE DRAWINGS

FIG. 1: pptr-1, a regulatory subunit of the PP2A holoenzyme, was identified as a top candidate in a directed RNAi screen to identify serine/threonine phosphatases that regulate the IIS pathway. (A) depicts the different families and classes of the phosphatases included in the RNAi screen; (B) is a schematic representation of the RNAi screen. All the assays were performed in triplicate; (C) depicts the top two candidates that dramatically suppressed daf-2(e1370) dauer formation at 20° C. (fem-2, and pptr-1). Both fem-2 and pptr-1 RNAi were able to suppress daf-2 dauer formation to a similar level as daf-18 RNAi. Error bars indicate the standard deviations (Std. Dev.) among the different RNAi plates within one experiment. Data shown [% Dauers±Std. Dev. (n)] are from one representative experiment; (D) is a graph showing that pptr-1 is the only PP2A regulatory subunit family member that dramatically suppresses daf-2(1370) dauer formation. Error bars indicate the standard deviations among the different RNAi plates within one experiment. Data shown are from one representative experiment.

FIG. 2: pptr-1 regulates lifespan, thermotolerance and fat-storage through the IIS pathway. Data shown are from one representative experiment. (A) pptr-1 RNAi significantly reduces the lifespan of daf-2(e1370) mutants similar to daf-18 RNAi. Mean lifespan [Days±Std. Dev. (n)] of daf-2(e1370) is as follows: on vector RNAi is 33.9±0.7 days (n=77), on pptr-1 RNAi is 27.7±0.9 days (n=63) p<0.0001 and on daf-18 RNAi is 20.4±0.6 days (n=40), p<0.0001; (B) pptr-1 RNAi does not affect the lifespan of wild-type worms. Mean lifespan [Days±Std. Dev. (n)] of wild type is as follows: on vector RNAi is 22.8±0.4 days (n=61), on pptr-1 RNAi is 21.9±0.5 days (n=49) daf-18 RNAi reduces mean lifespan to 18.6±0.3 days (n=48), p<0.0001; (C) The thermotolerance of daf-2(e1370) worms is reduced by pptr-1 and daf-18 RNAi (mean survival [Hours±Std. Dev. (n)] at 37° C. on vector RNAi was 15.2±0.7 hrs (n=34), 13.8±0.5 hrs (p value<0.006) (n=36) on pptr-1 RNAi, and 10.3±0.7 hrs (p value<0.0001) (n=29) on daf-18 RNAi. pptr-1 RNAi did not affect the thermotolerance of wild type worms; (mean survival was 9.8±0.4 hrs on vector RNAi (n=32), 9.3±0.3 hrs on pptr-1 RNAi (n=35) and 9.7±0.4 hrs on daf-18 RNAi (n=32); (D) Sudan black staining showing that pptr-1 RNAi reduces the increased fat storage of daf-2(e1370) worms, similar to daf-18 RNAi but has no effect on wild type fat-storage. Arrows indicate the pharynx. A representative picture from one of three independent experiments (n=30) is shown.

FIG. 3: PPTR-1 co-localizes with AKT-1. akt-1::gfp;pptr-1::mC-flag, akt-2::gfp;pptr-1::mC-flag and sgk-1::gfp;pptr-1::mC-flag transgenic worms were mounted and visualized by fluorescence microscopy using Rhodamine (mCherry) and FITC (GFP) filters. PPTR-1 expression is observed mainly in the pharynx, vulva and spermatheca (A-C, mCherry). (A) PPTR-1::mC-FLAG (mCherry) and AKT-1::GFP (GFP) colocalize in a akt-1::gfp; pptr-1::mC-flag strain (Merge); (B) PPTR-1::mC-FLAG and AKT-2::GFP colocalize in some tissues in a akt-2::gfp; pptr-1::mC-flag strain (Merge); (C) SGK-1::GFP and PPTR-1::mC-FLAG do not colocalize in sgk-1::gfp;pptr-1::mC-flag transgenic worms (Merge).

Arrows indicate the following tissues: p-pharynx, v-vulva, s-spermatheca, i-intestine

FIG. 4: PPTR-1 interacts with and modulates AKT-1 phosphorylation. Data shown are from one representative experiment. (A) PPTR-1 directly interacts with AKT-1 in C. elegans. AKT-1::GFP and MYO-3::GFP were immunoprecipated (IP) using anti-GFP antibody and were analyzed by western blotting (WB) using anti-Ds-Red or anti-GFP antibodies. In addition, PPTR-1::mC-FLAG was immunoprecipitated with anti-FLAG antibody and analysed by WB using using anti-Ds-Red or anti-GFP antibodies. Lysates were used for WB analysis; (B) PPTR-1 overexpression reduces AKT-1 phosphorylation in C. elegans. AKT-1::GFP and MYO-3::GFP were immunoprecipitated from akt-1::gfp, akt-1::gfp;pptr-1::mC-flag and myo-3::gfp;pptr-1::mC-flag followed by western blotting using pThr 350 or pSer 517 antibodies (upper panels). Total lysates were analyzed by western blotting (lower panels). Quantification of fold changes in AKT-1::GFP phosphorylation upon PPTR-1 overexpression is shown below each lane. (C) Knockdown of the mammalian B56β regulatory subunit by siRNA in 3T3-L1 adipocytes increases insulin-stimulated AKT phosphorylation at Thr 308. 3T3-L1 adipocytes transfected with scrambled (Scr), PP2Acα/β, B56α, B56β or B56α/β siRNA were treated with increasing concentrations of insulin and Akt phosphorylation was analyzed by western blotting using pThr 308 (left) and pSer 473 antibodies (middle). Total Akt antibody was used as a loading control (right). Quantification of fold changes in Akt phosphorylation is shown below each lane.

FIG. 5: PPTR-1 regulates DAF-16 localization and activity. Data shown are from one representative experiment. (A) Over-expression of PPTR-1 promotes DAF-16 nuclear translocation. On vector RNAi, DAF-16 is more enriched in the nucleus in a pptr-1::mC-flag;daf-16::gfp strain, compared to a daf-16::gfp strain. This effect is specific to the functional transgene, as knocking down pptr-1::mC-flag with mCherry RNAi decreases the extent of nuclear DAF-16. (B) Overexpression of PPTR-1 significantly increases the lifespan of wild type worms. Mean lifespan [Days±Std. Dev. (n)] of wild type worms is 23.9±0.3 days (n=154), pptr-1::mC-flag is 30.1±0.5 days (n=202), p<0.0001, and the unc-119(+); unc-119(ed3) control strain is 22.6±0.3 days (n=145). (C) In a daf-2(e1370);daf-16::gfp strain, DAF-16 is enriched in the nucleus on vector RNAi, whereas on pptr-1 RNAi as well as daf-18 RNAi, DAF-16 is mostly cytosolic. (D) pptr-1 RNAi affects transcriptional activity of sod-3, a direct target of DAF-16. pptr-1 RNAi reduces Psod-3::GFP expression in a daf-2(e1370);Psod-3::gfp(muIs84) strain, similar to daf-18 RNAi. (E) Transcript abundance of known DAF-16 target genes decrease when daf-2(e1370) worms are grown on pptr-1 RNAi similar to daf-18 RNAi, as detected by real-time PCR. (F) Proposed model illustrating the role of PPTR-1 in the insulin/IGF-1 signaling pathway. Signals from DAF-2 are processed by PI3-kinase leading to the phosphorylation and activation of the downstream serine/threonine kinases PDK-1, AKT-1, AKT-2 and SGK-1. PPTR-1, a PP2A holoenzyme regulatory subunit, regulates the dephosphorylation and activation status of AKT-1 at T350. This in turn affects the nuclear translocation of DAF-16 and the expression of genes involved in lifespan, dauer formation.

FIG. 6: PPTR-1 directly interacts with AKT-1 in C. elegans but not with DAF-16, AKT-2 or GFP (control). A biochemical interaction between PPTR-1 and SGK-1 was observed in our co-IP experiments, but neither a genetic interaction nor any tissue overlap was observed between these proteins. PPTR-1::mC-FLAG was immunoprecipated (IP) from akt-1::gfp;pptr-1::mC-flag, akt-2::gfp;pptr-1::mC-flag, sgk-1::gfp;pptr-1::mC-flag, daf-16::gfp;pptr-1::mC-flag and myo-3::gfp;pptr-1::mC-flag using anti-FLAG antibody and interactions with AKT-1::GFP, AKT-2::GFP, SGK-1::GFP, DAF-16::GFP or MYO-3::GFP (control) and were analyzed by western blotting (WB) using anti-GFP or anti-Ds-Red antibodies.

FIG. 7: A) Determination of the specificity of the C. elegans phospho-AKT antibodies. L2/L3 (Lanes 1, 3 and 5) or mixed stage (Lanes 2, 4 and 6) akt-1::gfp worms were lysed and AKT-1::GFP was immunoprecipitated using anti-GFP antibody (Lanes 3-6). The immunoprecipitated proteins were resolved by SDS-PAGE and blotted to PVDF membrane. Lanes 5 and 6 of the membrane were cut out and treated with Lambda phosphatase. The treated as well as the untreated blots were probed with anti-phospho Thr 350 or Ser 517 antibodies. (B) Determination of specificity of siRNA knockdown. The 3T3-L1 adipocytes were transfected with scrambled (Scr), PP2Acα/β, B56α, B56β or B56α/β siRNA (indicated at the bottom of each graph). Total RNA was isolated from the adipocytes and quantitative RT-PCR analysis was performed to determine the efficiency of knock down of each gene (indicated at the top of each graph).

FIG. 8: Summary of growth experimental results.

FIG. 9: Schematic summarizing conservation of Insulin/IGF-1 signaling pathway.

FIG. 10: Schematic summarizing PP2A phosphatase subunits.

### DETAILED DESCRIPTION OF THE INVENTION

The present invention is based, at least in part, on the discovery of a central role for the PP2A B56 regulatory subunit (e.g., PPTR-1 or B56β) in regulating insulin signaling. In particular, the invention is based on the discovery that the PP2A B56 regulatory subunit (e.g., PPTR-1 or B56β) directly and negatively regulates phosphorylation of C. elegans AKT-1 at Thr350. The invention is based on the further discovery that mammalian PPTR-1/B56 regulates Akt phosphorylation at Thr308, thus highlighting the remarkable conservation of this interaction. In C. elegans, this modulation ultimately results in the increased activity of the forkhead transcription factor DAF-16 and upregulation of genes required for increased longevity and stress resistance. The invention is based on the further discovery that overexpression of the PP2A B56 regulatory subunit in C. elegans extends lifespan.

So that the invention may be more readily understood, certain terms are first defined.

“Longevity” and “life-extension”, used interchangeably herein, also include delay and/or stabilizing the aging process. Preferably, the longevity is due to an extension of the mature life phase, as opposed to an extension of the immature life phase (i.e., delay in maturity).

A “function” of a polynucleotide can be on any level, including DNA binding, transcription, translation, processing and/or secretion of expression product, interaction (such as binding) of expression product with another moiety, and regulation (whether repression or de-repression) of other genes. It is understood that a life-extension polynucleotide or polypeptide includes fragments, or regions, of a polynucleotide or polypeptide, as long as the requisite life-extension phenotype is observed.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

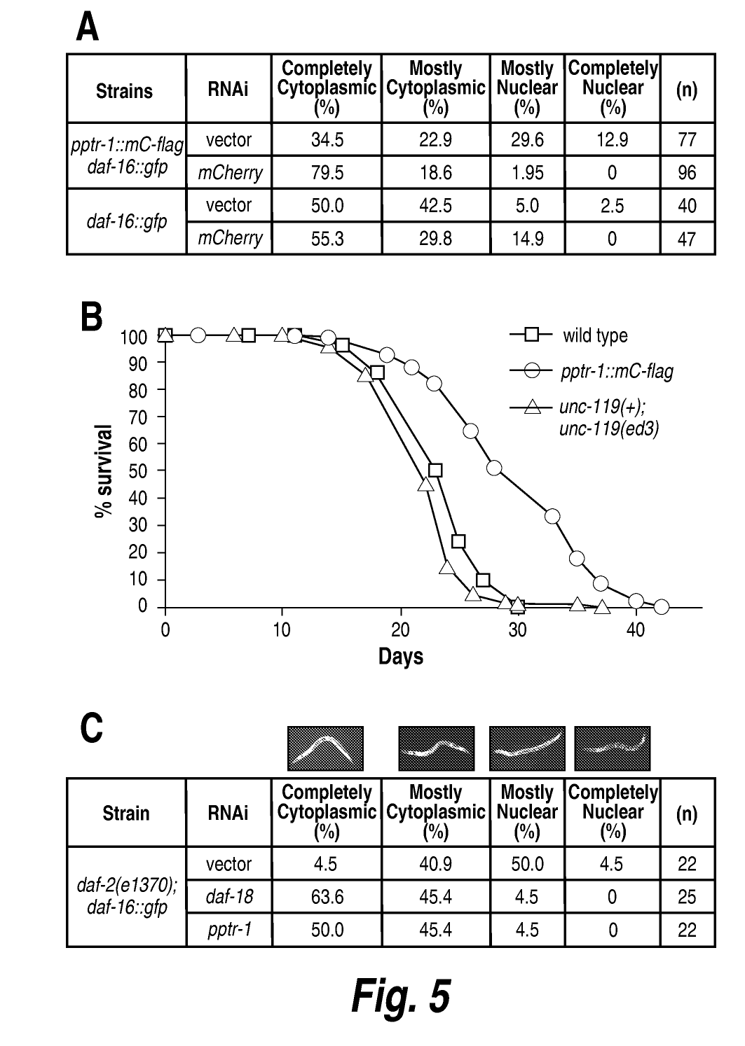

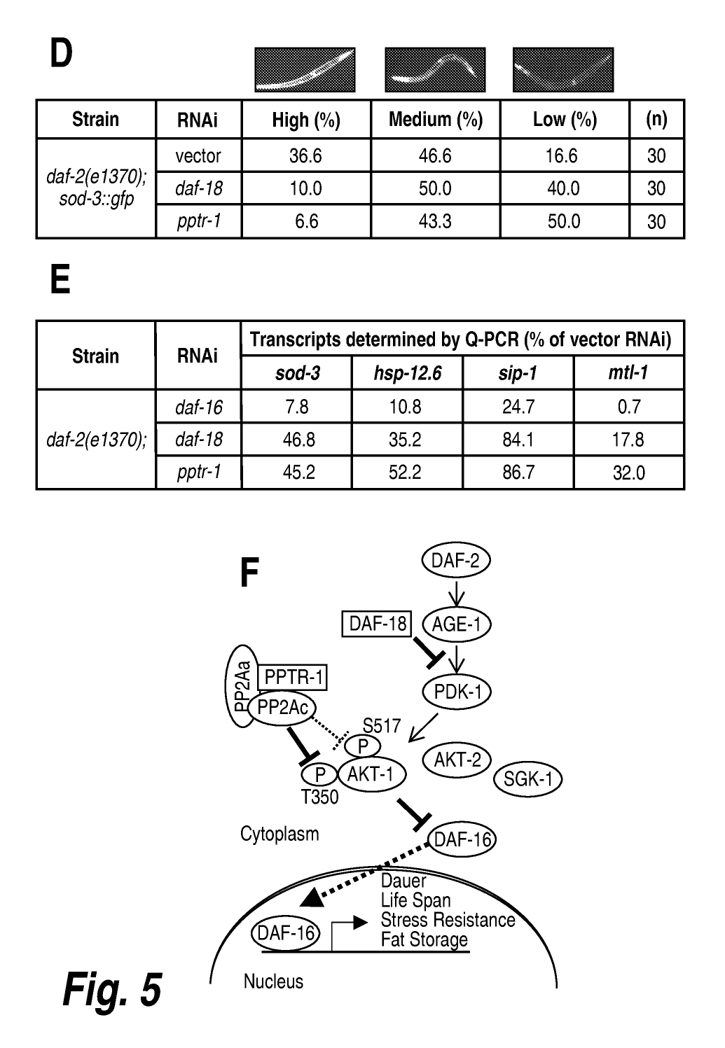

The term “insulin signaling pathway” (or “insulin-like signaling pathway”) refers to the signaling pathway involving proteins (e.g., enzymes) and other non-protein molecules (e.g., precursors, substrates, intermediates or products), utilized in transmission of an intracellular signal from a cell membrane to the nucleus, in particular, from an insulin receptor (IR) or insulin-like growth factor (IGF) receptor at the cell surface to the nucleus. Additional signaling molecules in the insulin signaling pathway in mammals, for example, include insulin receptor substrate (IRS), phosphatidylinositol 3-kinase (PI3-K), PTEN phosphatase, phosphoinositide kinase 1 (PDK1), protein kinase B (PKB) and forkhead transcription factors (FKHR). Such signaling molecules in C. elegans, for example, include IST-1, DAF-2, AAP-1, AGE-1, PDK-1, AKT-1, DAF-18 and DAF-16 (and the corresponding genes encoding these molecules, i.e., ist-1, daf-2, aap-1, age-1, pdk-1, akt-1, akt-2, daf-18, and daf-16, respectively. FIG. 5F includes a schematic representation of the insulin signaling pathway.

As used herein, the term “PP2A B56 regulatory subunit” includes PP2A B56 regulatory subunit (wherein the PP2A B56 regulatory subunit family is also referred to herein and in the art as the PR61 or B′ family) nucleic acid molecules, or biologically active fragments thereof, that share structural features with the nucleic acid molecules corresponding to known genes of the PP2A B56 family (e.g., B56α, B56β, B56γ, B56δ or B56ε in mammals) and PP2A B56 regulatory subunit proteins that share the distinguishing structural and functional features of the PP2A B56 regulatory subunit proteins, or biologically active fragments thereof, encoded by PP2A B56 regulatory subunit genes (e.g., B56α, B56β, B56γ, B56δ or B56ε in mammals). In a preferred embodiment, a “PP2A B56 regulatory subunit” of the invention is a PP2A B56β (or PPTR-1 in C. elegans) nucleic acid molecule, or biologically active fragment thereof, or a PP2A B56β (or PPTR-1 in C. elegans) protein, or biologically active fragment thereof.

As used herein, the term “PP2A B56 regulatory subunit gene” refers to the coding sequence of a PP2A B56 regulatory subunit, e.g., B56α, B56β, B56γ, B56δ or B56ε, found in genomic DNA, as well as the intronic sequences and 5′ and 3′ untranslated/regulatory regions of said PP2A B56 regulatory subunit gene. In a preferred embodiment, the term “PP2A B56 regulatory subunit gene” refers to the coding sequence of the B56 regulatory subunit B56β or PPTR-1 found in genomic DNA, as well as the intronic sequences and 5′ and 3′ untranslated/regulatory regions of the gene. For example, in one embodiment, a PP2A B56 regulatory subunit gene includes, for example, about 5 kb, about 4 kb, about 3 kb, about 2 kb, about 1 kb of genomic DNA upstream of the PP2A B56 regulatory subunit ATG initiation codon or downstream of the PP2A B56 regulatory subunit termination codon.

As used herein “selectively increasing PP2A B56 regulatory subunit activity” refers to directly enhancing or increasing the activity of a PP2A B56 regulatory subunit, e.g., B56β or PPTR-1 (e.g., thereby enhancing or increasing the activity of a PP2A holoenzyme comprising the PP2A B56 regulatory subunit, e.g., B56β or PPTR-1) using a “stimulatory agent”. In one embodiment, PP2A B56 regulatory subunit activity, e.g., B56β or PPTR-1 activity, is selectively increased in adipocyte cells.

As used herein, the term “a stimulatory agent” (“an agent that selectively increases PP2A B56 regulatory subunit activity”) includes agents that enhance PP2A B56 regulatory subunit, e.g., B56β or PPTR-1, expression, processing, post-translational modification, and/or activity. The term includes agents, for example a compound or compounds which increase transcription of a PP2A B56 regulatory subunit (e.g., B56β or PPTR-1) gene, processing of a PP2A B56 (e.g., B56β or PPTR-1) regulatory subunit mRNA, translation of PP2A B56 regulatory subunit (e.g., B56β or PPTR-1) mRNA, post-translational modification of a PP2A B56 regulatory subunit (e.g., B56β or PPTR-1) protein (e.g., glycosylation, ubiquitinization or phosphorylation) or activity of a PP2A B56 regulatory subunit (e.g., B56β or PPTR-1) protein. Examples of agents that directly increase PP2A B56 regulatory subunit (e.g., B56β or PPTR-1) activity include e.g., nucleic acid molecules that encode PP2A B56 regulatory subunit (e.g., B56β or PPTR-1), or biologically active portions thereof, PP2A B56 regulatory subunit (e.g., B56β or PPTR-1) polypeptides, or biologically active portions thereof, expression vectors encoding PP2A B56 regulatory subunit (e.g., B56β or PPTR-1) that allow for increased expression of PP2A B56 regulatory subunit (e.g., B56β or PPTR-1) activity in a cell, chemical compounds, or small molecules, that act to specifically enhance the activity of PP2A B56 regulatory subunit (e.g., B56β or PPTR-1).

As used herein “selectively decreasing PP2A B56 regulatory subunit activity” refers to directly inhibiting or decreasing the activity of a PP2A B56 regulatory subunit, e.g., B56β or PPTR-1 (e.g., thereby inhibiting or decreasing the activity of a PP2A holoenzyme comprising the PP2A B56 regulatory subunit, e.g., B56β or PPTR-1) using an “inhibitory agent”. In one embodiment, PP2A B56 regulatory subunit activity, e.g., B56β or PPTR-1 activity, is selectively decreased in adipocyte cells.

As used herein, the term “an inhibitory agent” (“an agent that selectively decreases PP2A B56 regulatory subunit activity”) includes agents that decrease, diminish or inhibit PP2A B56 regulatory subunit, e.g., B56β or PPTR-1, expression, processing, post-translational modification, and/or activity. The term includes agents, for example a compound or compounds which decrease transcription of a PP2A B56 regulatory subunit (e.g., B56β or PPTR-1) gene, processing of a PP2A B56 (e.g., B56β or PPTR-1) regulatory subunit mRNA, translation of PP2A B56 regulatory subunit (e.g., B56β or PPTR-1) mRNA, post-translational modification of a PP2A B56 regulatory subunit (e.g., B56β or PPTR-1) protein (e.g., glycosylation, ubiquitinization or phosphorylation) or activity of a PP2A B56 regulatory subunit (e.g., B56β or PPTR-1) protein. Examples of agents that directly decrease PP2A B56 regulatory subunit (e.g., B56β or PPTR-1) activity include e.g., antisense oligonucleotides or siRNAs directed to the PP2A B56 regulatory subunit (e.g., B56β or PPTR-1), and chemical compounds, or small molecules, that act to specifically inhibit or decrease the activity of a PP2A B56 regulatory subunit (e.g., B56β or PPTR-1).

As used herein, a “transgenic animal” refers to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, a “homologous recombinant animal” refers to a type of transgenic non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

The term “deregulated” or “deregulation” includes the alteration or modification of at least one molecule in a signaling pathway, such that signal transmission by the pathway is altered or modified. Preferably, the activity or expression of at least one enzyme in the pathway is altered or modified such that signal transmission by the pathway is altered or modified.

The term “upmodulated” refers to an increase or enhancement of the activity or expression of a molecule. The term “downmodulated” refers to a decrease or inhibition of the activity or expression of a molecule.

The term “diabetes” or “diabetic disorder” or “diabetes mellitus,” as used interchangeably herein, refers to a disease which is marked by elevated levels of sugar (glucose) in the blood. Diabetes can be caused by too little insulin (a chemical produced by the pancreas to regulate blood sugar), resistance to insulin, or both.

The term “type II diabetes” refers to a chronic, life-long disease that results when the body's insulin does not work effectively. A main component of type 2 diabetes is “insulin resistance,” wherein the insulin produced by the pancreas cannot connect with fat and muscle cells to allow glucose inside to produce energy, causing hyperglycemia (high blood glucose). To compensate, the pancreas produces more insulin, and cells, sensing this flood of insulin, become even more resistant, resulting in a vicious cycle of high glucose levels and often high insulin levels.

The phrase “disorders associated with diabetes” or “diabetes associated disorders”or “diabetes related disorders,” as used herein, refers to conditions and other diseases which are commonly associated with or related to diabetes. Example of disorders associated with diabetes include, for example, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, and osteoporosis.

As used herein, the term “agent” means a biological or chemical compound such as a simple or complex organic or inorganic molecule, a peptide, protein, oligonucleotide, polynucleotide, carbohydrate, or lipoprotein. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. Compounds can be tested singly or in combination with one another.

As used herein, “expression” refers to the process by which polynucleotides are transcribed into mRNA and translated into peptides, polypeptides, or proteins.

An agent that “modulates” life-extension is an agent that affects life-extension, or lifespan, whether directly or indirectly, whether negatively or positively.

Various aspects of the invention are described in further detail in the following subsections.

I. Insulin-Like Signaling Pathway

The insulin-like signaling pathway in C. elegans regulates lifespan, fat storage, stress resistance and dauer formation. Interestingly, several of the genes in this pathway were first isolated based on their effects on development. The normal lifecycle of C. elegans follows development from an egg, through four larval stages, and a final molt into a fertile, adult hermaphrodite. When nutrition is low or population density is high, the worms can undergo an alternative developmental program to form “dauer” larvae (Cassada R. C. & Russell R. (1975) Dev. Biology 46:326-342). The dauer larvae is a diapause stage that does not feed or reproduce, is stress resistant and is apparently non-aging, wherein worms can remain as dauer larvae for months (Klass M. R. & Hirsh D. I. (1976) Nature 260:523-525). When conditions improve, worms can re-enter the life cycle and develop into a normal reproductive hermaphrodite. The dauer formation genes (daf) were first isolated on the basis that they promote dauer arrest under plentiful growth conditions (dauer constitutive) or prevent dauer formation under crowded conditions (dauer defective) (Riddle D. L. et al. in C. elegans II, (1997) 739-768, Cold Spring Harbor Laboratory Press). Several of these genes, including daf-2, age-1 and daf-16, were subsequently identified as part of an insulin-like signaling pathway and shown to regulate life span.

The insulin-like signaling pathway in C. elegans contains 37 family members, of which daf-2 is the only insulin receptor-like gene (Pierce S. B. et al. (2001) Genes and Dev. 15:672-686; Gregoire F. M. et al. (1998) Biochem. Biophys. Res. Com. 249:385-390). DAF-2 resembles both insulin receptor (IR) and the related insulin growth factor-1 receptor (IGF1-R) (Kimura K. et al. (1997) Science 277:942-946). Activation of DAF-2 by an as yet unidentified ligand leads to activation of PI-3 kinase, the catalytic subunit of which is encoded in C. elegans by the age-1 gene (Morris J. Z. et al. (1996) Nature 382:536-539). Decrease-in-function mutations in either daf-2 or age-1 result in many phenotypes including constitutive dauer formation during development, resistance to stresses, and extension of life span in adults (Lithgow G. J. et al., (1994) J. Gerontol. 49:B270-276; Lithgow G. J. et al., (1995) PNAS USA 92:7540-4; Murakami S. & Johnson T. E. A Genetics 143:1207-1218; Honda Y. & Honda S., (1999) FASED J 13:1385-1393; Baryste D., (2001) FASEB J 15:627-634; Friedman D. B. & Johnson T. E., (1988) Genetics 118:75-86; Klass M. R., (1983) Mech of Ageing and Dev. 22:279-286). Activation of PI-3 kinase results in the generation of the 3-phosphoinositide second messengers PIP2 and PIP₃, which in turn activate the downstream kinases PDK-1, AKT-1, and AKT-2. In particular, in mammals, Akt is activated by PDK phosphorylation at Thr 308 and PDK-2/TORC-2 protein complex at Ser 473 (Brazil and Hemmings, 2001; Jacinto et al., 2006; Sarbassov et al., 2005). In C. elegans AKT-1, these sites correspond to Thr 350 and Ser 517, respectively. These kinases, including AKT-1, ultimately antagonize the final output of the pathway, DAF-16, a homolog of the HNF-3/forkhead transcription factors (Kimura K. et al. (1997) Science 277:942-946; Ogg S. et al. (1997) Nature 389:994-9; Lin K. et al. (1997) Science 278:1319-1322). Null mutations of daf-16 decrease life span and completely suppress all phenotypes in double mutant combinations with daf-2 or age-1. The final targets of DAF-16 are presumed to regulate metabolism and fat storage (Kimura K. et al. (1997) Science 277:942-6; Lithgow G. J. et al. (1995) PNAS USA 92:7540-4). In particular, DAF-16 is known to regulate the transcription of many downstream genes such as sod-3, hsp-12.6, sip-1 and mtl-1 (Furuyama et al., 2000; Lee et al., 2003; McElwee et al., 2003; Murphy et al., 2003; Oh et al., 2006).

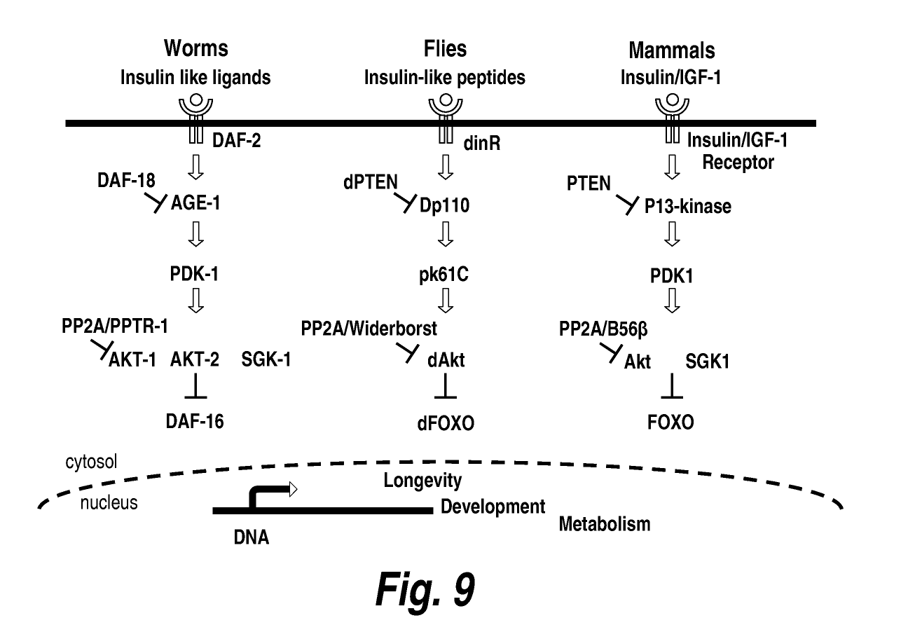

The insulin/IGF signaling pathway is highly conserved across a broad range of species, including nematodes, fruit flies, rodents, e.g., mice, and humans (FIG. 9).
Importantly, the influence of the insulin/IGF signaling pathway on lifespan has been conserved across large evolutionary distances. For example, in the fruit fly Drosophila, reduced insulin/IGF signaling also mediates life-span extensions (Clancy D. J. (2001) Science 292:104-106; Tatar M. & Yin C. (2001) Exp. Gerontol. 36:723-738). Indeed, insulin signaling has been demonstrated to play a role in regulating lifespan across a broad range of species, including yeast, fruit flies and rodents (see, e.g., Bluher et al. 2003 Science 299; 572-574; Al-Regaiey et al. 2005 Endocrinology 146(2):851-860; Kloting and Bluher 2005 Experimental Gerontology 40:878-883; and Barbieri et al. 2003 Am. J. Physiol. Endocrinol Metab. 285:E10640E1071). This conservation indicates that information on the aging of simple animals is likely to be similarly important for mammalian aging.

The present invention is based upon experiments carried out to identify novel regulators of the IIS pathway. In particular, a directed RNAi screen of serine/threonine protein phosphatases that affect phenotypes regulated by the IIS pathway was performed. C. elegans development proceeds from an egg, through 4 larval stages into a self-fertilizing, hermaphrodite adult. However, under unfavorable growth conditions such as crowding and low food availability, worms enter a stage of diapause known as as dauer (Riddle D., 1997). Upon favorable growth conditions, dauers are able to form reproductive adults. Since worms form dauers constitutively when the function of IIS pathway is reduced by mutations, a temperature-sensitive (ts) allele of daf-2 was taken advantage of for the RNAi screen (Riddle et al., 1981). A screen was carried out for genes that suppressed dauer formation in daf-2(e1370) mutants.

The PP2A phosphatases are comprised of three subunits, including a scaffold subunit (A subunit), a catalytic subunit (C subunit) and a regulatory subunit (B subunit) (FIG. 9). There are three known families of PP2A regulatory subunits, including the B55 (also referred to as B) family, B56 (also referred to as B′ or PR61) family and the B72 (also referred to as B″) family. The mammalian B56 family of PP2A regulatory subunits has 8 members encoded by 5 genes that express in different tissues, including B56α, B56β, B56γ, B56δ or B56ε (Eichhorn et al., 2008). The studies described herein characterize a B56 family regulatory subunit of the PP2A holoenzyme, PPTR-1 in C. elegans and B56β in mammals, as an important regulator of development, longevity, metabolism and stress response in C. elegans. In particular, the studies described herein show that PPTR-1 acts by modulating AKT-1 phosphorylation and as a consequence controls DAF-16 activity.

II. Screening Assays

The methods of the invention are suitable for use in methods to identify and/or characterize potential pharmacological agents, e.g. identifying new pharmacological agents from a collection of test substances, in particular, pharmacological agents for use in increasing life span and/or enhancing quality of life in aged individuals. Pharmacological agents identified according to the methodologies of the invention are also useful, for example, in enhancing stress resistance in individuals, and increasing the cytoprotective abilities of cells.

Pharmacological agents identified according to the methodologies of the invention are additionally useful in preventing or reducing obesity in individuals who suffer from obesity or in individuals who are predisposed to developing obesity, e.g., by reducing the adiposity of cells. Pharmacological agents identified according to the methodologies of the invention are also useful in treating individuals suffering from type II diabetes and preventing the onset of type II diabetes in individuals at risk of developing type II diabetes. Pharmacological agents are also useful in promoting apoptosis in cells, e.g., promoting apoptosis in tumor cells in individuals undergoing cancer therapies, e.g., cancer chemotherapy or radiation therapy. Pharmacological agents are additionally useful in inhibiting or downmodulating apoptosis in cells, e.g., in cells during development.

The methods described herein are in vitro and in vivo cell- and animal (e.g., nematode)-based screening assays.

A. Screening in Whole Organisms

The invention provides screening assays in whole organisms.

In one aspect, the invention provides method for evaluating the ability of a test compound to treat type II diabetes, comprising administering the test compound to an organism in which PP2A B56 regulatory subunit activity or expression is upmodulated, said organism having a phenotype, relative to a wild-type phenotype, associated with said upmodulation of PP2A B56 regulatory subunit activity or expression; and determining the ability of the test compound to effect said phenotype, to thereby evaluate the ability of the test compound to treat type II diabetes in a subject.

In another aspect, the invention provides method for evaluating the ability of a test compound to treat obesity, comprising administering the test compound to an organism in which PP2A B56 regulatory subunit activity or expression is upmodulated, said organism having a phenotype, relative to a wild-type phenotype, associated with said upmodulation of PP2A B56 regulatory subunit activity or expression; and determining the ability of the test compound to effect said phenotype, to thereby evaluate the ability of the test compound to treat obesity in a subject.

In another aspect, the invention provides method for evaluating the ability of a test compound to treat cancer, comprising administering the test compound to an organism in which PP2A B56 regulatory subunit activity or expression is upmodulated, said organism having a phenotype, relative to a wild-type phenotype, associated with said upmodulation of PP2A B56 regulatory subunit activity or expression; and determining the ability of the test compound to effect said phenotype, to thereby evaluate the ability of the test compound to treat cancer in a subject.

In another aspect, the invention provides method for evaluating the ability of a test compound to enhance lifespan, comprising administering the test compound to an organism in which PP2A B56 regulatory subunit activity or expression is upmodulated, said organism having a phenotype, relative to a wild-type phenotype, associated with said upmodulation of PP2A B56 regulatory subunit activity or expression; and determining the ability of the test compound to effect said phenotype, to thereby evaluate the ability of the test compound to enhance lifespan in a subject.

In one embodiment, the phenotype is nuclear localization of DAF-16 and, e.g., the ability of the compound to modulate the nuclear localization of DAF-16 is measured.

In one embodiment, the phenotype is decreased phosphorylation of DAF-16. In one embodiment, the ability of the compound to modulate the decreased phosphorylation of DAF-16 is measured. In one embodiment, the phenotype is decreased phosphorylation of AKT-1. In one embodiment, the ability of the compound to modulate the decreased phosphorylation of AKT-1 is measured.

In another aspect, the invention provides method for evaluating the ability of a test compound to treat type II diabetes, comprising administering the test compound to an organism in which PP2A B56 regulatory subunit activity or expression is downmodulated, said organism having a phenotype, relative to a wild-type phenotype, associated with said downmodulation of PP2A B56 regulatory subunit activity or expression; and determining the ability of the test compound to effect said phenotype, to thereby evaluate the ability of the test compound to treat type II diabetes in a subject.

In another aspect, the invention provides method for evaluating the ability of a test compound to treat obesity, comprising administering the test compound to an organism in which PP2A B56 regulatory subunit activity or expression is downmodulated, said organism having a phenotype, relative to a wild-type phenotype, associated with said downmodulation of PP2A B56 regulatory subunit activity or expression; and determining the ability of the test compound to effect said phenotype, to thereby evaluate the ability of the test compound to treat obesity in a subject.

In another aspect, the invention provides method for evaluating the ability of a test compound to treat cancer, comprising administering the test compound to an organism in which PP2A B56 regulatory subunit activity or expression is downmodulated, said organism having a phenotype, relative to a wild-type phenotype, associated with said downmodulation of PP2A B56 regulatory subunit activity or expression; and determining the ability of the test compound to effect said phenotype, to thereby evaluate the ability of the test compound to treat cancer in a subject.

In another aspect, the invention provides method for evaluating the ability of a test compound to enhance longevity, comprising administering the test compound to an organism in which PP2A B56 regulatory subunit activity or expression is downmodulated, said organism having a phenotype, relative to a wild-type phenotype, associated with said downmodulation of PP2A B56 regulatory subunit activity or expression, and determining the ability of the test compound to effect said phenotype, to thereby evaluate the ability of the test compound to enhance longevity in a subject. In one embodiment of these aspects, said organism further has a deregulated insulin signaling pathway, wherein said detectable phenotype is associated with said downmodulation of PP2A B56 regulatory subunit activity or expression and with said deregulated insulin signaling pathway.

In one embodiment, the phenotype is cytoplasmic localization of DAF-16 and, e.g., the ability of the compound to modulate the cytoplasmic localization of DAF-16 is measured.

In one embodiment, the phenotype is phosphorylation of DAF-16 and, e.g., the ability of the compound to modulate the phosphorylation of DAF-16 is measured.

In a preferred embodiment, the phenotype is phosphorylation of AKT-1 and , e.g., the ability of the compound to modulate the phosphorylation of AKT-1 is measured. In a particularly preferred embodiment, the phenotype is phosphorylation of AKT-1 at T350 (T308 in mammals) and the ability of the compound to modulate the phosphorylation of AKT-1 at T350 (T308 in mammals), e.g., to modulate the dephosphorylation of AKT-1 at T350 (T308 in mammals) by PPTR-1 (or B56β in mammals) is measured.

In a preferred embodiment of the invention, the roundworm Caenorhabditis elegans is employed. C. elegans is a simple soil nematode species that has been extensively described at the cellular and molecular level, and is a model organism for biological studies. C. elegans can develop through a normal life cycle that involves four larval stages and a final molt into an adult hermaphrodite. The dauer pathway is an alternative life cycle stage common to many nematode species which is normally triggered by environmental stresses such as starvation, temperature extremes, or overcrowding. Genetically, the dauer pathway has been most intensively studied in C. elegans. The response to overcrowding in C. elegans is mediated by a substance known as dauer pheromone, which is secreted by the animals. When dauer pheromone becomes sufficiently concentrated, it triggers commitment to the dauer alternative life cycle stage. A strong correlation exists between a constitutive dauer and the long-lived phenotype.

In preferred embodiments of the invention, the detectable phenotype is increased or decreased life span. In another embodiment, the detectable phenotype is constitutive dauer formation or defective dauer formation. In other embodiments, the phenotype is increased or decreased body size, or increased or decreased stress resistance, wherein stress resistance is selected from, but not limited to, the group consisting of oxidative stress, ultraviolet (UV) stress, hypoxic stress, heavy metal stress and heat stress. In other embodiments, the detectable phenotype is fat storage. In other embodiments, the detectable phenotype is increased or decreased rate of growth, e.g., fast growth or slow growth.

When screening for an effect of dauer formation, the assay population of C. elegans is preferably exposed to test agent during the portion of the life cycle at which commitment to the dauer pathway is made. Measurement of dauer formation has been previously described. See e.g., Riddle et al., Genetic and Environmental Regulation of Dauer Larva Development, In Riddle, Blumenthal, Meyer, and Priess (eds), C. ELEGANS II., Cold Spring Harbor Laboratory Press, Cold Spring Harbor, N.Y. (1997). In mutant strains containing deregulated JNK and/or insulin signaling and exhibiting a constitutive dauer phenotype, an agent is identified based on its ability to reverse that phenotype.

Life span assays have also been well described (Apfeld J. & Kenyon C. (1998) Cell 95: 199-210). In strains that exhibit an extended life span phenotype, an agent is identified based on its ability to either further extend or shorten the lifespan. Resistance to ultraviolet (UV) stress is determined by exposing the organism to UV light and measuring life span from the day of UV treatment. Oxidative stress resistance is determined by exposing the animals to paraquat, which produces superoxide when taken up by cells, and determining survival from the day of treatment (Feng et al. (2001) Dev. Cell 1:1-20.). Heat tolerance is measured by exposing adult animals to a 35° C. heat shock for 24 hours, and then scoring the animals for viability. Fat storage assays, e.g., in C. elegans, have been well described and are also described in the experiments provided herein. Growth assays have been well described (Gems et al., 1998; Jensen et al., 2007) and are further described in the experiments provided herein. In strains that exhibit a slow growth phenotype, an agent is identified based on its ability to either further slow growth or suppress the slow growth phenotype, e.g., increase growth.

In assay formats featuring indicator phenotypes, the phenotype of the animals may be detected by direct observation. An alternative to direct observation is mechanical detection of the animals. For instance, such detection could involve the determination of optical density across the test surface by a machine. The animals would be detected by changes in density at the location where an animal was located. Alternatively, if the animals are expressing a reporter gene that can be detected in living animals (e.g., GFP), a machine could monitor the animals using a suitable reporter gene detection protocol.

If desired, additional tests may be conducted using the agent identified to further characterize the nature of the agent's function with respect to longevity. For example, egg laying may also be measured to determine whether the longevity occurs by delaying maturity. As another example, other phenotypes associated with other gerontogenes could be tested to determine whether the identified agent affects functional pathways associated with these other genes. In such assays, the organism is a nematode. In a preferred embodiment, the nematode is C. elegans. In a further embodiment, the organism is a parasitic nematode. In one embodiment, the organism is not an insect.

In such assays, the indicator may be a downstream target of DAF-16, e.g., the sod-3, hsp-12.6, sip-1 or mtl-1 genes. In one embodiment, the indicator is selected from, but not limited to, the group consisting of DAF-16, superoxide dismutase (SOD), glucose transporter 4 (GLUT4) and glucose transporter 1 (GLUT1). In one embodiment, the indicator is DAF-16 or a mammalian orthologue thereof. Recent publications indicate that two other members of the insulin-like signaling pathway in C. elegans, DAF-9 and DAF-12, function downstream of DAF-16 (Gerisch B. et al. (2001) Dev. Cell, 1(6):841-51; Jia K. et al. (2002) Development 129:221-231). In C. elegans, daf-9 encodes a cytochrome P450 related to vertebrate steroidogenic hydroxylases, suggesting it could metabolize a DAF-12 ligand. In another embodiment, therefore, the indicator may be either DAF-9 or DAF-12.

In such an assay, the agent may be identified based on its ability to increase or decrease the indicator. The agent may alter expression of the indicator, wherein the expression is nucleic acid expression or polypeptide expression. The alteration of expression may be a change in the rate of expression or steady state expression.

In one embodiment, the agent alters the activity of the indicator. In a preferred embodiment, the agent may alter the post-translational modification state of the indicator, e.g. the phosphorylation state of the indicator, e.g., the phosphorylation state of DAF-16 or a mammalian orthologue thereof. In a particularly preferred embodiment, the indicator is the phosphorylation state of AKT-1 at specific phosphorylation sites in AKT-1, or a mammalian orthologue thereof (e.g., the phosphorylation sites T350 or S517 in C. elegans, or T308 or S473 in mammals), that is regulated by the activity of a PP2A B56 regulatory subunit or a mammalian orthologue thereof. In a preferred embodiment of the invention, the indicator is the phosphorylation state of the specific phosphorylation site T350 in AKT-1 in C. elegans, or the corresponding phosphorylation site in a mammalian orthologue thereof, e.g., the phosphorylation site T308 in mammals. It will be understood by the ordinary skilled artisan that the specific phosphorylation sites in a particular AKT-1 orthologue can be determined using methodologies well known to one of ordinary skill in the art and, for example, as described in the Examples herein. Techniques are well known in the art for analyzing phosphorylation and other post-translational modification states. For example, phosphorylation may be determined by the use of antibodies to phospho-epitopes to detect a phosphorylated polypeptide by Western analysis.

In another embodiment, the agent may alter the cellular localization of the indicator, such as from cytoplasmic to nuclear. In a preferred embodiment, the agent alters the nuclear translocation of DAF-16 or a mammalian orthologue thereof, e.g., the nuclear translocation of DAF-16 regulated by AKT-1. In one embodiment, nuclear translocation of DAF-16 is upmodulated. In one embodiment, nuclear translocation of DAF-16 is downmodulated. Changes in cellular localization can be determined by introducing a chimeric form of the indicator containing a reporter gene. Plasmid constructs can be introduced into C. elegans using described transformation methods. See e.g., Mello et al., (1991) EMBO J. 10:3959-3970. Preferably, the plasmid constructs are linear constructs. An important aspect of transformation in C. elegans is that plasmid constructs can be easily cotransformed, thus allowing for assay formats in which C. elegans are engineered to express, for example, non-C. elegans signaling pathway molecules and reporter genes. Preferably, a reporter gene is used that can be scored in a living animal, but does not affect the indicator phenotype of the animal. For example, green fluorescent protein (herein referred to as “GFP”) is a widely used reporter molecule in living systems. Ellenberg (1999) Trends Cell Biol. 9:52-56; Chalfie et al., (1994) Science 263:802-805.

B. Cell-Based Screening Assays

The invention further features cell-based assays for the identification of an agent capable of treating obesity, an agent capable of treating diabetes, an agent capable of treating cancer or an agent capable of enhancing longevity.

In particular, the invention features a method for evaluating the ability of a test compound to treat type II diabetes, comprising contacting a cell in which PP2A B56 regulatory subunit activity or expression is upmodulated with a test compound, wherein a detectable indicator is associated with said upmodulation of PP2A B56 regulatory subunit activity or expression, and determining the ability of the test compound to effect said indicator, to thereby evaluate the ability of the test compound to treat type II diabetes.

In yet another aspect, the invention provides method for evaluating the ability of a test compound to treat obesity, comprising contacting a cell in which PP2A B56 regulatory subunit activity or expression is upmodulated with a test compound, wherein a detectable indicator is associated with said upmodulation of PP2A B56 regulatory subunit activity or expression, and determining the ability of the test compound to effect said indicator, to thereby evaluate the ability of the test compound to treat obesity.

In yet another aspect, the invention provides method for evaluating the ability of a test compound to treat cancer, comprising contacting a cell in which PP2A B56 regulatory subunit activity or expression is upmodulated with a test compound, wherein a detectable indicator is associated with said upmodulation of PP2A B56 regulatory subunit activity or expression, and determining the ability of the test compound to effect said indicator, to thereby evaluate the ability of the test compound to treat cancer.

In another aspect, the invention provides method for evaluating the ability of a test compound to enhance longevity, comprising contacting a cell in which PP2A B56 regulatory subunit activity or expression is upmodulated with a test compound, wherein a detectable indicator is associated with said upmodulation of PP2A B56 regulatory subunit activity or expression, and determining the ability of the test compound to effect said indicator, to thereby evaluate the ability of the test compound to enhance longevity.

In one embodiment, the phenotype is nuclear localization of DAF-16 and, e.g., the ability of the compound to modulate the nuclear localization of DAF-16 is measured.

In one embodiment, the phenotype is decreased phosphorylation of DAF-16 and, e.g., the ability of the compound to modulate the decreased phosphorylation of DAF-16 is measured.

In one embodiment, the phenotype is decreased phosphorylation of AKT-1 and, e.g., the ability of the compound to modulate the decreased phosphorylation of AKT-1 is measured. In a particular embodiment, the phenotype is decreased phosphorylation of AKT-1 at the specific phosphorylation site T350 (T308 in mammals), e.g., decreased phosphorylation at T350 caused by or as a result of PP2A phosphatase PPTR-1 (B56B) regulatory subunit activity.

In another aspect, the invention provides a method for evaluating the ability of a test compound to treat type II diabetes, comprising contacting a cell in which PP2A B56 regulatory subunit activity or expression is downmodulated with a test compound, wherein a detectable indicator is associated with said downmodulation of PP2A B56 regulatory subunit activity or expression, and determining the ability of the test compound to effect said indicator, to thereby evaluate the ability of the test compound to treat type II diabetes.

In another aspect, the invention provides method for evaluating the ability of a test compound to treat obesity, comprising contacting a cell in which PP2A B56 regulatory subunit activity or expression is downmodulated with a test compound, wherein a detectable indicator is associated with said downmodulation of PP2A B56 regulatory subunit activity or expression, and determining the ability of the test compound to effect said indicator, to thereby evaluate the ability of the test compound to treat obesity.

In another aspect, the invention provides method for evaluating the ability of a test compound to treat cancer, comprising contacting a cell in which PP2A B56 regulatory subunit activity or expression is downmodulated with a test compound, wherein a detectable indicator is associated with said downmodulation of PP2A B56 regulatory subunit activity or expression, and determining the ability of the test compound to effect said indicator, to thereby evaluate the ability of the test compound to treat cancer.

In another aspect, the invention provides method for evaluating the ability of a test compound to enhance longevity, comprising contacting a cell in which PP2A B56 regulatory subunit activity or expression is downmodulated with a test compound, wherein a detectable indicator is associated with said downmodulation of PP2A B56 regulatory subunit activity or expression, and determining the ability of the test compound to effect said indicator, to thereby evaluate the ability of the test compound to enhance longevity.

In one embodiment, the phenotype is cytoplasmic localization of DAF-16 and, e.g., the ability of the compound to modulate the cytoplasmic localization of DAF-16 is measured.

In one embodiment, the phenotype is phosphorylation of DAF-16 and, e.g., the ability of the compound to modulate the phosphorylation of DAF-16 is measured.

In one embodiment, the phenotype is phosphorylation of AKT-1 and, e.g., the ability of the compound to modulate the phosphorylation of AKT-1 is measured. In a particular embodiment, the phenotype is modulated phosphorylation of AKT-1 at the specific phosphorylation site T350 (T308 in mammals), e.g., decreased or increased phosphorylation at T350 caused by or as a result of PP2A phosphatase PPTR-1 (B56B) regulatory subunit activity.

In one embodiment, the cell is a mammalian cell, e.g., a human cell. In another embodiment, the cell is a cell derived from a nematode.

The cell-based screening assays described herein have several advantages over conventional drug screening assays: 1) if an agent must enter a cell to achieve a desired therapeutic effect, a cell-based assay can give an indication as to whether the agent can enter a cell; 2) a cell-based screening assay can identify agents that, in the state in which they are added to the assay system are ineffective to modulate the PP2A B56 regulatory subunit and/or insulin signaling polynucleotide and/or polypeptide function, but that are modified by cellular components once inside a cell in such a way that they become effective agents; 3) most importantly, a cell-based assay system allows identification of agents affecting any component of a pathway that ultimately results in characteristics that are associated with PP2A B56 regulatory subunit and/or insulin signaling polynucleotide and/or polypeptide function.

In preferred embodiments, the indicator is altered cellular localization of DAF-16 or a mammalian orthologue thereof, e.g., FOXO, e.g., nuclear localization of DAF-16 or a mammalian orthologue thereof, e.g., FOXO. In other preferred embodiments, the indicator is altered phosphorylation state of AKT-1 or a mammalian orthologue thereof, e.g., phosphorylation of AKT-1 at the threonine 308 position in mammalian AKT-1 (e.g., the threonine 350 position of AKT-1 in C. elegans).

In one embodiment, suitable host cells include, but are not limited to, fungi (including yeast), bacterial, insect and mammalian cells. In a preferred embodiment, the host cell is a mammalian cell, e.g., a human cell. In another embodiment, the cells are derived from a nematode.

Functional characteristics of indicators include, but are not limited to, transcription, translation (including levels of precursor and/or processed polypeptide), location of protein product (such as nuclear or membrane localization), post-translational modification of protein product (such as phosphorylation or acetylation), any enzymatic activities, such as kinase activity, structural and/or functional phenotypes (such as stress resistance or life cycle), and expression (including repression or de-repression) of any other genes known to be controlled (modulated) by the polynucleotide. Any measurable change in any of these and other parameters indicate that the agent may be useful.

Modulation of function of a PP2A B56 regulatory subunit molecule, polynucleotide and/or polypeptide, may occur at any level. An agent may modulate function by reducing or preventing transcription of a PP2A B56 regulatory subunit polynucleotide. An example of such an agent is one that binds to the upstream controlling region, including a polynucleotide sequence or polypeptide. An agent may modulate translation of mRNA. An example of such an agent is one that binds to the mRNA, such as an anti-sense polynucleotide, or an agent which selectively degrades or stabilizes the mRNA. An agent may modulate function by binding to the PP2A B56 regulatory subunit polypeptide. An example of such an agent is a polypeptide or a chelator.

In preferred embodiments, to identify agents that modulate PP2A B56 regulatory subunit activity, the skilled artisan will look for modulation of phosphorylation of AKT-1. In a particular embodiment, the skilled artisan will look for modulation of phosphorylation of AKT-1 at the specific phosphorylation site T350 (T308 in mammals), e.g., modulation of the dephosphorylation of AKT-1 at T350 caused by or as a result of PP2A phosphatase PPTR-1 (B56B) regulatory subunit activity.

In each of these instances, the indication may involve an endogenous gene or protein. Alternatively, the indication could involve a reporter gene or protein.

Measuring all of these parameters (such as those using reporter genes) involve methods known in the art and need not be discussed herein. For example, degree of transcription can be measured using standard Northern analysis. Amount of expression product may be measured simply by Western analysis (if an antibody is available) or by a functional assay that detects the amount of protein, such as kinase activity.

Cell-based screening assays of the present invention can be designed, e.g., by constructing cell lines or strains of animals in which the expression of a reporter protein, i.e., an easily assayable protein, such as β-galactosidase, chloramphenicol acetyltransferase (CAT), green fluorescent protein (GFP) or hiciferase, is dependent on JNK and/or insulin signaling polynucleotide and/or polypeptide function. The cell is exposed to a test agent, and, after a time sufficient to effect β-galactosidase expression and sufficient to allow for depletion of previously expressed β-galactosidase, the cells are assayed for the production of β-galactosidase under standard assaying conditions.

Reporter genes include, but are not limited to, alkaline phosphatase, chloramphenicol acetyl transferase, galactosidase, luciferase and green fluorescent protein. Identification methods for the products of reporter genes include, but are not limited to, enzymatic assays and fluorimetric assays. Reporter genes and assays to detect their products are well known in the art and are described, for example in Current Protocols in Molecular Biology, eds. Ausubel et al., Greene Publishing and Wiley-Interscience: New York (1987) and periodic updates. Reporter genes, reporter gene assays and reagent kits are also readily available from commercial sources (Stratagene, Invitrogen and etc.).

Introduction of PP2A B56 regulatory subunit oligonucleotides (or reporter gene polynucleotides) depend on the particular host cell used and may be by any of the many methods known in the art, such as microinjection, spheroplasting, electroporation, CaCl, precipitation, lithium acetate treatment, and lipofectamine treatment.

Polynucleotides introduced into a suitable host cell(s) are polynucleotide constructs comprising a PP2A B56 regulatory subunit polynucleotide. These constructs contain elements (i.e., functional sequences) which, upon introduction of the construct, allow expression (i.e., transcription, translation, and post-translational modifications, if any) of PP2A B56 regulatory subunit polypeptide amino acid sequence in the host cell. The composition of these elements will depend upon the host cell being used. For introduction into C. elegans, polynucleotide constructs will generally contain the PP2A B56 regulatory subunit polynucleotide operatively linked to a suitable promoter and will additionally contain a selectable marker such as rol-6 (su1006). Other suitable host cells and/or whole animals include, for example, insect, yeast and mammalian cells. In one embodiment, the host cells and/or whole animals are not insects, e.g., Drosophila. Suitable selectable markers for nematode cells are those that enable the identification of cells that have taken up the nucleic acid, such as morphologic and behavioral markers such as rol-6 or visual markers such as green fluorescent protein. Screening of the transfectants identifies cells or animals that have taken up and express the polynucleotide.

The screening assay formats and components utilized in the screening assays featured in the instant invention are also useful for screening for agents that can be used to treat disorders, e.g., metabolic disorders, e.g., diabetes, e.g., type II diabetes. Type 2 diabetes is a disease of peripheral insulin resistance combined with pancreatic beta-cell dysfunction, and current evidence indicates that disruption of insulin/insulin-like growth factor (IGF)-1 signaling mechanisms may contribute to both defects. Based on the discoveries provided herein, which reveal that the PP2A B56 regulatory subunit (e.g., PPRT-1 or B56β) negatively regulates insulin signaling by directly regulating phosphorylation of AKT-1, the PP2A B56 regulatory subunit (e.g., PPRT-1 or B56β) is an attractive therapeutic targets for treatment of metabolic disorders, e.g., diabetes, e.g., type 2 diabetes and disorders associated with diabetes. The skilled artisan will appreciate that assay formats or combinations of assay components as described herein may be modified for the above described purpose.

III. Test Compounds

A variety of test compounds can be evaluated using the screening assays described herein. Exemplary compounds which can be screened for activity include, but are not limited to, peptides, nucleic acids, carbohydrates, small organic molecules, and natural product extract libraries. In one embodiment, an agent or compound that modulates PP2A B56 regulatory subunit activity in a cell is an agent that has not been previously identified as one that modulates PP2A B56 regulatory subunit activity. In another embodiment, an agent or compound that modulates PP2A B56 regulatory subunit activity in a cell is a known agent, e.g., a PP2A B56 regulatory subunit nucleic acid molecule, or biologically active fragment thereof or PP2A B56 regulatory subunit polypeptides, or biologically active fragments thereof.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms or PP2A B56 regulatory subunit molecules, e.g., dominant negative mutant forms of the molecules.

Other agents that can be used to modulate the activity of a PP2A B56 regulatory subunit include chemical compounds that directly modulate PP2A B56 regulatory subunit activity or compounds that modulate the interaction between the B56 regulatory subunit and the catalytic and/or structural subunits of the PP2A holoenzyme, or compounds that enhance the ability of the PP2A B56 regulatory subunit (e.g., as part of the PP2A holoenzyme) to directly regulate phosphorylation of AKT-1. Such compounds can be identified using screening assays that select for such compounds, as described in detail above.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 10⁴-10⁵ as been described (Carell et al. (1994). Angew. Chem. Int. Ed. Engl. 33:2059; Carell et al. (1994) Angew. Chem. Int. Ed. Engl. 33:2061-2064).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Compound Des. 12:145). Other exemplary methods for the synthesis of molecular libraries can be found in the art, for example in: Erb et al. (1994). Proc. Natl. Acad. Sci. USA 91:11422; Horwell et al. (1996) Immunopharmacology 33:68-; and in Gallop et al. (1994); J. Med. Chem. 37:1233-.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); In still another embodiment, the combinatorial polypeptides are produced from a cDNA library.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Compound Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

In one embodiment, an agent that selectively modulates PP2A B56 regulatory subunit activity is a small molecule which interacts with the PP2A B56 regulatory subunit protein to thereby modulate the activity of PP2A B56 regulatory subunit. Small molecule modulators of PP2A B56 regulatory subunit can be identified using database searching programs capable of scanning a database of small molecules of known three-dimensional structure for candidates which fit into the target protein site known in the art. Suitable software programs include, for example, CATALYST (Molecular Simulations Inc., San Diego, Calif.), UNITY (Tripos Inc., St Louis, Mo.), FLEXX (Rarey et al., J. Mol. Biol. 261: 470-489 (1996)), CHEM-3DBS (Oxford Molecular Group, Oxford, UK), DOCK (Kuntz et al., J. Mol. Biol 161: 269-288 (1982)), and MACCS-3D (MDL Information Systems Inc., San Leandro, Calif.).

The molecules found in the search may not necessarily be leads themselves, however, such candidates might act as the framework for further design, providing molecular skeletons to which appropriate atomic replacements can be made. The scaffold, functional groups, linkers and/or monomers may be changed to maximize the electrostatic, hydrogen bonding, and hydrophobic interactions with the target protein. Goodford (Goodford J Med Chem 28:849-857 (1985)) has produced a computer program, GRID, which seeks to determine regions of high affinity for different chemical groups (termed probes) on the molecular surface of the binding site. GRID hence provides a tool for suggesting modifications to known ligands that might enhance binding. A range of factors, including electrostatic interactions, hydrogen bonding, hydrophobic interactions, desolvation effects, conformational strain or mobility, chelation and cooperative interaction and motions of ligand and enzyme, all influence the binding effect and should be taken into account in attempts to design small molecule enhancers.

Small molecule modulators of a PP2A B56 regulatory subunit can also be identified using computer-assisted molecular design methods comprising searching for fragments which fit into a binding region subsite and link to a predefined scaffold can be used. The scaffold itself may be identified in such a manner. Programs suitable for the searching of such functional groups and monomers include LUDI (Boehm, J Comp. Aid. Mol. Des. 6:61-78 (1992)), CAVEAT (Bartlett et al. in “Molecular Recognition in Chemical and Biological Problems”, special publication of The Royal Chem. Soc., 78:182-196 (1989)) and MCSS (Miranker et al. Proteins 11:29-34 (1991)).

Yet another computer-assisted molecular design method for identifying small molecule modulators of the protein comprises the de novo synthesis of potential modulators by algorithmic connection of small molecular fragments that will exhibit the desired structural and electrostatic complementarity with the active binding site of the PP2A B56 regulatory subunit protein. The methodology employs a large template set of small molecules with are iteratively pieced together in a model of the PP2A B56 regulatory subunit binding site. Programs suitable for this task include GROW (Moon et al. Proteins 11:314-328 (1991)) and SPROUT (Gillet et al. J Comp. Aid. Mol. Des. 7:127 (1993)).

The suitability of small molecule candidates can be determined using an empirical scoring function, which can rank the binding affinities for a set of enhancers. For an example of such a method see Muegge et al. and references therein (Muegge et al., J Med. Chem. 42:791-804 (1999)). Other modeling techniques can be used in accordance with this invention, for example, those described by Cohen et al. (J. Med. Chem. 33: 883-894 (1994)); Navia et al. (Current Opinions in Structural Biology 2: 202-210 (1992)); Baldwin et al. (J. Med. Chem. 32: 2510-2513 (1989)); Appelt et al. (J. Med. Chem. 34: 1925-1934 (1991)); and Ealick et al. (Proc. Nat. Acad. Sci. USA 88: 11540-11544 (1991)).

A. Compounds that Increase PP2A B56 Regulatory Subunit Activity

The methods of the invention using compounds which modulate, e.g., increase, the expression and/or activity of a PP2A B56 regulatory subunit can be used in the prevention and/or treatment of disorders in which cell proliferation is undesirable or undesirably enhanced, stimulated, upregulated or the like, e.g., cancer. The methods of the invention using compounds which modulate, e.g., increase the expression and/or activity of a PP2A B56 regulatory subunit can also be used to enhance longevity in a subject.

1. Known Agents that Increase PP2A B56 Regulatory Subunit Activity

In one embodiment, an agent that is known to increase PP2A B56 regulatory subunit activity may be selectively targeted to cells as described below.

(i) PP2A B56 Regulatory Subunit Nucleic Acid Molecules

In one embodiment, isolated nucleic acid molecules that encode a PP2A B56 regulatory subunit or a biologically active portion thereof, may be used to increase activity in a cell. In one embodiment, the nucleic acid molecule of the invention enodes all or a portion of an amino acid sequences shown below, or is complementary to a nucleic acid molecule encoding an amino acid shown below.

Pptr-1 (C. elegans)
>gi|115534753|ref|NP_507133.3| Protein Phosphatase
2A (Two A) Regulatory subunit family member
(pptr-1) [Caenorhabditis elegans]
MHGSGHSLTGAPHQIPPPRTQGAATGGQQLSATANQFVDKIDPFHNKR
GTSRRLRINNSSRYNVDSAQELVQLALIKDTAANEQPALVIEKLVQCQ
HVFDFYDPVAQLKCKEIKRAALNELIDHITSTKGAIVETIYPAVIKMV
AKNIFRVLPPSENCEFDPEEDEPTLEVSWPHLQLVYELFLRFLESPDF
QASIGKKYIDQRFVLKLLDLFDSEDPRERDFLKTVLHRIYGKFLGLRA
FIRKHINNMFLRFVYETDSFNGVGELLEILGSIINGFALPLKQEHKVF
LVKVLLPLHKPKCLSLYHAQLAYCVVQFIEKDSSLTPQVFEALLKFWP
RTCSSKEVMFLGEVEEILDIIEPEQFKKIIDPLFRQLAKCVSSPHFQV
AERALYFWNNEYILSLIEDTSSLVMPIMFPALYRISKEHWNQTIVALV
YNVLKTFMEMNGKLFDELTSTYKGERLREKQREKDRDAFWKKMEALEL
NPPAEGKEVTPSLFPEKLTDYLKKDGPNMTPLPVATAGGGDKSPSWKK
SSTGSETTTPAKNSYQSFLILLLQKKKLDINGDIYDDKHLFYFSKMLS
VSASPSYTHSLVFCCWIETILFEIKSIDNCTHTNPRKPAKSHTLPTPP
QPKNKRHH
>gi|115534752|ref|NM_074732.4| Caenorhabditis
elegans Protein Phosphatase 2A (Two A) Regulatory
subunit family member (pptr-1) (pptr-1) mRNA,
complete cds
AAGCGCCCAGAAATCTCTCTCTCGCCGTCTGCTTCTCTGCTCCCTCCT
ACACGAAGTTTCAACACTTCGCTCGAACACAACCAGGGCGAGAAGACG
TTGAAGAAGATAAATTCTTTTAATCAAAAAAAAAAATGCACGGAAGCG
GGCACAGTCTAACGGGCGCTCCGCATCAAATTCCGCCGCCACGGACAC
AGGGAGCCGCCACCGGTGGCCAACAACTCTCGGCGACAGCTAATCAAT
TTGTTGATAAAATCGATCCGTTCCATAATAAAAGAGGAACTTCTAGAC
GATTGCGCATCAACAACAGCAGCCGATATAATGTTGATTCGGCTCAAG
AACTCGTACAATTGGCGCTCATCAAAGATACTGCTGCAAATGAGCAAC
CGGCTCTCGTTATCGAAAAGCTCGTCCAATGTCAGCACGTATTCGATT
TCTATGATCCAGTAGCTCAACTGAAATGCAAAGAAATCAAAAGAGCAG
CTCTAAATGAGCTCATCGATCACATTACATCGACAAAAGGAGCAATTG
TTGAGACAATTTATCCGGCTGTCATTAAAATGGTTGCCAAGAATATAT
TCCGTGTACTTCCACCATCAGAAAATTGTGAATTTGATCCAGAAGAGG
ATGAACCGACTTTAGAAGTCTCGTGGCCACATTTACAGCTTGTCTACG
AGCTCTTCCTGAGATTCCTTGAGTCGCCAGATTTCCAAGCTTCAATCG
GTAAAAAGTATATTGATCAGAGATTTGTGCTCAAGCTGCTCGATTTAT
TCGATTCGGAGGATCCACGTGAACGAGACTTTTTGAAGACGGTTCTTC
ATCGTATTTACGGAAAATTTTTGGGCCTTCGGGCTTTTATTCGCAAAC
ATATCAATAATATGTTTTTGAGATTCGTATACGAAACGGACTCATTCA
ACGGTGTCGGTGAGCTTCTCGAGATTCTTGGCTCAATTATAAACGGAT
TTGCTCTTCCATTGAAGCAAGAGCACAAGGTTTTCCTTGTAAAAGTTC
TTCTACCATTGCACAAACCGAAATGCTTATCACTGTATCATGCACAGC
TCGCCTACTGCGTCGTTCAATTCATCGAAAAAGACTCATCACTGACTC
CACAAGTTTTTGAGGCTCTGCTCAAATTTTGGCCTCGAACATGTAGCA
GCAAGGAGGTTATGTTCCTGGGTGAAGTGGAGGAAATTCTCGACATTA
TCGAACCGGAACAATTCAAAAAGATTATCGATCCATTATTCCGTCAAT
TGGCCAAATGTGTCAGTAGTCCACATTTTCAAGTTGCTGAACGGGCTC
TCTACTTTTGGAATAATGAATATATATTGTCATTAATTGAGGATACAA
GCAGTTTGGTGATGCCGATTATGTTCCCAGCGCTCTATCGGATTTCCA
AAGAGCACTGGAATCAAACAATTGTTGCACTTGTCTATAATGTACTCA
AAACTTTTATGGAAATGAATGGAAAACTGTTTGATGAGCTTACTTCTA
CGTACAAAGGTGAACGATTGCGGGAAAAGCAACGAGAAAAGGATCGTG
ATGCTTTTTGGAAGAAAATGGAAGCTCTTGAATTGAATCCACCGGCTG
AAGGAAAAGAAGTGACACCTTCGTTGTTTCCAGAGAAGTTGACTGATT
ATTTGAAGAAGGACGGTCCAAATATGACGCCACTGCCAGTTGCAACTG
CAGGTGGCGGTGATAAATCTCCATCTGTAGTGAAGAAATCGTCAACAG
GATCAGAAACGACGACGCCGGCTAAAAACTCCTATCAATCATTTCTTA
TTCTTCTTCTTCAAAAAAAAAAACTCGATATCAATGGTGATATATATG
ATGATAAGCACCTCTTCTATTTTTCCAAAATGCTCTCCGTCTCTGCGT
CTCCTTCTTATACACACTCTCTCGTCTTCTGCTGCTGGATCGAAACCA
TTTTATTTGAAATAAAATCAATCGATAATTGTACACACACAAATCCCC
GAAAACCCGCTAAATCACACACATTACCCACCCCACCCCAACCAAAAA
ATAAACGCCATCATTAATATTATTTTTCTCTTTTTTTATTATTTTTCC
TTTTTTCCCCCAAATTTTATTGAATTTCTTCCATTTTTCGCCCTCTTT
TCTGAAAATTCGTGTTTTTGCTCACCATGTCTTCGTGTTTTTCTTTTC
CCTCTCCACTTTTCCCTTTCTCTCTCTCTCTCGTCGTCGTTTTCCACA
TATTTTATTCCAGATTTTTCTCTATTCATAATATTTTTGTTGTATCCC
AAATTCTATTATCGAGTTGCGAACAAATGGTGATTTTTGGCGGAAAAT
TAGGGTCTCGCCGCGAAAAAACAATTTGCTACAGCACCCCTTTAAAGG
CGCACGATAGCGTCGAGACCTTTTTTAATCAAAAATCGATATTTTCTC
CGCAACTGGGTAATATTACTGAACTATTTCAGATTTTTAAATATACAT
TTTTTTGTGCCCCGTTTTATTTTTTTTTGTATGTCTGTTGTCTCTAGT
AGTATCAAATATCAATACCAAAAATAATGTATGGAATATTTTTTAATA
TTAGTGTTGTAAAGATCCCCCTCCTCCTCAAAATGTCCCAAAAATTGC
GAAGAAACAAAAAAATTCACTCTCTCCCCCTCTTTCTCTCTCTCTCTG
TATGAATTTTTTGTATTTATATACACAAACATACACACACACATACAC
ACCATA
Pptr-2 (C. elegans)
>gi|193207258|ref|NP_001122845.1| Protein
Phosphatase 2A (Two A) Regulatory subunit family
member (pptr-2) [Caenorhabditis elegans]
MLRSKKKDKENGKSEKKDKEKDKKSMKDDGAGSSKASTIPTIRTEDVG
GDMIPADAPPPTNIGRTNTYGGGPVIPRRERRQSSSMFNISQNRELQR
LPAIKDADPSERETLFIQKLRQCCVVFDFANDALSDLKFKEVKRAALN
ELVDHVSGAPKGSLSDAVYPEAIGMFSTNLFRPLSPPTNPIGAEFDPD
EDEPTLEAAWPHLQLVYEFFLRFLECPDFQSQVAKRYIDQNFILRLLM
IMDSEDPRERDFLKTTLHRIYGKFLGHRAYIRKQINNIFYSFIYETER
HNGIAELLEILGSIINGFALPLKEEHKTFLLRVLLPLHKVKSLSVYHP
QLAYCVVQFIEKDSSLTEPVISGMLREWPKQHSPKEVMELNELEEVLD
VIEPNEFQKIMTPLESQIARCVSSPHFQVAERALYYWNNEYVMSLVAD
NARVIIPIMFPVLEKNSKSHWNKTIHGLIYNALKMFMEMNQKLEDECS
QAYQKDRVQEKTLNEEKERIWNNIEKQAMGNPQYVEVKALFARFNPDE
IISSRQQNGVDENMKTSTVLSKDEILKNAVGVSSMKNDMDFGPNHKQS
DFPPDEQTTRALGEYKRHDPFLKKVTSTDEQ
>gi|193207257|ref|NM_001129373.1| Caenorhabditis
elegans Protein Phosphatase 2A (Two A) Regulatory
subunit family member (pptr-2) (pptr-2) mRNA,
complete cds
GCGGTCGGCTCCGCATCCGCAATGTTGAGATCAAAGAAGAAGGACAAG
GAAAATGGGAAATCAGAGAAAAAGGACAAAGAGAAGGATAAGAAATCA
ATGAAAGATGACGGTGCTGGAAGCAGTAAGGCATCTACAATTCCTACG
ATCCGAACAGAAGATGTTGGAGGAGATATGATTCCTGCAGATGCACCA
CCACCAACAAATATTGGCCGAACAAACACGTACGGAGGAGGGCCAGTG
ATACCGCGAAGAGAGCGCCGCCAATCAAGTAGCATGTTCAACATTTCA
CAAAACAGAGAGCTGCAAAGACTTCCAGCTATCAAAGATGCGGATCCG
AGCGAGAGAGAAACACTGTTCATTCAAAAACTTCGACAATGCTGCGTT
GTTTTTGACTTTGCCAATGATGCTCTCAGCGATTTAAAATTCAAAGAA
GTCAAACGGGCAGCTCTAAATGAACTCGTCGACCACGTTTCCGGAGCT
CCAAAAGGATCGCTATCTGATGCTGTCTATCCAGAAGCTATCGGCATG
TTCTCCACTAATCTTTTCCGTCCTTTGAGCCCACCGACCAATCCAATT
GGTGCTGAATTTGACCCCGATGAAGACGAACCAACATTAGAAGCTGCC
TGGCCACATCTGCAGCTCGTCTACGAGTTTTTCCTGAGATTCCTGGAG
TGCCCCGATTTTCAATCTCAAGTTGCAAAACGTTACATTGATCAAAAT
TTCATTCTTCGTCTTCTCATGATTATGGACAGTGAAGATCCACGTGAA
CGTGACTTTCTGAAGACAACTTTACATCGAATTTATGGAAAGTTCCTC
GGACATCGTGCTTACATTCGGAAGCAAATCAACAACATTTTCTACTCG
TTCATCTACGAAACTGAGAGGCACAACGGGATTGCTGAATTACTCGAG
ATTCTAGGTAGCATTATCAATGGATTCGCTCTTCCCTTGAAAGAGGAA
CACAAAACTTTCTTGCTCCGAGTTCTTCTTCCGCTACACAAGGTCAAA
TCATTATCTGTATATCATCCTCAACTCGCGTACTGTGTCGTACAGTTC
ATTGAAAAAGACTCATCACTGACAGAGCCTGTAATCAGTGGGATGCTA
CGCTTCTGGCCAAAACAACATAGTCCCAAGGAAGTGATGTTCCTGAAT
GAATTGGAAGAAGTCCTCGATGTGATAGAGCCGAATGAGTTCCAAAAA
ATCATGACTCCATTGTTCTCACAAATTGCTCGCTGTGTCAGCAGTCCA
CACTTCCAAGTTGCCGAACGAGCTCTCTACTATTGGAATAATGAATAT
GTGATGTCATTAGTAGCGGACAATGCTCGTGTGATAATTCCAATAATG
TTCCCTGTTCTTTTCAAAAACAGCAAGTCACATTGGAATAAAACGATT
CATGGACTTATCTACAACGCCCTCAAGATGTTCATGGAAATGAATCAA
AAACTGTTTGACGAGTGTTCCCAAGCTTATCAGAAAGATCGAGTTCAA
GAAAAGACATTGAATGAAGAAAAAGAACGCATCTGGAACAATATTGAG
AAACAGGCAATGGGAAACCCGCAATATGTTGAAGTTAAAGCACTATTT
GCTCGATTTAATCCAGATGAAATCATCAGCTCGCGTCAGCAGAATGGA
GTTGACGAGAATATGAAGACTTCAACTGTGCTTAGCAAGGATGAAATT
CTGAAAAATGCTGTAGGAGTGAGCTCGATGAAAAACGATATGGATTTC
GGACCCAACCACAAACAATCCGATTTCCCTCCAGATGAGCAAACGACG
AGAGCTTTAGGCGAGTACAAGCGTCACGATCCATTTTTGAAGAAAGTG
ACTAGCACCGACGAACAGTGA
Pptr-2 (C. elegans)
>gi|17557914|ref|NP_505807.1| Protein Phosphatase
2A (Two A) Regulatory subunit family member
(pptr-2) [Caenorhabditis elegans]
MIPADAPPPTNIGRTNTYGGDLISGPVIPRRERRQSSSMFNISQNREL
QRLPAIKDADPSERETLFIQKLRQCCVVEDFANDALSDLKEKEVKRAA
LNELVDHVSGAPKGSLSDAVYPEAIGMESTNLFRPLSPPTNPIGAEFD
PDEDEPTLEAAWPHLQLVYEFFLRFLECPDFQSQVAKRYIDQNFILRL
LMIMDSEDPRERDFLKTTLHRIYGKFLGHRAYIRKQINNIFYSFIYET
ERHNGIAELLEILGSIINGFALPLKEEHKTFLLRVLLPLHKVKSLSVY
HPQLAYCVVQFIEKDSSLTEPVISGMLREWPKQHSPKEVMELNELEEV
LDVIEPNEFQKIMTPLFSQIARCVSSPHFQVAERALYYWNNEYVMSLV
ADNARVIIPIMFPVLEKNSKSHWNKTIHGLIYNALKMFMEMNQKLEDE
CSQAYQKDRVQEKTLNEEKERIWNNIEKQAMGNPQYVEVKALFARFNP
DEIISSRQQNGVDENMKTSTVLSKDEILKNAVGVSSMKNDMDFGPNHK
QSDFPPDEQTTRALGEYKRHDPFLKKVTSTDEQ
>gi|133951887|ref|NM_073406.3| Caenorhabditis
elegans Protein Phosphatase 2A (Two A) Regulatory
subunit family member (pptr-2) (pptr-2) mRNA,
complete cds
GGATAAATTTTTTTTTGAATCACAATCATTGATTTTTCCATTTTCGGC
CCTCATATTTATCATTTCTAGGAGATATGATTCCTGCAGATGCACCAC
CACCAACAAATATTGGCCGAACAAACACGTACGGAGGAGATTTAATTT
CAGGGCCAGTGATACCGCGAAGAGAGCGCCGCCAATCAAGTAGCATGT
TCAACATTTCACAAAACAGAGAGCTGCAAAGACTTCCAGCTATCAAAG
ATGCGGATCCGAGCGAGAGAGAAACACTGTTCATTCAAAAACTTCGAC
AATGCTGCGTTGTTTTTGACTTTGCCAATGATGCTCTCAGCGATTTAA
AATTCAAAGAAGTCAAACGGGCAGCTCTAAATGAACTCGTCGACCACG
TTTCCGGAGCTCCAAAAGGATCGCTATCTGATGCTGTCTATCCAGAAG
CTATCGGCATGTTCTCCACTAATCTTTTCCGTCCTTTGAGCCCACCGA
CCAATCCAATTGGTGCTGAATTTGACCCCGATGAAGACGAACCAACAT
TAGAAGCTGCCTGGCCACATCTGCAGCTCGTCTACGAGTTTTTCCTGA
GATTCCTGGAGTGCCCCGATTTTCAATCTCAAGTTGCAAAACGTTACA
TTGATCAAAATTTCATTCTTCGTCTTCTCATGATTATGGACAGTGAAG
ATCCACGTGAACGTGACTTTCTGAAGACAACTTTACATCGAATTTATG
GAAAGTTCCTCGGACATCGTGCTTACATTCGGAAGCAAATCAACAACA
TTTTCTACTCGTTCATCTACGAAACTGAGAGGCACAACGGGATTGCTG
AATTACTCGAGATTCTAGGTAGCATTATCAATGGATTCGCTCTTCCCT
TGAAAGAGGAACACAAAACTTTCTTGCTCCGAGTTCTTCTTCCGCTAC
ACAAGGTCAAATCATTATCTGTATATCATCCTCAACTCGCGTACTGTG
TCGTACAGTTCATTGAAAAAGACTCATCACTGACAGAGCCTGTAATCA
GTGGGATGCTACGCTTCTGGCCAAAACAACATAGTCCCAAGGAAGTGA
TGTTCCTGAATGAATTGGAAGAAGTCCTCGATGTGATAGAGCCGAATG
AGTTCCAAAAAATCATGACTCCATTGTTCTCACAAATTGCTCGCTGTG
TCAGCAGTCCACACTTCCAAGTTGCCGAACGAGCTCTCTACTATTGGA
ATAATGAATATGTGATGTCATTAGTAGCGGACAATGCTCGTGTGATAA
TTCCAATAATGTTCCCTGTTCTTTTCAAAAACAGCAAGTCACATTGGA
ATAAAACGATTCATGGACTTATCTACAACGCCCTCAAGATGTTCATGG
AAATGAATCAAAAACTGTTTGACGAGTGTTCCCAAGCTTATCAGAAAG
ATCGAGTTCAAGAAAAGACATTGAATGAAGAAAAAGAACGCATCTGGA
ACAATATTGAGAAACAGGCAATGGGAAACCCGCAATATGTTGAAGTTA
AAGCACTATTTGCTCGATTTAATCCAGATGAAATCATCAGCTCGCGTC
AGCAGAATGGAGTTGACGAGAATATGAAGACTTCAACTGTGCTTAGCA
AGGATGAAATTCTGAAAAATGCTGTAGGAGTGAGCTCGATGAAAAACG
ATATGGATTTCGGACCCAACCACAAACAATCCGATTTCCCTCCAGATG
AGCAAACGACGAGAGCTTTAGGCGAGTACAAGCGTCACGATCCATTTT
TGAAGAAAGTGACTAGCACCGACGAACAGTGA
Pptr-2 (C. elegans)
>gi|17557912|ref|NP_505808.1| Protein Phosphatase
2A (Two A) Regulatory subunit family member
(pptr-2) [Caenorhabditis elegans]
MIPADAPPPTNIGRTNTYGGGPVIPRRERRQSSSMFNISQNRELQRLP
AIKDADPSERETLFIQKLRQCCVVEDFANDALSDLKEKEVKRAALNEL
VDHVSGAPKGSLSDAVYPEAIGMESTNLFRPLSPPTNPIGAEFDPDED
EPTLEAAWPHLQLVYEFFLRFLECPDFQSQVAKRYIDQNFILRLLMIM
DSEDPRERDFLKTTLHRIYGKFLGHRAYIRKQINNIFYSFIYETERHN
GIAELLEILGSIINGFALPLKEEHKTFLLRVLLPLHKVKSLSVYHPQL
AYCVVQFIEKDSSLTEPVISGMLREWPKQHSPKEVMELNELEEVLDVI
EPNEFQKIMTPLESQIARCVSSPHFQVAERALYYWNNEYVMSLVADNA
RVIIPIMFPVLEKNSKSHWNKTIHGLIYNALKMFMEMNQKLFDECSQA
YQKDRVQEKTLNEEKERIWNNIEKQAMGNPQYVEVKALFARFNPDEII
SSRQQNGVDENMKTSTVLSKDEILKNAVGVSSMKNDMDFGPNHKQSDF
PPDEQTTRALGEYKRHDPFLKKVTSTDEQ
>gi|86563734|ref|NM_073407.3| Caenorhabditis
elegans Protein Phosphatase 2A (Two A) Regulatory
subunit family member (pptr-2) (pptr-2) mRNA,
complete cds
GCGGTCGGCTCCGCATCCGCAATGTTGAGATCAAAGAAGAAGGACAAG
GAAAATGGGAAATCAGAGAAAAAGGACAAAGAGAAGGATAAGAAATCA
ATGAAAGATGACGGTGCTGGAAGCAGTAAGGCATCTACAATTCCTACG
ATCCGAACAGAAGATGTTGGAGGAGATATGATTCCTGCAGATGCACCA
CCACCAACAAATATTGGCCGAACAAACACGTACGGAGGAGGGCCAGTG
ATACCGCGAAGAGAGCGCCGCCAATCAAGTAGCATGTTCAACATTTCA
CAAAACAGAGAGCTGCAAAGACTTCCAGCTATCAAAGATGCGGATCCG
AGCGAGAGAGAAACACTGTTCATTCAAAAACTTCGACAATGCTGCGTT
GTTTTTGACTTTGCCAATGATGCTCTCAGCGATTTAAAATTCAAAGAA
GTCAAACGGGCAGCTCTAAATGAACTCGTCGACCACGTTTCCGGAGCT
CCAAAAGGATCGCTATCTGATGCTGTCTATCCAGAAGCTATCGGCATG
TTCTCCACTAATCTTTTCCGTCCTTTGAGCCCACCGACCAATCCAATT
GGTGCTGAATTTGACCCCGATGAAGACGAACCAACATTAGAAGCTGCC
TGGCCACATCTGCAGCTCGTCTACGAGTTTTTCCTGAGATTCCTGGAG
TGCCCCGATTTTCAATCTCAAGTTGCAAAACGTTACATTGATCAAAAT
TTCATTCTTCGTCTTCTCATGATTATGGACAGTGAAGATCCACGTGAA
CGTGACTTTCTGAAGACAACTTTACATCGAATTTATGGAAAGTTCCTC
GGACATCGTGCTTACATTCGGAAGCAAATCAACAACATTTTCTACTCG
TTCATCTACGAAACTGAGAGGCACAACGGGATTGCTGAATTACTCGAG
ATTCTAGGTAGCATTATCAATGGATTCGCTCTTCCCTTGAAAGAGGAA
CACAAAACTTTCTTGCTCCGAGTTCTTCTTCCGCTACACAAGGTCAAA
TCATTATCTGTATATCATCCTCAACTCGCGTACTGTGTCGTACAGTTC
ATTGAAAAAGACTCATCACTGACAGAGCCTGTAATCAGTGGGATGCTA
CGCTTCTGGCCAAAACAACATAGTCCCAAGGAAGTGATGTTCCTGAAT
GAATTGGAAGAAGTCCTCGATGTGATAGAGCCGAATGAGTTCCAAAAA
ATCATGACTCCATTGTTCTCACAAATTGCTCGCTGTGTCAGCAGTCCA
CACTTCCAAGTTGCCGAACGAGCTCTCTACTATTGGAATAATGAATAT
GTGATGTCATTAGTAGCGGACAATGCTCGTGTGATAATTCCAATAATG
TTCCCTGTTCTTTTCAAAAACAGCAAGTCACATTGGAATAAAACGATT
CATGGACTTATCTACAACGCCCTCAAGATGTTCATGGAAATGAATCAA
AAACTGTTTGACGAGTGTTCCCAAGCTTATCAGAAAGATCGAGTTCAA
GAAAAGACATTGAATGAAGAAAAAGAACGCATCTGGAACAATATTGAG
AAACAGGCAATGGGAAACCCGCAATATGTTGAAGTTAAAGCACTATTT
GCTCGATTTAATCCAGATGAAATCATCAGCTCGCGTCAGCAGAATGGA
GTTGACGAGAATATGAAGACTTCAACTGTGCTTAGCAAGGATGAAATT
CTGAAAAATGCTGTAGGAGTGAGCTCGATGAAAAACGATATGGATTTC
GGACCCAACCACAAACAATCCGATTTCCCTCCAGATGAGCAAACGACG
AGAGCTTTAGGCGAGTACAAGCGTCACGATCCATTTTTGAAGAAAGTG
ACTAGCACCGACGAACAGTGA
PPP2R5B (Human)
>gi|5453952|ref|NP_006235.1| beta isoform of
regulatory subunit B56, protein phosphatase 2A
[Homo sapiens]
METKLPPASTPTSPSSPGLSPVPPPDKVDGFSRRSLRRARPRRSHSSS
QFRYQSNQQELTPLPLLKDVPASELHELLSRKLAQCGVMFDFLDCVAD
LKGKEVKRAALNELVECVGSTRGVLIEPVYPDIIRMISVNIFRTLPPS
ENPEFDPEEDEPNLEPSWPHLQLVYEFFLRFLESPDFQPSVAKRYVDQ
KFVLMLLELFDSEDPREREYLKTILHRVYGKFLGLRAYIRKQCNHIFL
RFIYEFEHFNGVAELLEILGSIINGFALPLKTEHKQFLVRVLIPLHSV
KSLSVFHAQLAYCVVQFLEKDATLTEHVIRGLLKYWPKTCTQKEVMFL
GEMEEILDVIEPSQFVKIQEPLFKQVARCVSSPHFQVAERALYFWNNE
YILSLIEDNCHTVLPAVFGTLYQVSKEHWNQTIVSLIYNVLKTEMEMN
GKLEDELTASYKLEKQQEQQKAQERQELWQGLEELRLRRLQGTQGAKE
APLQRLTPQVAASGGQS
>gi|30795206|ref|NM_006244.2| Homo sapiens protein
phosphatase 2, regulatory subunit B′, beta isoform
(PPP2R5B), mRNA
GGGCGGCGGGTGCCGGTGCGCACGGAGCCGAGCCGGGGCTCCCGTTGC
GCTGCACCGCGTTGGGTCGGAGTCCCAGGACTTCAGCGGAGATCCGCG
CGCTGCGACGGCCGGTGCAGAGCCCGCCGAGCGCCCAGTCCCGGCCCG
GGGCTGAGTTGGGGGCATGCTCTAGCCGCCCCCCCGGAGCCCGGGAGA
GAACCCAGGAGCGCCGCCGCCCAGCCCCAGCGCCCCGAGCGGAACCGC
TGCGAAGGGGCCCTGAACGGCCGTCGCCCTCCCTACGGGCAGCCCCCG
GGGGTTGGCGACCGAAGTCTAGGTTTTCGAGAAAGCCAGGGTGGGAAC
CCTAACTGGACTCTTCGGGACCCCCAGGAAGGATCTGAGGCCTGAGCC
ATCCTCCTTTCTACCCTGTCTGCCCCCCAGGACTGGGCAGTTGCAGGA
GGCCCTGGGGGGGGGCCCAGGACTGTGGTTGTGCCCCCCCCCCAAAGG
CCGGACAGGATGGGACCAAGTTAGTCTGTCCAGTCTCACCCAGCACCT
CCCAGGCCCAGAGAGAACCCCCGGGGCTCTGAAAGCTTGCCCTGCCGC
CTGACCGCCATGGAGACGAAGCTGCCCCCTGCAAGCACCCCCACTAGC
CCCTCCTCCCCCGGGCTGTCGCCTGTGCCCCCACCCGACAAGGTGGAC
GGCTTCTCCCGCCGTTCCCTCCGCAGAGCCCGGCCCCGCCGCTCCCAC
AGCTCCTCTCAGTTCCGCTATCAGAGCAACCAGCAAGAGCTCACACCG
CTGCCCCTGCTCAAAGATGTGCCGGCTTCCGAGCTGCACGAGCTGCTG
AGCCGGAAGCTGGCCCAGTGTGGGGTGATGTTTGACTTCTTGGACTGT
GTGGCCGACCTCAAGGGGAAGGAGGTGAAGCGGGCAGCCCTCAACGAG
CTGGTGGAGTGTGTGGGGAGCACCCGGGGTGTCCTCATCGAGCCCGTC
TACCCAGACATCATCCGCATGATCTCAGTGAATATCTTCCGGACTCTG
CCGCCCAGTGAGAACCCTGAATTTGACCCTGAAGAGGATGAGCCCAAT
CTTGAGCCTTCGTGGCCACACCTGCAGCTGGTATATGAGTTTTTCCTG
CGTTTCTTGGAGAGCCCAGACTTCCAGCCCTCCGTGGCCAAGAGATAT
GTGGATCAAAAGTTTGTCCTGATGCTCCTGGAGCTATTTGATAGTGAG
GATCCCCGGGAGCGTGAGTACCTCAAGACCATCCTGCACCGGGTCTAT
GGCAAGTTCCTGGGTCTCCGGGCCTACATCCGCAAACAGTGCAACCAC
ATCTTCCTCCGGTTCATCTATGAATTCGAGCACTTCAATGGTGTGGCT
GAGCTGCTGGAGATCCTAGGAAGCATCATCAATGGCTTTGCGCTGCCC
CTGAAGACGGAGCACAAGCAGTTCCTGGTTCGCGTCCTGATCCCCCTG
CACTCTGTCAAGTCGCTGTCTGTCTTCCATGCCCAGCTGGCATACTGT
GTGGTGCAGTTCCTGGAGAAGGATGCCACTCTGACAGAGCACGTGATC
CGGGGGCTGCTCAAATACTGGCCAAAAACCTGCACCCAGAAGGAGGTG
ATGTTTCTGGGGGAGATGGAAGAGATTCTTGATGTCATCGAGCCCTCC
CAGTTTGTGAAGATCCAGGAGCCCCTTTTTAAGCAGGTGGCTCGCTGT
GTTTCCAGCCCCCATTTCCAGGTTGCAGAGCGGGCTCTGTATTTCTGG
AACAATGAGTATATCCTAAGCCTCATTGAGGACAACTGCCACACTGTG
CTGCCTGCTGTGTTTGGGACCCTCTACCAAGTCTCCAAGGAGCACTGG
AACCAAACCATCGTATCACTGATCTACAATGTGCTCAAGACCTTCATG
GAGATGAATGGGAAGCTGTTTGATGAGCTCACAGCCTCCTACAAGCTG
GAAAAGCAGCAGGAGCAGCAGAAGGCCCAGGAGCGTCAGGAGTTATGG
CAAGGTCTGGAGGAGCTGCGGCTACGCCGGCTACAGGGGACCCAGGGG
GCCAAGGAGGCCCCCCTCCAGCGGCTTACACCCCAGGTGGCCGCCAGT
GGGGGTCAGAGCTAGACAGCACCTCAGAAGGGGAAAAGCTAAACCCAG
AGCTGTCAGTCCCTCTATCCCTTCTCCTGTCCAGGGGCCCAGAGAGAA
ACACACCTACCCCTGGCCTTGCCAGAGTGGCTTCTGAGGACTCCCTGC
CCAGCCCAGCTTTCACTGGGGGGAGACGAGGAGAGGCAATGGTGGTCT
TGGCAACAGAATGCTCAGCCCCTCGTGGCAGGACTTGACAAGGGCAAG
CTTGACCAGGAAGCTGCCATCAGGGATCTTCCCCTGCCCCGCAAAGCT
AGGCTCCAGCTGCAGGCGGGCTCCCACCCTCTGCTCCTGGCCTTGGGC
AAGGGCACTCAGCGCCTCGCCTGCCCCTGCCTTGGCCAATGCGAGGTC
CTTCCTTATCCCCACCATGGGGTCCATGGTCTATTTATTCTCGCCCAG
CTCACCCTCTACACAGACACTGTCCTGGGTGCACACTCCTCCCTTCCC
TCGCTGTGTACTTCCTTGTCCCCTTTTTATTTATTGGGCAGGGGGAGG
GGGAGGGCACAGGCAAGAAGAGATTCACAGTGTCCTGGGGTAAGGGGG
GGTTCACAGTAATCATGGTCTACTCCTCTTTCCGTGGCTGGGGGTAGA
CTTAATAAAGAGAGAAATTCAA
PPP2R5B (Mouse)
>gi|37718993|ref|NP_937811.1| protein phosphatase
2, regulatory subunit B (B56), beta isoform
[Mus musculus]
METKLPPASTPTSPSSPGLSPVPPPDKVDGFSRRSLRRARPRRSHSSS
QFRYQSNQQELTPLPLLKDVPASELHELLSRKLAQCGVMFDFLDCVAD
LKGKEVKRAALNELVECVGCTRGVLIEPVYPDIIRMISVNIFRTLPPS
ENPEFDPEEDEPNLEPSWPHLQLVYEFFLRFLESPDFQPSVAKRYVDQ
KFVLMLLELFDSEDPREREYLKTILHRVYGKFLGLRAYIRKQCNHIFL
RFIYELEHFNGVAELLEILGSIINGFALPLKTEHKQFLVRVLIPLHSV
KSLSVFHAQLAYCVVQFLEKDATLTEHVIRGLLKYWPKTCTQKEVMFL
GEMEEILDVIEPSQFVKIQEPLFKQVARCVSSPHFQVAERALYFWNNE
YILSLIEDNCHTVLPAVFGTLYQVSKEHWNQTIVSLIYNVLKTFMEMN
GKLFDELTASYKLEKQQEQQKAQERQELWRGLEELRLRRLQGTQGAKE
APVPRPTPQVAASGGQS
>gi|142364854|ref|NM_198168.3| Mus musculus protein
phosphatase 2, regulatory subunit B (B56), beta
isoform (Ppp2r5b), mRNA
GTCATCCTGAGCAGCTGGGCGGCGGGTGCCGGTGCGCAGCGAGCCGGG
GCTCCCGCTGCGCTGCACCGCGCTGATCTGAGTCCCGGGATATCTGCC
GAGGGTCGCGTGCTGCCGGGGCCCAGCCCAGAGCCCACCGAGTGCCCG
ACCCCTTTTGGGGCTGAGCTGGGGGCATGCTCCAGCACCCCCAGAGCC
TGGGAGCGAACCCAGGAGCGCCGCCGCCCAGCCCCAGCGCCCCGAGCG
GCAGAACCACCGCTAAGGACCCTTGAACCGTCGTCTCTTCCACCGAGG
CAACCTCTAGGGTCGGCGACCAAAGTCTGGGTTTCTGAGAAAAGAGCC
AGCGTGGGAACCCTGACTGGAACTCTTCTGGATCCCCAGGAAAGACCT
GAGCCATTGCCTTTCTACCTTACCTGCCCCCCCAGGACTGGGCAGTTG
CCAGAGGCCCTGGGGGGGTCAGGACTGTGGCCGTGCCTCCCCCACCCC
CTGAAACATGCTGGACAGGATGGGTCCCAGTTAGTGTGTGTGGCCTCA
TTCAACTTACTCCAGACCCACAAGGAGCCCCCTGGGCTCTGAAAGATT
GACCCGTGTTGTCCACTGGCCATGGAGACGAAGCTGCCCCCTGCGAGC
ACCCCCACAAGCCCCTCCTCCCCAGGGCTGTCTCCAGTGCCACCACCA
GACAAGGTGGATGGCTTCTCCCGCAGGTCCCTCCGCAGGGCCCGGCCC
CGTCGCTCACACAGCTCTTCTCAGTTCCGCTATCAGAGCAACCAGCAA
GAGCTCACTCCACTGCCCCTGCTCAAAGATGTGCCAGCCTCTGAGTTG
CATGAGTTGCTGAGCCGGAAACTGGCCCAATGTGGGGTGATGTTTGAC
TTCTTGGACTGCGTGGCTGACCTCAAGGGGAAGGAGGTGAAGCGTGCA
GCCCTCAATGAACTGGTGGAATGTGTGGGTTGCACCCGGGGTGTGCTC
ATCGAGCCCGTCTACCCAGACATCATCCGCATGATATCAGTAAATATC
TTCCGGACCCTGCCACCCAGCGAGAACCCTGAGTTTGACCCTGAAGAA
GATGAGCCCAACCTTGAGCCCTCGTGGCCACATCTGCAGCTGGTATAT
GAGTTTTTCCTGCGTTTCTTGGAGAGTCCAGATTTCCAGCCCTCTGTG
GCCAAGAGATACGTGGATCAAAAGTTTGTCCTAATGCTCCTGGAGCTA
TTTGACAGCGAGGACCCCCGGGAACGTGAGTACCTCAAGACCATCTTG
CATCGGGTGTACGGCAAGTTCCTGGGTCTCCGGGCCTACATCCGCAAA
CAGTGCAACCACATCTTCCTCCGGTTCATCTACGAGCTGGAACACTTC
AATGGTGTGGCTGAGCTCTTAGAGATCTTAGGAAGCATCATCAATGGC
TTTGCGCTGCCCCTGAAGACTGAGCACAAGCAGTTCCTGGTTCGAGTC
CTGATCCCCTTGCACTCTGTCAAGTCACTATCTGTTTTTCATGCTCAG
CTGGCATACTGTGTGGTGCAGTTCCTGGAGAAGGATGCAACCTTGACA
GAGCATGTTATCCGGGGGCTTCTCAAATACTGGCCTAAAACCTGCACC
CAGAAGGAGGTGATGTTCCTGGGGGAGATGGAAGAGATTCTTGATGTC
ATCGAGCCCTCCCAGTTTGTGAAGATCCAGGAGCCCCTCTTCAAGCAG
GTGGCTCGCTGTGTCTCCAGCCCCCATTTCCAGGTTGCAGAGCGGGCT
CTGTATTTCTGGAACAATGAGTACATCCTGAGCCTCATTGAGGACAAC
TGCCACACTGTGCTGCCTGCGGTATTTGGGACCCTCTACCAAGTGTCC
AAGGAGCACTGGAATCAAACCATCGTGTCCCTGATCTACAACGTGCTC
AAGACTTTCATGGAGATGAACGGAAAGCTGTTTGACGAGCTCACAGCC
TCCTACAAGCTGGAAAAACAACAGGAGCAGCAGAAGGCCCAGGAGCGG
CAGGAGCTATGGCGAGGCTTGGAGGAACTGCGGCTACGCCGGCTACAG
GGGACCCAAGGGGCCAAGGAAGCCCCCGTCCCACGGCCTACGCCCCAG
GTGGCTGCCAGTGGGGGTCAGAGCTAGATACACCTAGAACAGGAGATG
CTAAACCCAGAGCTATCAGCCCCTCCATCCCTCTGCCCAGGGGCCCAG
TGAGGCCCACGCCTCCCCGTGGCCTTGCCGGAGTGGCTCTAGGACTCC
CTACCAGCCCCGTGGGAACAGCTTTCACGGAGGGGGAAACAAGAAGGC
AAGATGGTAGTCTTGGCAGCAGAACTCTCAGGCCTTTGTGGCAAGATT
CTGGCAAGACTAGACCAGGGCAAGTATGCAACTGGGAAGCTGCCATCA
GGGATCCTCCCCTGCCCTACACAGCCAGGCTCCAGGCGGCAGCTAGGC
CTCTACCCCTGCTCCCAGCCTGGGGCATGGACACTCAGCGCCTGGCCA
GGGCCTTCCTCATCCCCACCATGGGGGCACAGTCTATTTATTCTGCCC
AGCTCACCCCCAACAAGACCTTGTCCAGGGACATTCTCCTATCCTCTC
CCTGGCCCTGACTCCCTTATCCCCTTTTTATTTATTGGGCAGGGGGAG
GGGTGAGGGCACAGGCAAGAAGATTCACATTGTCCTGGGGTGAGGGGG
GAGGTCACAGTAATCATGGTCTGCCCCCTTCACCTGGCTGGGGGCAGA
TTTAATAAAGAGCGAAACTC
PPP2R5B (Rat)
>gi|31077118|ref|NP_852044.1| protein phosphatase
2, regulatory subunit B (B56), beta isoform
[Rattus norvegicus]
METKLPPASTPTSPSSPGLSPVPPPDKVDGFSRRSLRRARPRRSHSSS
QFRYQSNQQELTPLPLLKDVPASELHELLSRKLAQCGVMFDFLDCVAD
LKGKEVKRAALNELVECVGSTRGVLIEPVYPDIIRMISVNIFRTLPPS
ENPEFDPEEDEPNLEPSWPHLQLVYEFFLRFLESPDFQPSVAKRYVDQ
KFVLMLLELFDSEDPREREYLKTILHRVYGKFLGLRAYIRKQCNHIFL
RFIYELEHFNGVAELLEILGSIINGFALPLKTEHKQFLVRVLIPLHSV
KSLSVFHAQLAYCVVQFLEKDATLTEHVIRGLLKYWPKTCTQKEVMFL
GEMEEILDVIEPSQFVKIQEPLFKQVARCVSSPHFQVAERALYFWNNE
YILSLIEDNCHTVLPAVFGTLYQVSKEHWNQTIVSLIYNVLKTFMEMN
GKLFDELTASYKLEKQQEQQKAQERQELWRGLEELRLRRLQGTQGAKE
APVPRPTPQVAASGGQS
>gi|60593006|ref|NM_181379.2| Rattus norvegicus
protein phosphatase 2, regulatory subunit B (B56),
beta isoform (Ppp2r5b), mRNA
CAAGGAGCCCCTTGGGCTCTGAAAGATTGACTTGCTGCCCTCTGGCCA
TGGAGACGAAGCTGCCCCCTGCAAGCACCCCCACAAGCCCCTCCTCCC
CGGGGCTGTCTCCAGTGCCACCACCAGACAAGGTGGATGGCTTCTCCC
GCCGATCCCTCCGCAGGGCCCGGCCCCGACGCTCACACAGCTCTTCTC
AGTTCCGCTATCAGAGCAACCAGCAAGAGCTCACTCCACTGCCCCTGC
TCAAAGATGTGCCAGCCTCTGAGTTACATGAGTTGCTAAGCCGGAAAC
TGGCCCAATGTGGGGTGATGTTTGACTTCTTGGACTGTGTGGCTGACC
TCAAGGGCAAGGAGGTGAAGCGTGCAGCCCTCAATGAACTTGTGGAAT
GTGTGGGGAGCACCCGGGGGGTCCTCATTGAGCCTGTCTACCCAGACA
TCATCCGCATGATATCAGTAAATATCTTCCGGACCCTGCCGCCCAGTG
AGAACCCTGAATTTGACCCTGAAGAAGATGAGCCCAACCTTGAGCCTT
CGTGGCCACATCTACAGCTGGTATATGAGTTTTTCCTGCGGTTCTTGG
AGAGTCCAGATTTCCAGCCCTCTGTGGCCAAGAGATACGTGGATCAAA
AGTTTGTTCTGATGCTCCTGGAGCTATTTGACAGCGAGGACCCCCGGG
AACGTGAGTACCTCAAGACCATCTTGCATCGGGTATACGGCAAGTTCC
TGGGTCTCCGGGCCTACATCCGCAAACAGTGCAACCACATCTTCCTCC
GGTTCATCTATGAGCTGGAGCACTTTAATGGTGTGGCTGAGCTGTTAG
AGATCTTAGGAAGCATCATCAATGGCTTTGCGCTGCCCCTGAAGACTG
AGCACAAACAGTTCCTGGTTCGAGTCCTCATCCCCTTGCACTCTGTCA
AGTCACTGTCTGTTTTTCATGCTCAGCTGGCATACTGTGTGGTGCAGT
TCCTGGAGAAGGATGCGACCTTGACAGAGCATGTTATCCGGGGGCTTC
TCAAATACTGGCCTAAAACCTGCACCCAGAAGGAGGTGATGTTCCTGG
GGGAGATGGAAGAGATTCTCGATGTGATCGAGCCCTCCCAGTTTGTGA
AGATCCAGGAGCCTCTCTTCAAGCAGGTGGCTCGCTGTGTCTCCAGCC
CCCATTTCCAGGTTGCAGAGCGGGCTCTGTATTTCTGGAACAATGAGT
ACATCCTGAGCCTCATTGAGGACAACTGCCACACTGTGCTGCCTGCGG
TATTTGGGACCCTCTACCAAGTGTCCAAGGAGCACTGGAATCAAACCA
TCGTGTCCCTGATCTACAACGTGCTCAAGACTTTCATGGAGATGAATG
GGAAGCTGTTTGATGAGCTTACAGCCTCCTACAAGCTAGAAAAACAGC
AGGAGCAGCAGAAGGCCCAGGAGCGGCAGGAACTTTGGCGAGGCTTGG
AGGAACTGCGGTTACGCCGGCTACAGGGGACCCAAGGGGCCAAGGAAG
CCCCTGTCCCAAGGCCTACGCCCCAGGTGGCTGCCAGTGGGGGTCAAA
GCTAGATAAATCTAGAACAGGAGAAGCTAGACCCAGAGCTATCAGCCC
CTCCATCCCTTCTGCCCAGGGGCCCAGGGAGGCCCATGCCTCCCCGTG
GCCTTGCCAGCGTGGCTCTAGGACTCCCTACCAGCCCCATGGGAACAG
CTTTCATGGAGGGGAGACCAGAAGGCAAGATGGTAGTCTTGGCAGCAG
AACTCTCAGGCCCTTGTGGCAAGATTCTGGCAAGACTAGACCAGGGCA
AGTCTTCGACTGGGAAGCTGCCATCGGGGATCCTCCCCTGCCCTACAC
AGCTAGGCTCCAGGCGGCAGCTGGGCTTCTCCCCCTGCTCCTGGCTTG
GGCCATGGACACTCAGCACCTGGCTTGGCCTGCCTTGGTCTGGTCAGG
GCCTTCCTCATCCCCATCATGGGGGCACAGTCTATTTATTCTGCCCAG
CTCACCTTCAACAAGACATTGTCCAGGGACATCTTCCTCTCCTCCCCC
TTGCCCTGAACTTCCTTATCCCCTTTTTATTTATTGGGCAGGGGGAGG
GGTGAGGGCACAGGCAAGAAGAGAGTCACATTGTCCTGGGGTGAGGGG
GGGTCACAGTAATCATGGTCTGCTCCCTTCACCTGGCTAGGGGGCAGA
CTTAATAAAGAGCGAAACTCAAAAAAAAAAAAAAAAAAAAAAAAAAAA
AAAGG
PPP2R5A (Human)
>gi|5453950|ref|NP_006234.1| protein phosphatase
2, regulatory subunit B (B56), alpha isoform
[Homo sapiens]
MSSSSPPAGAASAAISASEKVDGFTRKSVRKAQRQKRSQGSSQFRSQG
SQAELHPLPQLKDATSNEQQELFCQKLQQCCILEDFMDSVSDLKSKEI
KRATLNELVEYVSTNRGVIVESAYSDIVKMISANIFRTLPPSDNPDFD
PEEDEPTLEASWPHIQLVYEFFLRFLESPDFQPSIAKRYIDQKFVQQL
LELFDSEDPRERDFLKTVLHRIYGKFLGLRAFIRKQINNIFLRFIYET
EHFNGVAELLEILGSIINGFALPLKAEHKQFLMKVLIPMHTAKGLALF
HAQLAYCVVQFLEKDTTLTEPVIRGLLKFWPKTCSQKEVMFLGEIEEI
LDVIEPTQFKKIEEPLFKQISKCVSSSHFQVAERALYFWNNEYILSLI
EENIDKILPIMFASLYKISKEHWNPTIVALVYNVLKTLMEMNGKLFDD
LTSSYKAERQREKKKELEREELWKKLEELKLKKALEKQNSAYNMHSIL
SNTSAE
>gi|30795205|ref|NM_006243.2| Homo sapiens protein
phosphatase 2, regulatory subunit B′, alpha
isoform (PPP2R5A), mRNA
CGCAGAGGGCCGGGGCTACGGGGCAGCGCCCCGGGCGATGAGGGGCCG
GCGTTGACCGGGAAGAGCGGGCACCGCGGCAGTGGCTCCGAGGGGACC
CGCGATGGCAGCGCCCTGAGAGGAGGCTCCAGGCAGGGCGGGCTGCGC
TGGCAGCGGCCGCTGAGGTGCTGGCCGGCCGGCTGGCTGGCGACGGGG
GCAGAAGCGACGAGAGGCGCGCTCGGCACCCGCACCCCCGTGCCCCCG
CCTCAGTTGTCTAAACTTCGGGCTCTCTTCCACCCGCTCTGCGCGCCC
AGAGTCAACAACTTCTTCACCCCCCTCCGCCCCCGCCCTTCCCTCCGT
CAGCCCCGGGAGCTCGCCGCGCGCCGGGGACCAGGAACCTCCAGCGCT
GAGATGTGGCCGTGAGGCGTTGGCGGGCGGCGAGGAGAAGCTCGGCGG
CGTCCCGGGGCCGGAGGGCCGTGGGGCCGGGGCGCAGGGGCGCGAGCA
CCCCGCGCCTCTCCCCCGCCTCCTCCTGCCGTCTCCGCCGCTGCCCGT
GCCTTGCAAGCAGCAGCCGGAGCTGCCAAGCGTCAGGGCCGCGGAGAT
GTCGTCGTCGTCGCCGCCGGCGGGGGCTGCCAGCGCCGCCATCTCGGC
CTCGGAGAAAGTGGACGGCTTCACCCGGAAATCGGTCCGCAAGGCGCA
GAGGCAGAAGCGCTCCCAGGGCTCGTCGCAGTTTCGCAGCCAGGGCAG
CCAGGCAGAGCTGCACCCGCTGCCCCAGCTCAAAGATGCCACTTCAAA
TGAACAACAAGAGCTTTTCTGTCAGAAGTTGCAGCAGTGTTGTATACT
GTTTGATTTCATGGACTCTGTTTCAGACTTGAAGAGCAAAGAAATTAA
AAGAGCAACACTGAATGAACTGGTTGAGTATGTTTCAACTAATCGTGG
TGTAATTGTTGAATCAGCGTATTCTGATATAGTAAAAATGATCAGTGC
TAACATCTTCCGTACACTTCCTCCAAGTGATAATCCAGATTTTGATCC
AGAAGAGGATGAACCCACGCTTGAGGCCTCTTGGCCTCACATACAGTT
GGTATATGAATTCTTCTTGAGATTTTTGGAGAGCCCTGATTTCCAGCC
TAGCATTGCAAAACGATACATTGATCAGAAATTCGTACAACAGCTCCT
GGAGCTTTTTGATAGTGAAGATCCCAGAGAACGTGACTTCCTGAAGAC
TGTTCTGCACCGAATTTATGGGAAATTTCTTGGATTAAGAGCATTCAT
CAGAAAACAAATTAACAACATTTTCCTCAGGTTTATATATGAAACAGA
ACATTTCAATGGTGTTGCTGAACTTCTTGAAATATTAGGAAGTATTAT
CAATGGCTTTGCATTGCCACTGAAAGCAGAACATAAACAATTTCTAAT
GAAGGTTCTTATTCCTATGCATACTGCAAAAGGATTAGCTTTGTTTCA
TGCTCAGCTAGCATATTGTGTTGTACAGTTCCTGGAGAAAGATACAAC
ACTAACAGAGCCAGTGATCAGAGGACTGCTGAAATTTTGGCCAAAAAC
CTGCAGTCAGAAAGAGGTGATGTTTTTAGGAGAAATTGAAGAAATCTT
AGATGTCATTGAACCAACACAGTTCAAAAAAATTGAAGAGCCACTTTT
CAAGCAGATATCCAAGTGTGTATCCAGTTCTCATTTTCAGGTTGCAGA
AAGGGCATTGTACTTCTGGAATAACGAATATATTCTTAGTTTGATTGA
GGAGAACATTGATAAAATTCTGCCAATTATGTTTGCCAGTTTGTACAA
AATTTCCAAAGAACACTGGAATCCGACCATTGTAGCACTGGTATACAA
TGTGCTGAAAACCCTAATGGAAATGAATGGCAAGCTTTTCGATGACCT
TACTAGCTCATACAAAGCTGAAAGACAGAGAGAGAAAAAGAAGGAATT
GGAACGTGAAGAATTATGGAAAAAATTAGAGGAGCTAAAGCTAAAGAA
AGCTCTAGAAAAACAGAATAGTGCTTACAACATGCACAGTATTCTCAG
CAATACAAGTGCCGAATAAAAAAAAAGCCTCCCACCTCTGCCGGATAG
GCAGAGTTTTGTATGCTTTTTTGAAATATGTAAAAATTACAAAACAAA
CCTCATCAGTATAATATAATTAAAAGGCCAATTTTTTCTGGCAACTGT
AAATGGAAAAATATATGGACTAAACGTAGCCCTGTGCTGTATCATGGC
CATAGTATATTGTAACCTTTGTCTAATCATTGGATTTATTGTGTCACT
TCTGAAGTTTCACAGAAATGAATGAATTTTATCATCTATGATATGAGT
GAGATAATTATGGGAGTGGTAAGAATTATGACTTGAATTCTTCTTTGA
TTGTGTTGCACATAGATATGGTAGTCTGCTCTGTATATTTTTCCCTTT
TATAATGTGCTTTTCACACTGCTGCAAACCTTAGTTACATCCTAGGAA
AAAATACTTCCTAAAATAAAACTAAGGTATCATCCTTACCCTTCTCTT
TGTCTCACCCAGAAATATGATGGGGGGAATTACCTGCCCTAACCCCTC
CCTCAATAAATACATTACTGTACTCTGGAATTTAGGCAAAACCTTAAA
TCTCCAGGCTTTTTAAAGCACAAAATATAAATAAAAGCTGGGAAAGTA
AACCAAAATTCTTCAGATTGTTCCTCATGAATATCCCCCTTCCTCTGC
AATTCTCCAGAGTGGTAACAGATGGGTAGAGGCAGCTCAGGTGAATTA
CCCAGCTTGCCTCTCAATTCATTCCTCCTCTTCCTCTCAAAGGCTGAA
GGCAGGGCCTTTCCAGTCCTCACAACCTGTCCTTCACCTAGTCCCTCC
TGACCCAGGGATGGAGGCTTTGAGTCCCACAGTGTGGTGATACAGAGC
ACTAGTTGTCACTGCCTGGCTTTATTTAAAGGAACTGCAGTAGGCTTC
CTCTGTAGAGCTCTGAAAAGGTTGACTATATAGAGGTCTTGTATGTTT
TTACTTGGTCAAGTATTTCTCACATCTTTTGTTATCAGAGTACCATTC
CAATCTCTTAACTTGCAGTTGTGTGGAAAACTGTTTTGTAATGAAAGA
TCTTCATTGGGGGATTGAGCAGCATTTAATAAAGTCTATGTTTGTATT
TTGCCTTAAAAAAAAAAAAAAAAAA
PPP2R5A (Mouse)
>gi|47059051|ref|NP_659129.2| protein phosphatase
2, regulatory subunit B (B56), alpha isoform
[Mus musculus]
MSSPSPPAPVACAAISASEKVDGFTRKSVRKAQRQKRSQGSSQFRSQG
SQAELHPLPQLKDATSNEQQELFCQKLQQCCVLEDFMDSVSDLKSKEI
KRATLNELVEYVSTNRGVIVESAYSDIVKMISANIFRTLPPSDNPDFD
PEEDEPTLEASWPHIQLVYEFFLRFLESPDFQPSIAKRYIDQKFVQQL
LELFDSEDPRERDFLKTVLHRIYGKFLGLRAFIRKQINNIFLRFIYET
EHFNGVAELLEILGSIINGFALPLKAEHKQFLMKVLIPMHTAKGLALF
HAQLAYCVVQFLEKDTTLTEPVIRGLLKFWPKTCSQKEVMFLGEIEEI
LDVIEPTQFKKIEEPLFKQISKCVSSSHFQVAERALYFWNNEYILSLI
EENIDKILPIMFASLYKISKEHWNQTIVALVYNVLKTLMEMNGKLFDD
LTSSYKAERQREKKKELEREELWKKLEELQLKKALEKQNNAYNMHSIR
SSTSAK
>gi|118130061|ref|NM_144880.4| Mus musculus protein
phosphatase 2, regulatory subunit B (B56), alpha
isoform (Ppp2r5a), mRNA
CCCCCCTCACCCGAACCAGCCACCCTCTCAAGTTGTAGCAGTTGCTTC
CCGGGCGTGCTCCGTGGGCGGCCGGTGGGCGCGGGAGGCTGAGCGAGG
GCGACGCTCCAGGGATCCAAGGATCCAAGGATCGGGGTACGGGTGGCC
GCCCCGGGCGATGAGGGGCCATCGCTGACAGGAGAACGAGCGCTCGGC
AGCGGCTCGCGCGGACCCGCGATGGAAGCGCCCCGGGAGGAGGCTCCC
GGCCGGGCGGACAGCGCGGGCGGCAGCCGCTGAAGACCTGTCCGGACT
CCGCGCGCTAAGGGGCCGCGAGGCGCGCCCGGCGTCCGCACCCGCGCG
CTCGCCCCAGCCGTCCAAACTTCGGGCTCGCCTCCGCCCGCCCTCGTC
GCCGGGCGTCAGCAACTTGTGCGGCCCGCGCGCCCCCGCCCTCCCCTC
CGCCAGCCCCGGGAGGCGGCCGCGCGGCGCGGGGATGCGGCCGTGAGG
CGCTGTCGGGTGAGGAGCAGCTCGCGGAGCGCGAGGGCGGCGCGGCCG
GGGCCCGGGGCGCAGGGGCGCAGGCCCGCCGTGCCCGTCCCCCGCCGA
TTGCCCGGGCCAGCCGCCGCGGGAGGCGCCGATCGCCCGGGTCGCCGA
GATGTCGTCGCCGTCGCCGCCCGCACCAGTTGCCTGCGCCGCCATCTC
GGCCTCGGAGAAAGTGGACGGCTTCACCCGGAAATCGGTGCGCAAGGC
GCAGAGGCAGAAGCGCTCTCAGGGCTCGTCGCAGTTCCGCAGCCAGGG
CAGCCAGGCGGAGCTGCACCCCCTGCCCCAGCTCAAAGATGCCACTTC
AAATGAACAGCAAGAGCTTTTCTGCCAGAAGCTACAACAGTGCTGTGT
ACTGTTTGATTTCATGGACTCTGTCTCAGACTTGAAGAGCAAAGAAAT
TAAAAGAGCGACGCTGAATGAACTGGTTGAGTATGTTTCAACTAATCG
TGGTGTAATTGTTGAATCAGCGTATTCTGATATAGTAAAAATGATCAG
TGCTAACATCTTCCGGACACTTCCTCCAAGTGATAATCCAGATTTTGA
CCCGGAAGAGGATGAGCCCACACTTGAGGCCTCTTGGCCTCACATACA
GTTGGTGTATGAATTCTTCTTGAGATTCTTGGAGAGTCCTGATTTCCA
GCCCAGCATTGCAAAGCGATACATTGATCAGAAGTTTGTCCAACAGCT
CCTGGAGCTTTTTGATAGTGAAGATCCACGGGAGCGCGACTTCCTGAA
GACCGTCCTGCATCGGATTTACGGCAAGTTCCTTGGCCTGAGAGCGTT
CATCAGAAAGCAAATTAACAACATTTTCCTCAGGTTTATATATGAAAC
GGAACATTTCAATGGTGTGGCTGAGCTCCTGGAAATATTGGGAAGTAT
CATCAATGGCTTTGCATTGCCACTGAAAGCAGAGCATAAGCAGTTTCT
AATGAAGGTTCTTATTCCTATGCATACTGCAAAAGGATTGGCCTTGTT
TCACGCACAGCTGGCGTACTGTGTTGTGCAGTTCCTGGAGAAAGACAC
AACGCTGACAGAGCCGGTGATCAGAGGACTGCTGAAATTTTGGCCAAA
AACATGCAGTCAGAAAGAGGTGATGTTTTTAGGAGAAATTGAAGAGAT
CTTAGATGTCATTGAACCAACACAATTCAAAAAAATTGAAGAGCCGCT
TTTTAAGCAGATATCCAAGTGCGTCTCCAGCTCTCATTTCCAGGTTGC
AGAAAGAGCACTGTACTTCTGGAATAATGAATATATTCTTAGTTTGAT
TGAAGAGAATATTGATAAAATCCTGCCAATCATGTTTGCCAGCTTGTA
TAAAATTTCCAAAGAACACTGGAATCAGACTATTGTAGCACTGGTGTA
CAATGTGCTGAAAACCCTCATGGAGATGAACGGCAAGCTTTTTGACGA
CCTTACTAGTTCCTACAAAGCCGAAAGACAGAGAGAGAAGAAGAAAGA
ACTGGAACGGGAAGAGTTGTGGAAAAAACTAGAGGAGCTGCAGCTGAA
GAAGGCTCTAGAGAAACAGAACAATGCTTACAACATGCACAGTATTCG
CAGCAGTACCAGTGCCAAATAAAGATCAGCTCCCCCTGCTGGGCGGTC
GTTTTGTACACTTTTTTTTTTTTTTGAATATATAAAAACTTCAGAGCA
GACCTCATCAGTATAATATAATTAGGAGGCCAGTTTTTCCTGGCAAGC
GTAAAAGCGAAAGAATTATGGACTAAAACATAGCCCTGTGCTGTATCA
CGGCCACAGTATATTGTAAACTCTGTCTAATCATGGATTGTGTCACTG
TCTCTGTTGAGTGAGGTGATCGTGGGAGTGGCAAGCGTGTGTTGCGAC
TTGAGCCCGGTTGTGCTGCACACACAGATGAAGCCGTCCTCTGCACAC
TTCCTTTATCATGTGTTTTCACCGTGCTGCACACCTTGGTGCTGCACA
CCTTGAGTACATCTGAGGAAAGAGCCTCGTAAGATAAGCGGAGGGGTT
GCCCTTCCCTCACCTCTCCTAGAGAGGTGTGGGCAGGGGACAAGAGCC
CAGCCTCATTAAAGACACTGCCATACTCTGGGTTTTACAACATCTGAC
ATTTCCAGGCTTCTGAAGCACAAAGTATTAAAGTTGGGGGGGGGAAGT
AAACCAAAATTCTGATGTTCCCAAATCCCCCCTTCAGCAGCGGCTCCC
CGGAGCGTGTGCGGAGCAGCACAGGCCACGGGTGGACCCGAGGCTCAC
CTCCTTCATTCCTCTCCTCCAAGGCTGGAGGCAGGGCCTTCCCAGTCC
TCACCCTGCCAGTCCCCAGGCCTGCCTGCCTGCAGGGTGGAGCTCTGG
GTCCCTCCCACAGTGTGATGCAGACTGCTAGCTGTCACTGCCTGGCTT
TATTTAAAGGAACTGCAGCAGGTGTCCTCAGAGCTGACTATGTAGAAG
CTTTGTCTGTTTTTACCTGGTCAGGTATTTTTCACACTGTTGTTACCA
GTACCATTCCAGCCTCTTGCCTTGCAGTTGTATGGAAAACTGTTTTAT
AATGAGAGATCTTTACTGAGGATTGAGCAGCATTTAATAAAGTCTATG
TTTGTATTTT
PPP2R5C (Human)
>gi|31083259|ref|NP_002710.2| gamma isoform of
regulatory subunit B56, protein phosphatase 2A
isoform a [Homo sapiens]
MLTCNKAGSRMVVDAANSNGPFQPVVLLHIRDVPPADQEKLFIQKLRQ
CCVLEDEVSDPLSDLKWKEVKRAALSEMVEYITHNRNVITEPIYPEVV
HMFAVNMERTLPPSSNPTGAEFDPEEDEPTLEAAWPHLQLVYEFFLRF
LESPDFQPNIAKKYIDQKFVLQLLELFDSEDPRERDFLKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQYTVYSQASTMSIPVAMETDGPLFEDVQMLRKTVKDEAH
QAQKDPKKDRPLARRKSELPQDPHTKKALEAHCRADELASQDGR
>gi|31083258|ref|NM_002719.2| Homo sapiens protein
phosphatase 2, regulatory subunit B′, gamma isoform
(PPP2R5C), transcript variant 1, mRNA
AGTTCCCTCCAGCTGCAGAGAGCTTCAGTTTGTCTTTTTTTTTTTAAA
CTAAAATGGAGGCTGGTTTCTTGCCTTAAGGAGCCCATTGCCTTTCCC
GCTGAAGTCTAGATGTTGACATGTAATAAAGCGGGCAGCAGGATGGTG
GTGGATGCGGCCAACTCCAATGGGCCTTTCCAGCCCGTGGTCCTTCTC
CATATTCGAGATGTTCCTCCTGCTGATCAAGAGAAGCTTTTTATCCAG
AAGTTACGTCAGTGTTGCGTCCTCTTTGACTTTGTTTCTGATCCACTA
AGTGACCTAAAGTGGAAGGAAGTAAAACGAGCTGCTTTAAGTGAAATG
GTAGAATATATCACCCATAATCGGAATGTGATCACAGAGCCTATTTAC
CCAGAAGTAGTCCATATGTTTGCAGTTAACATGTTTCGAACATTACCA
CCTTCCTCCAATCCTACGGGAGCGGAATTTGACCCGGAGGAAGATGAA
CCAACGTTAGAAGCAGCCTGGCCTCATCTACAGCTTGTTTATGAATTT
TTCTTAAGATTTTTAGAGTCTCCAGATTTCCAACCTAATATAGCGAAG
AAATATATTGATCAGAAGTTTGTATTGCAGCTTTTAGAGCTCTTTGAC
AGTGAAGATCCTCGGGAGAGAGATTTTCTTAAAACCACCCTTCACAGA
ATCTATGGGAAATTCCTAGGCTTGAGAGCTTACATCAGAAAACAGATA
AATAATATATTTTATAGGTTTATTTATGAAACAGAGCATCATAATGGC
ATAGCAGAGTTACTGGAAATATTGGGAAGTATAATTAATGGATTTGCC
TTACCACTAAAAGAAGAGCACAAGATTTTCTTATTGAAGGTGTTACTA
CCTTTGCACAAAGTGAAATCTCTGAGTGTCTACCATCCCCAGCTGGCA
TACTGTGTAGTGCAGTTTTTAGAAAAGGACAGCACCCTCACGGAACCA
GTGGTGATGGCACTTCTCAAATACTGGCCAAAGACTCACAGTCCAAAA
GAAGTAATGTTCTTAAACGAATTAGAAGAGATTTTAGATGTCATTGAA
CCATCAGAATTTGTGAAGATCATGGAACCCCTCTTCCGGCAGTTGGCC
AAATGTGTCTCCAGCCCACACTTCCAGGTGGCAGAGCGAGCTCTCTAT
TACTGGAATAATGAATACATCATGAGTTTAATCAGTGACAACGCAGCG
AAGATTCTGCCCATCATGTTTCCTTCCTTGTACCGCAACTCAAAGACC
CATTGGAACAAGACAATACATGGCTTGATATACAACGCCCTGAAGCTC
TTCATGGAGATGAACCAAAAGCTATTTGATGACTGTACACAACAGTTC
AAAGCAGAGAAACTAAAAGAGAAGCTAAAAATGAAAGAACGGGAAGAA
GCATGGGTTAAAATAGAAAATCTAGCCAAAGCCAATCCCCAGTACACA
GTGTATAGTCAAGCCAGCACCATGAGCATTCCGGTTGCAATGGAGACA
GATGGGCCTTTATTTGAAGATGTGCAGATGCTGAGAAAGACAGTGAAG
GACGAGGCTCATCAGGCACAGAAAGATCCGAAGAAGGACCGTCCTCTT
GCACGCCGCAAGTCCGAGCTGCCTCAGGACCCCCACACCAAGAAAGCC
TTGGAAGCTCACTGCAGGGCCGATGAGCTGGCCTCCCAGGACGGCCGC
TAGCCTCCGGGGCGCCGCGTCGGGGCCGGGCCCGCCAGTTCTTTTCCG
GATTCTGTAGAAAATACATACTTCCTGTGCCATACCAATCAGTTACAC
TCAAAGCTTTCTTGGACCCCGTTCCGTAGGCAATAACGTGCGTCCGCC
TCAGCGCGAGATTAGGAGTTCAAACAATGGTGACTTCCCAGAGCCCGC
TGGCAGAGCCGCGGGTTGACGACGGTGTCCTCGCAGTGTCGCCGCCAC
CCCAGCGTAGTCCAAGTCAGACTATTTCACAAAGTCAGAGCGATAGGA
AAGCACCCTGCCCTTCATCTTCATGTTCTCCCAAATGGAACTTAGGAT
CTTTTAACATAGGTGGTTCTGTGATAACATCAGTGTTTTCCAAATCAA
AGGAACGCTTTAAAAAATAGGACCTATTTTTTAAGACTTTACAGCCTT
TGAAATGGTTTCCACGTGATTGTTACGCCAGCAGTTCTCGTTTTGTTT
GTTTTTCAATCTCAGTGAAATGGCTCTTTGCTTTCGAGTTCTCACGCA
ACGTACTGGGCAAATGACAATCCTCAGCCGCTGGTATTTTCTAAGGGG
TCTCTTCACTTTGATGAGTGACATGAACACCGTGTCTCCTTCTCTTGT
GTGTACCTAAAGCCATATTTCCAAGTCTGTGGTACTCCAGGATTCCAG
GAGTAAGCCTGTAGAAGAGATTTATTTTAAAAGAGATTGCTCTGAAAT
TTATCTTAAAAGAGCTTGCTCTGTCTACCTTGACAGAAATTGGAGTTT
TAAAATTATGTGTTAATATTTTTATTTGCAGATTTCGTTTCCGTCAAC
TTAAACATTGTTGCCCTTCAACAAGGCTCTTGAATTAATAAAATTATA
GTCTCTAAGAATTCCACATTTTATGGAAAGTTAGAGCAAAATCATTTT
GAGTTAAGCCAGTTCTTAGCCTAATGCAAACTGCAGCGCCTTTAAGCA
TAAAGTAACACAACAGCATTGCACGGGGCCGGCACTGCCGCTGCCTTC
ACTGAAGGCTGCAGTGCTGTTCTGAGAGCTTGGAGGAGGCACCAGCGA
GGATGACGTTTAGTGGAGCTCTTTCTGTTGAAAAGAGCTCACGTTATC
AACACCTTGTAAGGAAAATACAGTGTCTGAGTTTTCATCGGTCTTCAC
ATGCTGCTATATATTCCACAGAGTTCCTTGCATGTACTGAGCTTTTGT
TTTAGATGGAATAGCACAAGGAGAAAAATCTTTAAACTTAGTGCTTTG
TCTATTCTTTATTTCTCTCAGGGTGGCCAGTATTTTGACTTATTTATC
CTGCTTGAAAGCTACTTGAGATGTGTACTGCTATTCTAAACACGTGAT
CTAGTTTCTTTCATCTCTGGCATAAGATTATATAACTTAATGTTAAGT
GTCTTGAGGCATAAAAGACAAAATGTGGCTTATTTTAGGATCTGTTTT
TTCATCGAGGTCTCGGGTATCCTTTCAAAGATAGTGAGAAGCAGACAC
TGCTCCTTGTGCAGCTCTGGTACCTCCTGCCCACTGCTGTCACTTCAA
GCCACTGGCAATGCTTCTGTCCTCGTGTCTTGGAGGAAAATCACCTGG
GGGGAGGGGACTTCTTGTGGTAAGAGCAAGTGCAGGTATGAAATGCGA
AGATTGCCCCAGCTAAAAGTGGACAAGTCCGCTTTGTGAGATGAATAC
TTCCTGAGAAACTTGACAAGTATCTCTCCATTTTACCATTATGAAAAC
TATCATTAAAAAAAACAGTTTAGATGCCTTCTCCTTTTGAGGGAAAAA
GGGTGCTTTTTATTGTATAAAGCAGCGTCTTATGTATTTTGATATACC
ATTGTTTGAACTTCCGTCTTTAGCTGATAGATTCTCAAATATCCTTGA
TTTTGGATGTTCAGTATGTTTGTGAGAGAGGTTTCTGGGAAGACTCTC
TTTTTGCCCTCGGGAAAAAGCAAAATATCAATGTTTGGGTGACTGTGT
AAAGCTCAGTGTGTAAGAACATCTTTTTGTCTAGGTTTTCTTTCTGCT
CTTTATTGAAGACAAACACTCACCAAAAAGAAAAATAAAAGTTTTCAG
AGAAACTAATTTTCTTTGGCAAGAGTATTACTTAATATTTTGGCCTCC
TAAAGTTTCCCTAGTTAGTACTCGGACTCCTGTGCTAATTGTCAGCTT
ACATATCATTGTATAGAGACTGTTTATTCTGTACCAAACTGATTTCAA
AAGTACTACATTGAAAATAAACCGGTGACTGTTTTTCTTCATAAAGTT
CTGCGTTTGGCATCTTCACTCTTTCCAAAATGTATCTGTACATCAGAA
ATGTCACTATTCCAAGTGTCTTTTTAGTGTGGCTTTAGTATGGCTTCC
TTTTAATATTGTACATACATTGTATCTTTGTTTTATGGTAATAAGTAA
TAAAAATGTAGACTTCATATTTTGTACAAAATGTCCTATGTACAGAAT
AAAAAAGTTCATAGAAACAGCAAAAAAAAAAAAAAAAAA
>gi|31083250|ref|NP_848703.1| gamma isoform of
regulatory subunit B56, protein phosphatase 2A
isoform d [Homo sapiens]
MLTCNKAGSRMVVDAANSNGPFQPVVLLHIRDVPPADQEKLFIQKLRQ
CCVLEDEVSDPLSDLKWKEVKRAALSEMVEYITHNRNVITEPIYPEVV
HMFAVNMERTLPPSSNPTGAEFDPEEDEPTLEAAWPHLQLVYEFFLRF
LESPDFQPNIAKKYIDQKFVLQLLELFDSEDPRERDELKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
>gi|31083249|ref|NM_178588.1| Homo sapiens protein
phosphatase 2, regulatory subunit B′, gamma isoform
(PPP2R5C), transcript variant 4, mRNA
AGTTCCCTCCAGCTGCAGAGAGCTTCAGTTTGTCTTTTTTTTTTTAAA
CTAAAATGGAGGCTGGTTTCTTGCCTTAAGGAGCCCATTGCCTTTCCC
GCTGAAGTCTAGATGTTGACATGTAATAAAGCGGGCAGCAGGATGGTG
GTGGATGCGGCCAACTCCAATGGGCCTTTCCAGCCCGTGGTCCTTCTC
CATATTCGAGATGTTCCTCCTGCTGATCAAGAGAAGCTTTTTATCCAG
AAGTTACGTCAGTGTTGCGTCCTCTTTGACTTTGTTTCTGATCCACTA
AGTGACCTAAAGTGGAAGGAAGTAAAACGAGCTGCTTTAAGTGAAATG
GTAGAATATATCACCCATAATCGGAATGTGATCACAGAGCCTATTTAC
CCAGAAGTAGTCCATATGTTTGCAGTTAACATGTTTCGAACATTACCA
CCTTCCTCCAATCCTACGGGAGCGGAATTTGACCCGGAGGAAGATGAA
CCAACGTTAGAAGCAGCCTGGCCTCATCTACAGCTTGTTTATGAATTT
TTCTTAAGATTTTTAGAGTCTCCAGATTTCCAACCTAATATAGCGAAG
AAATATATTGATCAGAAGTTTGTATTGCAGCTTTTAGAGCTCTTTGAC
AGTGAAGATCCTCGGGAGAGAGATTTTCTTAAAACCACCCTTCACAGA
ATCTATGGGAAATTCCTAGGCTTGAGAGCTTACATCAGAAAACAGATA
AATAATATATTTTATAGGTTTATTTATGAAACAGAGCATCATAATGGC
ATAGCAGAGTTACTGGAAATATTGGGAAGTATAATTAATGGATTTGCC
TTACCACTAAAAGAAGAGCACAAGATTTTCTTATTGAAGGTGTTACTA
CCTTTGCACAAAGTGAAATCTCTGAGTGTCTACCATCCCCAGCTGGCA
TACTGTGTAGTGCAGTTTTTAGAAAAGGACAGCACCCTCACGGAACCA
GTGGTGATGGCACTTCTCAAATACTGGCCAAAGACTCACAGTCCAAAA
GAAGTAATGTTCTTAAACGAATTAGAAGAGATTTTAGATGTCATTGAA
CCATCAGAATTTGTGAAGATCATGGAACCCCTCTTCCGGCAGTTGGCC
AAATGTGTCTCCAGCCCACACTTCCAGGTGGCAGAGCGAGCTCTCTAT
TACTGGAATAATGAATACATCATGAGTTTAATCAGTGACAACGCAGCG
AAGATTCTGCCCATCATGTTTCCTTCCTTGTACCGCAACTCAAAGACC
CATTGGAACAAGTAAGAAAGAACTGGCTGCCATCTTTTTCAGTCATTT
TAAAATATGGCACGTTTTACTGCTACTTCAGTAAGAATAAATATCAGA
ATTTTAAATATCAATTAAAAAACAAGAAGGTCAAAAAAAAAAAAAAAA
AAAAAAAAAAAAAAAAAAAAAAAAAAAAAAAAAAAAAAAAAAAAAAAA
AAAAAAAAAAAAAAAAAAAA
>gi|31083243|ref|NP_848702.1| gamma isoform of
regulatory subunit B56, protein phosphatase 2A
isoform c [Homo sapiens]
MLTCNKAGSRMVVDAANSNGPFQPVVLLHIRDVPPADQEKLFIQKLRQ
CCVLEDEVSDPLSDLKWKEVKRAALSEMVEYITHNRNVITEPIYPEVV
HMFAVNMERTLPPSSNPTGAEFDPEEDEPTLEAAWPHLQLVYEFFLRF
LESPDFQPNIAKKYIDQKFVLQLLELFDSEDPRERDFLKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQVLKKRIT
>gi|31083242|ref|NM_178587.1| Homo sapiens protein
phosphatase 2, regulatory subunit B′, gamma isoform
(PPP2R5C), transcript variant 3, mRNA
AGTTCCCTCCAGCTGCAGAGAGCTTCAGTTTGTCTTTTTTTTTTTAAA
CTAAAATGGAGGCTGGTTTCTTGCCTTAAGGAGCCCATTGCCTTTCCC
GCTGAAGTCTAGATGTTGACATGTAATAAAGCGGGCAGCAGGATGGTG
GTGGATGCGGCCAACTCCAATGGGCCTTTCCAGCCCGTGGTCCTTCTC
CATATTCGAGATGTTCCTCCTGCTGATCAAGAGAAGCTTTTTATCCAG
AAGTTACGTCAGTGTTGCGTCCTCTTTGACTTTGTTTCTGATCCACTA
AGTGACCTAAAGTGGAAGGAAGTAAAACGAGCTGCTTTAAGTGAAATG
GTAGAATATATCACCCATAATCGGAATGTGATCACAGAGCCTATTTAC
CCAGAAGTAGTCCATATGTTTGCAGTTAACATGTTTCGAACATTACCA
CCTTCCTCCAATCCTACGGGAGCGGAATTTGACCCGGAGGAAGATGAA
CCAACGTTAGAAGCAGCCTGGCCTCATCTACAGCTTGTTTATGAATTT
TTCTTAAGATTTTTAGAGTCTCCAGATTTCCAACCTAATATAGCGAAG
AAATATATTGATCAGAAGTTTGTATTGCAGCTTTTAGAGCTCTTTGAC
AGTGAAGATCCTCGGGAGAGAGATTTTCTTAAAACCACCCTTCACAGA
ATCTATGGGAAATTCCTAGGCTTGAGAGCTTACATCAGAAAACAGATA
AATAATATATTTTATAGGTTTATTTATGAAACAGAGCATCATAATGGC
ATAGCAGAGTTACTGGAAATATTGGGAAGTATAATTAATGGATTTGCC
TTACCACTAAAAGAAGAGCACAAGATTTTCTTATTGAAGGTGTTACTA
CCTTTGCACAAAGTGAAATCTCTGAGTGTCTACCATCCCCAGCTGGCA
TACTGTGTAGTGCAGTTTTTAGAAAAGGACAGCACCCTCACGGAACCA
GTGGTGATGGCACTTCTCAAATACTGGCCAAAGACTCACAGTCCAAAA
GAAGTAATGTTCTTAAACGAATTAGAAGAGATTTTAGATGTCATTGAA
CCATCAGAATTTGTGAAGATCATGGAACCCCTCTTCCGGCAGTTGGCC
AAATGTGTCTCCAGCCCACACTTCCAGGTGGCAGAGCGAGCTCTCTAT
TACTGGAATAATGAATACATCATGAGTTTAATCAGTGACAACGCAGCG
AAGATTCTGCCCATCATGTTTCCTTCCTTGTACCGCAACTCAAAGACC
CATTGGAACAAGACAATACATGGCTTGATATACAACGCCCTGAAGCTC
TTCATGGAGATGAACCAAAAGCTATTTGATGACTGTACACAACAGTTC
AAAGCAGAGAAACTAAAAGAGAAGCTAAAAATGAAAGAACGGGAAGAA
GCATGGGTTAAAATAGAAAATCTAGCCAAAGCCAATCCCCAGGTACTA
AAAAAGAGAATAACATGAAAACGCCCAGGGTTACTTGAATGTTTTTAT
AAGATAGGAATATATGTCTTCACCATGGGGGGGGTCTCGATTTCACTA
ACGTTGTATATGAAAATGTCTGCAATAAAAAGTACTTTTAAACTTTGT
AA
>gi|31083236|ref|NP_848701.1| gamma isoform of
regulatory subunit B56, protein phosphatase 2A
isoform b [Homo sapiens]
MLTCNKAGSRMVVDAANSNGPFQPVVLLHIRDVPPADQEKLFIQKLRQ
CCVLEDEVSDPLSDLKWKEVKRAALSEMVEYITHNRNVITEPIYPEVV
HMFAVNMERTLPPSSNPTGAEFDPEEDEPTLEAAWPHLQLVYEFFLRF
LESPDFQPNIAKKYIDQKFVLQLLELFDSEDPRERDELKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQAQKDPKKDRPLARRKSELPQDPHTKKALEAHCRADELA
SQDGR
>gi|31083235|ref|NM_178586.1|Homo sapiens protein
phosphatase 2, regulatory subunit B′, gamma isoform
(PPP2R5C), transcript variant 2, mRNA
AGTTCCCTCCAGCTGCAGAGAGCTTCAGTTTGTCTTTTTTTTTTTAAA
CTAAAATGGAGGCTGGTTTCTTGCCTTAAGGAGCCCATTGCCTTTCCC
GCTGAAGTCTAGATGTTGACATGTAATAAAGCGGGCAGCAGGATGGTG
GTGGATGCGGCCAACTCCAATGGGCCTTTCCAGCCCGTGGTCCTTCTC
CATATTCGAGATGTTCCTCCTGCTGATCAAGAGAAGCTTTTTATCCAG
AAGTTACGTCAGTGTTGCGTCCTCTTTGACTTTGTTTCTGATCCACTA
AGTGACCTAAAGTGGAAGGAAGTAAAACGAGCTGCTTTAAGTGAAATG
GTAGAATATATCACCCATAATCGGAATGTGATCACAGAGCCTATTTAC
CCAGAAGTAGTCCATATGTTTGCAGTTAACATGTTTCGAACATTACCA
CCTTCCTCCAATCCTACGGGAGCGGAATTTGACCCGGAGGAAGATGAA
CCAACGTTAGAAGCAGCCTGGCCTCATCTACAGCTTGTTTATGAATTT
TTCTTAAGATTTTTAGAGTCTCCAGATTTCCAACCTAATATAGCGAAG
AAATATATTGATCAGAAGTTTGTATTGCAGCTTTTAGAGCTCTTTGAC
AGTGAAGATCCTCGGGAGAGAGATTTTCTTAAAACCACCCTTCACAGA
ATCTATGGGAAATTCCTAGGCTTGAGAGCTTACATCAGAAAACAGATA
AATAATATATTTTATAGGTTTATTTATGAAACAGAGCATCATAATGGC
ATAGCAGAGTTACTGGAAATATTGGGAAGTATAATTAATGGATTTGCC
TTACCACTAAAAGAAGAGCACAAGATTTTCTTATTGAAGGTGTTACTA
CCTTTGCACAAAGTGAAATCTCTGAGTGTCTACCATCCCCAGCTGGCA
TACTGTGTAGTGCAGTTTTTAGAAAAGGACAGCACCCTCACGGAACCA
GTGGTGATGGCACTTCTCAAATACTGGCCAAAGACTCACAGTCCAAAA
GAAGTAATGTTCTTAAACGAATTAGAAGAGATTTTAGATGTCATTGAA
CCATCAGAATTTGTGAAGATCATGGAACCCCTCTTCCGGCAGTTGGCC
AAATGTGTCTCCAGCCCACACTTCCAGGTGGCAGAGCGAGCTCTCTAT
TACTGGAATAATGAATACATCATGAGTTTAATCAGTGACAACGCAGCG
AAGATTCTGCCCATCATGTTTCCTTCCTTGTACCGCAACTCAAAGACC
CATTGGAACAAGACAATACATGGCTTGATATACAACGCCCTGAAGCTC
TTCATGGAGATGAACCAAAAGCTATTTGATGACTGTACACAACAGTTC
AAAGCAGAGAAACTAAAAGAGAAGCTAAAAATGAAAGAACGGGAAGAA
GCATGGGTTAAAATAGAAAATCTAGCCAAAGCCAATCCCCAGGCACAG
AAAGATCCGAAGAAGGACCGTCCTCTTGCACGCCGCAAGTCCGAGCTG
CCTCAGGACCCCCACACCAAGAAAGCCTTGGAAGCTCACTGCAGGGCC
GATGAGCTGGCCTCCCAGGACGGCCGCTAGCCTCCGGGGCGCCGCGTC
GGGGCCGGGCCCGCCAGTTCTTTTCCGGATTCTGTAGAAAATACATAC
TTCCTGTGCCATACCAATCAGTTACACTCAAAGCTTTCTTGGACCCCG
TTCCGTAGGCAATAACGTGCGTCCGCCTCAGCGCGAGATTAGGAGTTC
AAACAATGGTGACTTCCCAGAGCCCGCTGGCAGAGCCGCGGGTTGACG
ACGGTGTCCTCGCAGTGTCGCCGCCACCCCAGCGTAGTCCAAGTCAGA
CTATTTCACAAAGTCAGAGCGATAGGAAAGCACCCTGCCCTTCATCTT
CATGTTCTCCCAAATGGAACTTAGGATCTTTTAACATAGGTGGTTCTG
TGATAACATCAGTGTTTTCCAAATCAAAGGAACGCTTTAAAAAATAGG
ACCTATTTTTTAAGACTTTACAGCCTTTGAAATGGTTTCCACGTGATT
GTTACGCCAGCAGTTCTCGTTTTGTTTGTTTTTCAATCTCAGTGAAAT
GGCTCTTTGCTTTCGAGTTCTCACGCAACGTACTGGGCAAATGACAAT
CCTCAGCCGCTGGTATTTTCTAAGGGGTCTCTTCACTTTGATGAGTGA
CATGAACACCGTGTCTCCTTCTCTTGTGTGTACCTAAAGCCATATTTC
CAAGTCTGTGGTACTCCAGGATTCCAGGAGTAAGCCTGTAGAAGAGAT
TTATTTTAAAAGAGATTGCTCTGAAATTTATCTTAAAAGAGCTTGCTC
TGTCTACCTTGACAGAAATTGGAGTTTTAAAATTATGTGTTAATATTT
TTATTTGCAGATTTCGTTTCCGTCAACTTAAACATTGTTGCCCTTCAA
CAAGGCTCTTGAATTAATAAAATTATAGTCTCTAAGAATTCCACATTT
TATGGAAAGTTAGAGCAAAATCATTTTGAGTTAAGCCAGTTCTTAGCC
TAATGCAAACTGCAGCGCCTTTAAGCATAAAGTAACACAACAGCATTG
CACGGGGCCGGCACTGCCGCTGCCTTCACTGAAGGCTGCAGTGCTGTT
CTGAGAGCTTGGAGGAGGCACCAGCGAGGATGACGTTTAGTGGAGCTC
TTTCTGTTGAAAAGAGCTCACGTTATCAACACCTTGTAAGGAAAATAC
AGTGTCTGAGTTTTCATCGGTCTTCACATGCTGCTATATATTCCACAG
AGTTCCTTGCATGTACTGAGCTTTTGTTTTAGATGGAATAGCACAAGG
AGAAAAATCTTTAAACTTAGTGCTTTGTCTATTCTTTATTTCTCTCAG
GGTGGCCAGTATTTTGACTTATTTATCCTGCTTGAAAGCTACTTGAGA
TGTGTACTGCTATTCTAAACACGTGATCTAGTTTCTTTCATCTCTGGC
ATAAGATTATATAACTTAATGTTAAGTGTCTTGAGGCATAAAAGACAA
AATGTGGCTTATTTTAGGATCTGTTTTTTCATCGAGGTCTCGGGTATC
CTTTCAAAGATAGTGAGAAGCAGACACTGCTCCTTGTGCAGCTCTGGT
ACCTCCTGCCCACTGCTGTCACTTCAAGCCACTGGCAATGCTTCTGTC
CTCGTGTCTTGGAGGAAAATCACCTGGGGGGAGGGGACTTCTTGTGGT
AAGAGCAAGTGCAGGTATGAAATGCGAAGATTGCCCCAGCTAAAAGTG
GACAAGTCCGCTTTGTGAGATGAATACTTCCTGAGAAACTTGACAAGT
ATCTCTCCATTTTACCATTATGAAAACTATCATTAAAAAAAACAGTTT
AGATGCCTTCTCCTTTTGAGGGAAAAAGGGTGCTTTTTATTGTATAAA
GCAGCGTCTTATGTATTTTGATATACCATTGTTTGAACTTCCGTCTTT
AGCTGATAGATTCTCAAATATCCTTGATTTTGGATGTTCAGTATGTTT
GTGAGAGAGGTTTCTGGGAAGACTCTCTTTTTGCCCTCGGGAAAAAGC
AAAATATCAATGTTTGGGTGACTGTGTAAAGCTCAGTGTGTAAGAACA
TCTTTTTGTCTAGGTTTTCTTTCTGCTCTTTATTGAAGACAAACACTC
ACCAAAAAGAAAAATAAAAGTTTTCAGAGAAACTAATTTTCTTTGGCA
AGAGTATTACTTAATATTTTGGCCTCCTAAAGTTTCCCTAGTTAGTAC
TCGGACTCCTGTGCTAATTGTCAGCTTACATATCATTGTATAGAGACT
GTTTATTCTGTACCAAACTGATTTCAAAAGTACTACATTGAAAATAAA
CCGGTGACTGTTTTTCTTCATAAAGTTCTGCGTTTGGCATCTTCACTC
TTTCCAAAATGTATCTGTACATCAGAAATGTCACTATTCCAAGTGTCT
TTTTAGTGTGGCTTTAGTATGGCTTCCTTTTAATATTGTACATACATT
GTATCTTTGTTTTATGGTAATAAGTAATAAAAATGTAGACTTCATATT
TTGTACAAAATGTCCTATGTACAGAATAAAAAAGTTCATAGAAACAGC
AAAAAAAAAAAAAAAAAA
PPP2R5C (Mouse)
>gi|125346154|ref|NP_001074927.1| gamma isoform
of regulatory subunit B56, protein phosphatase
2A isoform c [Mus musculus]
MLTCNKAGSGMVVDAASSNGPFQPVALLHIRDVPPADQEKLFIQKLRQ
CCVLEDEVSDPLSDLKWKEVKRAALSEMVEYITHNRNVITEPIYPEAV
HMFAVNMERTLPPSSNPTGAEFDPEEDEPTLEAAWPHLQLVYEFFLRF
LESPDFQPNIAKKYIDQKFVLQLLELFDSEDPRERDELKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQVLKKRVTREC
>gi|125346153|ref|NM_001081458.1| Mus musculus
protein phosphatase 2, regulatory subunit B (B56),
gamma isoform (Ppp2r5c), transcript variant 3, mRNA
CGAAGCAGCCAGTTCCCTCCAGCTGCAGAGAGCTTCAGTTTGTCTTTT
TTTTTTTTTAAACTAAAATGGAGGCTGGTTTCTTGCCTTAAGGAGTAC
AGCGCCCTTCCCGCTGGAGCCTAGATGTTGACATGTAATAAAGCGGGC
AGCGGGATGGTGGTGGATGCGGCCAGCTCCAACGGGCCTTTCCAGCCC
GTGGCCCTTCTCCACATTCGAGATGTTCCTCCTGCGGATCAAGAGAAG
CTTTTTATCCAGAAGCTACGCCAGTGTTGTGTCCTCTTTGACTTTGTC
TCTGACCCACTGAGTGACCTGAAGTGGAAGGAAGTAAAGCGCGCTGCG
CTGAGCGAGATGGTGGAGTATATCACCCACAACCGGAACGTGATCACG
GAGCCCATTTACCCCGAGGCCGTCCACATGTTTGCAGTTAACATGTTC
CGAACCTTGCCACCTTCCTCCAATCCCACGGGAGCAGAATTCGACCCA
GAAGAGGATGAACCAACGTTAGAAGCAGCCTGGCCTCATCTGCAGCTT
GTTTATGAATTTTTCTTAAGATTTTTAGAGTCTCCAGATTTCCAACCC
AATATAGCAAAGAAATATATTGATCAGAAGTTTGTATTGCAGCTTCTA
GAGCTGTTTGACAGCGAGGATCCTCGGGAGAGAGATTTTCTAAAAACC
ACCCTGCACAGAATCTATGGGAAGTTCTTAGGCCTGCGTGCTTACATC
AGGAAACAGATCAATAATATATTTTATAGGTTTATCTATGAGACAGAG
CATCACAATGGCATAGCGGAGTTACTGGAGATCCTGGGAAGTATAATT
AATGGATTTGCCTTACCACTGAAGGAGGAACACAAGATTTTCCTGCTG
AAGGTGTTGCTGCCCTTGCACAAAGTGAAGTCCCTGAGTGTCTACCAT
CCCCAGCTGGCGTACTGTGTCGTGCAGTTTTTAGAGAAGGACAGCACC
CTCACTGAACCAGTGGTAATGGCACTTCTCAAATACTGGCCAAAGACT
CACAGTCCAAAAGAAGTAATGTTCTTAAATGAATTAGAAGAAATTTTA
GATGTAATTGAACCATCAGAGTTTGTGAAGATCATGGAGCCTCTTTTC
CGACAGTTAGCCAAATGTGTTTCCAGCCCTCACTTCCAGGTGGCCGAG
CGGGCGCTCTATTACTGGAACAACGAGTACATCATGAGTTTAATCAGT
GACAACGCAGCGAAGATTCTGCCCATCATGTTTCCGTCCTTATACCGC
AACTCAAAGACCCACTGGAACAAGACAATACACGGCTTGATATACAAC
GCCCTGAAACTCTTCATGGAGATGAACCAAAAACTCTTCGATGACTGC
ACTCAGCAGTTCAAAGCAGAGAAACTCAAAGAGAAGCTAAAAATGAAA
GAGCGAGAAGAAGCATGGGTTAAAATAGAAAATCTAGCCAAAGCGAAT
CCCCAGGTACTAAAAAAGAGAGTAACTCGGGAGTGTTGAGGCTTTGCG
TGAATGTCTGAGATAGGGCCTGGCTCCACCCCAGGAAGGGAGGCCAAC
GTCACTAACACTGTATGTGCAAATGTCCGCAATAAAACACTTTCCAAC
TTTGTAACTTCCTCTTGTATAAGTACTTATTTGCCACACGTAACTTTT
ACCACAGAATCGATTTTTTTCTCTTCTTTTTAAAAGTAAGATGTGATG
TGGTAAAGAGAACACCAGGATGTAACCTCTAAGATTGTAATGTCCTTT
CTTGCTCGAATGTCATAGATGCTGTCACTTGAACCGTGTTCCTCCGTT
TTATTCTCATACATGAGAGGGATGGGGGGGAGGCAGATGAAGAATGCT
GAAAACTAACTGAATTCTGCCCTGCTCACACTAACTGTTCCTCTACCC
TAGCCGATGCAGTGTGCTCCCGCAAGGCGAAGCCAGGCAGATGGTCCT
GTCTGTCAGAGCTGCAGGTGACTCAGCAGCCTCTGTCCAGGGTTGGCT
TTTAGGCTGGCAGAGCCTGGGACAGCTTTGCAGCCATAGCTCTTTGGT
GTTGCCTCCGCTGGTCCGAATGATCAGTTATTGGGGTGTTGCCTCTGC
TGGTCCGAATGATCAGTTATTGGGGTGTTGCCTCTGCTGCTCCGAATG
ATCAATGTTACTGGGTGTTTCTTTTCTCCTTGTTGCGTGTGTCTGATT
ATAACAGCCACTTGATGAAATGTCTGACCTTCTCCACACTAAGATCTC
TCAGGCTTCTCTCAGCTCTCCAGGGAAGAAAATATCTTACTAAACAGT
GTATTTCTTTTTGGTTGAGAAGTGTGTATTAAGTGTGTACATAAATTA
TTATGTAAGTTGGATGTGGGTTTTTTTAATCTTTGTCATTTAATAAAA
ACAATATATGAAGAT
>gi|125346020|ref|NP_036153.2| gamma isoform of
regulatory subunit B56, protein phosphatase 2A
isoform a [Mus musculus]
MLTCNKAGSGMVVDAASSNGPFQPVALLHIRDVPPADQEKLFIQKLRQ
CCVLEDEVSDPLSDLKWKEVKRAALSEMVEYITHNRNVITEPIYPEAV
HMFAVNMERTLPPSSNPTGAEFDPEEDEPTLEAAWPHLQLVYEFFLRF
LESPDFQPNIAKKYIDQKFVLQLLELFDSEDPRERDELKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQYAVYSQASAVSIPVAMETDGPQFEDVQMLKKTVSDEAR
QAQKELKKDRPLVRRKSELPQDPHTEKALEAHCRASELLSQDGR
>gi|125346019|ref|NM_012023.2| Mus musculus protein
phosphatase 2, regulatory subunit B (B56), gamma
isoform (Ppp2r5c), transcript variant 1, mRNA
CGAAGCAGCCAGTTCCCTCCAGCTGCAGAGAGCTTCAGTTTGTCTTTT
TTTTTTTTTAAACTAAAATGGAGGCTGGTTTCTTGCCTTAAGGAGTAC
AGCGCCCTTCCCGCTGGAGCCTAGATGTTGACATGTAATAAAGCGGGC
AGCGGGATGGTGGTGGATGCGGCCAGCTCCAACGGGCCTTTCCAGCCC
GTGGCCCTTCTCCACATTCGAGATGTTCCTCCTGCGGATCAAGAGAAG
CTTTTTATCCAGAAGCTACGCCAGTGTTGTGTCCTCTTTGACTTTGTC
TCTGACCCACTGAGTGACCTGAAGTGGAAGGAAGTAAAGCGCGCTGCG
CTGAGCGAGATGGTGGAGTATATCACCCACAACCGGAACGTGATCACG
GAGCCCATTTACCCCGAGGCCGTCCACATGTTTGCAGTTAACATGTTC
CGAACCTTGCCACCTTCCTCCAATCCCACGGGAGCAGAATTCGACCCA
GAAGAGGATGAACCAACGTTAGAAGCAGCCTGGCCTCATCTGCAGCTT
GTTTATGAATTTTTCTTAAGATTTTTAGAGTCTCCAGATTTCCAACCC
AATATAGCAAAGAAATATATTGATCAGAAGTTTGTATTGCAGCTTCTA
GAGCTGTTTGACAGCGAGGATCCTCGGGAGAGAGATTTTCTAAAAACC
ACCCTGCACAGAATCTATGGGAAGTTCTTAGGCCTGCGTGCTTACATC
AGGAAACAGATCAATAATATATTTTATAGGTTTATCTATGAGACAGAG
CATCACAATGGCATAGCGGAGTTACTGGAGATCCTGGGAAGTATAATT
AATGGATTTGCCTTACCACTGAAGGAGGAACACAAGATTTTCCTGCTG
AAGGTGTTGCTGCCCTTGCACAAAGTGAAGTCCCTGAGTGTCTACCAT
CCCCAGCTGGCGTACTGTGTCGTGCAGTTTTTAGAGAAGGACAGCACC
CTCACTGAACCAGTGGTAATGGCACTTCTCAAATACTGGCCAAAGACT
CACAGTCCAAAAGAAGTAATGTTCTTAAATGAATTAGAAGAAATTTTA
GATGTAATTGAACCATCAGAGTTTGTGAAGATCATGGAGCCTCTTTTC
CGACAGTTAGCCAAATGTGTTTCCAGCCCTCACTTCCAGGTGGCCGAG
CGGGCGCTCTATTACTGGAACAACGAGTACATCATGAGTTTAATCAGT
GACAACGCAGCGAAGATTCTGCCCATCATGTTTCCGTCCTTATACCGC
AACTCAAAGACCCACTGGAACAAGACAATACACGGCTTGATATACAAC
GCCCTGAAACTCTTCATGGAGATGAACCAAAAACTCTTCGATGACTGC
ACTCAGCAGTTCAAAGCAGAGAAACTCAAAGAGAAGCTAAAAATGAAA
GAGCGAGAAGAAGCATGGGTTAAAATAGAAAATCTAGCCAAAGCGAAT
CCCCAGTATGCAGTGTATAGTCAAGCCAGCGCCGTGAGCATTCCGGTC
GCAATGGAGACAGATGGGCCTCAGTTTGAAGATGTGCAGATGCTGAAA
AAGACAGTGAGCGACGAGGCTCGTCAGGCACAGAAGGAGCTGAAGAAG
GATCGTCCCCTCGTGCGCCGCAAGTCCGAGCTGCCTCAGGACCCCCAC
ACCGAGAAAGCCTTGGAAGCTCACTGCAGAGCCAGTGAGCTGCTCTCC
CAGGACGGGCGCTAGCGTCTGGAGCAGCACGCCGAGCTGGGCCTGTCG
GTTCTCTTCTGGATGCTGTAGAAAAGACATACTGTGTGTGCCATACCA
GTCAGTTACACTCAATGTCAAAGCTTCCTCTGACCCCGTTCTGTAGGC
AATAATGCACGTCCGCCTCAGCTCGAGATTAGGAGTTCAAACAATGGT
GGCTTCTCTGGCCCTGCTGGCCAAGCAGGGGGCTGGGGGAGTCGGTGA
CAGTGTCATCACCATGTTGTCACCACCCCAGCACAGCCCTGAGTGAGA
TCCAAAGTCAGAGCTGTAGGAAAGCACCCTGACTGTCACCTTCTCGTA
CCCAGTAGACCCTGTGGTCCTCTGAAATAGGGATTCTGTGGTAGGTAA
CACCGATGCTGTGGCAAATCAAACGCACATGGTAAAAGGTGTCTCCTC
CCTTTACAACCCTGACGCTGTCCCCACGCTTGCTGTGCCAGCCGCCCG
TGGTGGTTGGTTAGGTCTCTCAGAGATGGCTCCTCGCTTTCCCGTTCT
AGCTGCCTGCCGGGAAAGAGGCCAACTTTTGCGCTGGGGCCGTCCACA
GTGTTTCTTTCCTTTGGTTAATGGCACGGTCACCATGTCCTGCTTACC
TTAATGTCTACCGAAAGCCATATTTCATAGTCTGTGGTAGAGGCCAGG
GTTCCACCTCAGCGCAAGGGAGGGTTCTGAGAAGTGCTTCCTCAGCCC
ACCTTGAGGAGAACCGCCACGTTGGAATGACGTGTTCATGTTAGTTGC
AGAATCAGTTTTTCACCTACTTACACATGATCCTTCAACAAGGCTCTC
GAGTGGATAAAGATCACAGGATCTAAGAATTCCACATTGGTAGAAAGT
GACAGTAAACTCAGCCCCTTGGCCTAGTGCAGCCTGCAGCTGCCTGTG
GCCTGACTGCGGGCCGCACTGTGTGGGCGCCCTCAGTGAGCCTTTCTT
TCTGTTGGAAAAAGCACATGTCACCAAGCTCGCGAGGAAAGGCGCGGT
GCCCGCGTTCGCACCAGTCTCCACATGCTGCTCCGGCCTTCACAGAAT
TCTTGCATGGATTAAGCTTCTGTTTTAGATGGGATAGCACGAGGAGAT
ACATCCTTAAACTTAGTGCTTTGTCTTCTTGTCCTCTCAGGGTGGCCA
GTATTTTGACTTATTTATCCTGCTCAAAAGCTGCTTGAAATGTGTGCT
GCTCTCTGAACAGTGGCGAGTCTTCTCTTGTCTCCAGCATATATATAA
CTCAGTGTTACATGTCTTGATGCATAAGATAGGTTAAAAAAAAAGAAG
AAGAAGAAAAATGGGGCTTCTTTTAAAACCTGATTTTAATCGTGGTCT
CAGGGATCCGTGAAAAAGTTGTGAAATCAGACATTGCTCTCTCGCGGC
CTTGGAGGCGGGTCCCACGGGGAAGAAGGGCATGTGCAGGGAGGGAGA
GCCAGCACGGCTAGTCAAAAAGGCGGCCCCAGCTGAGGGTGAACAGGT
TGGCCACTTGGGTGAGAACACTTCCTGAGAAACTTGACAAGTACCTAT
CCACTTTACCATTATGAAATCTATAATTAAAAAAAAAAAGTTGAGATG
CCTTCTCCTTTTGAGGGAAAAAGGGTGCTTTTATTGTATAAAGCAGTG
TCTCTGTGTTTTGATAGCCCACTGTTTGAACTCTCGTCTTTAGCTGGT
AGAGTCTCAGATATCCCTGACGTGTGGGGCATTCCGAGTGTGGTGAGA
GGTCCCAAGAGGCTCCTTTGCCCTGCGTGAAGAGCATGCTATCAGTGT
ACGGGTGATTGTGCAAAGCTCAGCGCGGGGGAGCATCTTCTGCTCTAG
GTTTTATTTCTGCTCTTTATTGAAGACAAACATTCGCCAATAAAGAAA
AGGGAAAAAAATGTTTTGAGAGAAACTAATTTTCTTTGGCAAAAGTAT
TACTTAAATTTTTGGCCTATTAAGGTTCCCCTAGTTAGTACTCGGATT
CCCCATGCTAATTGTTCAGCTTGTATGTTGTTAAGACACTGTTGATTC
TGTACCAGACTGGTTTCAAAACAAAAAAAGTACCACGTTGAAAATAAA
CTGGTGACTGTTTTTCTTCATATGGCTCTAGTTTGGCACCTTCACTCT
TTCCAAAACGTGTCTGTGCGCCAGAGATGTCACAGTTCAAGTGTCTTT
CTAGTGTGGCTTTGTATGGCTTCCTTTGAACATTGTACATACATTGTA
TCTTTGTTTTATGGTAATAAGTAATAAAAAATGTAGACTTCGTATTTT
GTACAAAATGTCCTATGTACAGAATAAAAAAAAGTTCATAGAAACAGC
AAAAATAGGTTAAGTGGCACAGTTATTTTTCTTTAGAAAATATCTGTA
ACTTTATGCTTTAGTGAAACGTTAAGTACCAACATATTTTTTAACATT
TTGTAATTCAAAACTTTTTTGTTTTGACATTGTTTATGAAGAAAGACT
TCATGCACTTGCCATTTAATATGCTCTTTTATCTAATTTTAAAGAACT
CTTTAAAATGGTGTATTATATGGACTAAATAAAGAACATGTGAATTTT
>gi|125346006|ref|NP_001074926.1| gamma isoform of
regulatory subunit B56, protein phosphatase 2A
isoform b [Mus musculus]
MLTCNKAGSGMVVDAASSNGPFQPVALLHIRDVPPADQEKLFIQKLRQ
CCVLEDEVSDPLSDLKWKEVKRAALSEMVEYITHNRNVITEPIYPEAV
HMFAVNMERTLPPSSNPTGAEFDPEEDEPTLEAAWPHLQLVYEFFLRF
LESPDFQPNIAKKYIDQKFVLQLLELFDSEDPRERDELKTTLHRIYGK
FLGLRAYIRKQINNIFYRFIYETEHHNGIAELLEILGSIINGFALPLK
EEHKIFLLKVLLPLHKVKSLSVYHPQLAYCVVQFLEKDSTLTEPVVMA
LLKYWPKTHSPKEVMFLNELEEILDVIEPSEFVKIMEPLFRQLAKCVS
SPHFQVAERALYYWNNEYIMSLISDNAAKILPIMFPSLYRNSKTHWNK
TIHGLIYNALKLEMEMNQKLEDDCTQQFKAEKLKEKLKMKEREEAWVK
IENLAKANPQAQKELKKDRPLVRRKSELPQDPHTEKALEAHCRASELL
SQDGR
>gi|125346005|ref|NM_001081457.1| Mus musculus
protein phosphatase 2, regulatory subunit B (B56),
gamma isoform (Ppp2r5c), transcript variant 2, mRNA
CGAAGCAGCCAGTTCCCTCCAGCTGCAGAGAGCTTCAGTTTGTCTTTT
TTTTTTTTTAAACTAAAATGGAGGCTGGTTTCTTGCCTTAAGGAGTAC
AGCGCCCTTCCCGCTGGAGCCTAGATGTTGACATGTAATAAAGCGGGC
AGCGGGATGGTGGTGGATGCGGCCAGCTCCAACGGGCCTTTCCAGCCC
GTGGCCCTTCTCCACATTCGAGATGTTCCTCCTGCGGATCAAGAGAAG
CTTTTTATCCAGAAGCTACGCCAGTGTTGTGTCCTCTTTGACTTTGTC
TCTGACCCACTGAGTGACCTGAAGTGGAAGGAAGTAAAGCGCGCTGCG
CTGAGCGAGATGGTGGAGTATATCACCCACAACCGGAACGTGATCACG
GAGCCCATTTACCCCGAGGCCGTCCACATGTTTGCAGTTAACATGTTC
CGAACCTTGCCACCTTCCTCCAATCCCACGGGAGCAGAATTCGACCCA
GAAGAGGATGAACCAACGTTAGAAGCAGCCTGGCCTCATCTGCAGCTT
GTTTATGAATTTTTCTTAAGATTTTTAGAGTCTCCAGATTTCCAACCC
AATATAGCAAAGAAATATATTGATCAGAAGTTTGTATTGCAGCTTCTA
GAGCTGTTTGACAGCGAGGATCCTCGGGAGAGAGATTTTCTAAAAACC
ACCCTGCACAGAATCTATGGGAAGTTCTTAGGCCTGCGTGCTTACATC
AGGAAACAGATCAATAATATATTTTATAGGTTTATCTATGAGACAGAG
CATCACAATGGCATAGCGGAGTTACTGGAGATCCTGGGAAGTATAATT
AATGGATTTGCCTTACCACTGAAGGAGGAACACAAGATTTTCCTGCTG
AAGGTGTTGCTGCCCTTGCACAAAGTGAAGTCCCTGAGTGTCTACCAT
CCCCAGCTGGCGTACTGTGTCGTGCAGTTTTTAGAGAAGGACAGCACC
CTCACTGAACCAGTGGTAATGGCACTTCTCAAATACTGGCCAAAGACT
CACAGTCCAAAAGAAGTAATGTTCTTAAATGAATTAGAAGAAATTTTA
GATGTAATTGAACCATCAGAGTTTGTGAAGATCATGGAGCCTCTTTTC
CGACAGTTAGCCAAATGTGTTTCCAGCCCTCACTTCCAGGTGGCCGAG
CGGGCGCTCTATTACTGGAACAACGAGTACATCATGAGTTTAATCAGT
GACAACGCAGCGAAGATTCTGCCCATCATGTTTCCGTCCTTATACCGC
AACTCAAAGACCCACTGGAACAAGACAATACACGGCTTGATATACAAC
GCCCTGAAACTCTTCATGGAGATGAACCAAAAACTCTTCGATGACTGC
ACTCAGCAGTTCAAAGCAGAGAAACTCAAAGAGAAGCTAAAAATGAAA
GAGCGAGAAGAAGCATGGGTTAAAATAGAAAATCTAGCCAAAGCGAAT
CCCCAGGCACAGAAGGAGCTGAAGAAGGATCGTCCCCTCGTGCGCCGC
AAGTCCGAGCTGCCTCAGGACCCCCACACCGAGAAAGCCTTGGAAGCT
CACTGCAGAGCCAGTGAGCTGCTCTCCCAGGACGGGCGCTAGCGTCTG
GAGCAGCACGCCGAGCTGGGCCTGTCGGTTCTCTTCTGGATGCTGTAG
AAAAGACATACTGTGTGTGCCATACCAGTCAGTTACACTCAATGTCAA
AGCTTCCTCTGACCCCGTTCTGTAGGCAATAATGCACGTCCGCCTCAG
CTCGAGATTAGGAGTTCAAACAATGGTGGCTTCTCTGGCCCTGCTGGC
CAAGCAGGGGGCTGGGGGAGTCGGTGACAGTGTCATCACCATGTTGTC
ACCACCCCAGCACAGCCCTGAGTGAGATCCAAAGTCAGAGCTGTAGGA
AAGCACCCTGACTGTCACCTTCTCGTACCCAGTAGACCCTGTGGTCCT
CTGAAATAGGGATTCTGTGGTAGGTAACACCGATGCTGTGGCAAATCA
AACGCACATGGTAAAAGGTGTCTCCTCCCTTTACAACCCTGACGCTGT
CCCCACGCTTGCTGTGCCAGCCGCCCGTGGTGGTTGGTTAGGTCTCTC
AGAGATGGCTCCTCGCTTTCCCGTTCTAGCTGCCTGCCGGGAAAGAGG
CCAACTTTTGCGCTGGGGCCGTCCACAGTGTTTCTTTCCTTTGGTTAA
TGGCACGGTCACCATGTCCTGCTTACCTTAATGTCTACCGAAAGCCAT
ATTTCATAGTCTGTGGTAGAGGCCAGGGTTCCACCTCAGCGCAAGGGA
GGGTTCTGAGAAGTGCTTCCTCAGCCCACCTTGAGGAGAACCGCCACG
TTGGAATGACGTGTTCATGTTAGTTGCAGAATCAGTTTTTCACCTACT
TACACATGATCCTTCAACAAGGCTCTCGAGTGGATAAAGATCACAGGA
TCTAAGAATTCCACATTGGTAGAAAGTGACAGTAAACTCAGCCCCTTG
GCCTAGTGCAGCCTGCAGCTGCCTGTGGCCTGACTGCGGGCCGCACTG
TGTGGGCGCCCTCAGTGAGCCTTTCTTTCTGTTGGAAAAAGCACATGT
CACCAAGCTCGCGAGGAAAGGCGCGGTGCCCGCGTTCGCACCAGTCTC
CACATGCTGCTCCGGCCTTCACAGAATTCTTGCATGGATTAAGCTTCT
GTTTTAGATGGGATAGCACGAGGAGATACATCCTTAAACTTAGTGCTT
TGTCTTCTTGTCCTCTCAGGGTGGCCAGTATTTTGACTTATTTATCCT
GCTCAAAAGCTGCTTGAAATGTGTGCTGCTCTCTGAACAGTGGCGAGT
CTTCTCTTGTCTCCAGCATATATATAACTCAGTGTTACATGTCTTGAT
GCATAAGATAGGTTAAAAAAAAAGAAGAAGAAGAAAAATGGGGCTTCT
TTTAAAACCTGATTTTAATCGTGGTCTCAGGGATCCGTGAAAAAGTTG
TGAAATCAGACATTGCTCTCTCGCGGCCTTGGAGGCGGGTCCCACGGG
GAAGAAGGGCATGTGCAGGGAGGGAGAGCCAGCACGGCTAGTCAAAAA
GGCGGCCCCAGCTGAGGGTGAACAGGTTGGCCACTTGGGTGAGAACAC
TTCCTGAGAAACTTGACAAGTACCTATCCACTTTACCATTATGAAATC
TATAATTAAAAAAAAAAAGTTGAGATGCCTTCTCCTTTTGAGGGAAAA
AGGGTGCTTTTATTGTATAAAGCAGTGTCTCTGTGTTTTGATAGCCCA
CTGTTTGAACTCTCGTCTTTAGCTGGTAGAGTCTCAGATATCCCTGAC
GTGTGGGGCATTCCGAGTGTGGTGAGAGGTCCCAAGAGGCTCCTTTGC
CCTGCGTGAAGAGCATGCTATCAGTGTACGGGTGATTGTGCAAAGCTC
AGCGCGGGGGAGCATCTTCTGCTCTAGGTTTTATTTCTGCTCTTTATT
GAAGACAAACATTCGCCAATAAAGAAAAGGGAAAAAAATGTTTTGAGA
GAAACTAATTTTCTTTGGCAAAAGTATTACTTAAATTTTTGGCCTATT
AAGGTTCCCCTAGTTAGTACTCGGATTCCCCATGCTAATTGTTCAGCT
TGTATGTTGTTAAGACACTGTTGATTCTGTACCAGACTGGTTTCAAAA
CAAAAAAAGTACCACGTTGAAAATAAACTGGTGACTGTTTTTCTTCAT
ATGGCTCTAGTTTGGCACCTTCACTCTTTCCAAAACGTGTCTGTGCGC
CAGAGATGTCACAGTTCAAGTGTCTTTCTAGTGTGGCTTTGTATGGCT
TCCTTTGAACATTGTACATACATTGTATCTTTGTTTTATGGTAATAAG
TAATAAAAAATGTAGACTTCGTATTTTGTACAAAATGTCCTATGTACA
GAATAAAAAAAAGTTCATAGAAACAGCAAAAATAGGTTAAGTGGCACA
GTTATTTTTCTTTAGAAAATATCTGTAACTTTATGCTTTAGTGAAACG
TTAAGTACCAACATATTTTTTAACATTTTGTAATTCAAAACTTTTTTG
TTTTGACATTGTTTATGAAGAAAGACTTCATGCACTTGCCATTTAATA
TGCTCTTTTATCTAATTTTAAAGAACTCTTTAAAATGGTGTATTATAT
GGACTAAATAAAGAACATGTGAATTTT
PPP2R5D (Human)
>gi|5453954|ref|NP_006236.1| delta isoform of
regulatory subunit B56, protein phosphatase 2A
isoform 1 [Homo sapiens]
MPYKLKKEKEPPKVAKCTAKPSSSGKDGGGENTEEAQPQPQPQPQPQA
QSQPPSSNKRPSNSTPPPTQLSKIKYSGGPQIVKKERRQSSSRFNLSK
NRELQKLPALKDSPTQEREELFIQKLRQCCVLFDFVSDPLSDLKFKEV
KRAGLNEMVEYITHSRDVVTEAIYPEAVTMFSVNLFRTLPPSSNPTGA
EFDPEEDEPTLEAAWPHLQLVYEFFLRFLESPDFQPNIAKKYIDQKFV
LALLDLFDSEDPRERDFLKTILHRIYGKFLGLRAYIRRQINHIFYRFI
YETEHHNGIAELLEILGSIINGFALPLKEEHKMFLIRVLLPLHKVKSL
SVYHPQLAYCVVQFLEKESSLTEPVIVGLLKFWPKTHSPKEVMFLNEL
EEILDVIEPSEFSKVMEPLFRQLAKCVSSPHFQVAERALYYWNNEYIM
SLISDNAARVLPIMFPALYRNSKSHWNKTIHGLIYNALKLFMEMNQKL
FDDCTQQYKAEKQKGRFRMKEREEMWQKIEELARLNPQYPMFRAPPPL
PPVYSMETETPTAEDIQLLKRTVETEAVQMLKDIKKEKVLLRRKSELP
QDVYTIKALEAHKRAEEFLTASQEAL
>gi|31083266|ref|NM_006245.2| Homo sapiens protein
phosphatase 2, regulatory subunit B′, delta isoform
(PPP2R5D), transcript variant 1, mRNA
GAGACGCCGAGCGGGCCGAGTGCGGCCGAGCAAAGCCGGAGCCGGAGC
GGGGCCGCAGGAGACGGGCCGGGTCCGGACGGGCCGAGATGCCCTATA
AACTGAAAAAGGAGAAGGAGCCCCCCAAGGTTGCCAAATGCACAGCCA
AGCCTAGCAGCTCGGGCAAGGATGGTGGAGGCGAGAACACTGAGGAGG
CCCAGCCGCAGCCCCAGCCCCAGCCCCAGCCCCAAGCCCAGTCTCAGC
CACCGTCATCCAACAAGCGTCCCAGCAATAGCACGCCGCCCCCCACGC
AGCTCAGCAAAATCAAGTACTCAGGGGGGCCCCAGATTGTCAAGAAGG
AGCGACGGCAAAGCTCCTCCCGCTTCAACCTCAGCAAGAATCGGGAGC
TGCAGAAGCTTCCTGCCCTGAAAGATTCGCCAACCCAGGAGCGGGAGG
AGCTGTTTATCCAGAAGCTACGCCAGTGCTGTGTCCTCTTTGACTTCG
TGTCAGACCCACTCAGTGACCTCAAATTCAAGGAGGTGAAGCGGGCAG
GACTCAACGAGATGGTGGAGTACATCACCCATAGCCGTGATGTTGTCA
CTGAGGCCATTTACCCTGAGGCTGTCACCATGTTTTCAGTGAACCTCT
TCCGGACGCTGCCACCTTCATCGAATCCCACAGGGGCTGAGTTTGACC
CAGAGGAAGATGAGCCCACCCTGGAAGCTGCTTGGCCACATCTCCAGC
TCGTGTATGAGTTCTTCTTACGTTTCCTTGAGTCTCCTGATTTCCAGC
CAAACATAGCCAAGAAGTACATCGACCAGAAGTTTGTACTTGCTCTCC
TAGACCTATTTGACAGTGAGGATCCTCGAGAGCGGGACTTCCTCAAGA
CCATTTTGCATCGCATCTATGGCAAGTTTTTGGGGCTCCGGGCTTATA
TCCGTAGGCAGATCAACCACATCTTCTACAGGTTCATCTACGAGACGG
AGCATCACAACGGGATTGCTGAGCTCCTGGAGATCCTGGGCAGCATCA
TCAATGGCTTTGCCCTGCCCCTTAAAGAAGAGCACAAGATGTTCCTCA
TCCGTGTCCTACTTCCCCTTCACAAGGTCAAGTCCCTGAGTGTCTACC
ACCCTCAGCTGGCATACTGTGTGGTACAATTCCTGGAGAAGGAGAGCA
GTCTGACTGAGCCGGTAATTGTGGGACTTCTCAAGTTTTGGCCCAAGA
CCCACAGCCCCAAGGAGGTGATGTTCTTGAATGAGCTGGAGGAGATTC
TGGACGTCATTGAACCTTCTGAGTTCAGCAAAGTGATGGAACCCCTCT
TCCGCCAGCTGGCCAAGTGTGTCTCTAGCCCCCATTTCCAGGTGGCAG
AGCGTGCTCTCTATTACTGGAACAATGAGTACATCATGAGCCTGATAA
GTGACAATGCTGCCCGAGTCCTCCCCATCATGTTCCCTGCACTCTACA
GGAACTCCAAGAGCCACTGGAACAAGACAATCCATGGACTGATCTATA
ATGCCCTGAAGTTGTTTATGGAAATGAATCAGAAGCTGTTTGATGACT
GCACACAACAATACAAGGCAGAGAAGCAGAAGGGCCGGTTCCGAATGA
AGGAAAGGGAAGAGATGTGGCAAAAAATCGAGGAGCTGGCCCGGCTTA
ATCCCCAGTATCCCATGTTCCGAGCCCCTCCACCACTGCCCCCTGTGT
ACTCGATGGAGACAGAGACCCCCACAGCTGAGGACATCCAGCTTCTGA
AGAGGACTGTGGAGACTGAGGCTGTTCAGATGCTAAAAGACATCAAGA
AGGAGAAAGTGCTGCTGCGGAGGAAGTCGGAGCTGCCCCAGGACGTGT
ACACCATCAAGGCACTGGAGGCGCACAAGCGGGCGGAAGAGTTCCTAA
CTGCCAGCCAGGAGGCTCTCTGACCCCTCACGTTCCTACCACAGGGCC
ACAGCCCACACAGCCCTGGGACACTGCCCTGGCCCTCCATACTCTGCT
CCCTACTGGCTGTCTTGGGGGAAGGCAGCGCCTCTCTAGCTACTCAAG
GGAGGGGGATGTGGGCACTTGAAGCAGGGACACCCACAGAATGGTCCC
TCTTCTCCCCAAAAGGTGTTCATGCCTCCCTGTGGCTAGTACAGGCTG
AGCACTAAGATGCTTAGTGCTCAGACAACCTGGGGATGCCTGTCCCCT
ACCTGCTCCTCACCCACAGCTACCTGAGGCTGCTCTGAGAAGTACACA
CAGGAATACATACGCTCCTCTATTCTTCCCTTCATCCTCATTTGAACG
CCAGGTATCTCCCCTCCTCTCTCTCCCCTGCAGAGGCATGTAGGGAAC
AGCAGGAGATTATTCTCACCAAAGTTATGTCAAGCCCCATTGGTCCCA
GAGTAGCTGAAGGGAAGCCAACCCCCCTGCAGCACAAATAGGCCCCCC
AGTCCCAGCCGTGTGCTGGCAGATAGGGTTGTATTATTTCTTCTTACC
CCATGCCTGACCAAGGGAGGTCAAAAGGAGAAAAGTATAGGCTGTGGA
CAATAACTGATGAATATAGGGCCCAGATGGACCAAGTGGGGCCGGGGA
GGGATGAATAAACACCCTACCCCGTGCCCTGTCTTTGGTGAGCAGCAG
CCCTGGGGTCACAGACATGGAAGGGACCACCCTGGGGCTGACTGCTTT
TCTGTGCTGTTGGTTCCCAAAACTAGAAAGAAGGAAGCAGGGAGCGGT
GCCCCAAGCATGGCTCCTGCCAACACCTATTTATTTCCTTGTTTGTGC
TATGCTGGGCAGGCCTTCTCTTGTCCCTTATAGGTACCTTGGAGGGGC
CAGGGGCTGAGGAAGGCCGGACCCAGGTTCCAGGGGCGCAGGCAGTGC
GGCTTTTGGCTGTGTACATAGGGTGCTTTATTCTCCACAGAGTGATAC
ATGCTAAGGTGGGTTGGGCTTGGACCGATGTCCCCATATGTACAGAAC
TGAATAAAGTGGGTCTCTGAGAAAAAAAAAAAAAAAAAAAAAAAAAA
>gi|31083280|ref|NP851307.1| delta isoform of
regulatory subunit B56, protein phosphatase 2A
isoform 2 [Homo sapiens]
MPYKLKKEKEPPKVAKCTAKPSSSGKDGGGENTEEAQPQPQPQPQPQA
QSQPPSSNKRPSNSTPPPTQLSKIKYSGGPQIVKKELFIQKLRQCCVL
FDFVSDPLSDLKFKEVKRAGLNEMVEYITHSRDVVTEAIYPEAVTMFS
VNLFRTLPPSSNPTGAEFDPEEDEPTLEAAWPHLQLVYEFFLRFLESP
DFQPNIAKKYIDQKFVLALLDLFDSEDPRERDFLKTILHRIYGKFLGL
RAYIRRQINHIFYRFIYETEHHNGIAELLEILGSIINGFALPLKEEHK
MFLIRVLLPLHKVKSLSVYHPQLAYCVVQFLEKESSLTEPVIVGLLKF
WPKTHSPKEVMFLNELEEILDVIEPSEFSKVMEPLFRQLAKCVSSPHF
QVAERALYYWNNEYIMSLISDNAARVLPIMFPALYRNSKSHWNKTIHG
LIYNALKLFMEMNQKLFDDCTQQYKAEKQKGRFRMKEREEMWQKIEEL
ARLNPQYPMFRAPPPLPPVYSMETETPTAEDIQLLKRTVETEAVQMLK
DIKKEKVLLRRKSELPQDVYTIKALEAHKRAEEFLTASQEAL
>gi|31083279|ref|NM_180976.1| Homo sapiens protein
phosphatase 2, regulatory subunit B′, delta
isoform (PPP2R5D), transcript variant 2, mRNA
GAGACGCCGAGCGGGCCGAGTGCGGCCGAGCAAAGCCGGAGCCGGAGC
GGGGCCGCAGGAGACGGGCCGGGTCCGGACGGGCCGAGATGCCCTATA
AACTGAAAAAGGAGAAGGAGCCCCCCAAGGTTGCCAAATGCACAGCCA
AGCCTAGCAGCTCGGGCAAGGATGGTGGAGGCGAGAACACTGAGGAGG
CCCAGCCGCAGCCCCAGCCCCAGCCCCAGCCCCAAGCCCAGTCTCAGC
CACCGTCATCCAACAAGCGTCCCAGCAATAGCACGCCGCCCCCCACGC
AGCTCAGCAAAATCAAGTACTCAGGGGGGCCCCAGATTGTCAAGAAGG
AGCTGTTTATCCAGAAGCTACGCCAGTGCTGTGTCCTCTTTGACTTCG
TGTCAGACCCACTCAGTGACCTCAAATTCAAGGAGGTGAAGCGGGCAG
GACTCAACGAGATGGTGGAGTACATCACCCATAGCCGTGATGTTGTCA
CTGAGGCCATTTACCCTGAGGCTGTCACCATGTTTTCAGTGAACCTCT
TCCGGACGCTGCCACCTTCATCGAATCCCACAGGGGCTGAGTTTGACC
CAGAGGAAGATGAGCCCACCCTGGAAGCTGCTTGGCCACATCTCCAGC
TCGTGTATGAGTTCTTCTTACGTTTCCTTGAGTCTCCTGATTTCCAGC
CAAACATAGCCAAGAAGTACATCGACCAGAAGTTTGTACTTGCTCTCC
TAGACCTATTTGACAGTGAGGATCCTCGAGAGCGGGACTTCCTCAAGA
CCATTTTGCATCGCATCTATGGCAAGTTTTTGGGGCTCCGGGCTTATA
TCCGTAGGCAGATCAACCACATCTTCTACAGGTTCATCTACGAGACGG
AGCATCACAACGGGATTGCTGAGCTCCTGGAGATCCTGGGCAGCATCA
TCAATGGCTTTGCCCTGCCCCTTAAAGAAGAGCACAAGATGTTCCTCA
TCCGTGTCCTACTTCCCCTTCACAAGGTCAAGTCCCTGAGTGTCTACC
ACCCTCAGCTGGCATACTGTGTGGTACAATTCCTGGAGAAGGAGAGCA
GTCTGACTGAGCCGGTAATTGTGGGACTTCTCAAGTTTTGGCCCAAGA
CCCACAGCCCCAAGGAGGTGATGTTCTTGAATGAGCTGGAGGAGATTC
TGGACGTCATTGAACCTTCTGAGTTCAGCAAAGTGATGGAACCCCTCT
TCCGCCAGCTGGCCAAGTGTGTCTCTAGCCCCCATTTCCAGGTGGCAG
AGCGTGCTCTCTATTACTGGAACAATGAGTACATCATGAGCCTGATAA
GTGACAATGCTGCCCGAGTCCTCCCCATCATGTTCCCTGCACTCTACA
GGAACTCCAAGAGCCACTGGAACAAGACAATCCATGGACTGATCTATA
ATGCCCTGAAGTTGTTTATGGAAATGAATCAGAAGCTGTTTGATGACT
GCACACAACAATACAAGGCAGAGAAGCAGAAGGGCCGGTTCCGAATGA
AGGAAAGGGAAGAGATGTGGCAAAAAATCGAGGAGCTGGCCCGGCTTA
ATCCCCAGTATCCCATGTTCCGAGCCCCTCCACCACTGCCCCCTGTGT
ACTCGATGGAGACAGAGACCCCCACAGCTGAGGACATCCAGCTTCTGA
AGAGGACTGTGGAGACTGAGGCTGTTCAGATGCTAAAAGACATCAAGA
AGGAGAAAGTGCTGCTGCGGAGGAAGTCGGAGCTGCCCCAGGACGTGT
ACACCATCAAGGCACTGGAGGCGCACAAGCGGGCGGAAGAGTTCCTAA
CTGCCAGCCAGGAGGCTCTCTGACCCCTCACGTTCCTACCACAGGGCC
ACAGCCCACACAGCCCTGGGACACTGCCCTGGCCCTCCATACTCTGCT
CCCTACTGGCTGTCTTGGGGGAAGGCAGCGCCTCTCTAGCTACTCAAG
GGAGGGGGATGTGGGCACTTGAAGCAGGGACACCCACAGAATGGTCCC
TCTTCTCCCCAAAAGGTGTTCATGCCTCCCTGTGGCTAGTACAGGCTG
AGCACTAAGATGCTTAGTGCTCAGACAACCTGGGGATGCCTGTCCCCT
ACCTGCTCCTCACCCACAGCTACCTGAGGCTGCTCTGAGAAGTACACA
CAGGAATACATACGCTCCTCTATTCTTCCCTTCATCCTCATTTGAACG
CCAGGTATCTCCCCTCCTCTCTCTCCCCTGCAGAGGCATGTAGGGAAC
AGCAGGAGATTATTCTCACCAAAGTTATGTCAAGCCCCATTGGTCCCA
GAGTAGCTGAAGGGAAGCCAACCCCCCTGCAGCACAAATAGGCCCCCC
AGTCCCAGCCGTGTGCTGGCAGATAGGGTTGTATTATTTCTTCTTACC
CCATGCCTGACCAAGGGAGGTCAAAAGGAGAAAAGTATAGGCTGTGGA
CAATAACTGATGAATATAGGGCCCAGATGGACCAAGTGGGGCCGGGGA
GGGATGAATAAACACCCTACCCCGTGCCCTGTCTTTGGTGAGCAGCAG
CCCTGGGGTCACAGACATGGAAGGGACCACCCTGGGGCTGACTGCTTT
TCTGTGCTGTTGGTTCCCAAAACTAGAAAGAAGGAAGCAGGGAGCGGT
GCCCCAAGCATGGCTCCTGCCAACACCTATTTATTTCCTTGTTTGTGC
TATGCTGGGCAGGCCTTCTCTTGTCCCTTATAGGTACCTTGGAGGGGC
CAGGGGCTGAGGAAGGCCGGACCCAGGTTCCAGGGGCGCAGGCAGTGC
GGCTTTTGGCTGTGTACATAGGGTGCTTTATTCTCCACAGAGTGATAC
ATGCTAAGGTGGGTTGGGCTTGGACCGATGTCCCCATATGTACAGAAC
TGAATAAAGTGGGTCTCTGAGAAAAAAAAAAAAAAAAAA
>gi|31083288|ref|NP_851308.1| delta isoform of
regulatory subunit B56, protein phosphatase 2A
isoform 3 [Homo sapiens]
MPYKLKKEKELFIQKLRQCCVLFDFVSDPLSDLKFKEVKRAGLNEMVE
YITHSRDVVTEAIYPEAVTMFSVNLFRTLPPSSNPTGAEFDPEEDEPT
LEAAWPHLQLVYEFFLRFLESPDFQPNIAKKYIDQKFVLALLDLFDSE
DPRERDFLKTILHRIYGKFLGLRAYIRRQINHIFYRFIYETEHHNGIA
ELLEILGSIINGFALPLKEEHKMFLIRVLLPLHKVKSLSVYHPQLAYC
VVQFLEKESSLTEPVIVGLLKFWPKTHSPKEVMFLNELEEILDVIEPS
EFSKVMEPLFRQLAKCVSSPHFQVAERALYYWNNEYIMSLISDNAARV
LPIMFPALYRNSKSHWNKTIHGLIYNALKLFMEMNQKLFDDCTQQYKA
EKQKGRFRMKEREEMWQKIEELARLNPQYPMFRAPPPLPPVYSMETET
PTAEDIQLLKRTVETEAVQMLKDIKKEKVLLRRKSELPQDVYTIKALE
AHKRAEEFLTASQEAL
>gi|31083287|ref|NM_180977.1| Homo sapiens protein
phosphatase 2, regulatory subunit B′, delta isoform
(PPP2R5D), transcript variant 3, mRNA
GAGACGCCGAGCGGGCCGAGTGCGGCCGAGCAAAGCCGGAGCCGGAGC
GGGGCCGCAGGAGACGGGCCGGGTCCGGACGGGCCGAGATGCCCTATA
AACTGAAAAAGGAGAAGGAGCTGTTTATCCAGAAGCTACGCCAGTGCT
GTGTCCTCTTTGACTTCGTGTCAGACCCACTCAGTGACCTCAAATTCA
AGGAGGTGAAGCGGGCAGGACTCAACGAGATGGTGGAGTACATCACCC
ATAGCCGTGATGTTGTCACTGAGGCCATTTACCCTGAGGCTGTCACCA
TGTTTTCAGTGAACCTCTTCCGGACGCTGCCACCTTCATCGAATCCCA
CAGGGGCTGAGTTTGACCCAGAGGAAGATGAGCCCACCCTGGAAGCTG
CTTGGCCACATCTCCAGCTCGTGTATGAGTTCTTCTTACGTTTCCTTG
AGTCTCCTGATTTCCAGCCAAACATAGCCAAGAAGTACATCGACCAGA
AGTTTGTACTTGCTCTCCTAGACCTATTTGACAGTGAGGATCCTCGAG
AGCGGGACTTCCTCAAGACCATTTTGCATCGCATCTATGGCAAGTTTT
TGGGGCTCCGGGCTTATATCCGTAGGCAGATCAACCACATCTTCTACA
GGTTCATCTACGAGACGGAGCATCACAACGGGATTGCTGAGCTCCTGG
AGATCCTGGGCAGCATCATCAATGGCTTTGCCCTGCCCCTTAAAGAAG
AGCACAAGATGTTCCTCATCCGTGTCCTACTTCCCCTTCACAAGGTCA
AGTCCCTGAGTGTCTACCACCCTCAGCTGGCATACTGTGTGGTACAAT
TCCTGGAGAAGGAGAGCAGTCTGACTGAGCCGGTAATTGTGGGACTTC
TCAAGTTTTGGCCCAAGACCCACAGCCCCAAGGAGGTGATGTTCTTGA
ATGAGCTGGAGGAGATTCTGGACGTCATTGAACCTTCTGAGTTCAGCA
AAGTGATGGAACCCCTCTTCCGCCAGCTGGCCAAGTGTGTCTCTAGCC
CCCATTTCCAGGTGGCAGAGCGTGCTCTCTATTACTGGAACAATGAGT
ACATCATGAGCCTGATAAGTGACAATGCTGCCCGAGTCCTCCCCATCA
TGTTCCCTGCACTCTACAGGAACTCCAAGAGCCACTGGAACAAGACAA
TCCATGGACTGATCTATAATGCCCTGAAGTTGTTTATGGAAATGAATC
AGAAGCTGTTTGATGACTGCACACAACAATACAAGGCAGAGAAGCAGA
AGGGCCGGTTCCGAATGAAGGAAAGGGAAGAGATGTGGCAAAAAATCG
AGGAGCTGGCCCGGCTTAATCCCCAGTATCCCATGTTCCGAGCCCCTC
CACCACTGCCCCCTGTGTACTCGATGGAGACAGAGACCCCCACAGCTG
AGGACATCCAGCTTCTGAAGAGGACTGTGGAGACTGAGGCTGTTCAGA
TGCTAAAAGACATCAAGAAGGAGAAAGTGCTGCTGCGGAGGAAGTCGG
AGCTGCCCCAGGACGTGTACACCATCAAGGCACTGGAGGCGCACAAGC
GGGCGGAAGAGTTCCTAACTGCCAGCCAGGAGGCTCTCTGACCCCTCA
CGTTCCTACCACAGGGCCACAGCCCACACAGCCCTGGGACACTGCCCT
GGCCCTCCATACTCTGCTCCCTACTGGCTGTCTTGGGGGAAGGCAGCG
CCTCTCTAGCTACTCAAGGGAGGGGGATGTGGGCACTTGAAGCAGGGA
CACCCACAGAATGGTCCCTCTTCTCCCCAAAAGGTGTTCATGCCTCCC
TGTGGCTAGTACAGGCTGAGCACTAAGATGCTTAGTGCTCAGACAACC
TGGGGATGCCTGTCCCCTACCTGCTCCTCACCCACAGCTACCTGAGGC
TGCTCTGAGAAGTACACACAGGAATACATACGCTCCTCTATTCTTCCC
TTCATCCTCATTTGAACGCCAGGTATCTCCCCTCCTCTCTCTCCCCTG
CAGAGGCATGTAGGGAACAGCAGGAGATTATTCTCACCAAAGTTATGT
CAAGCCCCATTGGTCCCAGAGTAGCTGAAGGGAAGCCAACCCCCCTGC
AGCACAAATAGGCCCCCCAGTCCCAGCCGTGTGCTGGCAGATAGGGTT
GTATTATTTCTTCTTACCCCATGCCTGACCAAGGGAGGTCAAAAGGAG
AAAAGTATAGGCTGTGGACAATAACTGATGAATATAGGGCCCAGATGG
ACCAAGTGGGGCCGGGGAGGGATGAATAAACACCCTACCCCGTGCCCT
GTCTTTGGTGAGCAGCAGCCCTGGGGTCACAGACATGGAAGGGACCAC
CCTGGGGCTGACTGCTTTTCTGTGCTGTTGGTTCCCAAAACTAGAAAG
AAGGAAGCAGGGAGCGGTGCCCCAAGCATGGCTCCTGCCAACACCTAT
TTATTTCCTTGTTTGTGCTATGCTGGGCAGGCCTTCTCTTGTCCCTTA
TAGGTACCTTGGAGGGGCCAGGGGCTGAGGAAGGCCGGACCCAGGTTC
CAGGGGCGCAGGCAGTGCGGCTTTTGGCTGTGTACATAGGGTGCTTTA
TTCTCCACAGAGTGATACATGCTAAGGTGGGTTGGGCTTGGACCGATG
TCCCCATATGTACAGAACTGAATAAAGTGGGTCTCTGAGAAAAAAAAA
AAAAAAAAA
PPP2R5D (Mouse)
>gi|33942059|ref|NP033384.2| delta isoform of
regulatory subunit B56, protein phosphatase 2A
[Mus musculus]
MSYKLKKDKEPSKLAKGTAKPSSSSKDGGGENTDEAQPQPQSQSPSSN
KRPSNSTPPPTQLSKIKYSGGPQIVKKERRQSSFPFNLNKNRELQKLP
ALKDSPTQEREELFIQKLRQCCVLFDFVSDPLSDLKCKEVKRAGLNEM
VEYITHSRDVVTEAIYPEAVTMFSVNLFRTLPPSSNPTGAEFDPEEDE
PTLEAAWPHLQLVYEFFLRFLESPDFQPNIAKKYIDQKFVLALLDLFD
SEDPRERDFLKTILHRIYGKFLGLRAYIRRQINHIFYRFIYETEHHNG
IAELLEILGSIINGFALPLKEEHKVFLVRVLLPLHKVKSLSVYHPQLA
YCVVQFLEKESSLTEPVIVGLLKFWPKTHSPKEVMFLNELEEILDVIE
PSEFSKVMEPLFRQLAKCVSSPHFQVAERALYYWNNEYIMSLISDNAA
RILPIMFPALYRNSKSHWNKTIHGLIYNALKLFMEMNQKLFDDCTQQY
KAEKQKGRFRMKEREEMWQKIEELARLNPQYPMFRAPPPLPPVYSMET
ETPTAEDIQLLKRTVETEAVQMLKDIKKDKVLLRRKSELPQDVYTIKA
LEAHKRAEEFLTASQEAL
>gi|33942058|ref|NM_009358.2| Mus musculus protein
phosphatase 2, regulatory
subunit B (B56), delta isoform (Ppp2r5d), mRNA
GTGGCGAAGAGACGCCGAGCGGGCCGAGTGTGGCCGAGCAGAGCCGGA
GCGGGGCCGCAGGAGCCGGGCCGGGTGTGGACCGGCCGAGATGTCCTA
TAAACTGAAGAAGGATAAGGAACCCTCCAAGCTTGCCAAAGGCACAGC
CAAGCCCAGCAGCTCAAGCAAGGATGGTGGAGGGGAGAACACCGATGA
GGCCCAGCCCCAGCCCCAGTCTCAGTCACCATCATCCAACAAGCGACC
CAGCAACAGTACACCACCCCCAACACAACTCAGCAAAATCAAGTACTC
AGGGGGACCCCAGATTGTCAAGAAAGAACGACGCCAAAGCTCCTTCCC
CTTCAACCTCAACAAGAACCGGGAGCTACAAAAACTTCCTGCCTTGAA
AGACTCACCAACCCAGGAACGTGAGGAGCTGTTTATCCAGAAGCTACG
CCAGTGCTGTGTCCTCTTTGACTTCGTGTCAGACCCACTCAGTGACCT
CAAATGCAAAGAGGTGAAGCGGGCAGGACTCAATGAGATGGTGGAGTA
TATCACCCACAGCCGTGATGTTGTCACTGAGGCCATCTACCCTGAGGC
TGTCACCATGTTTTCAGTGAATCTCTTCCGGACGCTGCCTCCTTCATC
GAATCCCACAGGAGCAGAGTTTGACCCTGAGGAAGATGAGCCAACCTT
GGAAGCTGCCTGGCCGCATCTCCAGCTCGTGTACGAGTTTTTCTTACG
TTTCTTGGAGTCTCCAGATTTCCAGCCAAATATAGCCAAGAAGTACAT
TGACCAGAAGTTTGTACTTGCTCTCCTGGACCTATTCGATAGTGAAGA
CCCTAGAGAGCGGGATTTTCTCAAGACCATTTTGCATCGCATCTATGG
CAAGTTTTTGGGGCTCCGGGCTTATATTCGTAGGCAGATCAACCACAT
CTTCTACAGGTTTATCTATGAGACTGAGCATCACAATGGGATTGCGGA
GCTGCTGGAGATCCTGGGCAGCATCATCAATGGCTTTGCCTTGCCCCT
TAAGGAAGAACACAAGGTGTTTCTCGTCCGTGTCCTGCTTCCACTTCA
CAAAGTCAAGTCTCTGAGTGTATACCACCCTCAGTTGGCGTACTGTGT
GGTGCAGTTCCTGGAGAAGGAGAGCAGTCTCACTGAGCCGGTGATTGT
GGGACTTCTCAAGTTTTGGCCTAAGACCCACAGCCCCAAGGAGGTGAT
GTTTCTGAACGAGCTGGAGGAGATTCTAGATGTCATTGAACCCTCGGA
GTTCAGTAAAGTCATGGAGCCGCTCTTCCGCCAGCTTGCCAAATGTGT
TTCCAGCCCCCATTTCCAGGTGGCGGAGCGCGCCCTCTACTACTGGAA
CAATGAGTACATCATGAGCCTCATCAGCGACAATGCTGCCCGCATCCT
CCCCATCATGTTTCCTGCACTCTATAGGAACTCCAAAAGCCACTGGAA
CAAGACAATCCATGGACTCATCTACAATGCCCTGAAGCTCTTCATGGA
GATGAACCAGAAGCTGTTTGACGACTGCACACAGCAGTACAAGGCGGA
GAAGCAGAAGGGCAGGTTCCGAATGAAGGAGAGAGAGGAGATGTGGCA
GAAGATCGAGGAGCTGGCCCGGCTCAATCCCCAGTACCCTATGTTTCG
GGCTCCTCCGCCACTGCCCCCTGTGTACTCCATGGAGACAGAGACGCC
CACAGCAGAGGACATTCAGCTCCTGAAGAGGACGGTGGAGACAGAGGC
CGTGCAGATGCTGAAGGACATCAAGAAGGACAAAGTGCTGCTCCGGAG
GAAGTCAGAGCTGCCACAGGACGTGTACACCATCAAGGCACTAGAGGC
GCACAAGCGGGCAGAAGAGTTCCTGACTGCCAGCCAGGAGGCCCTCTG
ACCCCCTCACCCTTCCCTTCCCACAGGATTCCAGCCCACTCAGCCCTG
GGATGCAGCCCAGCCCTCCACCCTCTGCTCCTCATCGGCTGACCTGGG
GCAGAACGGCACCTCTCTGGCTACTCTAGAGGATGCAGGCACTGGAAG
CGGGATGCCCAGAGCGCTCCCTCTTCTCCCCTAAACGTGTTCATGCCT
′CCCTGTGGCTAGTACAGACAGGCTGAGCACTGAGTGCTCAGTGCTTA
GACAACCTGGGGGTGCCTGTCCCCCTGCTCCTAACCCCACAGCTGCCT
GAAACTGTTCTGAGAAATACACACAGGAATCACATGTCCCTCTCTACC
CCCTTAATCCTCATCTGAAGTTGAGGTGTCTCTCCTCTCCCTGCAGAG
TACAACAGGAGACTGTCACCAAAGTCACGTTAAGCTCCGTTGGCCCCT
TGAGCGAAGAGCTGGAGGGAACCAATCCCCAGCAGCACAAATAGGCCC
CTAGACCCAGCTTTATGCAGGCGGGGGTGGTAGTGTGTTTCTTTGTAT
CCTATACCTAACCAAGACAACACAGAGGACAAACAAGTTACGGACACT
AGATGACTAGTGGGCCCGGTGGGGAGAAAGAACCATCTGCCTTGCCCT
CTCTGGCAAGCAGCAGTCCTGGGATCACACATTTGCAAGGGACCACCC
TGTGGCTGACTGCTTTCCCTTGTGCTCTTGGTTCCCAGAGCTATAAAG
AAGGAGGCAGGAGCAGTGCTCCAAGCATGGCTCCCTGCTGTGCCTGTT
TATTTCCTGGTTTCTGCTGACGCTGGGCTGGGCGGGTCCTCACCTGTA
CCCTCTAGGCGCTCAGAAACAAAAGCCTGGGGAGGGCTGGACCAGGAT
TCAGGAGCACAGGCGGTGTGGCTTCTGGCTGTGTACACAGGGTGCTTT
TTCTCCACAGAGTGATACATGCTAAGGTGGGCTGGGCTTGGGCCGATG
TCCCCATATGTACAGAACTGAATAAAGTGGGTCTCTGAG
PPP2R5E (Human)
>gi|5453956|ref|NP_006237.1| epsilon isoform of
regulatory subunit B56, protein phosphatase 2A
[Homo sapiens]
MSSAPTTPPSVDKVDGFSRKSVRKARQKRSQSSSQFRSQGKPIELTPL
PLLKDVPSSEQPELFLKKLQQCCVIFDFMDTLSDLKMKEYKRSTLNEL
VDYITISRGCLTEQTYPEVVRMVSCNIFRTLPPSDSNEFDPEEDEPTL
EASWPHLQLVYEFFIRFLESQEFQPSIAKKYIDQKFVLQLLELFDSED
PRERDYLKTVLHRIYGKFLGLRAFIRKQINNIFLRFVYETEHFNGVAE
LLEILGSIINGFALPLKAEHKQFLVKVLIPLHTVRSLSLFHAQLAYCI
VQFLEKDPSLTEPVIRGLMKFWPKTCSQKEVMFLGELEEILDVIEPSQ
FVKIQEPLFKQIAKCVSSPHFQVAERALYYWNNEYIMSLIEENSNVIL
PIMFSSLYRISKEHWNPAIVALVYNVLKAFMEMNSTMFDELTATYKSD
RQREKKKEKEREELWKKLEDLELKRGLRRDGIIPT
>gi|31083295|ref| NM_006246.2| Homo sapiens
protein phosphatase 2, regulatory subunit B′,
epsilon isoform (PPP2R5E), mRNA
GGTTGCGCGTGTGCCATGGACTCAGCCGCCCGGTGATATTGACAATAG
GAGAGAGAAAGGGGCATTGACTGGGACCCACCGCGGGTAGCGAAAGGT
GGCTCTGGCAGCGGCGGCTCCAGCTCCTGCGGCTCCTCCTCCTTATTC
TGTCCCCTTCTCTTGCTGCCGCTGCAGATCCAGTCTTCCTCCCTCCCT
TCCCCCCCTCCCCACGTCGTCGCCGCCGCCGCCGGGTCCGGGGCAACG
AGCTGAGGCGCCGCCCGCCAGGAATGTGAGCGAGGAGCCACCGGCGGA
GCCGCAACGGGGTCGGTGCCGATTTGATGGGACGGGCCCGCGGGGGAG
GATCGTGAGGCCGCCGCCGCCACCGGAACGCTGAGGTTCGGGTCCGGC
CGTGAGGCCTAGAGGCTCCGCCGCCGCGGAACCGGAGGGACCCCGTAC
CGGACAGCCGTCGCCCCAGGCTCCCCGCAGCTGCCCGGACCTCCCCCT
GCACGTCCCGGTCCCGCCGCCCGCCCCCGCTGCGGCCACCTCGCCCGT
CTCCCGCCCCTCCAAGCCACAGATCATCTTTGGATTCTTCCCCAGAAG
CTTCAAGTAGGGATATGTCCTCAGCACCAACTACTCCTCCATCAGTGG
ATAAAGTAGACGGATTTTCTCGGAAGTCCGTCAGAAAAGCCAGACAGA
AGAGGTCGCAAAGTTCCTCACAGTTTAGGTCTCAAGGCAAGCCTATTG
AGTTAACACCTCTGCCGCTGCTAAAAGACGTTCCATCCTCAGAGCAGC
CTGAACTGTTCCTAAAGAAACTTCAGCAGTGCTGTGTCATTTTTGACT
TCATGGACACGCTATCTGATCTTAAAATGAAAGAATACAAGCGCTCCA
CTCTTAATGAACTGGTGGACTACATTACAATAAGCAGAGGCTGTTTGA
CAGAGCAGACTTACCCTGAAGTAGTTAGAATGGTATCTTGCAATATAT
TCAGAACTCTCCCTCCTAGTGACAGCAATGAATTTGATCCAGAAGAAG
ATGAACCTACCCTTGAGGCATCGTGGCCACACTTACAGCTTGTATATG
AATTTTTCATACGATTTTTGGAAAGCCAAGAATTCCAACCCAGCATTG
CCAAAAAATATATAGATCAGAAATTTGTATTACAGCTTCTGGAGCTAT
TTGACAGCGAAGACCCTCGGGAACGGGACTACTTAAAAACAGTCTTAC
ACAGAATTTATGGCAAGTTTCTTGGTCTTAGAGCATTTATCCGAAAAC
AGATTAACAATATTTTTCTAAGGTTTGTTTATGAAACAGAACACTTCA
ATGGTGTAGCTGAACTGCTGGAAATATTAGGAAGTATTATCAATGGCT
TTGCTTTACCTCTTAAGGCAGAACACAAACAGTTTCTGGTGAAAGTAT
TGATCCCTTTACACACTGTCAGGAGCTTATCACTCTTCCATGCACAGC
TGGCATATTGTATAGTACAGTTTCTGGAGAAAGATCCTTCACTCACAG
AACCAGTTATTAGGGGGTTAATGAAATTTTGGCCTAAAACATGTAGTC
AAAAAGAGGTCATGTTCCTTGGGGAACTGGAAGAAATATTGGATGTGA
TTGAACCTTCACAATTTGTTAAAATCCAAGAACCTTTGTTTAAACAAA
TCGCCAAGTGTGTATCTAGCCCCCATTTTCAGGTGGCAGAAAGAGCAC
TCTATTATTGGAATAATGAATACATCATGAGTTTGATAGAAGAAAACT
CTAACGTCATCCTTCCCATCATGTTTTCCAGCCTTTATAGGATTTCAA
AAGAACATTGGAATCCGGCTATTGTGGCGTTGGTGTACAATGTGTTGA
AGGCATTTATGGAAATGAACAGCACCATGTTTGACGAGCTGACAGCCA
CATACAAGTCAGATCGTCAGCGTGAGAAAAAGAAAGAAAAGGAGCGTG
AAGAATTGTGGAAAAAATTGGAGGATCTGGAGTTAAAGAGAGGTCTTA
GACGTGATGGAATAATTCCAACTTAACAAAAACAATGACAACAACATT
ACTAACCTGTGGAGTCACACGTTTATGTAGTAGAAGATGGAGCAACAG
TTTTCTGTATTGTGCAACTTTACAGTAGATTTCACCTTTGTTTCATTA
TTACAGCAGCACTGTATATACCTGTCTCTAAGTAAAGGAAAAAACAAA
ATAAGGACTTCAATCCAAAGTTTGGACAGTAGATGGACTTCTCAGAAC
TTTGCAAACATAATCATTGTTCTCACCCTCTTTTAAAAAAAAAAATCG
GTCTTCAAAGATCTGTTGATGAAATTGCTATGTTAAAATTCCATTATC
GGGAGTTCCTTATTTATCACTAGCAGAGAGTATGATACAATTTTCAAA
TGTGAACAATCTTAAATTTAGCTTGTCTTTCTGCTAAGCTGTTAAATG
TATTTATAGTAAAGGAAGAAAAAAAGACTGTCATTTCCTTATAAGTTT
GTGTAACATCCTCCTCTGGATAACTTGACTGTAATTTAACATCTTTTT
CTTTTGCACATCTTCCTGAGTTGAATGTCCACGTGGAATGGGGTCATG
AATTATAAAAGTCCCTGATAAAAGTTTTGTTTACTGGGGTGAACATCT
TTCCAGTAACCAGGTAGTCCTGGTACTCCTTTAGTTTTAAAATTAGGA
GTTAAGAGAGAAGAGGTGATAAACATAGTAGGGAAGGGAATATCGGAT
TCATGCATCAGTTTATGGTGAATCCAAATCAATGTCTTGAATCCTTTG
AAAACAGGCACTGGGACATCACAGGCTTCAGTACCTGACCAGTATTAG
TTGCATATATCATTGAACACACATACCAGAGATGTTTTAGAAATGTGA
GAAAAACATCCTTTTGGACCATTTGAAATAAGAAAGACAAACACTAAA
CAATACAACCATGAAATTGATCACCGGGATTGCAAATCTAATTGGGAA
AAGAGTTGAGCAAACAGCTTGGACTGTTTGGAGTTGTTGCCTTACTTT
TTAATATGTATTTATAAAGTATTCCAGCAAAAGAGGATGTAGCCTCTG
GGAAAAAACAAACATGTTACAGTGTTTTTTGTAGATTCTCGTTCTATA
TCTCATCACAGCGCCAGCCCTGTTTTTAGCCGGAAAGGATTCAGGATA
AACATTATTATGCATTCTGAATTGGATGCATATTCCTAACTACTGTAT
TTGTTACCAAAAGTGGTTCTACAAATGCTACTGAAAAAAATCTGGAAA
TTCCTAATGTCCTGAGTATTAATAATAAAGTTTAAAAATGCTTTTATA
TCAAAGGTGCATCGTGACCAAATTGTTTAAGAAAAAAAAACAAAAAAA
ACAAAATCTAGGGCTGTATTTTATATATATATATATATATATATATAT
ATATAT
PPP2R5E (Mouse)
>gi|33859660|ref|NP_036154.1| epsilon isoform of
regulatory subunit B56, protein phosphatase 2A
[Mus musculus]
MSSAPTTPPSVDKVDGFSRKSVRKARQKRSQSSSQFRSQGKPIELTPL
PLLKDVPTSEQPELFLKKLQQCCVIEDFMDTLSDLKMKEYKRSTLNEL
VDYITISRGCLTEQTYPEVVRMVSCNIFRTLPPSDSNEFDPEEDEPTL
EASWPHLQLVYEFFIRFLESQEFQPSIAKKYIDQKFVLQLLELFDSED
PRERDYLKTVLHRIYGKFLGLRAFIRKQINNIFLREVYETEHENGVAE
LLEILGSIINGFALPLKAEHKQFLVKVLIPLHTVRSLSLFHAQLAYCI
VQFLEKDPSLTEPVIRGLMKFWPKTCSQKEVMFLGELEEILDVIEPSQ
FVKIQEPLFKQIAKCVSSPHFQVAERALYYWNNEYIMSLIEENSNVIL
PIMFSSLYRISKEHWNPAIVALVYNVLKAFMEMNSTMFDELTATYKSD
RQREKKKEKEREELWKKLEDLELKRGLRRDGIIPT
>gi|55741699|ref|NM_012024.2| Mus musculus protein
phosphatase 2, regulatory subunit B (B56), epsilon
isoform (Ppp2r5e), mRNA
GTGATATTGACAATAGGAGAGAGAAAGGGGCATTGACGGGGACCCTGC
GCGGGTAGCGAACGGCGGCTCTGGCAGCGGCGGCTCAGGCGGCTCCAA
CTCTTCCTCCCGACTCCCGCGCTTGCTGCCGCTGCAAATCCAGGCTTC
CTCCCTCCTCCCCCTCCCCGCGTCGCCGCCGCCGCCGCCGCCGCCACC
ACCGCCGCCGGGTCCGGTGCAACGAGCAGAGGCGCCGCCCGCCGGGAA
TGTGAACGAAGAGCCACCGGCCGCGCCGCAACCGGGTCGGTGCCGATT
TGATGGGACGGGCCCGCGGGGGAGGCTCGTGAGGCCGCCGCTGCCACT
GCCGGAGCGCTGAGGTTAGGGGCCAGCCGAGAGGCCTAGAAACACTGC
CGCTACCGCGGAACCGGAGGGACGTCGCCCCGGACGGCCGTAGTAGCC
CGGGGCTCTCCGCCGCTGCCTGGACCTCCCCCCGTGCGTCCCGGGCCG
TCGCCCGCCCCCGCCGCGGCCCCTAGCTCGCAGCCCACTCCGCGCAGC
CACAGATCATCTTCCGATTCTTTCCCAGGAGCTTCAAGTAGGGATATG
TCCTCAGCACCAACTACTCCTCCATCAGTGGATAAAGTAGACGGATTT
TCTCGGAAGTCCGTCAGAAAAGCCAGACAGAAGAGGTCGCAAAGCTCC
TCCCAGTTCAGGTCTCAAGGCAAGCCCATTGAGCTCACGCCTCTGCCA
CTGCTGAAAGACGTTCCAACCTCAGAGCAGCCTGAACTGTTCCTAAAG
AAACTTCAGCAGTGCTGTGTCATTTTTGACTTCATGGACACGCTATCT
GATCTTAAAATGAAAGAATACAAGCGCTCCACTCTTAATGAACTGGTG
GACTACATTACAATAAGCAGAGGCTGTCTGACAGAGCAGACTTACCCT
GAAGTAGTTAGAATGGTATCTTGCAATATATTCAGAACTCTCCCTCCT
AGTGACAGCAATGAATTCGACCCAGAAGAAGATGAACCTACCCTCGAG
GCATCATGGCCCCACTTACAGCTTGTATATGAATTTTTCATAAGATTT
TTGGAAAGCCAAGAATTCCAACCCAGCATTGCCAAAAAATACATAGAT
CAGAAATTTGTATTACAGCTTTTGGAGCTATTTGACAGTGAAGACCCT
CGAGAACGGGACTATTTAAAAACAGTCTTGCACAGGATTTATGGCAAG
TTTCTCGGGCTTAGAGCATTTATCCGAAAACAAATTAACAATATTTTT
CTAAGGTTTGTTTATGAAACAGAACACTTCAATGGTGTAGCTGAACTG
CTGGAAATATTAGGAAGTATTATCAATGGCTTTGCTTTACCACTCAAG
GCAGAACACAAACAGTTCCTGGTGAAAGTGTTGATCCCTCTACACACT
GTCAGGAGCTTGTCACTCTTTCATGCACAGCTGGCGTATTGTATAGTA
CAGTTTCTGGAGAAAGACCCTTCCCTTACAGAACCAGTTATTAGGGGG
CTAATGAAATTTTGGCCTAAAACATGCAGTCAAAAAGAGGTCATGTTC
CTCGGGGAGCTGGAAGAAATCTTGGATGTGATTGAACCGTCACAATTT
GTTAAAATCCAAGAACCTTTGTTTAAACAAATTGCCAAGTGTGTTTCT
AGCCCCCATTTTCAGGTGGCAGAAAGGGCACTCTATTATTGGAATAAT
GAATACATCATGAGTTTGATAGAAGAAAACTCTAACGTCATCCTTCCC
ATCATGTTCTCCAGCCTTTATAGGATTTCAAAAGAGCATTGGAATCCG
GCCATTGTGGCATTGGTGTACAACGTGTTGAAGGCATTTATGGAAATG
AACAGCACTATGTTTGATGAGCTGACAGCCACATACAAGTCAGATCGT
CAGCGTGAGAAAAAGAAAGAAAAGGAGCGAGAAGAACTGTGGAAAAAA
TTGGAGGATCTGGAGTTAAAGAGAGGTCTTAGACGCGATGGGATAATT
CCAACTTAACAACAGCCTGACAGCGACACACTAACCCGTGGGTCACAC
GCTTATGTAGTAGAAGGATGGAGCAACAGTTTTCTGTATTGTGCAACT
TACAGTAGATTTCACATTTGTCTCATTATTACAGCAGCACTGTATATA
CCTGTCTCTAAGTAAAGGAAAACATAAGGACTTCAAAGTGTGGACAGT
AGATGGACTCTCAGAACTTTGCAAACATAATCATTGTTCTAACCCTCT
TTAAAAACAAACAAACAAACAAAAGGTCTACAGAGATCTGTCGGGGAA
ATTGCTATGTTAAAATTCCATTATCAGGAGTTCCTTATTTATCACTAG
CAGAGAGTATGATACAATTTTCAAATGTGAACAATCTTAAATTTAGCT
TGTCTTTCTGCTAAGCTGTTAAATGTATTTATAGTAAAGGAAGAAAAG
ATTGTCATTTCTTTATAAGTTTGTATAGCGCCCTCCTCTGGACATGAC
TGTAACGTGATATCCTTTCCTTTTGCACATCTTCATGAGTCAAGTGTC
CACATGGACCGGGGCTGTGAGTCAGAGGTCTCTGAGGAAAGGTTGTCT
GCTGGATGAACATCTTCCCAGCTGCCAGGTCCTGGTACTCCTTTAGTT
TTCAGATTAGGAGTAAAAAGAAGAGGTGATAAGCATGGTAGCGAAGGG
AATGTTGGACTTCATATCAGATATGGTGAGTCCAAATCAGTGTCTTGA
ATCCTTTGAAAACAGGCAGTGGGACGCGCAGGCTTCAGCACCTGCAGC
ATTAGCTGCAGACATAAGTGTGCATCCAGACCAGAGACAACTTAGAAA
TGTCAGAGAACATCCTTTCGGATCCTGTGGAGTAAGAGAGAGAGCACT
AAGCGATAGAACCGTGAAGCCGAGCACCAGGACCGCGCATCCACTTGG
GAACCGAGTTGAGCAAACAGCATGAACTGTTTGCAGTTGATGCCTTAC
TTCCTAATGTGTACTTACAAAGTATTTCAGCAGAAGAGGATGCAGGGT
GCAGCCTCTGGGAGGAACATCCTCCTTCTCTAGCTCACCACAGCGCCA
GCCCTGTTTTTAGCCAGAAAGGATTCAGGATCAACATTATGCGTTCTG
AATTGGATGCATATTCCTAACTACTGTATTTGTTACCAAAAGTGGTTC
TACAAATGCTACTGAAAAAAATCTGGAAATTCCTAATGTCCTGAGTAT
TAATAATAAAGTTTAAAAAATGCTTTTATATCAAAGGTGCATCGTGAC
CAAATTGTTTAAGTTAAAAACAAAACAAAACCAAACCAACCCGAGGGC
TGTGCTCTAGCTGGGCTTACTGTCTCTGTACTGGGTCCCCCTGGGTAG
GTAAGTTGCTGATGTACAGGTGTGCAGTATGTATTCACCCGATGTGTT
GCATCGCTGTGTATTGGCAAGTGTGTGTTGTGGGGTTTGTTTCCAGGA
AGCTTTTTCTTGTTAGAGACAGGACAGTGGTGAGCCCATAACCCCGAG
CCATCACATCCAGCAGTGTTCATGGCATAGAGCCGACTCTGTGTTCAT
ATGTTGAGTTGTGATTATAGCTAACATGAACACACATGAGCTATTGTT
GCTCTTAGTAGAAATACAGTATAAATATGGGCTTGTATATTTTCTTCC
TTCCTTTTAAATGTAAAGTAGAATATATAGAGCTTTGTTGTTTCCAAG
GAAAAGGAGGGGAAAAGCCACTGACCTATTTCACTTATGAAGTATTTC
CAGATTGTCAGTCACAATGTGTACATAAATGTAAGCTAATTATGGATA
TTTTCATTTAGGTACCTTTGTTCTCTTGTTTTTTAAAGGTATGCGTAC
CACCAAGTCCAAGATGGTTCAGTGGAATAAATAGGACTCTTAAAGAGC
GTTAAGGAGGGTTCCATCTGAGGACCTGGAAAGTGTGAGCAGAGCTGC
ATGTTCACATATTGATGCTAAGAAGCAAACCATAGCACATCTCCCTTA
GCTGTCTTAGCCGTTTCCATCTTCATTACACATATCTTTGATAGTGTA
GACCTTGCTTGCTGTGGTCCCTCTGGGGATGACATGTGCTCATTTGAC
ATGAGGAAGGTCAGGACCTCTTTCTGCACCGTGTCCTGTCCTCAGAGC
TAAGGGTCAGTGTTGTCTGTTGGTCATTCCCAACTGAGAGGAATGAAG
CTAAGCTTTAGACTATTTAAAGATGTGATGAGAGCTGAATGTCAGAAC
TCACAGGGCAATCTCTGAGAAGAAACACTCATTTCCCTTTTAACCAAA
AGTGACAATGTGACCTCTGTCATGTTGTGTGTGGTGAGGAGAAGCTAG
CCCAGGTGACTTGATTGTTGCACTATGTGACATATTTAAGGACATACT
TGCTGCATTTGCAGCAGCCCCCCTGTCTGACCTCCCTGGGCAATCCTG
CTGTCACTCAATGTGAGTTAACTGTGACAAACTCAGCTCTGTGTTATA
TTTTGTTTGCTTCATAGTTTCCAGAACTATATATAAATATATTTTTTC
AATAGCAAATATTGTAGTTAGTGAGTGGTGACCACTTCAACCTTATCC
CTACCATATTGGTTTCAGGGATGGGACTAAGAATGTCCTTGGAATAAA
AAAATTAAAATATAACCCTGTGATTTAGAATGATGCCGAGGTTGCAGG
TTGCAGCCTGCAGCAGCTGTGTGAGCGAAGTGACCGTCTCCTCTGGAT
CAAGCGTCCACTCCTCATTGTGCCCAGGCCCCAGTGCTCCTTCATCAC
TGTTTAGTTATTGTCCGCTGTGGTTTTGATTATATTTTGCTCTTCCTG
ACATTGACTGTTTTTTTTTAATAAAAGAAAATAAAAAGTAAATGAATA
ACCCCTGAAAAAAAAAAAAAAAAAAAAAAAAAAAAAAAAA

In other embodiments, the nucleic acid molecule has at least 70% identity, more preferably 80% identity, and even more preferably 90% identity with a nucleic acid molecule comprising: at least about 700, at least about 800, at least about 1000, at least about 1200, at least about 1400 or at least about 1600 contiguous nucleotides of the sequences shown above and includes the DNA binding domain. In other embodiments, the nucleic acid molecule has at least 70% identity, more preferably 80% identity, and even more preferably 90% nucleotide identity with a nucleic acid molecule comprising: at least about 600, at least about 800, at least about 1000, at least about 1200, or at least about 1400 contiguous nucleotides of the sequences shown above and includes the DNA binding domain.

Nucleic acid molecules that differ from the sequences shown above due to degeneracy of the genetic code, and thus encode the same PP2A B56 regulatory subunit protein as that encoded by the sequences shown above, are encompassed by the invention. In addition, nucleic acid molecules encoding PP2A B56 regulatory subunit proteins can be isolated from other sources using standard molecular biology techniques and the sequence information provided herein. For example, a PP2A B56 regulatory subunit DNA can be isolated from a human genomic DNA library using all or portion of the sequences shown above as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989). Moreover, a nucleic acid molecule encompassing all or a portion of a PP2A B56 regulatory subunit gene can be isolated by the polymerase chain reaction using oligonucleotide primers designed based upon the sequence of SEQ ID NO: 1 or 3. For example, mRNA can be isolated from cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO: 1 or 3. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis.

Furthermore, oligonucleotides corresponding to a PP2A B56 regulatory subunit nucleotide sequence can be prepared by standard synthetic techniques, e.g., using an automated DNA synthesizer.

In addition to the PP2A B56 regulatory subunit nucleotide sequences shown above, it will be appreciated by those skilled in the art that DNA sequence polymorphisms that lead to minor changes in the nucleotide or amino acid sequences of PP2A B56 regulatory subunit may exist within a population. Such genetic polymorphism in the PP2A B56 regulatory subunit gene may exist among individuals within a population due to natural allelic variation. Such natural allelic variations can typically result in 1-2% variance in the nucleotide sequence of a gene. Any and all such nucleotide variations and resulting amino acid polymorphisms in the PP2A B56 regulatory subunit that are the result of natural allelic variation and that do not alter the functional activity of are intended to be within the scope of the invention.

Nucleic acid molecules corresponding to natural allelic variants of the PP2A B56 regulatory subunit DNAs of the invention can be isolated based on their homology to the PP2A B56 regulatory subunit nucleic acid molecules disclosed herein using the human DNA, or a portion thereof, as a hybridization probe according to standard hybridization techniques under high stringency hybridization conditions. Exemplary high stringency conditions include hybridization in a hybridization buffer that contains 6× sodium chloride/sodium citrate (SSC) at a temperature of about 45° C. for several hours to overnight, followed by one or more washes in a washing buffer containing 0.2×SSC, 0.1% SDS at a temperature of about 50-65° C. Accordingly, in another embodiment, an isolated nucleic acid molecule of the invention hybridizes under high stringency conditions to a second nucleic acid molecule comprising the nucleotide sequences shown above. Preferably, an isolated nucleic acid molecule of the invention that hybridizes under high stringency conditions to the sequences shown above. In one embodiment, such a nucleic acid molecule is at least about 700, 800, 900, 1000, 1200, 1300, 1400, 1500, or 1600 nucleotides in length. In another embodiment, such a nucleic acid molecule comprises at least about 700, 800, 900, 1000, 1200, 1300, 1400, 1500, or 1600 contiguous nucleotides and includes the biologically active domain of the B56 regulatory subunit. Preferably, an isolated nucleic acid molecule corresponds to a naturally-occurring allelic variant of a PP2A B56 regulatory subunit nucleic acid molecule.

In addition to naturally-occurring allelic variants of the PP2A B56 regulatory subunit sequence that may exist in the population, the skilled artisan will further appreciate that minor changes may be introduced by mutation into the nucleotide sequences shown above, thereby leading to changes in the amino acid sequence of the encoded protein, without altering the functional activity of the PP2A B56 regulatory subunit protein. For example, nucleotide substitutions leading to amino acid substitutions at “non-essential” amino acid residues may be made in the sequences shown above. A “non-essential” amino acid residue is a residue that can be altered from the wild-type sequence of PP2A B56 regulatory subunit (e.g., the sequences shown above) without altering the functional activity of PP2A B56 regulatory subunit, such as its ability to interact with the catalytic and/or structural subunits of the PP2A holoenzyme and function to dephosphorylate AKT-1, whereas an “essential” amino acid residue is required for functional activity.

Accordingly, another aspect of the invention pertains to nucleic acid molecules encoding PP2A B56 regulatory subunit proteins that contain changes in amino acid residues that are not essential for PP2A B56 regulatory subunit activity. Such PP2A B56 regulatory subunit proteins differ in amino acid sequences shown above yet retain PP2A B56 regulatory subunit activity. An isolated nucleic acid molecule encoding a non-natural variant of a PP2A B56 regulatory subunit protein can be created by introducing one or more nucleotide substitutions, additions or deletions into the nucleotide sequences shown above such that one or more amino acid substitutions, additions or deletions are introduced into the encoded protein. Mutations can be introduced into the sequences by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a PP2A B56 regulatory subunit is preferably replaced with another amino acid residue from the same side chain family.

Alternatively, in another embodiment, mutations can be introduced randomly along all or part of the PP2A B56 regulatory subunit coding sequence, such as by saturation mutagenesis, and the resultant mutants can be screened for their ability to bind to DNA and/or activate transcription, to identify mutants that retain functional activity. Following mutagenesis, the encoded PP2A B56 regulatory subunit mutant protein can be expressed recombinantly in a host cell and the functional activity of the mutant protein can be determined using assays available in the art for assessing PP2A B56 regulatory subunit activity (e.g., by measuring the ability of the protein to bind to a T-box binding element present in DNA or by measuring the ability of the protein to modulate IL2 production).

Yet another aspect of the invention pertains to isolated nucleic acid molecules encoding PP2A B56 regulatory subunit fusion proteins. Such nucleic acid molecules, comprising at least a first nucleotide sequence encoding a PP2A B56 regulatory subunit protein, polypeptide or peptide operatively linked to a second nucleotide sequence encoding a non-PP2A B56 regulatory subunit protein, polypeptide or peptide, can be prepared by standard recombinant DNA techniques.

In one embodiment, a nucleic acid molecule encoding a PP2A B56 regulatory subunit or a biologically active portion thereof is present in an expression vector for expression in a host cell. Such expression vectors can be used to make recombinant PP2A B56 regulatory subunit or to increase PP2A B56 regulatory subunit activity in a cell, e.g., a cell of the innate immune system. The expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., PP2A B56 regulatory subunit proteins, mutant forms of PP2A B56 regulatory subunit proteins, PP2A B56 regulatory subunit fusion proteins and the like).

The recombinant expression vectors of the invention can be designed for expression of a PP2A B56 regulatory subunit protein in prokaryotic or eukaryotic cells. For example, PP2A B56 regulatory subunit can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector may be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors can serve one or more purposes: 1) to increase expression of recombinant protein; 2) to increase the solubility of the recombinant protein; 3) to aid in the purification of the recombinant protein by acting as a ligand in affinity purification; 4) to provide an epitope tag to aid in detection and/or purification of the protein; and/or 5) to provide a marker to aid in detection of the protein (e.g., a color marker using β-galactosidase fusions). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Recombinant proteins also can be expressed in eukaryotic cells as fusion proteins for the same purposes discussed above.

Examples of suitable inducible non-fusion E. coli expression vectors include pTrc (Amann et al., (1988) Gene 69:301-315) and pET lid (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nuc. Acids Res. 20:2111-2118). Such alteration of nucleic acid sequences of the invention can be carried out by standard DNA synthesis techniques.

In another embodiment, the PP2A B56 regulatory subunit expression vector is a yeast expression vector. Examples of vectors for expression in yeast S. cerivisae include pYepSec1 (Baldari. et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, PP2A B56 regulatory subunit can be expressed in insect cells using baculovirus expression vectors. Baculovirus vectors available for expression of proteins in cultured insect cells (e.g., Sf 9 cells) include the pAc series (Smith et al., (1983) Mol. Cell Biol. 3:2156-2165) and the pVL series (Lucklow, V. A., and Summers, M. D., (1989) Virology 170:31-39).

In yet another embodiment, a nucleic acid of the invention is expressed in mammalian cells using a mammalian expression vector. Examples of mammalian expression vectors include pMex-NeoI, pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), the albumin promoter (liver-specific; Pinkert et al. (1987) Genes Dev. 1:268-277), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546). In one embodiment, an adipocyte cell specific promoter is operably linked to a PP2A B56 regulatory subunit nucleic acid molecule.

Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Accordingly, in another embodiment, the invention provides a recombinant expression vector in which PP2A B56 regulatory subunit DNA is operatively linked to an inducible eukaryotic promoter, thereby allowing for inducible expression of a PP2A B56 regulatory subunit protein in eukaryotic cells.

Another aspect of the invention pertains to recombinant host cells into which a vector, preferably a recombinant expression vector, of the invention has been introduced. A host cell may be any prokaryotic or eukaryotic cell. For example, PP2A B56 regulatory subunit protein may be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to compounds, such as G418, hygromycin and methotrexate. Nucleic acid encoding a selectable marker may be introduced into a host cell on the same vector as that encoding PP2A B56 regulatory subunit or may be introduced on a separate vector. Cells stably transfected with the introduced nucleic acid can be identified by compound selection (e.g., cells that have incorporated the selectable marker gene will survive, while the other cells die).

A host cell of the invention, such as a prokaryotic or eukaryotic host cell in culture, can be used to produce (i.e., express) PP2A B56 regulatory subunit protein. Accordingly, the invention further provides methods for producing PP2A B56 regulatory subunit protein using the host cells of the invention. In one embodiment, the method comprises culturing the host cell of invention (into which a recombinant expression vector encoding PP2A B56 regulatory subunit has been introduced) in a suitable medium until PP2A B56 regulatory subunit is produced. In another embodiment, the method further comprises isolating PP2A B56 regulatory subunit from the medium or the host cell. In its native form the PP2A B56 regulatory subunit protein is an intracellular protein and, accordingly, recombinant PP2A B56 regulatory subunit protein can be expressed intracellularly in a recombinant host cell and then isolated from the host cell, e.g., by lysing the host cell and recovering the recombinant PP2A B56 regulatory subunit protein from the lysate. Alternatively, recombinant PP2A B56 regulatory subunit protein can be prepared as a extracellular protein by operatively linking a heterologous signal sequence to the amino-terminus of the protein such that the protein is secreted from the host cells. In this case, recombinant PP2A B56 regulatory subunit protein can be recovered from the culture medium in which the cells are cultured.

Certain host cells of the invention can also be used to produce nonhuman transgenic animals. For example, in one embodiment, a host cell of the invention is a fertilized oocyte or an embryonic stem cell into which PP2A B56 regulatory subunit-coding sequences have been introduced. Such host cells can then be used to create non-human transgenic animals in which exogenous PP2A B56 regulatory subunit sequences have been introduced into their genome or homologous recombinant animals in which endogenous PP2A B56 regulatory subunit sequences have been altered. Such animals are useful for studying the function and/or activity of PP2A B56 regulatory subunit and for identifying and/or evaluating modulators of PP2A B56 regulatory subunit activity. Accordingly, another aspect of the invention pertains to nonhuman transgenic animals which contain cells carrying a transgene encoding a PP2A B56 regulatory subunit protein or a portion of a PP2A B56 regulatory subunit protein. In a subembodiment, of the transgenic animals of the invention, the transgene alters an endogenous gene encoding an endogenous PP2A B56 regulatory subunit protein (e.g., homologous recombinant animals in which the endogenous PP2A B56 regulatory subunit gene has been functionally disrupted or “knocked out”, or the nucleotide sequence of the endogenous PP2A B56 regulatory subunit gene has been mutated or the transcriptional regulatory region of the endogenous PP2A B56 regulatory subunit gene has been altered).

A transgenic animal of the invention can be created by introducing PP2A B56 regulatory subunit-encoding nucleic acid into the male pronuclei of a fertilized oocyte, e.g., by microinjection, and allowing the oocyte to develop in a pseudopregnant female foster animal. The PP2A B56 regulatory subunit nucleotide sequences shown above can be introduced as a transgene into the genome of a non-human animal. Intronic sequences and polyadenylation signals can also be included in the transgene to increase the efficiency of expression of the transgene. A tissue-specific regulatory sequence(s) can be operably linked to the PP2A B56 regulatory subunit transgene to direct expression of PP2A B56 regulatory subunit protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the PP2A B56 regulatory subunit transgene in its genome and/or expression of PP2A B56 regulatory subunit mRNA in tissues or cells of the animals. A transgenic founder animal can then be used to breed additional animals carrying the transgene. Moreover, transgenic animals carrying a transgene encoding PP2A B56 regulatory subunit can further be bred to other transgenic animals carrying other transgenes.

To create a homologous recombinant animal, a vector is prepared which contains at least a portion of a PP2A B56 regulatory subunit gene into which a deletion, addition or substitution has been introduced to thereby alter, e.g., functionally disrupt, the endogenous PP2A B56 regulatory subunit gene. In one embodiment, a homologous recombination vector is designed such that, upon homologous recombination, the endogenous PP2A B56 regulatory subunit gene is functionally disrupted (i.e., no longer encodes a functional protein; also referred to as a “knock out” vector). Alternatively, the vector can be designed such that, upon homologous recombination, the endogenous PP2A B56 regulatory subunit gene replaced by the PP2A B56 regulatory subunit gene. In the homologous recombination vector, the altered portion of the PP2A B56 regulatory subunit gene is flanked at its 5′ and 3′ ends by additional nucleic acid of the PP2A B56 regulatory subunit gene to allow for homologous recombination to occur between the exogenous PP2A B56 regulatory subunit gene carried by the vector and an endogenous PP2A B56 regulatory subunit gene in an embryonic stem cell. The additional flanking PP2A B56 regulatory subunit nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced PP2A B56 regulatory subunit gene has homologously recombined with the endogenous PP2A B56 regulatory subunit gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In addition to the foregoing, the skilled artisan will appreciate that other approaches known in the art for homologous recombination can be applied to the instant invention. Enzyme-assisted site-specific integration systems are known in the art and can be applied to integrate a DNA molecule at a predetermined location in a second target DNA molecule. Examples of such enzyme-assisted integration systems include the Cre recombinase-lox target system (e.g., as described in Baubonis, W. and Sauer, B. (1993) Nucl. Acids Res. 21:2025-2029; and Fukushige, S. and Sauer, B. (1992) Proc. Natl. Acad. Sci. USA 89:7905-7909) and the FLP recombinase-FRT target system (e.g., as described in Dang, D. T. and Perrimon, N. (1992) Dev. Genet. 13:367-375; and Fiering, S. et al. (1993) Proc. Natl. Acad. Sci. USA 90:8469-8473). Tetracycline-regulated inducible homologous recombination systems, such as described in PCT Publication No. WO 94/29442 and PCT Publication No. WO 96/01313, also can be used.

Alternatively, null mutations can be generated by targeted mutagenesis in ES cells (Ranger, A. M., et al. 1998. Nature 392, 186; Hodge, M. R., et al. 1996. Immunity 4:1., 144; Grusby, M. J., et al. 1991. Science 253, 1417; Reimold, A. M., et al. 1996. Nature 379: 262; Kaplan, M. H., 1996. Immunity: 313; Kaplan, M. H., et al. 1996. Nature 382, 174; Smiley, S. T., et al. 1997. Science 275, 977). For example using techniques which are known in the art, a genomic PP2A B56 regulatory subunit clone can be isolated from a genomic library, the intron-exon organization delineated, and a targeting construct in the cre-lox vector (see discussion below) created which should delete the first exon and 450 bp of upstream promoter sequence. This construct can be electroporated into an ES cell line, and double compound resistant (e.g., neomycin, gancyclovir) clones identified by Southern blot analysis. Clones bearing homologous recombinant events in the PP2A B56 regulatory subunit locus can then be identified and injected into blastocysts obtained from day 3.5 BALB/c pregnant mice. Chimeric mice can then be produced and mated to wildtype BALB/c mice to generate germline transmission of the disrupted PP2A B56 regulatory subunit gene.

In another embodiment, implantation into RAG2-deficient blastocysts (Chen, J., et al. 1993. Proc. Natl. Acad. Sci. USA 90, 4528) or the cre-lox inducible deletion approach can be used to develop mice that are lacking PP2A B56 regulatory subunit only in the immune system. For example, the targeting construct can be made in the cre-lox vector. The blastocyst complementation system has been used to study NFATc, an embryonic lethal phenotype (Ranger, A. M., et al. 1998. Immunity 8:125). This approach requires disrupting the PP2A B56 regulatory subunit gene on both chromosomes in ES cells, which can be accomplished, e.g., by using a mutant neomycin gene and raising the concentration of G418 in the ES cultures, as described (Chen, J., 1993. Proc. Natl. Acad. Sci. USA 90;4528) or by flanking the neo gene with cre-lox sites. To disrupt the second allele, the neomycin gene can be deleted by transfecting the ES clone with the cre recombinase, and then the ES clone can be retransfected with the same targeting construct to select clones with PP2A B56 regulatory subunit deletions on both alleles. A third transfection with cre-recombinase yields the desired doubly-deficient ES cells. Such doubly targeted ES cells are then implanted into RAG2 blastocysts and the lymphoid organs of the chimeric mice thus generated will be entirely colonized by the transferred ES cells. This allows assessment of the effect of the absence of PP2A B56 regulatory subunit on cells of the lymphoid system without affecting other organ systems where the absence of PP2A B56 regulatory subunit might cause lethality.

The conditional ablation approach employing the cre-lox system can also be used. Briefly, a targeting construct is generated in which lox recombination sequences are placed in intronic regions flanking the exons to be deleted. This construct is then transfected into ES cells and mutant mice are generated as above. The resulting mutant mice are then mated to mice transgenic for the cre recombinase driven by an inducible promoter. When cre is expressed, it induces recombination between the introduced lox sites in the PP2A B56 regulatory subunit gene, thus effectively disrupting gene function. The key feature of this approach is that gene disruption can be induced in the adult animal at will by activating the cre recombinase.

A tissue-specific promoter can be used to avoid abnormalities in organs outside the immune system. The cre-expressing transgene may be driven by an inducible promoter. Several inducible systems are now being used in cre-lox recombination strategies, the most common being the tetracycline and ecdysone systems. A tissue-specific inducible promoter can be used if there is embryonic lethality in the PP2A B56 regulatory subunit null mouse.

An alternative approach is to generate a transgenic mouse harboring a regulated PP2A B56 regulatory subunit gene (for example using the tetracycline off promoter; e.g., St-Onge, et al. 1996. Nuc. Acid Res. 24, 3875-3877) and then breed this transgenic to the PP2A B56 regulatory subunit deficient mouse. This approach permits creation of mice with normal PP2A B56 regulatory subunit function; tetracycline can be administered to adult animals to induce disruption of PP2A B56 regulatory subunit function in peripheral T cells, and then the effect of PP2A B56 regulatory subunit deficiency can be examined over time. Repeated cycles of provision and then removal of compound (tetracycline) permits turning the PP2A B56 regulatory subunit gene on and off at will.

(ii). PP2A B56 Regulatory Subunit Proteins

In one embodiment, an isolated PP2A B56 regulatory subunit proteins or a biologically active portion thereof is used to increase PP2A B56 regulatory subunit activity in a cell. In one embodiment, the PP2A B56 regulatory subunit protein comprises the amino acid sequence encoded by the sequences shown above. In other embodiments, the protein has at least 60% amino acid identity, more preferably 70% amino acid identity, more preferably 80%, and even more preferably, 90% or 95% amino acid identity with the amino acid sequence shown in the sequences above and retains a PP2A B56 regulatory subunit biological activity, such as the ability to interact with the catalytic and/or structural subunits of the PP2A holoenzyme, and the ability to directly regulate phosphorylation of AKT-1, e.g., phosphorylation at the threonine 308 phosphorylation site of mammalian AKT-1 or the threonine 350 phosphorylation site in C. elegans AKT-1.

In other embodiments, the invention provides isolated portions of the PP2A B56 regulatory subunit protein or chimeric proteins comprising at least a biological active portion of PP2A B56 regulatory subunit and another non-PP2A B56 regulatory subunit polypeptide.

PP2A B56 regulatory subunit proteins of the invention are preferably produced by recombinant DNA techniques. For example, a nucleic acid molecule encoding the protein is cloned into an expression vector (as described above), the expression vector is introduced into a host cell (as described above) and the PP2A B56 regulatory subunit protein is expressed in the host cell. The PP2A B56 regulatory subunit protein can then be isolated from the cells by an appropriate purification scheme using standard protein purification techniques. Alternative to recombinant expression, a PP2A B56 regulatory subunit polypeptide can be synthesized chemically using standard peptide synthesis techniques. Moreover, native PP2A B56 regulatory subunit protein can be isolated from cells, for example by immunoprecipitation using an anti-PP2A B56 regulatory subunit antibody.

The present invention also pertains to variants of the PP2A B56 regulatory subunit proteins which function as PP2A B56 regulatory subunit agonists (mimetics). Variants of the PP2A B56 regulatory subunit proteins can be generated by mutagenesis, e.g., discrete point mutation or truncation of a PP2A B56 regulatory subunit protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PP2A B56 regulatory subunit protein. In one embodiment, the invention pertains to derivatives of PP2A B56 regulatory subunit which may be formed by modifying at least one amino acid residue of PP2A B56 regulatory subunit by oxidation, reduction, or other derivatization processes known in the art.

In one embodiment, variants of a PP2A B56 regulatory subunit protein which function as PP2A B56 regulatory subunit agonists (mimetics) can be identified by screening combinatorial libraries of mutants, e.g., truncation mutants, of a PP2A B56 regulatory subunit protein for PP2A B56 regulatory subunit protein agonist activity. In one embodiment, a variegated library of PP2A B56 regulatory subunit variants is generated by combinatorial mutagenesis at the nucleic acid level and is encoded by a variegated gene library. A variegated library of PP2A B56 regulatory subunit variants can be produced by, for example, enzymatically ligating a mixture of synthetic oligonucleotides into gene sequences such that a degenerate set of potential PP2A B56 regulatory subunit sequences is expressible as individual polypeptides, or alternatively, as a set of larger fusion proteins (e.g., for phage display) containing the set of PP2A B56 regulatory subunit sequences therein. There are a variety of methods which can be used to produce libraries of potential PP2A B56 regulatory subunit variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PP2A B56 regulatory subunit sequences. Methods for synthesizing degenerate oligonucleotides are known in the art (see, e.g., Narang, S. A., 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of a PP2A B56 regulatory subunit protein coding sequence can be used to generate a variegated population of PP2A B56 regulatory subunit fragments for screening and subsequent selection of variants of a PP2A B56 regulatory subunit protein. In one embodiment, a library of coding sequence fragments can be generated by treating a double stranded PCR fragment of a PP2A B56 regulatory subunit coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the PP2A B56 regulatory subunit protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PP2A B56 regulatory subunit proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify PP2A B56 regulatory subunit variants (Arkin and Yourvan, 1992, Proc. Natl. Acad. Sci. USA 89:7811-7815; Delgrave et al., 1993, Protein Engineering 6(3):327-331).

The invention also provides PP2A B56 regulatory subunit fusion proteins. As used herein, a PP2A B56 regulatory subunit “fusion protein” comprises a PP2A B56 regulatory subunit polypeptide operatively linked to a polypeptide other than PP2A B56 regulatory subunit. A “PP2A B56 regulatory subunit polypeptide” refers to a polypeptide having an amino acid sequence corresponding to PP2A B56 regulatory subunit protein, or a peptide fragment thereof which is unique to PP2A B56 regulatory subunit protein whereas a “polypeptide other than PP2A B56 regulatory subunit” refers to a polypeptide having an amino acid sequence corresponding to another protein. Within the fusion protein, the term “operatively linked” is intended to indicate that the PP2A B56 regulatory subunit polypeptide and the other polypeptide are fused in-frame to each other. The other polypeptide may be fused to the N-terminus or C-terminus of the PP2A B56 regulatory subunit polypeptide. For example, in one embodiment, the fusion protein is a GST-PP2A B56 regulatory subunit fusion protein in which the PP2A B56 regulatory subunit sequences are fused to the C-terminus of the GST sequences. In another embodiment, the fusion protein is a PP2A B56 regulatory subunit-HA fusion protein in which the PP2A B56 regulatory subunit nucleotide sequence is inserted in a vector such as pCEP4-HA vector (Herrscher, R. F. et al. (1995) Genes Dev. 9:3067-3082) such that the PP2A B56 regulatory subunit sequences are fused in frame to an influenza hemagglutinin epitope tag. Such fusion proteins can facilitate the purification of recombinant PP2A B56 regulatory subunit.

In certain embodiments of the invention a fusion protein comprises a protein transduction domain (PTD) operatively linked to a PP2A B56 regulatory subunit polypeptide. Examples of suitable protein transduction domains are discussed below.

Preferably, a PP2A B56 regulatory subunit fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A PP2A B56 regulatory subunit-encoding nucleic acid can be cloned into such an expression vector such that the fusion moiety is linked in-frame to the PP2A B56 regulatory subunit protein.

### B. Inhibitory Compounds

The methods of the invention using inhibitory compounds which inhibit the expression and/or activity of a PP2A B56 regulatory subunit can be used in the prevention and/or treatment of disorders in which PP2A B56 regulatory subunit activity is undesirable or undesirably enhanced, stimulated, upregulated or the like, e.g., Diabetes, e.g., Diabetes II, or a diabetes related disorder, e.g., obesity.

In one embodiment of the invention, an inhibitory compound can be used to inhibit (e.g., specifically inhibit) the expression or activity of a PP2A B56 regulatory subunit.

Inhibitory compounds of the invention can be, for example, intracellular binding molecules that act to specifically inhibit the expression or activity e.g., of a PP2A B56 regulatory subunit. As used herein, the term “intracellular binding molecule” is intended to include molecules that act intracellularly to inhibit the processing expression or activity of a protein by binding to the protein or to a nucleic acid (e.g., an mRNA molecule) that encodes the protein. Examples of intracellular binding molecules, described in further detail below, include antisense nucleic acids, peptidic compounds that inhibit the interaction of a PP2A B56 regulatory subunit with a target molecule, e.g., a catalytic and/or structural subunit of the PP2A holoenzyme, or a substrate of the holoenzyme, e.g., AKT-1, and chemical agents that specifically inhibit a PP2A B56 regulatory subunit activity.

i. Antisense or siRNA Nucleic Acid Molecules

In one embodiment, an inhibitory compound of the invention is an antisense nucleic acid molecule that is complementary to a gene encoding a PP2A B56 regulatory subunit, or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequence for the coding strand of the PP2A B56 regulatory subunit gene and thus the known sequence of the PP2A B56 regulatory subunit mRNA, antisense nucleic acids of the invention can be designed according to the rules of Watson and Crick base pairing. The antisense nucleic acid molecule can be complementary to the entire coding region of an mRNA, but more preferably is antisense to only a portion of the coding or noncoding region of an mRNA. For example, the antisense oligonucleotide can be complementary to the region surrounding the translation start site of an mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. To inhibit expression in cells, one or more antisense oligonucleotides can be used.

Alternatively, an antisense nucleic acid can be produced biologically using an expression vector into which all or a portion of a cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector can be introduced into cells using a standard transfection technique.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, an antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid molecule of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation mRNAs. A ribozyme having specificity e.g., for an XBP-1, IRE-1 alpha, or ATF6α-encoding nucleic acid can be designed based upon the nucleotide sequence of the cDNA. For example, a derivative of a Tetrahymena L-19 IVS RNA can be constructed in which the nucleotide sequence of the active site is complementary to the nucleotide sequence to be cleaved in, e.g., an XBP-1-encoding mRNA. See, e.g., Cech et al. U.S. Pat. No. 4,987,071 and Cech et al. U.S. Pat. No. 5,116,742. Alternatively, XBP-1 mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of a gene (e.g., a PP2A B56 regulatory subunit promoter and/or enhancer) to form triple helical structures that prevent transcription of a gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In another embodiment, a compound that promotes RNAi can be used to inhibit expression of a PP2A B56 regulatory subunit. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F. 2003. Mol Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of siRNA are commercially available from, e.g. New England Biolabsor Ambion. In one embodiment one or more of the chemistries described above or known in the art for use in antisense RNA can be employed in molecules that mediate RNAi. The ordinary skilled artisan would be able to generate, based on common knowledge in the art and using no more than routine experimentation, working examples of a PP2A B56 regulatory subunit specific siRNAs.

ii. Peptidic Compounds

In another embodiment, an inhibitory compound of the invention is a peptidic compound derived from the PP2A B56 regulatory subunit amino acid sequence. For example, in one embodiment, the inhibitory compound comprises a portion of, e.g., a PP2A B56 regulatory subunit (or a mimetic thereof) that mediates interaction of a PP2A B56 regulatory subunit with another PP2A subunit (e.g., a catalytic and/or structural subunit) or with a target molecule (e.g., AKT-1) such that contact of the PP2A B56 regulatory subunit with this peptidic compound competitively inhibits the interaction of the PP2A B56 regulatory subunit with the other PP2A subunit or target molecule.

The peptidic compounds of the invention can be made intracellularly in cells by introducing into the cells an expression vector encoding the peptide. Such expression vectors can be made by standard techniques using oligonucleotides that encode the amino acid sequence of the peptidic compound. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

In addition, dominant negative proteins (e.g., of a PP2A B56 regulatory subunit) can be made which include a PP2A B56 regulatory subunit molecules (e.g., portions or variants thereof) that compete with native (i.e., wild-type) molecules, but which do not have the same biological activity. Such molecules effectively decrease, e.g., a PP2A B56 regulatory subunit activity in a cell.

iii. Other Agents

In one embodiment, the expression of a PP2A B56 regulatory subunit can be inhibited using an agent that inhibits a signal that increases PP2A B56 regulatory subunit expression, processing, post-translational modification or activity in a cell.

Other inhibitory agents that can be used to specifically inhibit the activity of a PP2A B56 regulatory subunit are chemical compounds that directly inhibit expression, processing, post-translational modification, and/or activity of, e.g., an a PP2A B56 regulatory subunit target protein or inhibit the interaction between, e.g., a PP2A B56 regulatory subunit and the catalytic and/or structural subunit of PP2A, or between, e.g., a PP2A B56 regulatory subunit (e.g., as part of the PP2A holoenzyme) and target molecules, e.g., AKT-1. Such compounds can be identified using screening assays that select for such compounds, as described in detail above as well as using other art recognized techniques.

In one embodiment, an inhibitory compound is a chemical chaperone. As used herein, a “chemical chaperone” is a compound known to stabilize protein conformation against denaturation (e.g., chemical denaturation, thermal denaturation), thereby preserving protein structure and function (Welch et al. Cell Stress Chaperones 1:109-115, 1996; incorporated herein by reference). Chemical chaperones have been shown in certain instances to correct folding/trafficking defects seen in such diseases as cystic fibrosis (Fischer et al. Am. J. Physiol. Lung Cell Mol. Physiol. 281 :L52-L57, 2001; incorporated herein by reference), prion-associated diseases, nephrogenic diabetes insipidus, and cancer (Bai et al. Journal of Pharmacological and Toxicological Methods 40(1):39-45, July 1998; incorporated herein by reference).

In one embodiment, a “chemical chaperone” is a small molecule or low molecular weight compound. Preferably, the “chemical chaperone” is not a protein. Examples of “chemical chaperones include glycerol, deuterated water (D2O), dimethylsulfoxide (DMSO), trimethylamine N-oxide (TMAO), glycine betaine (betaine), glycerolphosphocholine (GPC) (Burg et al. Am. J. Physiol. (Renal Physiol. 43):F762-F765, 1998; incorporated herein by reference), 4-phenyl butyrate or 4-phenyl butyric acid (PBA), methylamines, and tauroursodeoxycholic acid (TUDCA), taurin, methylamine and deoxyspergualin (see Brown et al., Cell Stress Chaperones 1:117-125, 1996; Jiang et al., Amer J. Physiol.-Cell Physiol. 44:C171-C178, 1998). (Rubenstein et al., J. Clin. Invest. 100:2457-2465, 1997), sodium butyrate (Cheng et al., Am. J. Physiol. 268:L615-624, 1995) and S-Nitrosoglutathione (Zaman, et al., Biochem Biophys Res Commun 284: 65-70, 2001; Snyder, et al., American Journal of Respiratory and Critical Care Medicine 165: 922-6, 2002; Andersson, et al. Biochemical and Biophysical Research Communication 297(3): 552-557, 2002.).

In another embodiment of the invention, an inhibitory compound is an autophagy activator. As used herein, an “autophagy activator” is any compound that increases autphagy within a cell. An increase in autophagy may be determined as known in the art and described herein. Exemplary, non-limiting autophagy activators are known in the art and include, for example, proteasome inhibitor, tamoxifen, IFN-gamma, trehalose, vinblastine, rapamycin, or its analogues, that inhibit the mammalian target of rapamycin (mTOR) (a negative regulator of autophagy), ganima-benzene hexachloride, or of a derivative thereof which is obtainable by chemical substitution, but has retained said capacity of acting as an inducer or stimulator of autophagy maturation.

An mTor inhibitor may include a rapamycin macrolide such as rapamycin or a salt, analogue or derivatives of rapamycin. Suitable rapamycin macrolides are described in more detail below. An IMPase inhibitor may include a compound described above. mTOR inhibitors include rapamycin and other rapamycin macrolides. A macrolide is a macrocyclic lactone, for example a compound having a 12-membered or larger lactone ring. Lactam macrolides are macrocyclic compounds which have a lactam (amide) bond in the macrocycle in addition to a lactone (ester) bond.

Rapamycin is a lactam macrolide produced by Streptomyces hygroscopicus (McAlpine J. B. et al. J. Antibiotics (1991) 44: 688; Schreiber, S. L. et al. J. Am. Chem. Soc. (1991) 113:7433; U.S. Pat. No. 3,929,992). A rapamycin macrolide as described herein may include rapamycin or a salt, analogue or derivative of rapamycin.

Suitable rapamycin analogues well known in the art (see for example WO 94/09010 and Wa 96/41807) and include 40-0-(2-hydroxy)ethyl-rapamycin, 32-deoxo-rapamycin, 16-O-pent-2-ynyl-32-deoxo-rapamycin, 16-a-pent-2-ynyl-32-deoxO-40-0-(2-hydroxyethyl)-rapamycin, 16-O-pent-2-ynyl-32-CS)-dihydro-rapamycin and 16-a-pent-2-ynyl-32-(S)-dihydro-40-0-C2 hydroxyethyl)-rapamycin. Other rapamycin analogues include carboxylic acid esters as set out in WO 92/05179, amide esters as set out in U.S. Pat. No. 5,118,677, carbamates as set out in U.S. Pat. No. 5,118,678, fluorinated esters as set out in USS,100,883, acetals as set out in U.S. Pat. No. 5,151,413, silyl ethers as set out in U.S. Pat. No. 5,120,842 and arylsulfonates and sulfamates as set out in U.S. Pat. No. 5,177,203. Other rapamycin analogues which may be used in accordance with the invention may have the methoxy group at the position 16 replaced with alkynyloxy as set out in WO 95/16691. Rapamycin analogues are also disclosed in WO 93/11130, WO 94/02136, WO 94/02385 and Administration of a compound for the treatment of a disorder, as described herein, is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

E. Suitable Controls

Assay methods generally require comparison to a control sample to which no agent is added. The screening methods described above represent primary screens, designed to detect any agent that may exhibit activities indicating modulation of the expression or activity of the PP2A B56 regulatory subunit. The skilled artisan will recognize that secondary tests will likely be necessary in order to evaluate an agent further. For example, a cytotoxicity assay would be performed as a further corroboration that an agent which tested positive in a primary screen would be suitable for use in living organisms. Any assay for cytotoxicity would be suitable for this purpose, including, for example the MTT assay (Promega).

This invention further pertains to novel agents identified by the above-described screening assays. Accordingly, it is within the scope of this invention to further use an agent identified as described herein in an appropriate animal model, e.g., an animal model to determine the efficacy, toxicity, or side effects of treatment with such an agent.

IV. Therapeutic Methods of the Invention

The present invention provides methods of treating a subject in need thereof with an agent which modulates a PP2A B56 regulatory subunit, for example, an agent identified according to one of the above-described screening assays. “Treatment”, or “treating” as used herein, is defined as the application or administration of a pharmacological agent of the invention to a subject, or application or administration of said agent to an isolated tissue or cell line from a subject, such that the desired outcome is achieved.

Agents identified according to one of the above-described screening assays can be useful in the treatment of metabolic disorders, e.g., diabetes, e.g., type II diabetes, and diabetes associated disorders, e.g., obesity. Type 2 diabetes is a disease of peripheral insulin resistance combined with pancreatic beta-cell dysfunction. Current evidence indicates that disruption of insulin/insulin-like growth factor (IGF)-1 signaling mechanisms may contribute to defects in both peripheral insulin action and β-cell function. Thus components of the insulin/IGF signaling network and its downstream effector molecules have been identified as attractive therapeutic targets for the rationale treatment of of this disease. Based on the discoveries provided herein, which reveal that the PP2A B56 regulatory subunit (e.g., PPRT-1 or B56β) regulates phosphorylation of AKT-1, the PP2A B56 regulatory subunit is an attractive therapeutic targets for treatment of type 2 diabetes and other disorders associated with diabetes.

The present invention provides a method of preventing or treating type II diabetes in a subject, involving selecting a subject in need of prevention or treatment for type II diabetes, administering to said subject a pharmacologically effective dose of an agent that modulates, e.g., reduces, the activity or expression of a PP2A B56 regulatory subunit, wherein modulation, e.g., reduction, of the activity or expression of a PP2A B56 regulatory subunit in said subject prevents or reduces obesity in said subject. In one embodiment of this aspect, the activity or expression of a PP2A B56 regulatory subunit is downmodulated, reduced or inhibited. In various embodiments of this aspect, the agent can be, for example, a blocking antibody to the PP2A B56 regulatory subunit, a dominant-negative form of a PP2A B56 regulatory subunit, a small molecule that inhibits the PP2A B56 regulatory subunit, an agent that modulates the interaction of the PP2A B56 regulatory subunit with a PP2A B56 regulatory subunit-binding molecule (e.g., a catalytic and/or structural subunit of the PP2A holoenzyme), such that the activity or expression of the PP2A B56 regulatory subunit is modulated, e.g., reduced.

The present invention further provides a method of preventing or reducing obesity in a subject, involving selecting a subject in need of preventing or reducing obesity, administering to said subject a pharmacologically effective dose of an agent that modulates, e.g., reduces, the activity or expression of a PP2A B56 regulatory subunit, wherein modulation, e.g., reduction, of the activity or expression of a PP2A B56 regulatory subunit in said subject prevents or reduces obesity in said subject. In one embodiment of this aspect, the activity or expression of a PP2A B56 regulatory subunit is downmodulated, reduced or inhibited. In various embodiments of this aspect, the agent can be, for example, a blocking antibody to the PP2A B56 regulatory subunit, a dominant-negative form of a PP2A B56 regulatory subunit, a small molecule that inhibits the PP2A B56 regulatory subunit, an agent that modulates the interaction of the PP2A B56 regulatory subunit with a PP2A B56 regulatory subunit-binding molecule (e.g., a catalytic and/or structural subunit of the PP2A holoenzyme), such that the activity or expression of the PP2A B56 regulatory subunit is modulated, e.g., reduced.

Agents identified according to one of the above-described screening assays can additionally be useful in enhancing longevity. Accordingly, the present invention provides a method of enhancing longevity in a subject, involving selecting a subject in need of enhanced longevity, and administering to said subject a pharmacologically effective dose of an agent that modulates, e.g., increases, stimulates or enhances, the activity or expression of a PP2A B56 regulatory subunit, wherein modulation, e.g., stimulation of the activity or expression of the PP2A B56 regulatory subunit in said subject enhances longevity. In a preferred embodiments, the agent increases or enhances the activity or expression of a PP2A B56 regulatory subunit. Preferably, the agent increases the activity or expression of a PP2A B56 regulatory subunit such that AKT-1 phosphorylation is decreased.

Agents identified according to one of the above-described screening assays can also be useful in preventing or treating cancer or in inhibiting proliferation of cancer cells. Accordingly, the present invention provides a method of preventing or treating cancer in a subject, involving selecting a subject in need of treating cancer, and administering to said subject a pharmacologically effective dose of an agent that modulates, e.g., increases, stimulates or enhances, the activity or expression of a PP2A B56 regulatory subunit, wherein modulation, e.g., enhancement, of the activity or expression of said PP2A B56 regulatory subunit in said subject prevents or treats cancer. In a preferred embodiment, the agent increases, stimulates or enhances the activity or expression of a PP2A B56 regulatory subunit. Preferably, the agent increases, stimulates or enhances the activity or expression of a PP2A B56 regulatory subunit such that AKT-1 phosphorylation is decreased. The present invention further provides a method of inhibiting proliferation of cancer cells in a subject, involving selecting a subject in need thereof, and administering to said subject a pharmacologically effective dose of an agent that modulates, e.g., increases, stimulates or enhances, the activity or expression of a PP2A B56 regulatory subunit, wherein modulation, e.g., enhancement, of the activity or expression of said PP2A B56 regulatory subunit in said subject inhibits proliferation of cancer cells. In a preferred embodiment, the agent increases, stimulates or enhances the activity or expression of a PP2A B56 regulatory subunit. Preferably, the agent increases, stimulates or enhances the activity or expression of a PP2A B56 regulatory subunit such that AKT-1 phosphorylation is decreased.

Such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Compounds that can be used in the methods of the invention are described in further detail below.

V. Pharmaceutical Compositions

The modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

This invention is further illustrated by the experiments described in the following examples, which should not be construed as limiting. The contents of all references, patents and published patent applications cited throughout this application are incorporated herein by reference.

### EXAMPLES

### Example 1

### Materials and Methods for Examples 2-10

### A. Strains

All strains were maintained at 15° C. using standard C. elegans techniques (Stiernagle, 2006). Double mutants were made using standard genetic methods as described below. For all RNAi assays, the worms were grown for at least two full generations on the RNAi bacteria.

For making double mutants, daf-2(e1370) males were mated to either akt-1(ok525), akt-2(ok393) and sgk-1(ok538) hermaphrodites, respectively. A total of 40 F1 progeny were singled onto individual plates and allowed to have progeny at 25° C. From the F2 progeny on each plate, dauers were selected and allowed to recover at 15° C. The adult worms were singled and transferred to 25° C. and allowed to have progeny. The F3 progeny formed 100% dauers at 25° C., and parents were tested for akt-1(ok525), akt-2(ok393) or sgk-1(ok538) deletion by PCR. The primers used for the PCR analysis are listed in Table 1 below.

TABLE 1
List of Primers

| | Gene | | |
|---|---|---|---|
| Primer name | Amplified | Used for | Sequence (5′-3′) |
| AKT-1 INT2 LT | akt-1 | Detecting akt-1 deletion | AGAGCGTCTATGATTAGTATTG |
| AKT-1 DEL2 | akt-1 | Detecting akt-1 deletion | TACTTTCTGTGGAACTCCGGAG |
| AKT-1 INT2 RT | akt-1 | Detecting akt-1 deletion | AGTTTCTGACTGCACATTTGGC |
| FW(AKT-2) | akt-2 | Detecting akt-2 deletion | AGTACACAGAGTGTGATAATGC |
| RV-WT(AKT-2) | akt-2 | Detecting akt-2 deletion | GCAAACTCCATCACAAAACAG |
| RV-MUT(AKT-2) | akt-2 | Detecting akt-2 deletion | CACGTAGTATGACGCAAATTG |
| SGK-1(WT FW): | sgk-1 | Detecting sgk-1 deletion | TCAGGCTTCAAATCTCTGGAA |
| SGK-1(DEL-FW): | sgk-1 | Detecting sgk-1 deletion | AAACAGGCAAATACGGCATC |
| SGK-1(RV): | sgk-1 | Detecting sgk-1 deletion | CACGTCATGGCAGAAAGAAA |
| GRd3Fw | daf-3 | Detecting daf-3 deletion | CTGGCAGTCACTAACACACG |
| GRd3Rv | daf-3 | Detecting daf-3 deletion | ACCCTCATGCCTACTGTCAG |
| P1 | mCherry(fw- | mCherry cloning | AGCTCG GGTACCG |
| | KpnI) | | ATGGTCTCAAAGGGTGAAGAAG |
| P2 | mcherry(Rv- | mCherry cloning | TACGAATGAATTCTTAATCATCGTCCTTATAG |
| | EcoRI-CF-tag): | | TCCATTCCGCTAGCTCCACCCTGAAAATACAA |
| | | | ATTCTCTCCAGCTCCCTTATACAATTCATCCA |
| | | | TGCCAC |
| ccdb(R2-r4)Fw | ccdb | Ccdb amplification | CAACTTTGTATAGAAAAGTTGAACG |
| ccdb(R2-r4)Rv | ccdb | Ccdb amplification | ATAATGGTACCTCAACTTTGTACAAGAAAGTT |
| | | | GAACGA |
| unc-119(Prom-Fw): | unc-119 | unc-119 amplifcation | TGTTTTGTGCCAAGCTTCAG |
| | (genomic) | | |
| unc-119(Prom-rv): | unc-119 | unc-119 amplification | AGTTCCGTGTGCTCTTGCTC |
| | (genomic) | | |
| unc-119(cdna-fw) | unc-119 (cDNA) | unc-119 amplification | GGACGACCCCCATTAATTTT |
| unc-119(cdna-rv) | unc-119 (cDNA) | unc-119 amplification | ATCTGA AAGCTT |
| | | | TGAAAAATCATTTATTGGGATTTG |
| sod-3-52F | sod-3 | RTPCR | GGAGTTCTCGCCGTCCG |
| sod-3-102R | sod-3 | RTPCR | GTCGAATGGGAGATCTGGGAG |
| mt1-1-11F | mtl-1 | RTPCR | AGTGTGACTGCAAAAACAAGCAA |
| mtl-1-66R | mtl-1 | RTPCR | TCCACTGCATTCACATTTGTCTC |
| hsp-12.6-41F | hsp-12.6 | RTPCR | GGGATTGGCCACTTCAAAAG |
| hsp-12.6-91R | hsp-12.6 | RTPCR | CGTCGTCGAGGACATTGACA |
| sip-1-274F | sip-1 | RTPCR | AAGAGATCGTTCACTCGCCAG |
| sip-1-324R | sip-1 | RTPCR | AGCCAAGTCGACGTCCTTTG |
| ACT-1-5 | act-1 | RTPCR | CTCTTGCCCCATCAACCATG |
| ACT-1-3 | act-1 | RTPCR | CTTGCTTGGAGATCCACATC |
| Ppp2Ca F1 | PP2Aca | RTPCR | ATGGACGAGAAGTTGTTCACC |
| Ppp2Ca R1 | PP2Aca | RTPCR | CAGTGACTGGACATCGAACCT |
| Ppp2Cb F1 | PP2Acb | RTPCR | ATGAGTGCCTACGGAAGTATGG |
| Ppp2Cb R1 | PP2Acb | RTPCR | CAGGGCTCTTATGTGGTCCAG |
| Ppp2R5a F1 | 856a | RTPCR | ATTGAAGAGCCGCTTTTTAAGCA |
| Ppp2R5a R1 | 856a | RTPCR | TGAGGGTTTTCAGCACATTGT |
| Ppp2R5b F1 | 856b | RTPCR | GGGCCTACATCCGCAAACA |
| Ppp2R5b R1 | 856b | RTPCR | GGATCAGGACTCGAACCAGG |
| t22d1.5-160F | T22D1.5 | RTPCR | ACGTCTCAAGCAATCACTGGAG |
| t22d1.5-21OR | T22D1.5 | RTPCR | TTTCGTCGCTTCTATGTGCG |
| rsa-1-367F | rsa-1 | RTPCR | GATATCACTCTTTTAGAGGAGCTTGTACG |
| rsa-1-417R | rsa-1 | RTPCR | CTCCTCAGCGTGAATGACTGTT |
| W08G11.4-161F | pptr-1 | RTPCR | CCATTGCACAAACCGAAATG |
| W08G11.4-933R | pptr-1 | RTPCR | GTAGGCGAGCTGTGCATGATAC |
| C13G3.3-44F | pptr-2 | RTPCR | CAAACACGTACGGAGGAGGG |
| C13G3.3-94R | pptr-2 | RTPCR | ATTGGCGGCGCTCTCTT |
| sur-6-245F | sur-6 | RTPCR | CAACGGGAGACAAAGGAGGA |
| sur-6-295R | sur-6 | RTPCR | CACTCTGATCTCGTTGGAAAATTACA |

For making the daf-2(e1370);daf-3(mgDf90) double mutant, daf-2(e1370) males were crossed to daf-3(mgDf90) hermaphrodites. The F1 progeny males were selected and mated back to daf-3(mgDf90) hermaphrodites. Forty F2 progeny were transferred to individual plates and incubated at 25° C. After 4-5 days, parents were selected from plates where the F3 progeny were 100% dauers and the daf-3 deficiency was checked by PCR, as described previously (Patterson et al., 1997). Dauers were recovered to establish the strain.

For co-localization and immunoprecipitation experiments, pptr-1::mC-flag males were mated to myo-3::gfp (Fire et al., 1998), akt-1::gfp (Sp209) (Paradis and Ruvkun, 1998), akt-2::gfp;unc-119(+);unc-119(ed3), sgk-1::gfp (BR2773; kind gift from Ralf Baumeister) (Hertweck et al., 2004) and daf-16::gfp (kind gift from Ruvkun Lab) hermaphrodites, respectively. F1 progeny were selected and subsequently F2 worms homozygous for both GFP and mCherry were selected under a fluorescence microscope. The extrachromosomal lines akt-1::gfp (Sp209) and sgk-1::gfp (BR2777) were integrated into the genome by UV irradiation prior to making genetic doubles.

The daf-2(e1370);daf-16::gfp and daf-2(e1370);Psod-3::gfp(muIs84) strain was made by crossing daf-2(e1370) males to either daf-16::gfp (kind gift from Ruvkun Lab) or Psod-3::gfp(muIs84) hermaphrodites. About 40 F1 animals were transferred to individual plates and allowed to have progeny at 25° C. From the progeny, F2 dauers were selected from each plate and allowed to recover at 15° C. The recovered adult worms were then checked for the presence of GFP, and GFP-positive worms were transferred to individual plates and incubated at 25° C. Plates where 100% of the progeny were dauers and GFP positive were selected and established as the strain for the assays.

Strains used in Examples 2-10 in clued N2, P daf16a::daf16a::gfp, CB1370[daf-2)e1370)], DR1572 [daf-2(e1368)], RB759 [akt-1(ok525)], JT9609 [pdk-1 (sa680)], VC204 [sgk-1(ok538)] and PD4251. A number of additional strains were generated for these studies are listed below in Table 2.

TABLE 2
List of Strains Generated

| Strain | Strain # | Comment |
|---|---|---|
| daf-2(e1370); akt-1(ok525) | HT1547 | |
| daf-2(e1370); akt-2(ok393) | HT1548 | |
| daf-2(e1370); sgk-1(ok538) | HT1648 | |
| daf-2(e1370); daf-3(mgDf90) | HT1607 | |
| akt-1::gfp | HT1632 | Integrated SP209 Pakt-1::akt- |
| | | 1::gfp, rol-6 from Ruvkun Lab |
| | | (Paradis and Ruvkun, 1998) |
| akt-2::gfp; unc-119(+); | HT1604 | Integrated Line |
| unc-119 (ed3) | | |
| sgk-1::gfp | HT1634 | Integrated BR2777 Ex[sgk- |
| | | 1::gfp] from Baumeister Lab |
| | | (Hertweck et al., 2004) |
| daf-2(e1370); daf-16::gfp | HT1531 | Crossed Pdaf-16a::daf- |
| | | 16::gfp from Ruvkun Lab to |
| | | daf-2(e1370) (Lee et al., 2001) |
| pptr-1::mC-flag | HT1630 | Integrated Line, 4x backcrossed |
| | | to N2, contains unc-119(+) |
| | | marker |
| akt-1::gfp; pptr-1::mc-flag | HT1644 | |
| akt-2::gfp; pptr-1::mc-flag | HT1645 | |
| sgk-1::gfp; pptr-1::mc-flag | HT1635 | |
| daf-16::gfp; pptr-1::mc-flag | HT1646 | Crossed Pdaf-16a::daf- |
| | | 16a::gfp to pptr-1::mC-flag |
| myo-3::gfp; pptr-1::mc-flag | HT1647 | Crossed PD4251 to pptr- |
| | | 1 ::mc-flag (Fire et al., 1998) |
| daf-2(e1370); pptr-1::mc-flag | HT1641 | |
| daf-2(e1370); Psod-3::gfp | HT1643 | Crossed muIs84 [pAD76(sod- |
| | | 3::GFP)] to daf-2(e1370) |
| | | (Libina et al., 2003) |
| unc-119(+); unc-119(ed3) | HT1638 | Extrachromosomal Array Line |
| daf-2(e1370); unc-119(+); | HT1642 | Extrachromosomal Array Line |
| unc-119(ed3) | | |

### B. Preparation of RNAi Plates

RNAi plates were prepared by supplementing Nematode Growth Media (NGM) media with 100 μg/ml ampicillin and 1 mM IPTG. After pouring, the plates were kept at room temperature (RT) for 5 days to dry. RNAi bacteria were grown overnight at 37° C. in LB media supplemented with 100 μg/ml ampicillin and 12.5 μg/ml tetracycline. The next day, the cultures were diluted (1:50) in LB containing 100 μg/ml ampicillin and grown at 37° C. until an OD₆₀₀ of 0.9. The bacterial pellets were resuspended in 1× PBS (phosphate-buffered saline) containing 1 mM IPTG. About 200 μl of the bacterial suspension was seeded onto the RNAi plates. The seeded plates were dried at RT for 3 days and stored at 4° C.

C. C. elegans Assays

C. elegans assays were modified from previously published methods (Henderson and Johnson, 2001; Kimura et al., 1997; Libina et al., 2003; Oh et al., 2006; Oh et al., 2005) and are described in detail below.

(i) Dauer Assays

For the dauer assays, approximately 5 L4 or young adult worms were transferred to the RNAi bacteria and maintained at 15° C. F2 adult worms were then picked to a fresh RNAi plate and allowed to lay eggs. About 120 eggs were picked from these plates onto 3 fresh plates containing the RNAi bacteria and incubated at the indicated temperatures. The plates were scored for the presence of dauers or non-dauers after 3.5-4 days, unless indicated otherwise. For assays involving daf-2(e1370);sgk-1(ok538), the strain is slow growing with a prolonged L1/L2 arrest and only forms dauers on vector RNAi after 7-8 days. Similarly, daf-2(e1370);akt-1(ok525) worms grown on vector and pptr-1 RNAi were also scored after 7-8 days.

The pdk-1(sa680) worms have an Egl phenotype. For the pdk-1(sa680) dauer assays, eggs were obtained by hypochlorite treatment of gravid adults worms grown on vector, daf-18 and pptr-1 RNAi plates (Stiernagle, 2006).

(ii) Life Span Assays

All life span analyses were performed at 15° C. Strains were synchronized by picking eggs on to fresh RNAi or OP50 plates and allowed to grow for several days until they became young adults. Approximately 60 young adult worms were transferred to each of 3 RNAi plates for every RNAi clone tested (vector, daf-18 and pptr-1). For life span experiments with overexpression strains, approximately 60 young adult worms were transferred to 3 fresh OP50 plates for every strain tested. Life spans were performed on RNAi plates or OP50 plates overlaid with 5-fluorodeoxyuridine (FUDR) to a final concentration of 0.1 mg/ml of agar (Hosono et al., 1982). Significantly fewer worms were observed to burst at 15° C. by transferring young adult animals rather than L4 animals to FUDR plates. Worms were then scored as dead or alive by tapping them with a platinum wire every 2-3 days. Worms that died from vulval bursting were censored. Statistical analyses for survival were conducted using the standard chi-squared-based log rank test.

(iii) Heat Stress Assays

Wild type and daf-2(e1370) animals were maintained on RNAi bacteria at 15° C. From these plates, approximately 30 young adult worms were picked onto fresh vector, daf-18 and pptr-1 RNAi plates. These plates were shifted to 20° C. for 6 hrs. The plates were then transferred to 37° C. and heat stress-induced mortality was determined every few hours till all the animals were dead. Statistical analyses for survival were conducted using the standard chi-squared-based log rank test.

(iv) Fat Staining

Sudan black staining of stored fat was performed as previously described (Kimura et al., 1997). Briefly, wild type and daf-2(e1370) worms on RNAi plates were synchronized by picking eggs on to fresh RNAi plates and grown until the L3 stage. The worms were then washed off the plates and incubated in M9 buffer for 30 minutes on a shaker at RT. After 3 washes with M9 buffer, the worms were fixed in 1% paraformaldehyde. The worms were then sequentially dehydrated by washes in 25%, 50% and 70% ethanol. Saturated Sudan Black (Sigma, USA) solution was prepared fresh in 70% ethanol. The fixed worms were incubated overnight in 250 uL of Sudan Black solution, on a shaker at RT, mounted on slides and visualized using the Zeiss Axioscope 2+ microscope.

(v) daf-2(e1370) Growth Assay

daf-2(e1370) worms were maintained on vector, pptr-1, daf-18 and daf-16 RNAi plates for two generations at 15° C. Approximately 100-150 eggs were picked on to two fresh plates for every RNAi clone tested and the plates were incubated at 20° C. Worms were scored based upon their stages as larval stages 1/2 (L1/L2), dauers, larval stage 3 (L3), larval stage 4 (L4) or adults after 3.5 days.

(vi) DAF-16::GFP Localization Assay

The daf-2(e1370);daf-16::gfp strain was maintained at 15° C. on vector, daf-18 or pptr-1 RNAi plates. About 20-25 L4 or young adults were transferred to fresh RNAi bacteria and the plates were shifted to 25° C. for 1 hr. The worms were then visualized using Zeiss Axioscope 2+ microscope. Worms were classified into four categories based on the extent of DAF-16::GFP nuclear-cytoplasmic distribution, as follows: +: completely cytoplasmic; ++: nuclear in some tissues but cytoplasmic in majority of the tissues; +++: cytoplasmic in some tissues but nuclear in majority of the tissues; and ++++: nuclear localization in all tissues (Hertweck, 2004).

D. C. elegans Immunoprecipitation (IP) and Western Blotting

Transgenic worms were grown in three 100 mm plates seeded with OP50 bacteria at 20° C. Worms were harvested by washing with M9 buffer and pellet collected by centrifugation. The pellet was resuspended in 250 μl lysis buffer (20 mM Tris-Cl, 137 mM NaCl, 10% glycerol, 1% Triton X-100, 25 mM β-glycerophosphate, Protease inhibitor cocktail (Roche Biochemicals, USA), pH 7.4). The worms were sonicated with Bioruptor (Diagenode, USA) using maximum power output (1 min sonication, 2 min off-repeated 10 times). The lysate was cleared by centrifugation and protein content estimated by Bradford method. Lysate equivalent to 1.5 mg total protein was pre-cleared with 50 μl of protein-G agarose beads, fast flow (Upstate, USA) and then immunoprecipitated overnight at 4° C. using either anti-GFP monoclonal antibody (Sigma, USA) or anti-FLAG M2 gel (Sigma, USA). The following morning, 50 μl protein-G agarose beads, fast flow were added to the GFP IP to capture the immune complex. The agarose beads were then washed 5 times with lysis buffer. Following this step, the beads were boiled in Laemelli's buffer.

For western blot analysis, immunoprecipitated protein samples was resolved on a 10% SDS-PAGE and transferred to nitrocellulose membranes. Membranes were blocked in TBST (Tris Buffered Saline containing 0.05% Tween 20, pH 7.4) containing 5% non-fat milk at RT for 1 hour. Membranes were then washed with TBST and incubated overnight with 1:1000 dilutions of antibodies in TBST containing 5% non-fat milk 4° C. Membranes were washed 3 times with TBST and then incubated with TBST containing 5% non-fat milk containing a 1:10,000 dilution of the secondary antibody. Antibodies used for western were: Living Color DsRed antibody (Clontech, USA; Catalog no. 632496); Living Color Rabbit polyclonal GFP antibody (BD Biosciences, USA; Catalog no. 632460); Monoclonal mAb 3e6 GFP antibody (Invitrogen, USA; Catalog no. A11120); and Anti-FLAG M2 Affinity Gel (Sigma, USA; Catalog no. A2220).

E. C. elegans Phospho-AKT Western Blotting

Transgenic worms were grown at 20° C. in 3-4 large (100 mm) plates seeded with OP50. Worms were collected by washing with 1× PBS and the pellet was then immediately frozen in dry ice. Approximately 500 μl lysis buffer, supplemented by Sigma Phosphatase inhibitor cocktails I and II (50×) and Protease inhibitor cocktail (Roche Biochemicals, USA), was added to the pellet and sonicated using a Misonix (3000) sonicator (Misonix, USA; power output set at 4, 3 pulses of 10 secs each with 1 min interval between pulses). The lysates were clarified by centrifugation at 13000 rpm for 10 mins at 4° C. and the protein content estimated by Quick Bradford (Pierce). About 3.5 μg of anti-GFP monoclonal antibody (3E6, Invitrogen USA) was used for each IP from lysates containing 1.7 mg protein in a volume of 1 ml. IPs were performed overnight at 4° C. and antibody-protein complexes were captured using 50 μl of protein-G agarose beads, fast flow (Upstate, USA) for 2 hrs at 4° C. The pellets were washed 3 times with lysis buffer supplemented by protease and phosphatase inhibitors and boiled in Laemelli's buffer. The IP samples were then resolved on a 10% SDS-PAGE, western blotted and analyzed with phospho-specific antibodies (described below).

### F. Psod-3::GFP Expression

daf-2(e1370);Psod-3::gfp(muIs84) worms were grown at 15° C. on RNAi plates as described herein. About 25-30 L4/young adults were transferred to fresh RNAi plates and shifted to 25° C. for 2 hrs. The expression of GFP was visualized using Zeiss Axioscope 2+ microscope. Worms were classified into three categories based on the intensity of GFP expression as follows: High: bright GFP expression seen throughout the worm; Medium: Low GFP expression in the worm body; Low: weak or barely detectable GFP expression in the body. GFP expression in the head region does not change dramatically.

G. Construction of pSCFTdest

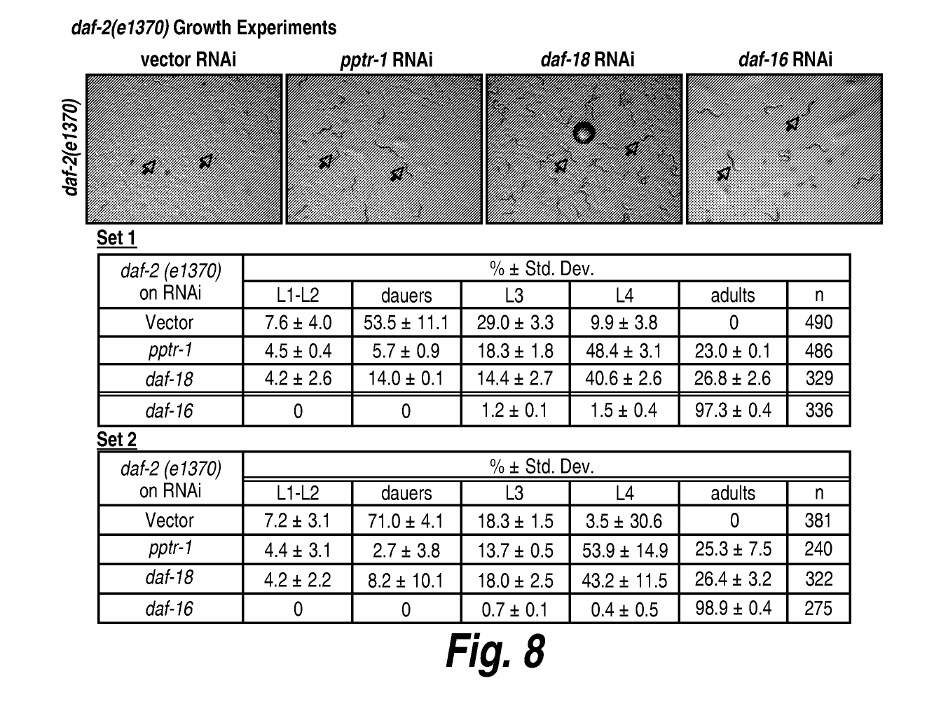

The pSCFTdest vector was derived from pPD95.75 vector (provided by Fire lab). Briefly, the mcherry gene was amplified from the mCherry vector (McNally et al., 2006) using primers listed in FIG. 8. The amplified product was restriction digested with KpnI and EcoRI and ligated to pPD95.75 at KpnI/EcoRI (replacing the gfp gene in the vector) to give pSCFT (CFT-Colocalization Flag tag) plasmid. The R4-R2 gateway cassette from pdestMB14 (Reboul et al., 2003) was PCR-amplified using ccdb primers (FIG. 8). The amplicon was restriction digested with KpnI, producing an insert with one blunt end and another KpnI-compatible end. pSCFT was then digested with SmaI and KpnI and the blunt end/KpnI insert was ligated into the cut vector to make pSCFTdest.

### H. Transgenic Worms

Transgenic worms were made by microparticle bombardment. Briefly, a 3 kb sequence of the pptr-1 promoter and the pptr-1 ORF were cloned into separate entry vectors (Reboul et al., 2003; Walhout et al., 2000) using Gateway Technology (Invitrogen, USA) and confirmed by DNA sequencing. The promoter and ORF were then combined using multi-site Gateway cloning into the pSCFTdest vector (for details, see Example 1 above) to create the pSCFT-pptr-1. An unc-119 promoter::ORF fusion mini-gene was constructed as described earlier (Maduro and Pilgrim, 1995) and cloned into pUC-19 vector between EcoRI sites. The unc-119 mini-gene insert was then excised using EcoRI restriction digestion, gel-purified, blunt ended with T4 DNA Polymerase (Roche Biochemicals, USA) and cloned into pSCFT-pptr-1 (at the filled-in SphI site) giving rise to the pSCFT-pptr-1-unc-119 vector. This construct was used in biolistic transformation (Biorad, USA) of unc-119(ed3) mutants (Maduro and Pilgrim, 1995; Praitis, 2006). Integrated lines were back-crossed four times to wild-type and used for further analysis.

For the akt-2::gfp construct, a 3 kb sequence of the akt-2 promoter was cloned into the corresponding entry vector and the akt-2 ORF from the ORFeome were combined using multi-site Gateway technology into the R4-R2 destination vector (Reboul et al., 2003) to create akt-2::gfp-unc-119(+) vector. This vector was verified by restriction digestion and integrated transgenic lines were obtained by micro-particle bombardment (Maduro and Pilgrim, 1995; Praitis, 2006).

I. C. elegans AKT-1 Phospho-Specific Antibodies

Phospho antibodies were prepared and affinity purified by 21st Century Biochemicals (Marlboro, Mass.). Briefly, rabbits were immunized with phospho-peptides (mixture of AKT pT350 PP1 and AKT pT350 PP2 for T350 and pS517PP1 for S517) and after 6 boosts, the rabbit serum was immunodepleted with a peptide column conjugated to non-phosphorylated peptide (AKT T350 NP or AKT S517 NP). Following immunodepletion, the sera was affinity purified with Phospho-peptide AKT pT350 PP1 (for T350 site) or AKT pS517 PP1 (for S517 site). The peptides used were: AKT pT350 PP1: Ac-TS[pT]FCGTPEYK-amide (injected); AKT pT350 PP2: CSYGDKTS[pT]FSGTPEY-amide (injected); AKT T350 NP: CSYGDKTSTF[C/S]GTPEY-amide-used for (immunodepletion); AKT pS517 PP1: Ac-CSNFTQF[pS]FHNVMGS-amide-conjugated (injected); and AKT S517 NP: Ac-CSNFTQFSFHNVMGS-amide-conjugated (immunodepletion).

J. Mammalian Cell Culture and siRNA Transfection

3T3-L1 adipocytes were cultured and differentiated as previously described (Tesz et al., 2007). Briefly, 3T3-L1 adipocytes were cultured and differentiated in complete Dulbecco's modified Eagle's medium (10% fetal bovine serum, 50 units/ml penicillin, and 50 g/ml streptomycin) as previously described (Tesz et al., 2007). For siRNA transfections, cells from 4 days post-induction of adipocyte differentiation were used as previously described (Tang et al., 2006). Briefly, 1.125×10⁶ cells were electroporated using 6 nmol of siRNA and then plated in 5 wells of a 12-well plate. Cells were recovered in complete DMEM and were cultured for 48 h after the transfection prior to the experiments.

### K. Insulin Stimulation and Phospho-Akt Western Blotting

3T3-L1 adipocytes transfected with siRNA were serum-starved for 18 hours. Cells were stimulated with increasing concentrations of insulin for a period of 30 minutes. Following insulin stimulation, the cells were washed with ice-cold PBS and harvested on ice as described previously (Tesz et al., 2007). Protein samples were resolved on 8% SDS-PAGE and transferred to a nitrocellulose (NC) membrane as mentioned above. Antibodies used were Phospho-Akt Ser 473 (Cell Signaling, USA; Catalog no. 9271), Phospho-Akt Thr 308 (Cell Signaling, USA; Catalog no. 9275), total Akt antibody (Cell Signaling, USA; Catalog no. 9272). Secondary antibody incubation was performed as above in 1% BSA. Changes in the phosphorylation of Akt pSer 473 and pThr 308 were quantified through densitometry using NIH ImageJ and normalized for loading against the non-phosphorylated total Akt levels.

### L. RNA Isolation and Real-Time PCR

Total RNA was isolated using Trizol (Invitrogen, USA). Briefly, worms grown on vector, daf-16, daf-18 or pptr-1 RNAi were washed off the plates using M9 buffer. Next, 0.3 ml of TRIzol reagent was added and vortexed vigorously. The RNA was then purified by phenol:chloroform:isoamylalcohol extraction and ethanol precipitation. The concentration and the purity of the RNA were determined by measuring the absorbance at 260/280 nm. To further determine the quality of the RNA, the quality of the ribosomal 28 S and 18 S was visually inspected on an agarose gel. cDNA was synthesized using 2 ug of RNA and the SuperScript cDNA synthesis kit (Invitrogen, USA). For the RNAi knockdown experiments shown in FIG. 12 (Supplemental Table 1A), daf-2(e1370) worms grown on vector, pptr-1, pptr-2, rsa-1, T22D1.5 and sur-6 RNAi plates were washed off using M9 and RNA was purified using the RNA Plus Mini Kit (Qiagen Cat #74134). Gene expression levels were determined by real time PCR using the SYBR® Green PCR Master Mix and 7000 Real-Time PCR System (Applied Biosystems, USA) according to the manufacturer's instructions. Relative gene expression was compared to actin as an internal loading control. All the primers used are listed in Table 1 (above).

### M. DAPI Staining

pptr-1::mC-flag worms were washed off an OP50 plate with PBS and washed three times by briefly spinning the worms at 3000 rpm for 1 minute. The supernatant was collected and 500 uL of 3% Formaldehyde (diluted with potassium phosphate buffer, KH2PO4) was added to the worm pellet. The samples were fixed for 15-20 mins with gentle shaking, and 500 uL of PBS-Tween (0.1%) was added and gently mixed. The samples were then spun at 3000 rpm for 1 minute and washed twice with PBS-Tween and the supernatant was removed. 2 uL of DAPI (1 mg/mL, Sigma D9542) added to 500 uL of PBS and the samples were incubated in this solution for 15-20 minutes before mounting.

### Example 2

### RNAi Screen to Identify Phosphatases in IIS Pathway

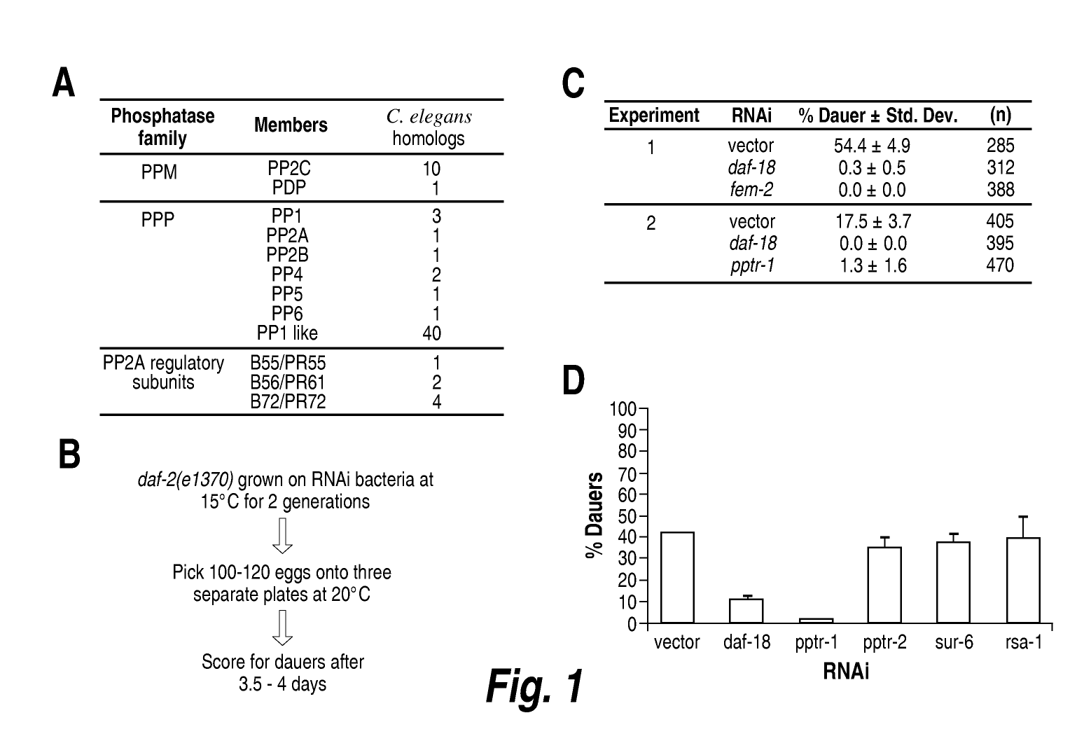

To identify the serine/threonine phosphatases in the C. elegans genome, in silico analyses was performed using both NCBI KOGs (clusters of euKaryotic Orthologous Groups) and WormBase (a C. elegans database: http://www.wormbase.org; WS152) annotations. A total of 60 genes were identified for further analysis (FIG. 1A). RNAi clones for these phosphatases were obtained from the Ahringer RNAi library (Kamath and Ahringer, 2003; Kamath et al., 2003), generated using available clones from the ORFeome library (Reboul et al., 2003) or cloned de-novo using Gateway Technology (Invitrogen, USA; Materials and Methods). Three of the phosphatase cDNAs were not able to be cloned and therefore a total of 57 candidates were screened.

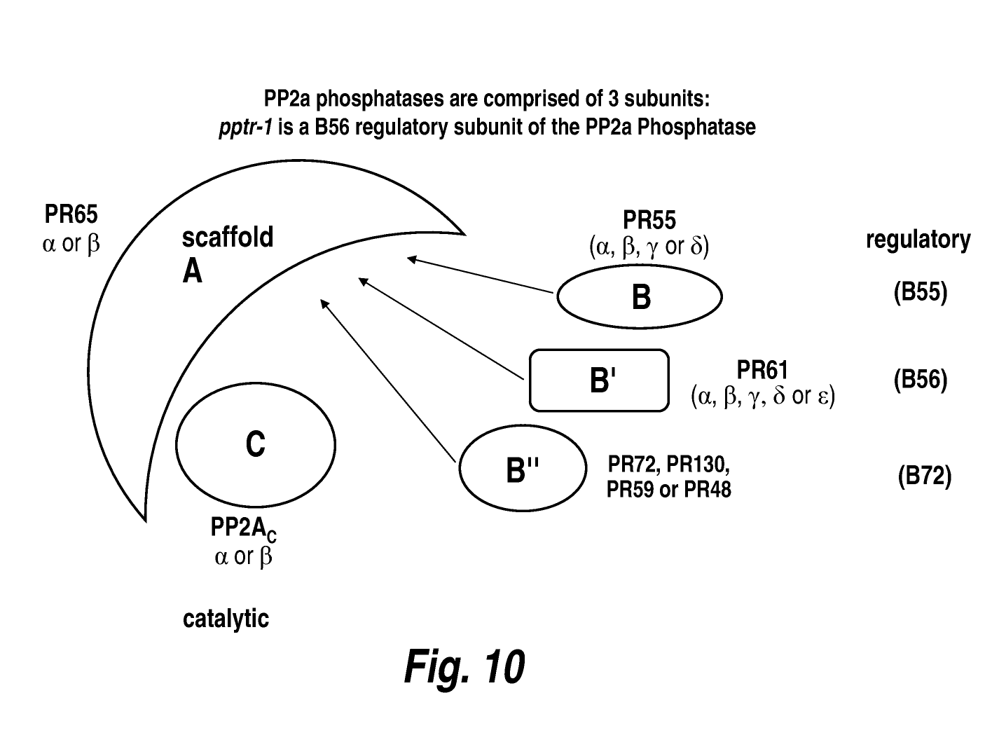

In addition, 6 of the 7 annotated PP2A holoenzyme regulatory subunits (one was not cloned) were included in the screen for two reasons. First, a preliminary chemical inhibitor screen identified the PP2A family of phosphatases as important regulators of DAF-16 nuclear translocation. Second, the PP2A holoenzyme is comprised of a catalytic, structural and a regulatory subunit (Janssens et al., 2008) (FIG. 10) and RNAi of the catalytic and structural subunits of PP2A resulted in lethality.

daf-2(e1370) carries a mutation in the insulin receptor tyrosine kinase domain that results in a is phenotype for dauer formation (Kimura et al., 1997). daf-2(e1370) worms arrest as 100% dauers at 25° C. whereas at 15° C. they have a normal reproductive cycle (Riddle D., 1997). At an intermediate temperature of 20° C., a significant percentage of daf-2(e1370) worms form dauers. Therefore, at this temperature, RNAi can be used to easily assess the contribution of any gene in modulating daf-2 dauer formation.

For the screen, daf-2(e1370) mutants were grown on RNAi-expressing bacteria for two generations, and eggs were picked onto 3 plates for each RNAi clone (FIG. 1B). The plates were incubated at 20° C. and scored 3.5-4 days later for the presence of dauers and non-dauers. Since DAF-18 is the only known phosphatase that negatively regulates the IIS in C. elegans, daf-18 RNAi was used as a positive control in all experiments. From a total of 63 RNAi clones (57 phosphatases and 6 regulatory subunits), two phosphatases were identified that dramatically decreased daf-2(e1370) dauer formation to a level similar to daf-18 RNAi (FIG. 1C).

A top candidate, fem-2 (T19C3.4), functions in C. elegans sex determination (Hansen and Pilgrim, 1998; Pilgrim et al., 1995). However, further analysis with an additional daf-2 allele, daf-2(e1368), revealed that fem-2 RNAi suppresses dauer formation in an allele-specific manner. fem-2 RNAi suppressed dauer formation of daf-2(e1370) but not daf-2(e1368) and therefore, fem-2 was not pursued further.

The next candidate, pptr-1 (W08G11.4), is a member of the B56 family of genes encoding regulatory subunits of the PP2A protein phosphatase holoenzyme. The C. elegans genome contains 7 known PP2A regulatory subunit genes (pptr-1 and pptr-2, B56 family; sur-6, B55 family; F47B8.3, C06G1.5, rsa-1 and T22D1.5, B72 family; currently F47B8.3 is not annotated as a PP2A regulatory subunit according to WormBase Release WS194). To determine the specificity of pptr-1 in regulating dauer formation, the six PP2A regulatory subunits included in the screen were re-tested for their ability to regulate dauer formation in daf-2(e1370) mutants. Knockdown efficiency of each RNAi clone was verified by RT PCR Table 3. As shown in FIG. 1D, only pptr-1 RNAi suppressed daf-2(e1370) dauer formation comparable to daf-18 RNAi.

TABLE 3
Quantitative PCR to show the knock down of PP2a regulatory subunits by RNAi

| | | Transcripts determined by Q-PCR | |
|---|---|---|---|
| | | (% of vector RNAi) | |
| Strain | RNAi | Set 1^{a} | Set 2 |
| daf-(e1370) | pptr-1 | 0.3 | 0.3 |
| | pptr-2 | 0.4 | 0.5 |
| | sur-6 | 0.4 | 0.7 |
| | rsa-1 | 0.1 | 0.1 |
| | T22D1.5 | * | 0.1 |

^{a}Plates used in Set 1 were used in The dauer assay in FIG. 1D and the Q-PCR analysis.
*Knock down of T22D1.5 was independently verified in Set 2
Additional experiments showing the specificity of dauer suppression
by RNAi of PP2A regulatory subunit family members. Only pptr-
1RNAi is able to suppresses dauer formation in daf-2(e1370)mutants.

| | % Dauer ± Std. Dev. (n) | | |
|---|---|---|---|
| daf-2(e1370) on RNAi | Repeat 1 | Repeat 2 | Repeat 3 |
| vector | 81.7 ± 6.8 (191) | 43.2 ± 15.5 (191) | 13.5 ± 3.4 (181) |
| daf-18 | 0.8 ± 1.2 (244) | 4.9 ± 3.5 (528) | 3.9 ± 0.1 (205) |
| pptr-1 | 2.8 ± 0.8 (253) | 0.8 ± 0.5 (492) | 0.9 ± 1.3 (186) |
| pptr-2 | 73.0 ± 15.1 (200) | 34.0 ± 12.8 (426) | 5.0 ± 1.7 (229) |
| sur-6 | 77.3 ± 7 (141) | 58.9 ± 18.1 (302) | nt |
| T22D1.5 | nt | 57.6 ± 5.6 (295) | 17.9 ± 6.6 (175) |
| F47B8.3* | 82.1 ± 7.4 (151) | 47.7 ± 3.5 (417) | 8.7 ± 1.7 (220) |
| rsa-1 | 75.6 ± 4.8 (164) | nt | 11.4 ± 3.1 (161) |

nt-not tested in this trial
*currently F47B8.3 is not annotated as a PP2A regulatory subunit according toWormBase Release
WS194

The effect of pptr-1 RNAi on dauer formation of daf-2(e1368) was next analyzed. pptr-1 RNAi significantly suppressed the dauer formation of daf-2(e1368) (69.2±9.4% on vector RNAi versus 3.8±4.4% on pptr-1 RNAi; Tables 4 and 5. Therefore the effect of pptr-1 RNAi on daf-2 mutants is not allele-specific. Together these results indicated that pptr-1 may function downstream of daf-2. In addition, pptr-1 was the only PP2A regulatory subunit to affect daf-2 dauer formation.

TABLE 4
Epistasis analysis of dauer formation using different IIS pathway mutants grown on RNAi clones.

| | % Dauer ± Std. Dev. (n) | | |
|---|---|---|---|
| Strains | vector RNAi | daf-18 RNAi | pptr-1 RNAi |
| daf-2(1368)^{a} | 69.2 ± 9.4 (202) | 0 (295) | 3.8 ± 4.4 (257) |
| daf-2(e1370)^{b} | 17.2 ± 5.9 (517) | 0.2 ± 0.3 (397) | 9.6 ± 7.3 (460) |
| daf2(e1370; daf-3(mgDf90) | 94.5 ± 0.8 (589) | 40.4 ± 14.0 (308) | 42.7 ± 14.6 (329) |
| pdk-1(sa680)^{a} | 95.6 ± 1.0 (490) | 80.8^{c} (52) | 9.5 ± 0.3 (180) |
| daf-2(e1370) | 73.0 ± 0.2 (525) | 3.5 ± 1.7 (279) | 4.1 ± 3.8 (344) |
| daf-2(e1370); akt-1(ok525)^{d} | 94.8 ± 3.1 (237) | 1.0 ± 0.3 (386) | 96.0 ± 1.7 (186) |
| daf-2(e1370); akt-2(ok393) | 36.8 ± 3.8 (336) | 5.0 ± 1.1 (610) | 10.8 ± 4.3 (583) |
| daf(1370) | 79.1 ± 5.4 (601) | 0.7 ± 1.0 (405) | 27.2 ± 14.8 (536) |
| daf-2(e1370); sgk-1(ok538) | 65.4 ± 4.9 (338)^{e} | 0.3 ± 0.4 (303) | 0 (364) |

All strains were maintained at 15° and assays were performed at 20° C., unless indicated otherwise.
Also, dauer formation of all strains were scored after 3.5-4 days, unless indicated otherwise.
Data shown is representative of one experiment.
^{a}The experiment was performed at 25° C.
^{b}Dauers were scored after 5 days.
^{c}In most experiments, the pdk-1(sa680) worms failed to hatch on daf-18 RNAi. This number represents 20% of the eggs picked for this assay.
^{d}Dauers were scored after 7-8 days. For daf-2(e1370); akt-1(ok525) worms on vector or pptr-1 RNAi, all the non-dauers were either partial dauers or dauer-like. They did not develop into adults even after 2 weeks
^{e}Dauers were scored after 7-8 days. The daf-2(e1370); sgk-1(ok538) strain shows no gro phenotype and worms reman at L1/L2 stage for 6-7 days at 20° C.

TABLE 5
Epistasis analysis of dauer formation using different IIS pathway
mutants grown on RNAi clones.

| | % Dauer ± Std. Dev. (n) | | |
|---|---|---|---|
| Strains | vector RNAi | daf-18 RNAi | pptr-1 RNAi |
| daf-2(e1368)^{a} | 84.6 ± 25.3 (326) | 0 (141) | 19.8 ± 10.7 (104) |
| daf-2(e1370)^{b} | 19.4 ± 2.7 (588) | 1.9 ± 0.9 (528) | 1.2 ± 1.0 (581) |
| daf-2(e1370); daf-3(mgDf90) | 87.6 ± 18.1 (394) | 41.6 ± 18.6 (345) | 28.4 ± 12.1 (284) |
| pdk-1(sa680)^{a} | 100.0 ± 0 (221) | na^{c} | 61.2 ± 2.6 (170) |
| daf-2(e1370) | 13.5 ± 3.4 (221) | 3.9 ± .1 (205) | 0.9 ± 1.3 (186) |
| daf-2(e1370); akt-1(ok525)^{d} | 85.3 ± 5.8 (136) | 10.5 ± 0.8 (349) | 89.9 ± 1.6 (246) |
| daf-2(e1370); akt-2(ok393) | 45.6 ± 6.7 (272) | 8.0 ± 4.1 (311) | 1.1 ± 0.8 (293) |
| daf-2(e1370); sgk-1(ok538) | 58.9 ± 3.6 (326)^{e} | 4.9 ± 2.8 (237) | 1.5 ± 0.9 (210) |

All strains were maintained at 15° C. and assays were performed at 20° C., unless indicated otherwise.
Also, dauer formation of all strains was scored after 3.5-4 days, unless indicated otherwise.
Data shown is representative of one experiment.
^{a}The experiment was performed at 25° C.
^{b}Dauers were scored after 5 days.
^{c}In most experiments, the pdk-1(sa680) worms failed to hatch on daf-18 RNAi.
^{d}Dauers were scored after 7-8 days. For daf-2(e1370); akt-1(ok525) worms on vector or pptr-1 RNAi, all the non-dauers were either partial dauers or dauer-like. They did not develop into adults even after 2 weeks.
^{e}Dauers were scored after 7-8 days. The daf-2(e1370); sgk-1(ok538) strain shows a gro phenotype and worms remain arrested in the L1/L2 stage for 6-7 days at 20° C.

### Example 3

### pptr-1 Regulates Dauer Formation through the IIS Pathway

To further investigate the role of pptr-1 in dauer formation, genetic epistasis analysis was performed. In addition to the C. elegans IIS pathway, a second parallel TGF-β pathway also regulates dauer formation (Patterson and Padgett, 2000; Savage-Dunn, 2005). In this pathway, loss of function mutations in daf-7 (TGF-β ligand), daf-1 and daf-4 (receptors) or daf-14 and daf-8 (R-Smads) lead to constitutive dauer formation; loss-of-function mutations in daf-3 (Co-Smad) or daf-5 (Sno/Ski) suppress these phenotypes (da Graca et al., 2004; Gunther et al., 2000; Inoue and Thomas, 2000; Patterson et al., 1997; Ren et al., 1996). However, mutations in daf-3 do not suppress daf-2(e1370) dauer formation (Vowels and Thomas, 1992). A daf-2(e1370);daf-3(mgDf90) double mutant which bears a null mutation in daf-3 (Patterson et al., 1997) was generated, which essentially removes the input from the TGF-β pathway for dauer formation. In this strain, the dauer formation of daf-2(e1370);daf-3(mgDf90) worms was suppressed by pptr-1 RNAi (94.5±0.8% dauers on vector RNAi to 42.7±14.6.% dauers on pptr-1 RNAi; Tables 4 and 5. This data suggested that pptr-1 controls dauer formation specifically through the IIS pathway and not the TGF-β pathway.

### Example 4

### pptr-1 Affects Longevity, Metabolism and Stress Response Downstream of the IIS Receptor

In addition to dauer formation, the C. elegans IIS pathway also regulates lifespan, fat storage and stress resistance (Antebi, 2007; Kenyon, 2005; Wolff and Dillin, 2006). Since pptr-1 regulates dauer formation specifically via the IIS pathway, it was next determined whether this gene could also affect these other important phenotypes.

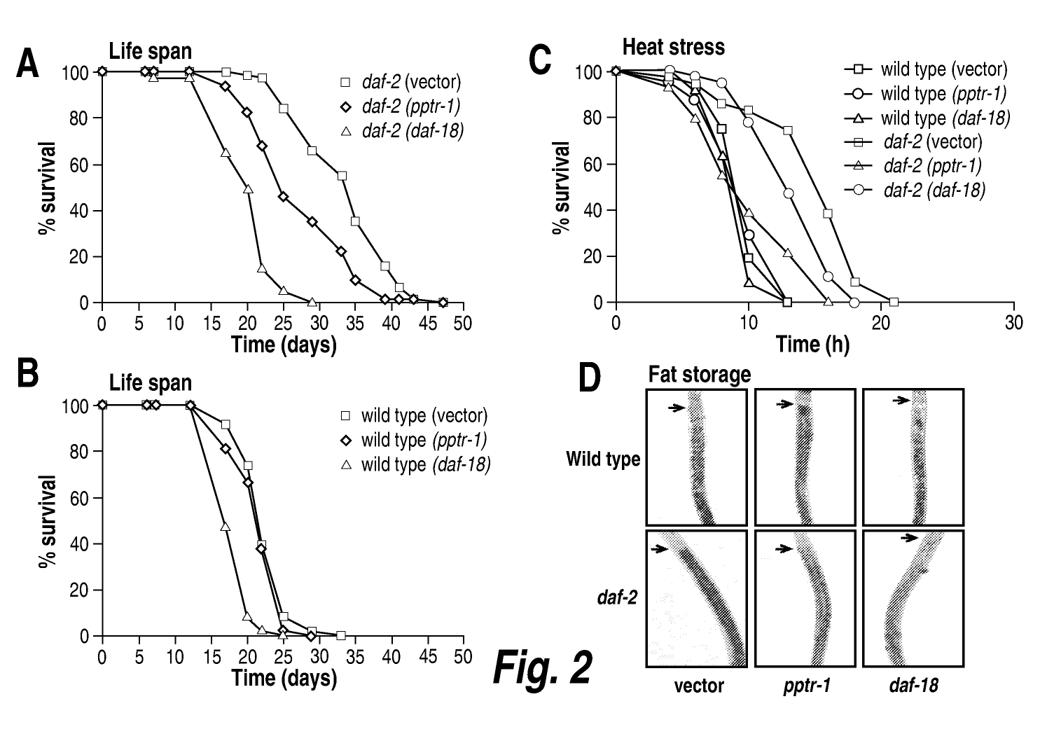

Mutations in daf-2 result in lifespan extension (Kenyon et al., 1993) that is suppressed by loss-of-function mutations in daf-18 (Dorman et al., 1995; Larsen et al., 1995). To investigate whether pptr-1 can regulate lifespan similar to daf-18, it was determined whether knocking down pptr-1 by RNAi could affect daf-2(e1370) lifespan. Wild type and daf-2(e1370) worms were grown on vector, daf-18 and pptr-1 RNAi and lifespan was measured (FIG. 2A). Similar to daf-18 RNAi, knockdown of pptr-1 resulted in a significant reduction in daf-2(e1370) lifespan (mean lifespan of daf-2(e1370) on vector RNAi is 33.9±0.7 days, on pptr-1 RNAi is 27.7±0.9 days and on daf-18 RNAi is 20.4±0.6 days, p value <0.0001; FIG. 2A and TABLE 6. In contrast, lifespan of wild type was unaffected by pptr-1 RNAi (mean lifespan of wild type on vector RNAi is 22.8±0.4 days, is 21.9±0.5 days on pptr-1 RNAi and 18.6±0.3 days on daf-18 RNAi; FIG. 2B and TABLE 7. Thus, pptr-1 affects lifespan in addition to dauer formation.

TABLE 6
Additional Life span Experiments on RNAi plates

| | | Mean Life Span | | |
|---|---|---|---|---|
| | | Days ± Std. Dev. (n) | | |
| Set # | Strain | vector RNAi | daf-18 RNAi | pptr-1 RNAi |
| 1. | wild type | 23.8 ± 0.4 (67) | 19.5 ± 0.3 (54)* | 22.8 ± 0.8 (40) |
| | daf-2(e1370) | 38.9 ± 0.9 (75) | 24.5 ± 0.6 (59)* | 29.8 ± 1.1 (50)* |
| 2. | wild type | 23.7 ± 0.5 (32) | 18.4 ± 1.1 (17)* | 22.3 ± 0.7 (44) |
| | daf-2(e1370) | 35.9 ± 0.5 (96) | 22.5 ± 0.5 (63)* | 30.5 ± 0.5 (124)* |

All life span assays were performed at 15° C.
*significance vs vector RNAi p < .0001
Additional PPTR-1 Overexpression Life span Experiments

| | Mean Life Span | | |
|---|---|---|---|
| | Days ± Std. Dev. (n) | | |
| Strain | Set 1 | Set 2 | Set 3 |
| wild type | 23.5 ± 0.3 (160) | 23.9 ± 0.3 (154) | 22.4 ± 0.3 (148) |
| daf-2(e1370) | 33.0 ± 0.5 (150) | 33.6 ± 0.5 (164) | 33.2 ± 0.6 (129) |
| pptr-1::mC-flag | 26.0 ± 0.5* (115) | 30.1 ± 0.5* (202) | 28.7 ± 0.4* (201) |
| daf-2(e1370); pptr-1::mC-flag | 33.2 ± 0.6 (150) | 35.5 ± 0.4 (190) | 33.1 ± 0.5 (142) |
| unc-119(+); unc-119(ed3) | 21.9 ± 0.3 (137) | 22.6 ± 0.3 (145) | 22.3 ± 0.3 (151) |
| daf-2(e1370); unc-119(+); | 30.0 ± 0.7 (109) | 30.5 ± 0.6 (117) | 31.1 ± 0.7 (108) |
| unc-119(ed3) | | | |

All life span assays were performed at 15° C.
*significance vs N2 p < .0001

TABLE 7
Additional Heat Stress Experiments

| Re- | | % Survival at 37° C. | | |
|---|---|---|---|---|
| peat | | Hours ± Std. Dev. (n) | | |
| # | Strain | vector RNAi | daf-18 RNAi | pptr-1 RNAi |
| 1. | wild | 12.1 ± 0.3 (36) | 10.2 ± 0.4 (33)* | 10.0 ± 0.3 (39) |
| | type | | | |
| | daf-2 | 18.6 ± 0.7 (45) | 15.5 ± 0.8 (40)* | 14.5 ± 0.6 (30)* |
| | (e1370) | | | |
| 2. | wild type | 10.7 ± 0.4 (49) | 9.9 ± 0.3 (43)* | 10.6 ± 0.4 (43) |

All strains were maintained at 15° C. and assays were performed at 37° C.
*significance vs vector RNAi-p < .0001

Lifespan extension correlates well with increased stress resistance (Lithgow and Walker, 2002; Oh et al., 2005). For example, daf-2(e1370) mutants are not only long-lived but are also extremely resistant to various stresses such as heat and oxidative stress (Honda and Honda, 1999; Lithgow et al., 1995; Munoz and Riddle, 2003). Therefore, the effect of pptr-1 RNAi on the thermotolerance of daf-2(e1370) mutants was analyzed. As anticipated, pptr-1 RNAi significantly reduced the thermotolerance of daf-2(e1370) mutants (on vector RNAi, daf-2(e1370) had a mean survival of 15.2±0.7 hrs, whereas on pptr-1 RNAi survival was 13.8±0.5 hrs (p value <0.006)). pptr-1 RNAi did not affect the thermotolerance of wild type worms (mean thermotolerance was 9.8±0.4 hrs on vector RNAi, versus 9.3±0.3 hrs on pptr-1 RNAi; FIG. 2C and Table 8.

TABLE 8

| | | | Completely | Mostly | Mostly | Completely | |
|---|---|---|---|---|---|---|---|
| | | | Cytoplasmic | Cytoplasmic | Nuclear | Nuclear | |
| A | Strains | RNAi | (%) | (%) | (%) | (%) | (n) |
| | pptr-1::mC-flag; | vector | 30.6 | 52.8 | 11.1 | 5.6 | 36 |
| | daf-16::gfp | mCherry | 62.5 | 27.1 | 6.3 | 4.2 | 48 |
| | daf-16::gfp | vector | 58.1 | 32.3 | 9.7 | 0 | 62 |
| | | mCherry | 57.4 | 27.8 | 13.0 | 1.9 | 47 |
| | | | Completely | Mostly | Mostly | Completely | |
| | | | Cytoplasmic | cytoplasmic | Nuclear | Nuclear | |
| B | Strain | RNAi | (%) | (%) | (%) | (%) | (n) |
| | daf-2(e1370); | vector | 0 | 20.0 | 35.0 | 45.0 | 20 |
| | daf-16::gfp | daf-18 | 20.0 | 50.0 | 30.0 | 0 | 20 |
| | | pptr-1 | 30.0 | 35.0 | 15.0 | 20.0 | 20 |

| C | Strain | RNAi | High (%) | Medium (%) | Low (%) | (n) |
|---|---|---|---|---|---|---|
| | daf-2(e1370); | vector | 56.0 | 40.0 | 4.0 | 25 |
| | sod-3::gfp | daf-18 | 10.0 | 20.0 | 70.0 | 10 |
| | | pptr-1 | 7.4 | 37.0 | 55.6 | 27 |

| | | | Transcripts determined by Q-PCR (% of vector RNAi) | | | |
|---|---|---|---|---|---|---|
| D | Strain | RNAi | sod-3 | hsp-12.6 | sip-1 | mti-1 |
| | daf-2(e1370) | daf-16 | 9.6 | 19.2 | 13.6 | 15.0 |
| | | daf-18 | 33.0 | 66.2 | 74.4 | 26.2 |
| | | pptr-1 | 50.0 | 62.2 | 35.8 | 48.3 |

In addition to enhanced lifespan and stress resistance, daf-2 mutants have increased fat storage (Ashrafi et al., 2003; Kimura et al., 1997). Accordingly, it was next asked whether pptr-1 could also affect fat storage in wild type and daf-2(e1370) worms using Sudan black staining. Consistent with the lifespan and stress resistance results described above, pptr-1 RNAi suppressed the increased fat storage of daf-2(e1370) without affecting wild type fat storage (FIG. 2D).

Finally, daf-2 mutants have a slow growth phenotype (Gems et al., 1998; Jensen et al., 2007) that is suppressed by knockdown of daf-16 by RNAi (FIG. 8). Similar to daf-16 RNAi and daf-18 RNAi, pptr-1 RNAi suppresses this slow growth phenotype. Together, the results of these experiments indicate that pptr-1 regulates multiple phenotypes associated with the IIS pathway in C. elegans.

### Example 5

### pptr-1 Functions at the Level of akt-1

Signals from DAF-2 are transduced to the PI 3-kinase AGE-1 to activate the downstream serine/threonine kinase PDK-1. PDK-1 activates three downstream serine/threonine kinases, AKT-1, AKT-2 and SGK-1 (Antebi, 2007; Kenyon, 2005; Wolff and Dillin, 2006). These kinases together regulate the transcription factor DAF-16 by direct phosphorylation (Hertweck et al., 2004). Mutations in daf-16 suppress the enhanced dauer formation of pdk-1 (Paradis et al., 1999) or akt-1/akt-2 mutants (Oh et al., 2005; Paradis and Ruvkun, 1998). The results described above in Examples 2-4 suggest that pptr-1 functions in the IIS pathway. Accordingly, genetic epistasis experiments were performed on components of the IIS pathway in order to identify the potential target of pptr-1.

First, the effect of pptr-1 RNAi on dauer formation of a pdk-1 mutant was analyzed. Dauer formation of pdk-1(sa680) was suppressed by pptr-1 RNAi (95.6±1.0% dauers on vector RNAi versus 9.5±0.3% dauers on pptr-1 RNAi, Tables 4 and 5. In contrast, daf-18 RNAi had no effect on pdk-1(sa680) dauer formation Tables 4 and 5. Therefore, these results placed pptr-1 downstream of pdk-1 and were consistent with the current understanding that daf-18 acts upstream of pdk-1.

Next, to investigate whether pptr-1 acts at the level of akt-1, akt-2 or sgk-1, dauer formation in akt-1(ok525), akt-2(ok393) and sgk-1(ok538) single mutants and the akt-1(ok525);akt-2(ok393) double mutant were analyzed. While akt-1(ok525), akt-2(ok393) and sgk-1(ok538) single mutants do not arrest as dauers at either 20 or 25° C., the akt-1(ok525);akt-2(ok393) double mutant forms 100% dauers at all temperatures (Oh et al., 2005). To circumvent this problem, double mutants of daf-2(e1370);akt-1(ok525), daf-2(e1370);akt-2(ok393) and daf-2(e1370);sgk-1(ok538) were generated and these strains were tested for dauer formation on vector, daf-18 and pptr-1 RNAi. It was reasoned that in a daf-2 mutant background, the akt-1, akt-2 and sgk-1 mutants would exhibit temperature-induced dauer formation. Indeed, all three double mutants were able to form dauers at 20° C.; Tables 4 and 5 (see panel for vector RNAi)). Importantly, pptr-1 RNAi significantly suppressed dauer formation in daf-2(e1370);akt-2(ok393) (36.8±3.8% dauers on vector RNAi versus 10.8±4.3% on pptr-1 RNAi; Tables 4 and 5. In addition, pptr-1 RNAi suppressed dauer formation of daf-2(e1370);sgk-1(ok538) worms (65.4±4.9% dauers on vector RNAi versus 0% on pptr-1 RNAi, Tables 4 and 5. In contrast, pptr-1 RNAi did not affect dauer formation of daf-2(e1370);akt-1(ok525) mutants (vector RNAi is 94.8.±3.1% versus 96.0±1.7% on pptr-1 RNAi; Tables 4 and 5. However, daf-18 RNAi could suppress daf-2(e1370) akt-1(ok525) dauer formation (reduced to 10.5±0.8%; Table 4. These observations genetically placed pptr-1 at the level or downstream of akt-1 in the IIS pathway.

### Example 6

### PPTR-1 and AKT-1 are Expressed in the Same Tissues

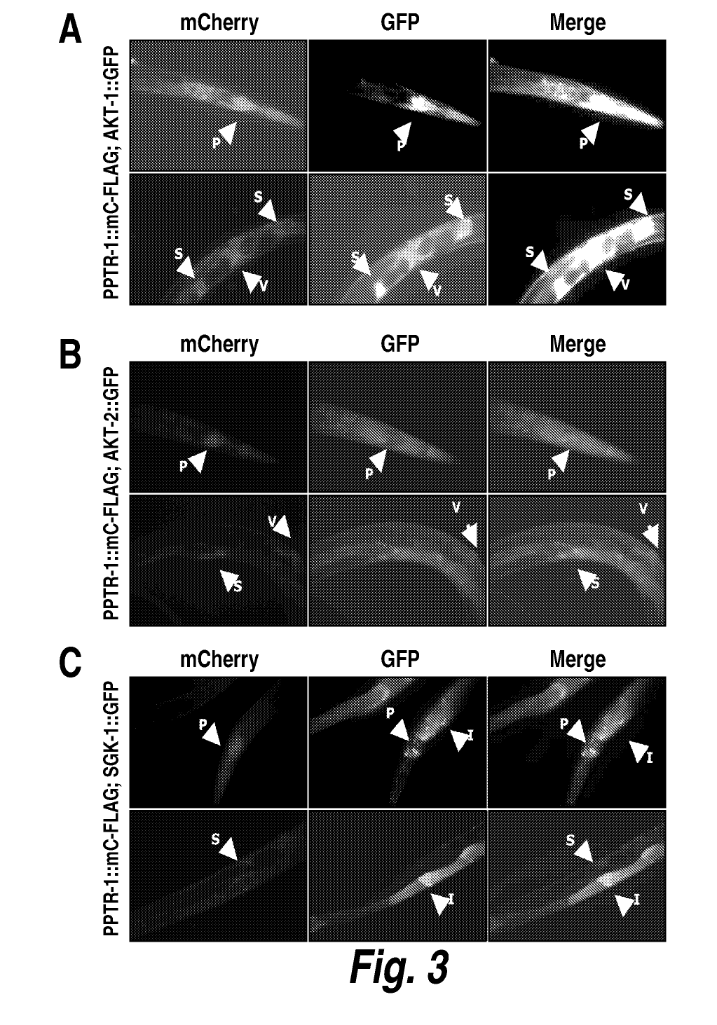

Since pptr-1 and akt-1 genetically interact, it was next investigated whether they have a common expression pattern. The strains akt-1::gfp, akt-2::gfp, sgk-1::gfp were generated or obtained, and pptr-1 was tagged with mCherry and a minimal flag tag to generate pptr-1::mCherry flag transgenic worms (hence referred to as pptr-1::mC-flag; see Example 1 above; GFP/mC-FLAG refers to protein while gfp/mC-flag stands for transgene). Double transgenic worms were made by crossing pptr-1::mC-flag worms to each of the above-mentioned GFP lines. Similar to published data, AKT-1::GFP was observed predominantly in the pharynx, several head neurons, the nerve ring, spermathecae and vulva (Paradis and Ruvkun, 1998); AKT-2::GFP in the pharynx (predominantly in the anterior region), somatic muscles, vulva muscles, spermathecae (Paradis and Ruvkun, 1998); SGK-1::GFP in amphid neurons, intestine and some tail neurons (Hertweck et al., 2004) (FIG. 3A, B, C middle panel) PPTR-1::mC-FLAG was also observed in the pharynx, head neurons, nerve ring, spermathecae and vulva (FIG. 3A, B, C left panel). To observe the sub-cellular localization of PPTR-1, pptr-1::mC-flag worms were stained with DAPI (see Example 1 above). PPTR-1 was found to be predominantly cytosolic with little DAPI overlap. As shown in FIG. 3A-C, there is remarkable overlap between the expression patterns of PPTR-1 and AKT-1. Partial overlap was also observed between AKT-2::GFP and PPTR-1::mC-FLAG, predominantly in the pharynx (FIG. 3B). SGK-1 and PPTR-1 are expressed in different tissues and no significant overlap was observed (FIG. 3C).

### Example 7

### PPTR-1 Regulates AKT-1 Phosphorylation

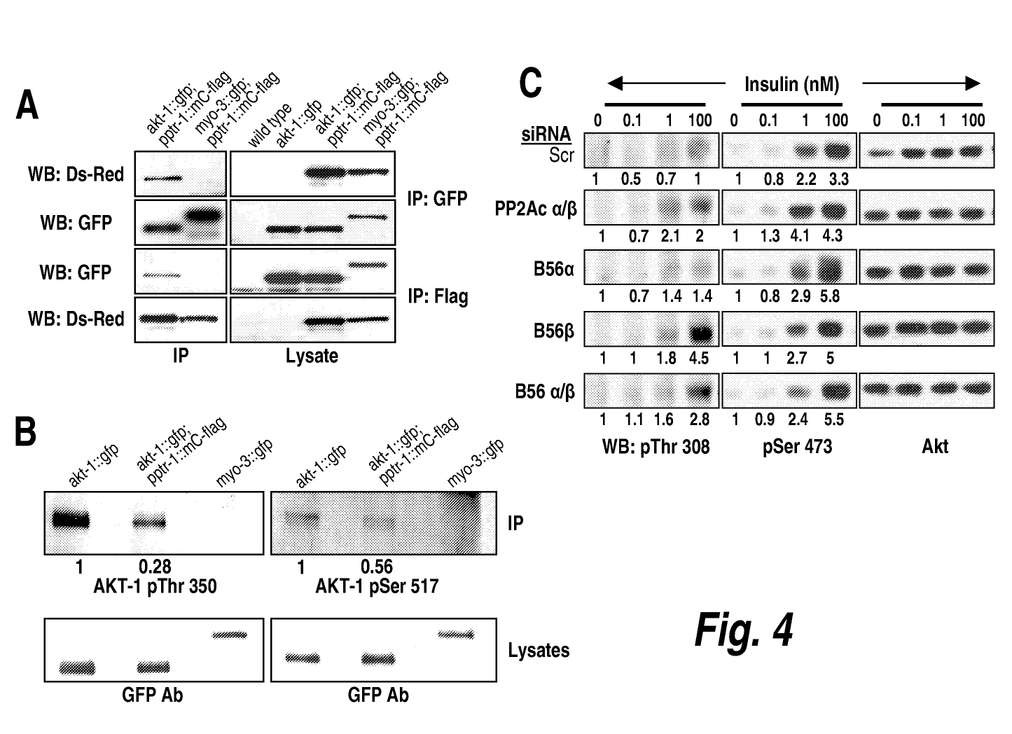

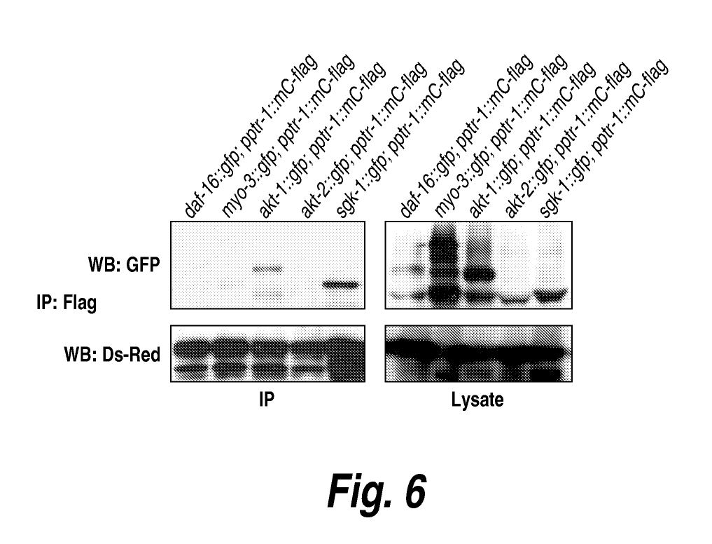

Given the genetic epistasis as well as the overlapping expression patterns described above, it was next examined whether PPTR-1 directly interacts with AKT-1 by co-immunoprecipitation (co-IP) in C. elegans. For all biochemical experiments, the PD4251 strain was used as a control. This strain contains Pmyo-3::gfp with a mitochondrial localization signal and Pmyo-3::lacZ-gfp with a nuclear localization signal (Fire et al., 1998). This strain is referred to herein as myo-3::gfp. Lysates were prepared from mixed-stage akt-1::gfp; pptr-1::mC-flag and myo-3::gfp; pptr-1::mcherry-flag transgenic worms. Following immunoprecipitation with either anti-FLAG or anti-GFP antibody, PPTR-1 was found to specifically interact with AKT-1 and not with MYO-3::GFP (FIG. 4A; for details see Example 1 above). Co-IP experiments were also performed to investigate whether PPTR-1 and AKT-2 interact, since partial overlap in expression pattern of these proteins was observed. The results indicate that PPTR-1 does not interact with AKT-2 (FIG. 6). Epistasis analyses showed no genetic interaction between pptr-1 and sgk-1. Moreover, no overlap in the expression pattern of these two proteins was observe using confocal microscop). However, PPTR-1::mC-FLAG and SGK-1::GFP were found to interact in the co-IP experiments (FIG. 6). This biochemical interaction was not believed to have a measurable functional output and as a consequence it was not pursued further.

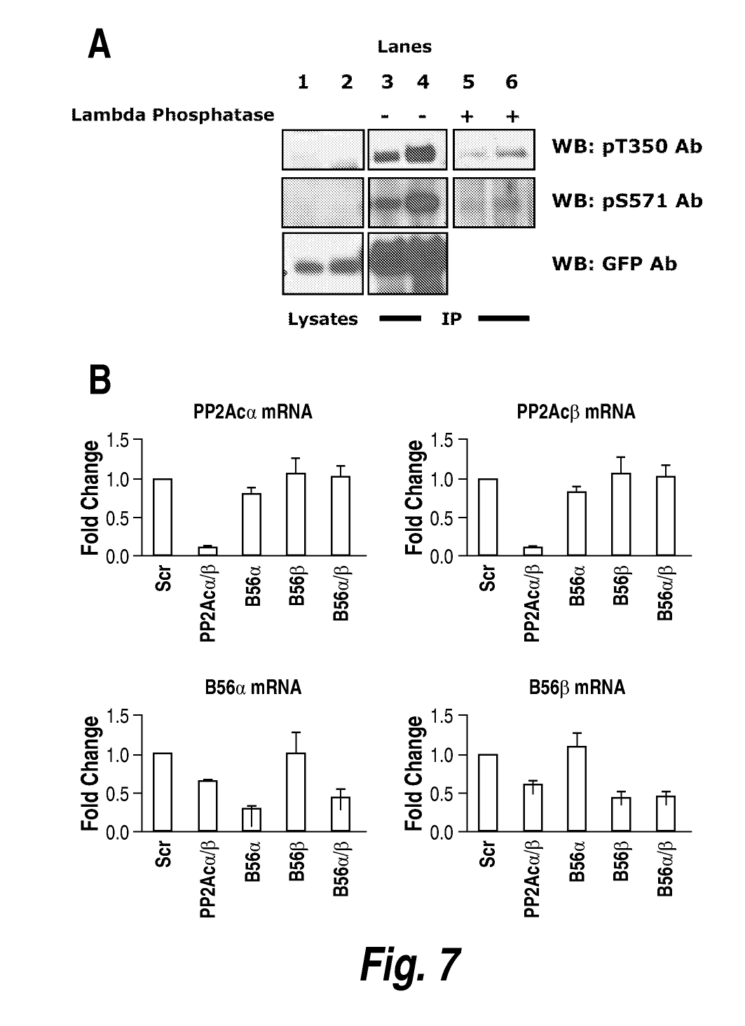

In mammals, Akt is activated by PDK phosphorylation at Thr 308 and PDK-2/TORC-2 protein complex at Ser 473 (Brazil and Hemmings, 2001; Jacinto et al., 2006; Sarbassov et al., 2005). In C. elegans AKT-1, these sites correspond to Thr 350 and Ser 517, respectively. Affinity-purified phospho-specific antibodies (21^{st} Century BioChemicals, USA; Materials and Methods) were generated against both sites to further investigate the role of PPTR-1 on AKT-1 phosphorylation. Following immunoprecipitation with anti-GFP antibody from either akt-1::gfp or akt-1::gfp;pptr-1::mC-flag strain, the phosphorylation status at these two sites was compared. Overexpressing PPTR-1 was found to dramatically decrease the phosphorylation of the T350 site while having a marginal effect on the Ser 517 site (FIG. 4B). As a control experiment, the immunoprecipitated AKT-1::GFP samples were treated with lambda phosphatase and loss of the Thr and Ser phosphorylation was observed, demonstrating the specificity of the phospho-AKT antibodies (FIG. 7A). In summary, these results demonstrate that in C. elegans, PPTR-1 functions by directly regulating the dephosphorylation of AKT-1 primarily at the Thr 350 (mammalian Thr 308) site.

### Example 8

### Mammalian PPTR-1 Homolog Regulates AKT-1 Phosphorylation

Given the evolutionary conservation of the C. elegans IIS pathway, it was next examined whether this mechanism of AKT-1 dephosphorylation mediated by PPTR-1 is also conserved in mammals. The mammalian B56 family of PP2A regulatory subunits has 8 members encoded by 5 genes that express in different tissues (Eichhorn et al., 2008). 3T3-L1 adipocytes were used to perform these studies since in this system, there is a well-characterized insulin signaling pathway that is responsive to changes in insulin levels (Ugi et al., 2004; Watson et al., 2004). First, microarray data from the expression profiles of fibroblasts was compared to differentiated 3T3-L1 adipocytes (Powelka et al., 2006) in order to determine which B56 members were expressed in the adipocytes. Two genes, PPP2R5A (B56α) and PPP2R5B (B56β), were identified as the top candidates. Either one or both these regulatory subunits was knocked down by designing Smartpool siRNAs (Dharmacon, USA) and the silencing was verified by quantitative RT PCR (FIG. 7B). Serum-starved siRNA-treated 3T3-L1 adipocytes were then stimulated with increasing concentrations of insulin. The cells were lysed and the proteins analyzed by western blotting using mammalian Akt phospho-specific antibodies (see Example 1 above). Knockdown of B56β resulted in a dramatic increase in phosphorylation at the Thr 308 site of Akt with relatively less changes in Ser 473 phosphorylation (FIG. 4C). However, silencing of B56α had no effect on the phosphorylation status of Akt at either site. Further, siRNA against both the PP2A catalytic subunits (PP2Acα/β) resulted in increased phosphorylation at Thr 308 but not at Ser 473. Taken together, these data demonstrated that PPTR-1/B56β regulatory subunits function to modulate AKT-1 phosphorylation in a conserved manner across phylogeny.

### Example 9

### PPTR-1 Positively Regulates DAF-16 Nuclear Localization and Activity

The consequences of modulating PPTR-1 dosage on the IIS pathway were next investigated. In C. elegans, one of the major targets of AKT-1 is the forkhead transcription factor, DAF-16. Active signaling through the IIS pathway results in the phosphorylation of DAF-16 by AKT-1, AKT-2 and SGK-1, leading to its nuclear exclusion (Antebi 2007). Experiments were thus carried out to determine whether pptr-1 regulates IIS pathway-specific phenotypes by modulating DAF-16 function. Since reduced phosphorylation of AKT-1 was observed upon overexpression of PPTR-1, the effect of PPTR-1 overexpression on DAF-16 nuclear localization was examined (Henderson and Johnson, 2001; Lee et al., 2001; Lin et al., 2001). A daf-16::gfp;pptr-1::mC-flag strain was generated and then the DAF-16 nuclear localization in these worms was compared with that of a daf-16::gfp strain (FIG. 5A and Table 8A). The DAF-16::GFP localization was categorized as completely cytosolic, mostly cytosolic, mostly nuclear or completely nuclear. DAF-16::GFP nuclear localization was found to be enhanced when PPTR-1 is overexpressed (FIG. 5A and Table 8A). To determine the specificity of this response, mCherry RNAi was used to effectively knockdown mCherry expression in pptr-1::mC-flag thereby reducing the expression of pptr-1 transgene. The results show that the enhanced nuclear localization upon PPTR-1 overexpression is suppressed when pptr-1::mC-flag;daf-16::gfp worms are grown on mCherry RNAi (FIG. 5A and Table 8A) and mCherry RNAi has little effect on DAF-16 localization in daf-16::gfp worms. These experiments indicate that increased dosage of pptr-1 affects DAF-16 nuclear localization. Consistent with its role in the C. elegans IIS pathway, overexpression of pptr-1 was found to significantly increase the lifespan of wild type worms but not to further enhance the lifespan of daf-2(e1370) worms (FIG. 5B and Table 6B); mean lifespan of wild type is 23.9±0.3 days, pptr-1::mC-flag is 30.1±0.5 days, p<0.0001, and the unc-119(+); unc-119(ed3) control strain is 22.6±0.3 days).

As a corollary to this experiment, the effect of pptr-1RNAi on DAF-16 nuclear localization was examined. For this, a daf-2(e1370);daf-16::gfp strain was generated. At the permissive temperature of 15° C., DAF-16::GFP is excluded from the nucleus in the daf-2(e1370);daf-16::gfp strain. However, at the non-permissive temperature of 25° C., progressive nuclear localization of DAF-16::GFP is observed. The daf-2(e1370);daf-16::gfp worms were grown on either vector, pptr-1 or daf-18 RNAi and the extent of nuclear localization at 25° C. was measured. The results of this experiment showed that pptr-1 RNAi significantly reduced DAF-16 nuclear localization, similar to the effect of daf-18 RNAi (FIG. 5C and Table 8B). Together, these experiments indicate that changes in PPTR-1 levels affect the activity of AKT-1 and as a result, modulate DAF-16 sub-cellular localization.

### Example 10

### DAF-16 Target Genes

DAF-16 regulates the transcription of many downstream genes such as sod-3, hsp-12.6, sip-1 and mtl-1 (Furuyama et al., 2000; Lee et al., 2003; McElwee et al., 2003; Murphy et al., 2003; Oh et al., 2006). The effects of pptr-1 RNAi on these DAF-16 transcriptional targets were next examined. The sod-3 gene has been shown to be a direct target of DAF-16 by chromatin immunoprecipitation (Oh et al., 2006) and its expression changes in response to modulation of the IIS pathway (Furuyama et al., 2000; Libina et al., 2003; Murphy et al., 2003). A daf-2(e1370);Psod-3::gfp(muIs84) strain was grown on either vector, daf-18 or pptr-1 RNAi to look at the effect on GFP expression. Similar to worms grown on daf-18 RNAi, pptr-1 RNAi reduces expression of GFP (FIG. 5D and Table 8C). Therefore, modulation in the levels of pptr-1 can affect the expression of direct DAF-16 target genes.

The expression of known DAF-16 target genes was further analyzed by quantitative RT-PCR in a daf-2(e1370) mutant background. As a control, it was analyzed whether each of these target genes is expressed in a daf-16-dependent manner, as previously reported (McElwee et al., 2003; Murphy et al., 2003; Oh et al., 2006). As shown in FIG. 5E and Table 8D, daf-16 RNAi dramatically suppressed the expression levels of these genes. Next, the effects of either pptr-1 or daf-18 RNAi on the expression of these genes was tested. The results of this experiment showed that pptr-1 RNAi also suppressed the expression of these genes to a level similar to daf-18 RNAi. Taken together, these data indicate that PPTR-1 positively regulates DAF-16 nuclear localization and thereby its activity.

### Summary of Examples 1-10

The insulin/IGF-1 (IIS) signaling pathway regulates growth, metabolism and longevity across phylogeny. Given the large number of cellular processes that this pathway controls, understanding the mechanisms that modulate IIS is of paramount importance. IIS is a well-studied kinase cascade but few phosphatases in the pathway are known. Identification of these phosphatases, especially those that counterbalance the activity of the kinases, will provide a better insight into the regulation of this important pathway. C. elegans is an excellent system amenable to genetic manipulations including RNAi. In addition, the worm IIS pathway controls several well-defined phenotypes such as lifespan and dauer formation that can be easily quantitated. Therefore, to identify novel phosphatases regulating the IIS pathway, a directed RNAi screen was performed using dauer formation as an output. Serine/threonine phosphatases were specifically examined, as the majority of phosphorylations in the cell, including the insulin signaling pathway, occur on serine or threonine residues (Moorhead et al., 2007).

The pptr-1 gene was identified as a top candidate in the initial screen. This gene encodes a protein that bears homology to the mammalian B56 family of PP2A regulatory subunits (Janssens et al., 2008). PP2A itself is a ubiquitously expressed phosphatase that is involved in multiple cellular processes including the regulation of insulin signaling by direct dephosphorylation of Akt (Andjelkovic et al., 1996; Resjo et al., 2002; Ugi et al., 2004). Specificity of PP2A to its various cellular targets is achieved by its association with distinct regulatory subunits. The studies described herein provide for the first time a mechanistic insight into how the C. elegans PP2A regulatory subunit PPTR-1 modulates insulin signaling by specifically regulating AKT-1 phosphorylation and activity in the context of a whole organism. Furthermore, these studies show that this mechanism of regulation is conserved in mammals.

The studies described herein identify PPTR-1 as a novel and integral component of the C. elegans IIS pathway. As depicted in a model presented in FIG. 5F, the studies described herein indicate that PPTR-1 acts to negatively regulate signals transduced through the IIS pathway, ultimately controlling the activity of the FOXO transcription factor DAF-16. Under low signaling conditions, DAF-16 is able to translocate to the nucleus and transactivate or repress its downstream targets. It is well established that AKT modulates DAF-16 sub-cellular localization. Thus, the activity of AKT-1, as governed by its phosphorylation status, directly translates into the activity of DAF-16. In the studies described herein, PPTR-1 has been shown to directly interact with AKT-1 and regulate its activity by modulating its phosphorylation, predominantly at the Thr 350 site. Less active AKT-1 results in increased DAF-16 nuclear localization. Indeed, DAF-16 is found to be more nuclear throughout the worm when PPTR-1 is overexpressed. As a corollary, knocking down pptr-1 by RNAi results in less nuclear DAF-16 as well as reduced expression of DAF-16 target genes such as sod-3, hsp-12.6, mtl-1 and sip-1. These genes are known to play a combinatorial role in adaptation to various stresses, leading to enhanced dauer formation and increased lifespan. Consistent with the decreased levels of these important genes, pptr-1 RNAi results in a significant decrease in the dauer formation, lifespan as well as thermotolerance of daf-2(e1370) worms. In addition, pptr-1 also regulates other DAF-16-dependent outputs of the IIS pathway such as fat storage. Thus, it was found in the studies described herein that normal levels of pptr-1 are important under low insulin signaling conditions. However, pptr-1 RNAi does not affect IIS pathway-associated phenotypes in wild type worms. There could be several reasons for this observation. Firstly, under normal signaling conditions, AKT-1, AKT-2 as well as SGK-1 are active and negatively regulate DAF-16. Therefore, changes in the AKT-1 activity alone brought about by pptr-1 RNAi may not have a significant effect on DAF-16-dependent phenotypes. Secondly, PPTR-1 itself may be negatively regulated by the IIS pathway, leading to increased AKT-1 phosphorylation. Along similar lines, in mammals, insulin signaling can downregulate the expression and activity of the PP2A catalytic subunit (Hojlund et al., 2002; Srinivasan and Begum, 1994; Ugi et al., 2004). Thus, under normal conditions, further down regulation of pptr-1 by RNAi may have no effect. While not wishing to be bound by theory, it is possible that in C elegans, in response to changing environmental cues, PPTR-1 helps to downregulate the insulin signaling pathway to promote DAF-16 activity, enabling the worm to either enter diapause or enhance its tolerance to stress as adults.

In mammals, Akt controls a myriad of secondary signaling cascades that regulate glucose transport, protein synthesis, genomic stability, cell survival and gene expression (Toker and Yoeli-Lerner, 2006). Previous studies have implicated roles for PP2A and PHLPP phosphatases in the negative regulation of Akt (Kuo et al., 2008). The PP2A inhibitor Okadaic acid can increase Akt phosphorylation predominantly at Thr 308 and enhance glucose transport in adipocytes (Rondinone et al., 1999). Consistent with this, the results described herein show that siRNA knockdown of the PP2A catalytic subunit and more importantly, the B56β regulatory subunit results in enhanced Akt phosphorylation at Thr 308 in 3T3-L1 adipocytes. Thus, these studies highlight the remarkable functional conservation of the B56/PPTR-1 regulatory subunit of PP2A in regulating AKT phosphorylation between C. elegans and higher mammals. In worms, a modest effect on Ser 517 (equivalent to mammalian Ser 473) phosphorylation by PPTR-1 overexpression was also observed. However, a difference in Ser 473 phosphorylation in adipocytes was not observe. This difference may be explained by the fact that in worms, the phosphorylation of AKT-1 in the context of a whole organism is determined. Additionally, in mammals, the phosphorylation state of one Akt site may influence the status of the other (Gao et al., 2005; Toker and Newton, 2000). The studies described herein do not indicate a role for the PP2A B55 subunit (sur-6) in the C. elegans IIS pathway. However, a recent report using cell culture has implicated the mammalian B55 in the regulation of AKT (Kuo et al., 2008).

Dysregulation of Akt has been implicated in diseases such as cancer and diabetes (Rondinone et al., 1999; Sasaoka et al., 2006; Smith et al., 1999; Zdychova and Komers, 2005). In fact, the onset of diabetes is often associated with changes in Akt phosphorylation (Zdychova and Komers, 2005). In several cancer models, loss of function mutations in the PTEN results in hyper-phosphorylated and activated Akt (Groszer et al., 2001; Hakem and Mak, 2001; Stiles et al., 2002; Testa and Bellacosa, 2001). The studies described herein show that like PTEN, PPTR-1 acts to negatively regulate the insulin/IGF-1 signaling. Given the important role of PPTR-1/B56 in modulating Akt activity, this protein is an important therapeutic target for the treatment of diabetes as well as cancer.

### REFERENCES

Andjelkovic, M., Jakubowicz, T., Cron, P., Ming, X. F., Han, J. W., and Hemmings, B. A. (1996). Activation and phosphorylation of a pleckstrin homology domain containing protein kinase (RAC-PK/PKB) promoted by serum and protein phosphatase inhibitors. Proc Natl Acad Sci USA 93, 5699-5704.
Antebi, A. (2007). Genetics of aging in Caenorhabditis elegans. PLoS Genet 3, 1565-1571.
Ashrafi, K., Chang, F. Y., Watts, J. L., Fraser, A. G., Kamath, R. S., Ahringer, J., and Ruvkun, G. (2003). Genome-wide RNAi analysis of Caenorhabditis elegans fat regulatory genes. Nature 421, 268-272.
Barbieri, M., Bonafe, M., Franceschi, C., and Paolisso, G. (2003). Insulin/IGF-I-signaling pathway: an evolutionarily conserved mechanism of longevity from yeast to humans. Am J Physiol Endocrinol Metab 285, E1064-1071.
Brazil, D. P., and Hemmings, B. A. (2001). Ten years of protein kinase B signalling: a hard Akt to follow. Trends Biochem Sci 26, 657-664.
Brunet, A., Bonni, A., Zigmond, M. J., Lin, M. Z., Juo, P., Hu, L. S., Anderson, M. J., Arden, K. C., Blenis, J., and Greenberg, M. E. (1999). Akt promotes cell survival by phosphorylating and inhibiting a Forkhead transcription factor. Cell 96, 857-868.
Brunet, A., Park, J., Tran, H., Hu, L. S., Hemmings, B. A., and Greenberg, M. E. (2001). Protein kinase SGK mediates survival signals by phosphorylating the forkhead transcription factor FKHRL1 (FOXO3a). Mol Cell Biol 21, 952-965.
Calnan, D. R., and Brunet, A. (2008). The FoxO code. Oncogene 27, 2276-2288.
da Graca, L. S., Zimmerman, K. K., Mitchell, M. C., Kozhan-Gorodetska, M., Sekiewicz, K., Morales, Y., and Patterson, G. I. (2004). DAF-5 is a Ski oncoprotein homolog that functions in a neuronal TGF beta pathway to regulate C. elegans dauer development. Development 131, 435-446.
Dorman, J. B., Albinder, B., Shroyer, T., and Kenyon, C. (1995). The age-1 and daf-2 Genes Function in a Common Pathway to Control the Lifespan of Caenorhabditis elegans. Genetics 141, 1399-1406.
Eichhorn, P. J., Creyghton, M. P., and Bernards, R. (2008). Protein phosphatase 2A regulatory subunits and cancer. Biochim Biophys Acta.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Furuyama, T., Nakazawa, T., Nakano, I., and Mori, N. (2000). Identification of the differential distribution patterns of mRNAs and consensus binding sequences for mouse DAF-16 homologues. Biochem J 349, 629-634.
Gao, T., Furnari, F., and Newton, A. C. (2005). PHLPP: a phosphatase that directly dephosphorylates Akt, promotes apoptosis, and suppresses tumor growth. Mol Cell 18, 13-24.
Gems, D., Sutton, A. J., Sundermeyer, M. L., Albert, P. S., King, K. V., Edgley, M. L., Larsen, P. L., and Riddle, D. L. (1998). Two pleiotropic classes of daf-2 mutation affect larval arrest, adult behavior, reproduction and longevity in Caenorhabditis elegans. Genetics 150, 129-155.
Gil, E. B., Malone Link, E., Liu, L. X., Johnson, C. D., and Lees, J. A. (1999). Regulation of the insulin-like developmental pathway of Caenorhabditis elegans by a homolog of the PTEN tumor suppressor gene. Proc Natl Acad Sci USA 96, 2925-2930.
Groszer, M., Erickson, R., Scripture-Adams, D. D., Lesche, R., Trumpp, A., Zack, J. A., Kornblum, H. I., Liu, X., and Wu, H. (2001). Negative regulation of neural stem/progenitor cell proliferation by the Pten tumor suppressor gene in vivo. Science 294, 2186-2189.
Gunther, C. V., Georgi, L. L., and Riddle, D. L. (2000). A Caenorhabditis elegans type I TGF beta receptor can function in the absence of type II kinase to promote larval development. Development 127, 3337-3347.

Hakem, R., and Mak, T. W. (2001). Animal models of tumor-suppressor genes. Annu Rev Genet 35, 209-241.
Hansen, D., and Pilgrim, D. (1998). Molecular evolution of a sex determination protein. FEM-2 (pp 2c) in Caenorhabditis. Genetics 149, 1353-1362.
Henderson, S. T., and Johnson, T. E. (2001). daf-16 integrates developmental and environmental inputs to mediate aging in the nematode Caenorhabditis elegans. Curr Biol 11, 1975-1980.
Hertweck, M., Gobel, C., and Baumeister, R. (2004). C. elegans SGK-1 is the critical component in the Akt/PKB kinase complex to control stress response and life span. Dev Cell 6, 577-588.
Hojlund, K., Poulsen, M., Staehr, P., Brusgaard, K., and Beck-Nielsen, H. (2002). Effect of insulin on protein phosphatase 2A expression in muscle in type 2 diabetes. Eur J Clin Invest 32, 918-923.
Honda, Y., and Honda, S. (1999). The daf-2 gene network for longevity regulates oxidative stress resistance and Mn-superoxide dismutase gene expression in Caenorhabditis elegans. FASEB J 13, 1385-1393.
Inoue, T., and Thomas, J. H. (2000). Targets of TGF-beta signaling in Caenorhabditis elegans dauer formation. Developmental Biology 217, 192-204.
Jacinto, E., Facchinetti, V., Liu, D., Soto, N., Wei, S., Jung, S. Y., Huang, Q., Qin, J., and Su, B. (2006). SIN1/MIP1 maintains rictor-mTOR complex integrity and regulates Akt phosphorylation and substrate specificity. Cell 127, 125-137.
Janssens, V., Longin, S., and Goris, J. (2008). PP2A holoenzyme assembly: in cauda venenum (the sting is in the tail). Trends Biochem Sci 33, 113-121.
Jensen, V. L., Albert, P. S., and Riddle, D. L. (2007). Caenorhabditis elegans SDF-9 enhances insulin/insulin-like signaling through interaction with DAF-2. Genetics 177, 661-666.
Kamath, R. S., and Ahringer, J. (2003). Genome-wide RNAi screening in Caenorhabditis elegans. Methods 30, 313-321.
Kamath, R. S., Fraser, A. G., Dong, Y., Poulin, G., Durbin, R., Gotta, M., Kanapin, A., Le Bot, N., Moreno, S., Sohrmann, M., et al. (2003). Systematic functional analysis of the Caenorhabditis elegans genome using RNAi. Nature 421, 231-237.
Kenyon, C. (2005). The plasticity of aging: insights from long-lived mutants. Cell 120, 449-460.
Kenyon, C., Chang, J., Gensch, E., Rudner, A., and Tabtiang, R. (1993). A C. elegans mutant that lives twice as long as wild type. Nature 366, 461-464.
Kimura, K. D., Tissenbaum, H. A., Liu, Y., and Ruvkun, G. (1997). daf-2, an insulin receptor-like gene that regulates longevity and diapause in Caenorhabditis elegans. Science 277, 942-946.
Kuo, Y. C., Huang, K. Y., Yang, C. H., Yang, Y. S., Lee, W. Y., and Chiang, C. W. (2008). Regulation of phosphorylation of Thr-308 of Akt, cell proliferation, and survival by the B55alpha regulatory subunit targeting of the protein phosphatase 2A holoenzyme to Akt. J Biol Chem 283, 1882-1892.
Larsen, P. L., Albert, P. S., and Riddle, D. L. (1995). Genes that regulate both development and longevity in Caenorhabditis elegans. Genetics 139, 1567-1583.
Lee, R. Y., Hench, J., and Ruvkun, G. (2001). Regulation of C. elegans DAF-16 and its human ortholog FKHRL1 by the daf-2 insulin-like signaling pathway. Curr Biol 11, 1950-1957.
Lee, S. S., Kennedy, S., Tolonen, A. C., and Ruvkun, G. (2003). DAF-16 target genes that control C. elegans life-span and metabolism. Science 300, 644-647.
Libina, N., Berman, J. R., and Kenyon, C. (2003). Tissue-specific activities of C. elegans DAF-16 in the regulation of lifespan. Cell 115, 489-502.
Lin, K., Dorman, J. B., Rodan, A., and Kenyon, C. (1997). daf-16: An HNF-3/forkhead family member that can function to double the life-span of Caenorhabditis elegans. Science 278, 1319-1322.
Lin, K., Hsin, H., Libina, N., and Kenyon, C. (2001). Regulation of the Caenorhabditis elegans longevity protein DAF-16 by insulin/IGF-1 and germline signaling. Nat Genet 28, 139-145.
Lithgow, G. J., and Walker, G. A. (2002). Stress resistance as a determinate of C. elegans lifespan. Mech Ageing Dev 123, 765-771.
Lithgow, G. J., White, T. M., Melov, S., and Johnson, T. E. (1995). Thermotolerance and extended life-span conferred by single-gene mutations and induced by thermal stress. Proc Natl Acad Sci USA 92, 7540-7544.
McElwee, J., Bubb, K., and Thomas, J. H. (2003). Transcriptional outputs of the Caenorhabditis elegans forkhead protein DAF-16. Aging Cell 2, 111-121.
Mihaylova, V. T., Borland, C. Z., Manjarrez, L., Stern, M. J., and Sun, H. (1999). The PTEN tumor suppressor homolog in Caenorhabditis elegans regulates longevity and dauer formation in an insulin receptor-like signaling pathway. Proc Natl Acad Sci USA 96, 7427-7432.
Moorhead, G. B., Trinkle-Mulcahy, L., and Ulke-Lemee, A. (2007). Emerging roles of nuclear protein phosphatases. Nat Rev Mol Cell Biol 8, 234-244.
Morris, J. Z., Tissenbaum, H. A., and Ruvkun, G. (1996). A phosphatidylinositol-3-OH kinase family member regulating longevity and diapause in Caenorhabditis elegans. Nature 382, 536-539.
Mukhopadhyay, A., Oh, S. W., and Tissenbaum, H. A. (2006). Worming pathways to and from DAF-16/FOXO. Exp Gerontol 41, 928-934.
Munoz, M. J., and Riddle, D. L. (2003). Positive selection of Caenorhabditis elegans mutants with increased stress resistance and longevity. Genetics 163, 171-180.
Murphy, C. T., McCarroll, S. A., Bargmann, C. I., Fraser, A., Kamath, R. S., Ahringer, J., Li, H., and Kenyon, C. (2003). Genes that act downstream of DAF-16 to influence the lifespan of Caenorhabditis elegans. Nature 424, 277-283.
Ogg, S., Paradis, S., Gottlieb, S., Patterson, G. I., Lee, L., Tissenbaum, H. A., and Ruvkun, G. (1997). The Fork head transcription factor DAF-16 transduces insulin-like metabolic and longevity signals in C. elegans. Nature 389, 994-999.
Ogg, S., and Ruvkun, G. (1998). The C. elegans PTEN homolog, DAF-18, acts in the insulin receptor-like metabolic signaling pathway. Molecular Cell 2, 887-893.
Oh, S. W., Mukhopadhyay, A., Dixit, B. L., Raha, T., Green, M. R., and Tissenbaum, H. A. (2006). Identification of direct DAF-16 targets controlling longevity, metabolism and diapause by chromatin immunoprecipitation. Nat Genet 38, 251-257.
Oh, S. W., Mukhopadhyay, A., Svrzikapa, N., Jiang, F., Davis, R. J., and Tissenbaum, H. A. (2005). JNK regulates lifespan in Caenorhabditis elegans by modulating nuclear translocation of forkhead transcription factor/DAF-16. Proc Natl Acad Sci USA 102, 4494-4499.
Paradis, S., Ailion, M., Toker, A., Thomas, J. H., and Ruvkun, G. (1999). A PDK1 homolog is necessary and sufficient to transduce AGE-1 PI3 kinase signals that regulate diapause in Caenorhabditis elegans. Genes Dev 13, 1438-1452.
Paradis, S., and Ruvkun, G. (1998). Caenorhabditis elegans Akt/PKB transduces insulin receptor-like signals from AGE-1 PI3 kinase to the DAF-16 transcription factor. Genes Dev 12, 2488-2498.
Patterson, G. I., Koweek, A., Wong, A., Liu, Y., and Ruvkun, G. (1997). The DAF-3 Smad protein antagonizes TGF-beta-related receptor signaling in the Caenorhabditis elegans dauer pathway. Genes Dev 11, 2679-2690.
Patterson, G. I., and Padgett, R. W. (2000). TGF beta-related pathways. Roles in Caenorhabditis elegans development. Trends Genet 16, 27-33.
Pilgrim, D., McGregor, A., Jackle, P., Johnson, T., and Hansen, D. (1995). The C. elegans sex-determining gene fem-2 encodes a putative protein phosphatase. Mol Biol Cell 6, 1159-1171.
Powelka, A. M., Seth, A., Virbasius, J. V., Kiskinis, E., Nicoloro, S. M., Guilherme, A., Tang, X., Straubhaar, J., Cherniack, A. D., Parker, M. G., and Czech, M. P. (2006). Suppression of oxidative metabolism and mitochondrial biogenesis by the transcriptional corepressor RIP140 in mouse adipocytes. J Clin Invest 116, 125-136.
Reboul, J., Vaglio, P., Rual, J. F., Lamesch, P., Martinez, M., Armstrong, C. M., Li, S., Jacotot, L., Bertin, N., Janky, R., et al. (2003). C. elegans ORFeome version 1.1: experimental verification of the genome annotation and resource for proteome-scale protein expression. Nat Genet 34, 35-41.
Ren, P., Lim, C., Johnsen, R., Albert, P. S., Pilgrim, D., and Riddle, D. L. (1996). Control of C. elegans Larval Development by Neuronal Expression of a TGF-β homologue. Science 274, 1389-1391.
Resjo, S., Goransson, O., Harndahl, L., Zolnierowicz, S., Manganiello, V., and Degerman, E. (2002). Protein phosphatase 2A is the main phosphatase involved in the regulation of protein kinase B in rat adipocytes. Cell Signal 14, 231-238.
Riddle D., B. T., Meyer B., Priess J., (1997). C. Elegans II, 1 edn (Cold Spring Harbor: Cold Spring Harbor Press).
Riddle, D. L., Swanson, M. M., and Albert, P. S. (1981). Interacting genes in nematode dauer larva formation. Nature 290, 668-671.
Rondinone, C. M., Carvalho, E., Wesslau, C., and Smith, U. P. (1999). Impaired glucose transport and protein kinase B activation by insulin, but not okadaic acid, in adipocytes from subjects with Type II diabetes mellitus. Diabetologia 42, 819-825.
Rouault, J. P., Kuwabara, P. E., Sinilnikova, O. M., Duret, L., Thierry-Mieg, D., and Billaud, M. (1999). Regulation of dauer larva development in Caenorhabditis elegans by daf-18, a homologue of the tumour suppressor PTEN. Current Biology 9, 329-332.
Sarbassov, D. D., Guertin, D. A., Ali, S. M., and Sabatini, D. M. (2005). Phosphorylation and regulation of Akt/PKB by the rictor-mTOR complex. Science 307, 1098-1101.
Sasaoka, T., Wada, T., and Tsuneki, H. (2006). Lipid phosphatases as a possible therapeutic target in cases of type 2 diabetes and obesity. Pharmacol Ther 112, 799-809.

Savage-Dunn, C. (2005). TGF-beta signaling. WormBook, 1-12.
Smith, U., Axelsen, M., Carvalho, E., Eliasson, B., Jansson, P. A., and Wesslau, C. (1999). Insulin signaling and action in fat cells: associations with insulin resistance and type 2 diabetes. Ann N Y Acad Sci 892, 119-126.
Srinivasan, M., and Begum, N. (1994). Regulation of protein phosphatase 1 and 2A activities by insulin during myogenesis in rat skeletal muscle cells in culture. J Biol Chem 269, 12514-12520.
Stiernagle, T. (2006). Maintenance of C. elegans. WormBook, 1-11.
Stiles, B., Gilman, V., Khanzenzon, N., Lesche, R., Li, A., Qiao, R., Liu, X., and Wu, H. (2002). Essential role of AKT-1/protein kinase B alpha in PTEN-controlled tumorigenesis. Mol Cell Biol 22, 3842-3851.
Tang, X., Guilherme, A., Chakladar, A., Powelka, A. M., Konda, S., Virbasius, J. V., Nicoloro, S. M., Straubhaar, J., and Czech, M. P. (2006). An RNA interference-based screen identifies MAP4K4/NIK as a negative regulator of PPARgamma, adipogenesis, and insulin-responsive hexose transport. Proc Natl Acad Sci USA 103, 2087-2092.
Testa, J. R., and Bellacosa, A. (2001). AKT plays a central role in tumorigenesis. Proc Natl Acad Sci USA 98, 10983-10985.
Tesz, G. J., Guilherme, A., Guntur, K. V., Hubbard, A. C., Tang, X., Chawla, A., and Czech, M. P. (2007). Tumor necrosis factor alpha (TNFalpha) stimulates Map4k4 expression through TNFalpha receptor 1 signaling to c-Jun and activating transcription factor 2. J Biol Chem 282, 19302-19312.
Toker, A., and Newton, A. C. (2000). Akt/protein kinase B is regulated by autophosphorylation at the hypothetical PDK-2 site. J Biol Chem 275, 8271-8274.
Toker, A., and Yoeli-Lerner, M. (2006). Akt signaling and cancer: surviving but not moving on. Cancer Res 66, 3963-3966.
Ugi, S., Imamura, T., Maegawa, H., Egawa, K., Yoshizaki, T., Shi, K., Obata, T., Ebina, Y., Kashiwagi, A., and Olefsky, J. M. (2004). Protein phosphatase 2A negatively regulates insulin's metabolic signaling pathway by inhibiting Akt (protein kinase B) activity in 3T3-L1 adipocytes. Mol Cell Biol 24, 8778-8789.
Vowels, J. J., and Thomas, J. H. (1992). Genetic analysis of chemosensory control of dauer formation in Caenorhabditis elegans. Genetics 130, 105-123.
Watson, R. T., Kanzaki, M., and Pessin, J. E. (2004). Regulated membrane trafficking of the insulin-responsive glucose transporter 4 in adipocytes. Endocr Rev 25, 177-204.
Wolff, S., and Dillin, A. (2006). The trifecta of aging in Caenorhabditis elegans. Exp Gerontol 41, 894-903.
Wolkow, C. A., Munoz, M. J., Riddle, D. L., and Ruvkun, G. (2002). Insulin receptor substrate and p55 orthologous adaptor proteins function in the Caenorhabditis elegans daf-2/insulin-like signaling pathway. J Biol Chem 277, 49591-49597.
Zdychova, J., and Komers, R. (2005). Emerging role of Akt kinase/protein kinase B signaling in pathophysiology of diabetes and its complications. Physiol Res 54, 1-16.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Hertweck, M., Gobel, C., and Baumeister, R. (2004). C. elegans SGK-1 is the critical component in the Akt/PKB kinase complex to control stress response and life span. Dev Cell 6, 577-588.
McNally, K., Audhya, A., Oegema, K., and McNally, F. J. (2006). Katanin controls mitotic and meiotic spindle length. J Cell Biol 175, 881-891.
Paradis, S., and Ruvkun, G. (1998). Caenorhabditis elegans Akt/PKB transduces insulin receptor-like signals from AGE-1 PI3 kinase to the DAF-16 transcription factor. Genes Dev 12, 2488-2498.
Patterson, G. I., Koweek, A., Wong, A., Liu, Y., and Ruvkun, G. (1997). The DAF-3 Smad protein antagonizes TGF-beta-related receptor signaling in the Caenorhabditis elegans dauer pathway. Genes Dev 11, 2679-2690.
Reboul, J., Vaglio, P., Rual, J. F., Lamesch, P., Martinez, M., Armstrong, C. M., Li, S., Jacotot, L., Bertin, N., Janky, R., et al. (2003). C. elegans ORFeome version 1.1: experimental verification of the genome annotation and resource for proteome-scale protein expression. Nat Genet 34, 35-41.
Stiernagle, T. (2006). Maintenance of C. elegans. WormBook, 1-11.

### EQUIVALENTS

Those skilled in the art will recognize, or be able to ascertain using no more than routine experimentation, many equivalents to the specific embodiments of the invention described herein. Such equivalents are intended to be encompassed by the following claims.

In addition, the contents of all patent publications discussed supra are incorporated in their entirety by this reference.

1. A method for identifying a test compound that modulates the expression or activity of a PP2A B56 regulatory subunit, comprising:
administering the test compound to an organism in which PP2A B56 regulatory subunit activity or expression is upmodulated, said organism having a phenotype, relative to a wild-type phenotype, associated with said upmodulation of PP2A B56 regulatory subunit activity or expression;
determining the ability of the test compound to effect said phenotype,
to thereby evaluate the ability of the test compound to modulate the expression or activity of a PP2A B56 regulatory subunit in said organism.

2. The method of claim 1, wherein the phenotype is decreased phosphorylation of AKT-1, or a mammalian homolog thereof.

3. A method for identifying a test compound that modulates the expression or activity of a PP2A B56 regulatory subunit, comprising:
administering the test compound to an organism in which PP2A B56 regulatory subunit activity or expression is downmodulated, said organism having a phenotype, relative to a wild-type phenotype, associated with said downmodulation of PP2A B56 regulatory subunit activity or expression;
determining the ability of the test compound to effect said phenotype,
to thereby evaluate the ability of the test compound to modulate the expression or activity of a PP2A B56 regulatory subunit in said organism.

4. The method of claim 3, wherein said organism further has a deregulated insulin signaling pathway, wherein said detectable phenotype is associated with said downmodulation of PP2A B56 regulatory subunit activity or expression and with said deregulated insulin signaling pathway.

5. The method of claim 3, wherein the phenotype is phosphorylation of AKT-1.

6. A method for evaluating the ability of a test compound to modulate the expression or activity of a PP2A B56 regulatory subunit comprising:
contacting a cell in which PP2A B56 regulatory subunit activity or expression is upmodulated with a test compound, wherein a detectable indicator is associated with said upmodulation of PP2A B56 regulatory subunit activity or expression;
determining the ability of the test compound to effect said indicator,
to thereby evaluate the ability of the test compound to modulate the expression or activity of a PP2A B56 regulatory subunit.

7. The method of claim 6, wherein the phenotype is decreased phosphorylation of AKT-1, or a mammalian homolog thereof.

8. A method for evaluating the ability of a test compound to modulate the expression or activity of a PP2A B56 regulatory subunit, comprising:
contacting a cell in which PP2A B56 regulatory subunit activity or expression is downmodulated with a test compound, wherein a detectable indicator is associated with said downmodulation of PP2A B56 regulatory subunit activity or expression;
determining the ability of the test compound to effect said indicator,
to thereby evaluate the ability of the test compound to modulate the expression or activity of a PP2A B56 regulatory subunit.

9. The method of claim 8, wherein the phenotype is phosphorylation of AKT-1, or a mammalian homolog thereof.

10. The method of claims 6 or 8, wherein the cell is a mammalian cell.

11. The method claim 10, wherein the cell is a human cell.

12. The method claims 6 or 8, wherein the cell is a cell derived from a nematode.

13. The method of any one of claims 2, 5, 7 and 9, wherein the phenotype is phosphorylation of mammalian Akt at threonine 308.

14-15. (canceled)

16. A method for preventing or treating type II diabetes in a subject, comprising administering to the subject an agent that selectively decreases PP2A B56 regulatory subunit activity, to thereby prevent or treat type II diabetes in the subject.

17. A method for preventing or treating obesity in a subject, comprising administering to the subject an agent that selectively decreases PP2A B56 regulatory subunit activity, to thereby prevent or treat obesity in the subject.

18-20. (canceled)

21. A method for preventing or treating cancer in a subject, comprising administering to the subject an agent that selectively increases PP2A B56 regulatory subunit activity, to thereby prevent or treat cancer in the subject.

22-23. (canceled)

24. A method for enhancing longevity in a subject, comprising administering to the subject an agent that selectively increases PP2A B56 regulatory subunit activity, to thereby enhance longevity in the subject.

25-27. (canceled)

28. The method of claim 1, wherein the ability of the test compound to effect said phenotype is indicative of the ability of the compound to treat type II diabetes.

29. The method of claim 1, wherein the ability of the test compound to effect said phenotype is indicative of the ability of the compound to treat obesity.

30. The method of claim 3, wherein the ability of the test compound to effect said phenotype is indicative of the ability of the compound to treat cancer.

31. The method of claim 3, wherein the ability of the test compound to effect said phenotype is indicative of the ability of the compound to enhance longevity.

32. The method of claims 1 or 3, wherein the phenotype is nuclear localization of DAF-16, or a mammalian homolog thereof.

33. The method of claims 1 or 3, wherein the phenotype is phosphorylation of DAF-16, or a mammalian homolog thereof.
